SEQUENCE LISTING (25 sequences)

SEQ ID NO: 1 - DNA, 7547 bp, synthetic construct
  source 1..7547: mol_type = other DNA
tattgcttggcagcgcctatcgccaggtattactccaatcccgaatatccgagatcgggatcacccgagagaagttcaacctacatcctcaatcccgatctatccgagatccgaggaatatcgaaatcggggcgcgccaacgaagcatctgtgcttcattttgtagaacaaaaatgcaacgcgagagcgctaatttttcaaacaaagaatctgagctgcatttttacagaacagaaatgcaacgcgaaagcgctattttaccaacgaagaatctgtgcttcatttttgtaaaacaaaaatgcaacgcgagagcgctaatttttcaaacaaagaatctgagctgcatttttacagaacagaaatgcaacgcgagagcgctattttaccaacaaagaatctatacttcttttttgttctacaaaaatgcatcccgagagcgctatttttctaacaaagcatcttagattactttttttctcctttgtgcgctctataatgcagtctcttgataactttttgcactgtaggtccgttaaggttagaagaaggctactttggtgtctattttctcttccataaaaaaagcctgactccacttcccgcgtttactgattactagcgaagctgcgggtgcattttttcaagataaaggcatccccgattatattctataccgatgtggattgcgcatactttgtgaacagaaagtgatagcgttgatgattcttcattggtcagaaaattatgaacggtttcttctattttgtctctatatactacgtataggaaatgtttacattttcgtattgttttcgattcactctatgaatagttcttactacaatttttttgtctaaagagtaatactagagataaacataaaaaatgtagaggtcgagtttagatgcaagttcaaggagcgaaaggtggatgggtaggttatatagggatatagcacagagatatatagcaaagagatacttttgagcaatgtttgtggaagcggtattcgcaatattttagtagctcgttacagtccggtgcgtttttggttttttgaaagtgcgtcttcagagcgcttttggttttcaaaagcgctctgaagttcctatacttcctagagaataggaacttcggaataggaacttcaaagcgtttccgaaaacgagcgcttccgaaaatgcaacgcgagctgcgcacatacagctcactgttcacgtcgcacctatatctgcgtgttgcctgtatatatatatacatgagaagaacggcatagtgcgtgtttatgcttaaatgcgtacttatatgcgtctatttatgtaggatgaaaggtagtctagtacctcctgtgatattatcccattccatgcggggtatcgtatgcttccttcagcactaccctttagctgttctatatgctgccactcctcaattggattagtctcatccttcaatgctatcatttcctttgatattggatcatacggcgcgcctggtgtaccgagaacgatcctctcagtgcgagtctcgacgatccatatcgttgcttggcagtcagccagtcggaatccagcttgggacccaggaagtccaatcgtcagatattgtactcaagcctggtcacggcagcgtaccgatctgtttaaacctagatattgatagtctgatcggtcaacgtataatcgagtcctagcttttgcaaacatctatcaagagacaggatcagcaggaggctttcgcatgattgaacaagatggattgcacgcaggttctccggcggcttgggtggagaggctattcggctatgactgggcacaacagacaatcggctgctctgatgccgccgtgttccggctgtcagcgcaggggcgtccggttctttttgtcaagaccgacctgtccggtgccctgaatgaactgcaagacgaggcagcgcggctatcgtggctggcgacgacgggcgttccttgcgcggctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgtctatgcccgacggcgaggatctcgtcgtgacccacggcgatgcctgcttgccgaatatcatggtggaaaatggccgcttttctggattcatcgactgtggccgtctgggtgtggcggaccgctatcaggacatagcgttggctacccgtgatattgctgaagagcttggcggcgaatgggctgaccgcttccttgtgctttacggtatcgccgcgcccgattcgcagcgcatcgccttctatcgccttcttgacgagttcttctgaccgattctaggtgcattggcgcagaaaaaaatgcctgatgcgacgctgcgcgtcttatactcccacatatgccagattcagcaacggatacggcttccccaacttgcccacttccatacgtgtcctccttaccagaaatttatccttaaggtcgtttaaactcgactctggctctatcgaatctccgtcgtttcgagcttacgcgaacagccgtggcgctcatttgctcgtcgggcatcgaatctcgtcagctatcgtcagcttacctttttggcagcgatcgccgcaattaaccctcactaaagggaacaaaagctggagctcggctcccgacatcttggaccattagctccacaggtatcttcttccctctagtggtcataacagcagcttcagctacctctcaatttcaatcgttgcgttacacacacaaaaaaccaacacacatccatcttcgatggatagcgattttattatctaactgctgatcgagtgtagccagatctcatagcttcaaaatgtttctactccttttttactcttccagattttctcggactccgcgcatcgccgtaccacttcaaaacacccaagcacagcatactaaatttcccctctttcttcctctagggtgtcgttaattacccgtactaaaggtttggaaaagaaaaaagagaccgcctcgtttctttttcttcgtcgaaaaaggcaataaaaatttttatcacgtttctttttcttgaaaattttttttttgatttttttctctttcgatgacctcccattgatatttaagttaataaacggtcttcaatttctcaagtttcagtttcatttttcttgttctattacaactttttttacttcttgctcattagaaagaaagcatagcaatctaatctaagagatctttgtcgatcctaccatccactcgacacacccgccagcggccgctgccaagcttccgagctctcgaattcaaaggaggtaccaaccatgaagttgagcaccgtattgctgagtgctgggttagcaagcactacccttgcgcaaggaagttcctatccctatgacgtgccagactacgcatccagcggaggaagttccgactcccctgatcgtccgtggaaccctccaaccttttccccggccttactagtagtaaccgagggtgacaacgcaacttttacctgttcattctctaacacaagcgagtcatttcacgtcgtatggcatagagaaagcccaagcggccaaaccgacacgctagcggccttccccgaagacaggtcacaaccaggacaagatgctagatttagagttacgcaactgcccaatggcagggatttccacatgtcagtggtgagagccaggagaaacgactcagggacgtatgtgtgcggcgtaatctctctggccccaaaaatccaaataaaagaatcattacgtgctgagttaagagttacagaacgtggcggcggaagtagcggtggcggaagcggtgggcagcccagggagcctcaagtctatacgctgcctccgtcaagagaagagatgactaagaaccaagtctctctaacttgtctagtaaaggggttttatccttcagatattgccgtcgagtgggagtcaaatgggcaaccggagaataattacaagacaactcctccggtcctagattccgatggcagtttcttcctttactcaaaactgactgttgataaaagccgttggcagcaaggaaatgttttttcttgtagcgtgatgcatgaagcgctgcataaccactatactcagaaatctttatccctgtcacctggcaaatgatctagaagttgtctcctcctgcactgactgactgatacaatcgatttctggatccgcaggcctctgctagcttgactgactgagatacagcgtaccttcagctcaagtgcttttaactaagaattattagtcttttctgcttattttttcatcatagtttagaacactttatattaacgaatagtttatgaatctatttaggtttaaaaattgatacagttttataagttactttttcaaagactcgtgctgtctattgcataatgcactggaaggggaaaaaaaaggtgcacacgcgtggctttttcttgaatttgcagtttgaaaaataaagcaagcaattcgccctatagtgagtcgtattacgcgcgctaaaacctctacaaatgtggtattggcccatctctatcggtatcgtagcataaccccttggggcctctaaacgggtcttgaggggttttttgtgcccctcgggccggattgctatctaccggcattggcgcagaaaaaaatgcctgatgcgacgctgcgcgtcttatactcccacatatgccagattcagcaacggatacggcttccccaacttgcccacttccatacgtgtcctccttaccagaaatttatccttaaggtcgtcagctatcctgcagggtgtttaaagattacggatatttaacttacttagaataatgccatttttttgagttataataatcctacgttagtgtgagcgggatttaaactgtgaggaccttaatacattcagacacttctgcggtatcaccctacttattcccttcgagattatatctaggaacccatcaggttggtggaagattacccgttctaagacttttcagcttcctctattgatgttacacctggacaccccttttctggcatccagtttttaatcttcagtggcatgtgagattctccgaaattaactaaagcaatcacacaattctctcggataccacctcggttgaaactgacaggtggtttgttacgcatgctaatgcaaaggagcctatatacctttggctcggctgctgtaacagggaatataaagggcagcataatttaggagtttagtgaacttgcaacatttactattttcccttcttacgtaaatatttttctttttaattctaaatcaatctttttcaattttttgtttgtattcttttcttgcttaaatctataactacaaaaaacacatacataaactaaaaatggccaaaaccttcaacatctctcagcaggatctggagctggtggaggtcgccactgagaagatcaccatgctctatgaggacaacaagcaccatgtcggggcggccatcaggaccaagactggggagatcatctctgctgtccacattgaagcctacattggcagggtcactgtctgtgctgaagccattgccattgggtctgctgtgagcaacgggcagaaggactttgacaccattgtggctgtcaggcacccctactctgatgaggtggacagatccatcagggtggtcagcccctgtggcatgtgtagagagctgatctctgactatgctcctgactgctttgtgctcattgagatgaatggcaagctggtcaaaaccaccattgaggaactcatccccctcaagtacaccaggaactaaggttaatataattatataaaaatattatcttcttttctttatatctagtgttatgtaaaataaattgatgactacggaaagcctttttatattgtttctttttcattctgagccacttaaatttcgtgaatgttcttgtaagggacggtagatttacaagtgatacaacaaaaagcaaggcgctttttctaataaaaagaagaaaagcatttaacaattgaacacctctatatcaacgaagaatattcctgcaggcgatctctcgatttcgatcaagacattcctttaatggtcttttctggacaccactaggggtcagaagtagttcatcaaactttcttccctccctaatctcattggttaccttgggctatcgaaacttaattaaccagtcaagtcagctacttggcgagatcgacttgtctgggtttcgactacgctcagaattgcgtcagtcaagttcgatctggtccttgctattgcacccgttctccgattacgagtttcatttaaatcatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcatttaaatttccgaactctccaaggccctcgtcggaaaatcttcaaacctttcgtccgatccatcttgcaggctacctctcgaacgaactatcgcaagtctcttggccggccttgcgccttggc

SEQ ID NO: 2 - DNA, 7574 bp, synthetic construct
  source 1..7574: mol_type = other DNA
tattgcttggcagcgcctatcgccaggtattactccaatcccgaatatccgagatcgggatcacccgagagaagttcaacctacatcctcaatcccgatctatccgagatccgaggaatatcgaaatcggggcgcgccaacgaagcatctgtgcttcattttgtagaacaaaaatgcaacgcgagagcgctaatttttcaaacaaagaatctgagctgcatttttacagaacagaaatgcaacgcgaaagcgctattttaccaacgaagaatctgtgcttcatttttgtaaaacaaaaatgcaacgcgagagcgctaatttttcaaacaaagaatctgagctgcatttttacagaacagaaatgcaacgcgagagcgctattttaccaacaaagaatctatacttcttttttgttctacaaaaatgcatcccgagagcgctatttttctaacaaagcatcttagattactttttttctcctttgtgcgctctataatgcagtctcttgataactttttgcactgtaggtccgttaaggttagaagaaggctactttggtgtctattttctcttccataaaaaaagcctgactccacttcccgcgtttactgattactagcgaagctgcgggtgcattttttcaagataaaggcatccccgattatattctataccgatgtggattgcgcatactttgtgaacagaaagtgatagcgttgatgattcttcattggtcagaaaattatgaacggtttcttctattttgtctctatatactacgtataggaaatgtttacattttcgtattgttttcgattcactctatgaatagttcttactacaatttttttgtctaaagagtaatactagagataaacataaaaaatgtagaggtcgagtttagatgcaagttcaaggagcgaaaggtggatgggtaggttatatagggatatagcacagagatatatagcaaagagatacttttgagcaatgtttgtggaagcggtattcgcaatattttagtagctcgttacagtccggtgcgtttttggttttttgaaagtgcgtcttcagagcgcttttggttttcaaaagcgctctgaagttcctatacttcctagagaataggaacttcggaataggaacttcaaagcgtttccgaaaacgagcgcttccgaaaatgcaacgcgagctgcgcacatacagctcactgttcacgtcgcacctatatctgcgtgttgcctgtatatatatatacatgagaagaacggcatagtgcgtgtttatgcttaaatgcgtacttatatgcgtctatttatgtaggatgaaaggtagtctagtacctcctgtgatattatcccattccatgcggggtatcgtatgcttccttcagcactaccctttagctgttctatatgctgccactcctcaattggattagtctcatccttcaatgctatcatttcctttgatattggatcatacggcgcgcctggtgtaccgagaacgatcctctcagtgcgagtctcgacgatccatatcgttgcttggcagtcagccagtcggaatccagcttgggacccaggaagtccaatcgtcagatattgtactcaagcctggtcacggcagcgtaccgatctgtttaaacctagatattgatagtctgatcggtcaacgtataatcgagtcctagcttttgcaaacatctatcaagagacaggatcagcaggaggctttcgcatgattgaacaagatggattgcacgcaggttctccggcggcttgggtggagaggctattcggctatgactgggcacaacagacaatcggctgctctgatgccgccgtgttccggctgtcagcgcaggggcgtccggttctttttgtcaagaccgacctgtccggtgccctgaatgaactgcaagacgaggcagcgcggctatcgtggctggcgacgacgggcgttccttgcgcggctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgtctatgcccgacggcgaggatctcgtcgtgacccacggcgatgcctgcttgccgaatatcatggtggaaaatggccgcttttctggattcatcgactgtggccgtctgggtgtggcggaccgctatcaggacatagcgttggctacccgtgatattgctgaagagcttggcggcgaatgggctgaccgcttccttgtgctttacggtatcgccgcgcccgattcgcagcgcatcgccttctatcgccttcttgacgagttcttctgaccgattctaggtgcattggcgcagaaaaaaatgcctgatgcgacgctgcgcgtcttatactcccacatatgccagattcagcaacggatacggcttccccaacttgcccacttccatacgtgtcctccttaccagaaatttatccttaaggtcgtttaaactcgactctggctctatcgaatctccgtcgtttcgagcttacgcgaacagccgtggcgctcatttgctcgtcgggcatcgaatctcgtcagctatcgtcagcttacctttttggcagcgatcgccgcaattaaccctcactaaagggaacaaaagctggagctcggctcccgacatcttggaccattagctccacaggtatcttcttccctctagtggtcataacagcagcttcagctacctctcaatttcaatcgttgcgttacacacacaaaaaaccaacacacatccatcttcgatggatagcgattttattatctaactgctgatcgagtgtagccagatctcatagcttcaaaatgtttctactccttttttactcttccagattttctcggactccgcgcatcgccgtaccacttcaaaacacccaagcacagcatactaaatttcccctctttcttcctctagggtgtcgttaattacccgtactaaaggtttggaaaagaaaaaagagaccgcctcgtttctttttcttcgtcgaaaaaggcaataaaaatttttatcacgtttctttttcttgaaaattttttttttgatttttttctctttcgatgacctcccattgatatttaagttaataaacggtcttcaatttctcaagtttcagtttcatttttcttgttctattacaactttttttacttcttgctcattagaaagaaagcatagcaatctaatctaagagatctttgtcgatcctaccatccactcgacacacccgccagcggccgctgccaagcttccgagctctcgaattcaaaggaggtaccaaccatgaagttgagcaccgtattgctgagtgctgggttagcaagcactacccttgcgcaaggaagttcctatccctatgacgtgccagactacgcatccagcggaggaagttccgactcccctgatcgtccgtggaaccctccaaccttttccccggccttactagtagtaaccgagggtgacaacgcaacttttacctgttcattctctaacacaagcgagtcatttcacgtcgtatggcatagagaaagcccaagcggccaaaccgacacgctagcggccttccccgaagacaggtcacaaccaggacaagatgctagatttagagttacgcaactgcccaatggcagggatttccacatgtcagtggtgagagccaggagaaacgactcagggacgtatgtgtgcggcgtaatctctctggccccaaaaatccaaataaaagaatcattacgtgctgagttaagagttacagaacgtggcggcggaagtagcggtggcggaagcggtgggcagcccagggagcctcaagtctatacgctgcctccgtcaagagaagagatgactaagaaccaagtctctctaacttgtctagtaaaggggttttatccttcagatattgccgtcgagtgggagtcaaatgggcaaccggagaataattacaagacaactcctccggtcctagattccgatggcagtttcttcctttactcaaaactgactgttgataaaagccgttggcagcaaggaaatgttttttcttgtagcgtgatgcatgaagcgctgcataaccactatactcagaaatctttatccctgtcacctggcaaatgatctagaagttgtctcctcctgcactgactgactgatacaatcgatttctggatccgcaggcctctgctagcttgactgactgagatacagcgtaccttcagctcatcatgtaattagttatgtcacgcttacattcacgccctccccccacatccgctctaaccgaaaaggaaggagttagacaacctgaagtctaggtccctatttatttttttatagttatgttagtattaagaacgttatttatatttcaaatttttcttttttttctgtacagacgcgtgtacgcatgtaacattatactgaaaaccttgcttgagaaggttttgggacgctcgaaggctttaatttgcggccaaagcaagcaattcgccctatagtgagtcgtattacgcgcgctaaaacctctacaaatgtggtattggcccatctctatcggtatcgtagcataaccccttggggcctctaaacgggtcttgaggggttttttgtgcccctcgggccggattgctatctaccggcattggcgcagaaaaaaatgcctgatgcgacgctgcgcgtcttatactcccacatatgccagattcagcaacggatacggcttccccaacttgcccacttccatacgtgtcctccttaccagaaatttatccttaaggtcgtcagctatcctgcagggtgtttaaagattacggatatttaacttacttagaataatgccatttttttgagttataataatcctacgttagtgtgagcgggatttaaactgtgaggaccttaatacattcagacacttctgcggtatcaccctacttattcccttcgagattatatctaggaacccatcaggttggtggaagattacccgttctaagacttttcagcttcctctattgatgttacacctggacaccccttttctggcatccagtttttaatcttcagtggcatgtgagattctccgaaattaactaaagcaatcacacaattctctcggataccacctcggttgaaactgacaggtggtttgttacgcatgctaatgcaaaggagcctatatacctttggctcggctgctgtaacagggaatataaagggcagcataatttaggagtttagtgaacttgcaacatttactattttcccttcttacgtaaatatttttctttttaattctaaatcaatctttttcaattttttgtttgtattcttttcttgcttaaatctataactacaaaaaacacatacataaactaaaaatggccaaaaccttcaacatctctcagcaggatctggagctggtggaggtcgccactgagaagatcaccatgctctatgaggacaacaagcaccatgtcggggcggccatcaggaccaagactggggagatcatctctgctgtccacattgaagcctacattggcagggtcactgtctgtgctgaagccattgccattgggtctgctgtgagcaacgggcagaaggactttgacaccattgtggctgtcaggcacccctactctgatgaggtggacagatccatcagggtggtcagcccctgtggcatgtgtagagagctgatctctgactatgctcctgactgctttgtgctcattgagatgaatggcaagctggtcaaaaccaccattgaggaactcatccccctcaagtacaccaggaactaaggttaatataattatataaaaatattatcttcttttctttatatctagtgttatgtaaaataaattgatgactacggaaagcctttttatattgtttctttttcattctgagccacttaaatttcgtgaatgttcttgtaagggacggtagatttacaagtgatacaacaaaaagcaaggcgctttttctaataaaaagaagaaaagcatttaacaattgaacacctctatatcaacgaagaatattcctgcaggcgatctctcgatttcgatcaagacattcctttaatggtcttttctggacaccactaggggtcagaagtagttcatcaaactttcttccctccctaatctcattggttaccttgggctatcgaaacttaattaaccagtcaagtcagctacttggcgagatcgacttgtctgggtttcgactacgctcagaattgcgtcagtcaagttcgatctggtccttgctattgcacccgttctccgattacgagtttcatttaaatcatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcatttaaatttccgaactctccaaggccctcgtcggaaaatcttcaaacctttcgtccgatccatcttgcaggctacctctcgaacgaactatcgcaagtctcttggccggccttgcgccttggc

SEQ ID NO: 3 - DNA, 7547 bp, synthetic construct
  source 1..7547: mol_type = other DNA
tattgcttggcagcgcctatcgccaggtattactccaatcccgaatatccgagatcgggatcacccgagagaagttcaacctacatcctcaatcccgatctatccgagatccgaggaatatcgaaatcggggcgcgccaacgaagcatctgtgcttcattttgtagaacaaaaatgcaacgcgagagcgctaatttttcaaacaaagaatctgagctgcatttttacagaacagaaatgcaacgcgaaagcgctattttaccaacgaagaatctgtgcttcatttttgtaaaacaaaaatgcaacgcgagagcgctaatttttcaaacaaagaatctgagctgcatttttacagaacagaaatgcaacgcgagagcgctattttaccaacaaagaatctatacttcttttttgttctacaaaaatgcatcccgagagcgctatttttctaacaaagcatcttagattactttttttctcctttgtgcgctctataatgcagtctcttgataactttttgcactgtaggtccgttaaggttagaagaaggctactttggtgtctattttctcttccataaaaaaagcctgactccacttcccgcgtttactgattactagcgaagctgcgggtgcattttttcaagataaaggcatccccgattatattctataccgatgtggattgcgcatactttgtgaacagaaagtgatagcgttgatgattcttcattggtcagaaaattatgaacggtttcttctattttgtctctatatactacgtataggaaatgtttacattttcgtattgttttcgattcactctatgaatagttcttactacaatttttttgtctaaagagtaatactagagataaacataaaaaatgtagaggtcgagtttagatgcaagttcaaggagcgaaaggtggatgggtaggttatatagggatatagcacagagatatatagcaaagagatacttttgagcaatgtttgtggaagcggtattcgcaatattttagtagctcgttacagtccggtgcgtttttggttttttgaaagtgcgtcttcagagcgcttttggttttcaaaagcgctctgaagttcctatacttcctagagaataggaacttcggaataggaacttcaaagcgtttccgaaaacgagcgcttccgaaaatgcaacgcgagctgcgcacatacagctcactgttcacgtcgcacctatatctgcgtgttgcctgtatatatatatacatgagaagaacggcatagtgcgtgtttatgcttaaatgcgtacttatatgcgtctatttatgtaggatgaaaggtagtctagtacctcctgtgatattatcccattccatgcggggtatcgtatgcttccttcagcactaccctttagctgttctatatgctgccactcctcaattggattagtctcatccttcaatgctatcatttcctttgatattggatcatacggcgcgcctggtgtaccgagaacgatcctctcagtgcgagtctcgacgatccatatcgttgcttggcagtcagccagtcggaatccagcttgggacccaggaagtccaatcgtcagatattgtactcaagcctggtcacggcagcgtaccgatctgtttaaacctagatattgatagtctgatcggtcaacgtataatcgagtcctagcttttgcaaacatctatcaagagacaggatcagcaggaggctttcgcatgattgaacaagatggattgcacgcaggttctccggcggcttgggtggagaggctattcggctatgactgggcacaacagacaatcggctgctctgatgccgccgtgttccggctgtcagcgcaggggcgtccggttctttttgtcaagaccgacctgtccggtgccctgaatgaactgcaagacgaggcagcgcggctatcgtggctggcgacgacgggcgttccttgcgcggctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgtctatgcccgacggcgaggatctcgtcgtgacccacggcgatgcctgcttgccgaatatcatggtggaaaatggccgcttttctggattcatcgactgtggccgtctgggtgtggcggaccgctatcaggacatagcgttggctacccgtgatattgctgaagagcttggcggcgaatgggctgaccgcttccttgtgctttacggtatcgccgcgcccgattcgcagcgcatcgccttctatcgccttcttgacgagttcttctgaccgattctaggtgcattggcgcagaaaaaaatgcctgatgcgacgctgcgcgtcttatactcccacatatgccagattcagcaacggatacggcttccccaacttgcccacttccatacgtgtcctccttaccagaaatttatccttaaggtcgtttaaactcgactctggctctatcgaatctccgtcgtttcgagcttacgcgaacagccgtggcgctcatttgctcgtcgggcatcgaatctcgtcagctatcgtcagcttacctttttggcagcgatcgccgcaattaaccctcactaaagggaacaaaagctggagctcggctcccgacatcttggaccattagctccacaggtatcttcttccctctagtggtcataacagcagcttcagctacctctcaatttcaatcgttgcgttacacacacaaaaaaccaacacacatccatcttcgatggatagcgattttattatctaactgctgatcgagtgtagccagatctcatagcttcaaaatgtttctactccttttttactcttccagattttctcggactccgcgcatcgccgtaccacttcaaaacacccaagcacagcatactaaatttcccctctttcttcctctagggtgtcgttaattacccgtactaaaggtttggaaaagaaaaaagagaccgcctcgtttctttttcttcgtcgaaaaaggcaataaaaatttttatcacgtttctttttcttgaaaattttttttttgatttttttctctttcgatgacctcccattgatatttaagttaataaacggtcttcaatttctcaagtttcagtttcatttttcttgttctattacaactttttttacttcttgctcattagaaagaaagcatagcaatctaatctaagagatctttgtcgatcctaccatccactcgacacacccgccagcggccgctgccaagcttccgagctctcgaattcaaaggaggtaccaaccatgaagttgagcaccgtattgctgagtgctgggttagcaagcactacccttgcgcaaggaagttcctatccctatgacgtgccagactacgcatccagcggaggaagttccgactcccctgatcgtccgtggaaccctccaaccttttccccggccttactagtagtaaccgagggtgacaacgcaacttttacctgttcattctctaacacaagcgagtcatttcacgtcgtatggcatagagaaagcccaagcggccaaaccgacacgctagcggccttccccgaagacaggtcacaaccaggacaagatgctagatttagagttacgcaactgcccaatggcagggatttccacatgtcagtggtgagagccaggagaaacgactcagggacgtatgtgtgcggcgtaatctctctggccccaaaaatccaaataaaagaatcattacgtgctgagttaagagttacagaacgtggcggcggaagtagcggtggcggaagcggtgggcagcccagggagcctcaagtctatacgctgcctccgtcaagagaagagatgactaagaaccaagtctctctaacttgtctagtaaaggggttttatccttcagatattgccgtcgagtgggagtcaaatgggcaaccggagaataattacaagacaactcctccggtcctagattccgatggcagtttcttcctttactcaaaactgactgttgataaaagccgttggcagcaaggaaatgttttttcttgtagcgtgatgcatgaagcgctgcataaccactatactcagaaatctttatccctgtcacctggcaaatgatctagaagttgtctcctcctgcactgactgactgatacaatcgatttctggatccgcaggcctctgctagcttgactgactgagatacagcgtaccttcagctcaataaagcaatcttgatgaggataatgatttttttttgaatatacataaatactaccgtttttctgctagattttgtgatgacgtaaataagtacatattactttttaagccaagacaagattaagcattaactttacccttttctttctaagtttcaatattagttatcactgtttaaaagttatggcgagaacgtcggcggttaaaatatattaccctgaacgaaaagcaagcaattcgccctatagtgagtcgtattacgcgcgctaaaacctctacaaatgtggtattggcccatctctatcggtatcgtagcataaccccttggggcctctaaacgggtcttgaggggttttttgtgcccctcgggccggattgctatctaccggcattggcgcagaaaaaaatgcctgatgcgacgctgcgcgtcttatactcccacatatgccagattcagcaacggatacggcttccccaacttgcccacttccatacgtgtcctccttaccagaaatttatccttaaggtcgtcagctatcctgcagggtgtttaaagattacggatatttaacttacttagaataatgccatttttttgagttataataatcctacgttagtgtgagcgggatttaaactgtgaggaccttaatacattcagacacttctgcggtatcaccctacttattcccttcgagattatatctaggaacccatcaggttggtggaagattacccgttctaagacttttcagcttcctctattgatgttacacctggacaccccttttctggcatccagtttttaatcttcagtggcatgtgagattctccgaaattaactaaagcaatcacacaattctctcggataccacctcggttgaaactgacaggtggtttgttacgcatgctaatgcaaaggagcctatatacctttggctcggctgctgtaacagggaatataaagggcagcataatttaggagtttagtgaacttgcaacatttactattttcccttcttacgtaaatatttttctttttaattctaaatcaatctttttcaattttttgtttgtattcttttcttgcttaaatctataactacaaaaaacacatacataaactaaaaatggccaaaaccttcaacatctctcagcaggatctggagctggtggaggtcgccactgagaagatcaccatgctctatgaggacaacaagcaccatgtcggggcggccatcaggaccaagactggggagatcatctctgctgtccacattgaagcctacattggcagggtcactgtctgtgctgaagccattgccattgggtctgctgtgagcaacgggcagaaggactttgacaccattgtggctgtcaggcacccctactctgatgaggtggacagatccatcagggtggtcagcccctgtggcatgtgtagagagctgatctctgactatgctcctgactgctttgtgctcattgagatgaatggcaagctggtcaaaaccaccattgaggaactcatccccctcaagtacaccaggaactaaggttaatataattatataaaaatattatcttcttttctttatatctagtgttatgtaaaataaattgatgactacggaaagcctttttatattgtttctttttcattctgagccacttaaatttcgtgaatgttcttgtaagggacggtagatttacaagtgatacaacaaaaagcaaggcgctttttctaataaaaagaagaaaagcatttaacaattgaacacctctatatcaacgaagaatattcctgcaggcgatctctcgatttcgatcaagacattcctttaatggtcttttctggacaccactaggggtcagaagtagttcatcaaactttcttccctccctaatctcattggttaccttgggctatcgaaacttaattaaccagtcaagtcagctacttggcgagatcgacttgtctgggtttcgactacgctcagaattgcgtcagtcaagttcgatctggtccttgctattgcacccgttctccgattacgagtttcatttaaatcatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcatttaaatttccgaactctccaaggccctcgtcggaaaatcttcaaacctttcgtccgatccatcttgcaggctacctctcgaacgaactatcgcaagtctcttggccggccttgcgccttggc

SEQ ID NO: 4 - DNA, 8082 bp, synthetic construct
  source 1..8082: mol_type = other DNA
atccttttgttgtttccgggtgtacaatatggacttcctcttttctggcaaccaaacccatacatcgggattcctataataccttcgttggtctccctaacatgtaggtggcggaggggagatatacaatagaacagataccagacaagacataatgggctaaacaagactacaccaattacactgcctcattgatggtggtacataacgaactaatactgtagccctagacttgatagccatcatcatatcgaagtttcactaccctttttccatttgccatctattgaagtaataataggcgcatgcaacttcttttctttttttttcttttctctctcccccgttgttgtctcaccatatccgcaatgacaaaaaaatgatggaagacactaaaggaaaaaattaacgacaaagacagcaccaacagatgtcgttgttccagagctgatgaggggtatctcgaagcacacgaaactttttccttccttcattcacgcacactactctctaatgagcaacggtatacggccttccttccagttacttgaatttgaaataaaaaaaagtttgctgtcttgctatcaagtataaatagacctgcaattattaatcttttgtttcctcgtcattgttctcgttccctttcttccttgtttctttttctgcacaatatttcaagctataccaagcatacaatcaactagatctttgtcgatcctaccatccactcgacacacccgccagcggccgctgccaagcttccgagctctcgaattcaaaggaggtaccaaccatggggagatttccttcaatttttactgcagttttattcgcagcatcctccgcattagctgctccagtcaacactacaacagaagatgaaacggcacaaattccggctgaagctgtcatcggttacttagatttagaaggggatttcgatgttgctgttttgccattttccaacagcacaaataacgggttattgtttataaatactactattgccagcattgctgctaaagaagaaggggtatctttggataaaagagagggaagttcctatccctatgacgtgccagactacgcatccagcggaggaagttccgactcccctgatcgtccgtggaaccctccaaccttttccccggccttactagtagtaaccgagggtgacaacgcaacttttacctgttcattctctaacacaagcgagtcatttcacgtcgtatggcatagagaaagcccaagcggccaaaccgacacgctagcggccttccccgaagacaggtcacaaccaggacaagatgctagatttagagttacgcaactgcccaatggcagggatttccacatgtcagtggtgagagccaggagaaacgactcagggacgtatgtgtgcggcgtaatctctctggccccaaaaatccaaataaaagaatcattacgtgctgagttaagagttacagaacgtggcggcggaagtagcggtggcggaagcggtgggcagcccagggagcctcaagtctatacgctgcctccgtcaagagaagagatgactaagaaccaagtctctctaacttgtctagtaaaggggttttatccttcagatattgccgtcgagtgggagtcaaatgggcaaccggagaataattacaagacaactcctccggtcctagattccgatggcagtttcttcctttactcaaaactgactgttgataaaagccgttggcagcaaggaaatgttttttcttgtagcgtgatgcatgaagcgctgcataaccactatactcagaaatctttatccctgtcacctggcaaatgatctagaagttgtctcctcctgcactgactgactgatacaatcgatttctggatccgcaggcctctgctagcttgactgactgagatacagcgtaccttcagctcatgaacccgatgcaaatgagacgatcgtctattcctggtccggttttctctgccctctcttctattcactttttttatactttatataaaattatataaatgacataactgaaacgccacacgtcctctcctattcgttaacgcctgtctgtagcgctgttactgaagctgcgcaagtagttttttcaccgtataggccctctttttctctctctttctttctctcccgcgctgatctcttcttcgaaacaaaagcaagcaattcgccctatagtgagtcgtattacgcgcgctaaaacctctacaaatgtggtattggcccatctctatcggtatcgtagcataaccccttggggcctctaaacgggtcttgaggggttttttgtgcccctcgggccggattgctatctaccggcattggcgcagaaaaaaatgcctgatgcgacgctgcgcgtcttatactcccacatatgccagattcagcaacggatacggcttccccaacttgcccacttccatacgtgtcctccttaccagaaatttatccttaaggtcgtcagctatcctgcagggtgtttaaagattacggatatttaacttacttagaataatgccatttttttgagttataataatcctacgttagtgtgagcgggatttaaactgtgaggaccttaatacattcagacacttctgcggtatcaccctacttattcccttcgagattatatctaggaacccatcaggttggtggaagattacccgttctaagacttttcagcttcctctattgatgttacacctggacaccccttttctggcatccagtttttaatcttcagtggcatgtgagattctccgaaattaactaaagcaatcacacaattctctcggataccacctcggttgaaactgacaggtggtttgttacgcatgctaatgcaaaggagcctatatacctttggctcggctgctgtaacagggaatataaagggcagcataatttaggagtttagtgaacttgcaacatttactattttcccttcttacgtaaatatttttctttttaattctaaatcaatctttttcaattttttgtttgtattcttttcttgcttaaatctataactacaaaaaacacatacataaactaaaaatggccaaaaccttcaacatctctcagcaggatctggagctggtggaggtcgccactgagaagatcaccatgctctatgaggacaacaagcaccatgtcggggcggccatcaggaccaagactggggagatcatctctgctgtccacattgaagcctacattggcagggtcactgtctgtgctgaagccattgccattgggtctgctgtgagcaacgggcagaaggactttgacaccattgtggctgtcaggcacccctactctgatgaggtggacagatccatcagggtggtcagcccctgtggcatgtgtagagagctgatctctgactatgctcctgactgctttgtgctcattgagatgaatggcaagctggtcaaaaccaccattgaggaactcatccccctcaagtacaccaggaactaaggttaatataattatataaaaatattatcttcttttctttatatctagtgttatgtaaaataaattgatgactacggaaagcctttttatattgtttctttttcattctgagccacttaaatttcgtgaatgttcttgtaagggacggtagatttacaagtgatacaacaaaaagcaaggcgctttttctaataaaaagaagaaaagcatttaacaattgaacacctctatatcaacgaagaatattcctgcaggcgatctctcgatttcgatcaagacattcctttaatggtcttttctggacaccactaggggtcagaagtagttcatcaaactttcttccctccctaatctcattggttaccttgggctatcgaaacttaattaaccagtcaagtcagctacttggcgagatcgacttgtctgggtttcgactacgctcagaattgcgtcagtcaagttcgatctggtccttgctattgcacccgttctccgattacgagtttcatttaaatcatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcatttaaatttccgaactctccaaggccctcgtcggaaaatcttcaaacctttcgtccgatccatcttgcaggctacctctcgaacgaactatcgcaagtctcttggccggccttgcgccttggctattgcttggcagcgcctatcgccaggtattactccaatcccgaatatccgagatcgggatcacccgagagaagttcaacctacatcctcaatcccgatctatccgagatccgaggaatatcgaaatcggggcgcgccaacgaagcatctgtgcttcattttgtagaacaaaaatgcaacgcgagagcgctaatttttcaaacaaagaatctgagctgcatttttacagaacagaaatgcaacgcgaaagcgctattttaccaacgaagaatctgtgcttcatttttgtaaaacaaaaatgcaacgcgagagcgctaatttttcaaacaaagaatctgagctgcatttttacagaacagaaatgcaacgcgagagcgctattttaccaacaaagaatctatacttcttttttgttctacaaaaatgcatcccgagagcgctatttttctaacaaagcatcttagattactttttttctcctttgtgcgctctataatgcagtctcttgataactttttgcactgtaggtccgttaaggttagaagaaggctactttggtgtctattttctcttccataaaaaaagcctgactccacttcccgcgtttactgattactagcgaagctgcgggtgcattttttcaagataaaggcatccccgattatattctataccgatgtggattgcgcatactttgtgaacagaaagtgatagcgttgatgattcttcattggtcagaaaattatgaacggtttcttctattttgtctctatatactacgtataggaaatgtttacattttcgtattgttttcgattcactctatgaatagttcttactacaatttttttgtctaaagagtaatactagagataaacataaaaaatgtagaggtcgagtttagatgcaagttcaaggagcgaaaggtggatgggtaggttatatagggatatagcacagagatatatagcaaagagatacttttgagcaatgtttgtggaagcggtattcgcaatattttagtagctcgttacagtccggtgcgtttttggttttttgaaagtgcgtcttcagagcgcttttggttttcaaaagcgctctgaagttcctatacttcctagagaataggaacttcggaataggaacttcaaagcgtttccgaaaacgagcgcttccgaaaatgcaacgcgagctgcgcacatacagctcactgttcacgtcgcacctatatctgcgtgttgcctgtatatatatatacatgagaagaacggcatagtgcgtgtttatgcttaaatgcgtacttatatgcgtctatttatgtaggatgaaaggtagtctagtacctcctgtgatattatcccattccatgcggggtatcgtatgcttccttcagcactaccctttagctgttctatatgctgccactcctcaattggattagtctcatccttcaatgctatcatttcctttgatattggatcatacggcgcgcctggtgtaccgagaacgatcctctcagtgcgagtctcgacgatccatatcgttgcttggcagtcagccagtcggaatccagcttgggacccaggaagtccaatcgtcagatattgtactcaagcctggtcacggcagcgtaccgatctgtttaaacctagatattgatagtctgatcggtcaacgtataatcgagtcctagcttttgcaaacatctatcaagagacaggatcagcaggaggctttcgcatgattgaacaagatggattgcacgcaggttctccggcggcttgggtggagaggctattcggctatgactgggcacaacagacaatcggctgctctgatgccgccgtgttccggctgtcagcgcaggggcgtccggttctttttgtcaagaccgacctgtccggtgccctgaatgaactgcaagacgaggcagcgcggctatcgtggctggcgacgacgggcgttccttgcgcggctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgtctatgcccgacggcgaggatctcgtcgtgacccacggcgatgcctgcttgccgaatatcatggtggaaaatggccgcttttctggattcatcgactgtggccgtctgggtgtggcggaccgctatcaggacatagcgttggctacccgtgatattgctgaagagcttggcggcgaatgggctgaccgcttccttgtgctttacggtatcgccgcgcccgattcgcagcgcatcgccttctatcgccttcttgacgagttcttctgaccgattctaggtgcattggcgcagaaaaaaatgcctgatgcgacgctgcgcgtcttatactcccacatatgccagattcagcaacggatacggcttccccaacttgcccacttccatacgtgtcctccttaccagaaatttatccttaaggtcgtttaaactcgactctggctctatcgaatctccgtcgtttcgagcttacgcgaacagccgtggcgctcatttgctcgtcgggcatcgaatctcgtcagctatcgtcagcttacctttttggcagcgatcgccgcaattaaccctcactaaagggaacaaaagctggagctcggctcccgacatcttggaccattagctccacaggtatcttcttccctctagtggtcataacagcagcttcagctacctctcaatttcaatcgttgcgttacacacacaaaaaaccaacacacatccatcttcgatggatagcgattttattatctaactgctgatcgagtgtagccagatct

SEQ ID NO: 5 - DNA, 7731 bp, synthetic construct
  source 1..7731: mol_type = other DNA
tcattatcaatactcgccatttcaaagaatacgtaaataattaatagtagtgattttcctaactttatttagtcaaaaaattagccttttaattctgctgtaacccgtacatgcccaaaatagggggcgggttacacagaatatataacatcgtaggtgtctgggtgaacagtttattcctggcatccactaaatataatggagcccgctttttaagctggcatccagaaaaaaaaagaatcccagcaccaaaatattgttttcttcaccaaccatcagttcataggtccattctcttagcgcaactacagagaacaggggcacaaacaggcaaaaaacgggcacaacctcaatggagtgatgcaacctgcctggagtaaatgatgacacaaggcaattgacccacgcatgtatctatctcattttcttacaccttctattaccttctgctctctctgatttggaaaaagctgaaaaaaaaggttgaaaccagttccctgaaattattcccctacttgactaataagtatataaagacggtaggtattgattgtaattctgtaaatctatttcttaaacttctagatctttgtcgatcctaccatccactcgacacacccgccagcggccgctgccaagcttccgagctctcgaattcaaaggaggtaccaaccatgttgttacaggcattcttgtttctgttggccggttttgctgcgaagatttctgccggaagttcctatccctatgacgtgccagactacgcatccagcggaggaagttccgactcccctgatcgtccgtggaaccctccaaccttttccccggccttactagtagtaaccgagggtgacaacgcaacttttacctgttcattctctaacacaagcgagtcatttcacgtcgtatggcatagagaaagcccaagcggccaaaccgacacgctagcggccttccccgaagacaggtcacaaccaggacaagatgctagatttagagttacgcaactgcccaatggcagggatttccacatgtcagtggtgagagccaggagaaacgactcagggacgtatgtgtgcggcgtaatctctctggccccaaaaatccaaataaaagaatcattacgtgctgagttaagagttacagaacgtggcggcggaagtagcggtggcggaagcggtgggcagcccagggagcctcaagtctatacgctgcctccgtcaagagaagagatgactaagaaccaagtctctctaacttgtctagtaaaggggttttatccttcagatattgccgtcgagtgggagtcaaatgggcaaccggagaataattacaagacaactcctccggtcctagattccgatggcagtttcttcctttactcaaaactgactgttgataaaagccgttggcagcaaggaaatgttttttcttgtagcgtgatgcatgaagcgctgcataaccactatactcagaaatctttatccctgtcacctggcaaatgatctagaagttgtctcctcctgcactgactgactgatacaatcgatttctggatccgcaggcctctgctagcttgactgactgagatacagcgtaccttcagctcaataaagcaatcttgatgaggataatgatttttttttgaatatacataaatactaccgtttttctgctagattttgtgatgacgtaaataagtacatattactttttaagccaagacaagattaagcattaactttacccttttctttctaagtttcaatattagttatcactgtttaaaagttatggcgagaacgtcggcggttaaaatatattaccctgaacgaaaagcaagcaattcgccctatagtgagtcgtattacgcgcgctaaaacctctacaaatgtggtattggcccatctctatcggtatcgtagcataaccccttggggcctctaaacgggtcttgaggggttttttgtgcccctcgggccggattgctatctaccggcattggcgcagaaaaaaatgcctgatgcgacgctgcgcgtcttatactcccacatatgccagattcagcaacggatacggcttccccaacttgcccacttccatacgtgtcctccttaccagaaatttatccttaaggtcgtcagctatcctgcagggtgtttaaagattacggatatttaacttacttagaataatgccatttttttgagttataataatcctacgttagtgtgagcgggatttaaactgtgaggaccttaatacattcagacacttctgcggtatcaccctacttattcccttcgagattatatctaggaacccatcaggttggtggaagattacccgttctaagacttttcagcttcctctattgatgttacacctggacaccccttttctggcatccagtttttaatcttcagtggcatgtgagattctccgaaattaactaaagcaatcacacaattctctcggataccacctcggttgaaactgacaggtggtttgttacgcatgctaatgcaaaggagcctatatacctttggctcggctgctgtaacagggaatataaagggcagcataatttaggagtttagtgaacttgcaacatttactattttcccttcttacgtaaatatttttctttttaattctaaatcaatctttttcaattttttgtttgtattcttttcttgcttaaatctataactacaaaaaacacatacataaactaaaaatggccaaaaccttcaacatctctcagcaggatctggagctggtggaggtcgccactgagaagatcaccatgctctatgaggacaacaagcaccatgtcggggcggccatcaggaccaagactggggagatcatctctgctgtccacattgaagcctacattggcagggtcactgtctgtgctgaagccattgccattgggtctgctgtgagcaacgggcagaaggactttgacaccattgtggctgtcaggcacccctactctgatgaggtggacagatccatcagggtggtcagcccctgtggcatgtgtagagagctgatctctgactatgctcctgactgctttgtgctcattgagatgaatggcaagctggtcaaaaccaccattgaggaactcatccccctcaagtacaccaggaactaaggttaatataattatataaaaatattatcttcttttctttatatctagtgttatgtaaaataaattgatgactacggaaagcctttttatattgtttctttttcattctgagccacttaaatttcgtgaatgttcttgtaagggacggtagatttacaagtgatacaacaaaaagcaaggcgctttttctaataaaaagaagaaaagcatttaacaattgaacacctctatatcaacgaagaatattcctgcaggcgatctctcgatttcgatcaagacattcctttaatggtcttttctggacaccactaggggtcagaagtagttcatcaaactttcttccctccctaatctcattggttaccttgggctatcgaaacttaattaaccagtcaagtcagctacttggcgagatcgacttgtctgggtttcgactacgctcagaattgcgtcagtcaagttcgatctggtccttgctattgcacccgttctccgattacgagtttcatttaaatcatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcatttaaatttccgaactctccaaggccctcgtcggaaaatcttcaaacctttcgtccgatccatcttgcaggctacctctcgaacgaactatcgcaagtctcttggccggccttgcgccttggctattgcttggcagcgcctatcgccaggtattactccaatcccgaatatccgagatcgggatcacccgagagaagttcaacctacatcctcaatcccgatctatccgagatccgaggaatatcgaaatcggggcgcgccaacgaagcatctgtgcttcattttgtagaacaaaaatgcaacgcgagagcgctaatttttcaaacaaagaatctgagctgcatttttacagaacagaaatgcaacgcgaaagcgctattttaccaacgaagaatctgtgcttcatttttgtaaaacaaaaatgcaacgcgagagcgctaatttttcaaacaaagaatctgagctgcatttttacagaacagaaatgcaacgcgagagcgctattttaccaacaaagaatctatacttcttttttgttctacaaaaatgcatcccgagagcgctatttttctaacaaagcatcttagattactttttttctcctttgtgcgctctataatgcagtctcttgataactttttgcactgtaggtccgttaaggttagaagaaggctactttggtgtctattttctcttccataaaaaaagcctgactccacttcccgcgtttactgattactagcgaagctgcgggtgcattttttcaagataaaggcatccccgattatattctataccgatgtggattgcgcatactttgtgaacagaaagtgatagcgttgatgattcttcattggtcagaaaattatgaacggtttcttctattttgtctctatatactacgtataggaaatgtttacattttcgtattgttttcgattcactctatgaatagttcttactacaatttttttgtctaaagagtaatactagagataaacataaaaaatgtagaggtcgagtttagatgcaagttcaaggagcgaaaggtggatgggtaggttatatagggatatagcacagagatatatagcaaagagatacttttgagcaatgtttgtggaagcggtattcgcaatattttagtagctcgttacagtccggtgcgtttttggttttttgaaagtgcgtcttcagagcgcttttggttttcaaaagcgctctgaagttcctatacttcctagagaataggaacttcggaataggaacttcaaagcgtttccgaaaacgagcgcttccgaaaatgcaacgcgagctgcgcacatacagctcactgttcacgtcgcacctatatctgcgtgttgcctgtatatatatatacatgagaagaacggcatagtgcgtgtttatgcttaaatgcgtacttatatgcgtctatttatgtaggatgaaaggtagtctagtacctcctgtgatattatcccattccatgcggggtatcgtatgcttccttcagcactaccctttagctgttctatatgctgccactcctcaattggattagtctcatccttcaatgctatcatttcctttgatattggatcatacggcgcgcctggtgtaccgagaacgatcctctcagtgcgagtctcgacgatccatatcgttgcttggcagtcagccagtcggaatccagcttgggacccaggaagtccaatcgtcagatattgtactcaagcctggtcacggcagcgtaccgatctgtttaaacctagatattgatagtctgatcggtcaacgtataatcgagtcctagcttttgcaaacatctatcaagagacaggatcagcaggaggctttcgcatgattgaacaagatggattgcacgcaggttctccggcggcttgggtggagaggctattcggctatgactgggcacaacagacaatcggctgctctgatgccgccgtgttccggctgtcagcgcaggggcgtccggttctttttgtcaagaccgacctgtccggtgccctgaatgaactgcaagacgaggcagcgcggctatcgtggctggcgacgacgggcgttccttgcgcggctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgtctatgcccgacggcgaggatctcgtcgtgacccacggcgatgcctgcttgccgaatatcatggtggaaaatggccgcttttctggattcatcgactgtggccgtctgggtgtggcggaccgctatcaggacatagcgttggctacccgtgatattgctgaagagcttggcggcgaatgggctgaccgcttccttgtgctttacggtatcgccgcgcccgattcgcagcgcatcgccttctatcgccttcttgacgagttcttctgaccgattctaggtgcattggcgcagaaaaaaatgcctgatgcgacgctgcgcgtcttatactcccacatatgccagattcagcaacggatacggcttccccaacttgcccacttccatacgtgtcctccttaccagaaatttatccttaaggtcgtttaaactcgactctggctctatcgaatctccgtcgtttcgagcttacgcgaacagccgtggcgctcatttgctcgtcgggcatcgaatctcgtcagctatcgtcagcttacctttttggcagcgatcgccgcaattaaccctcactaaagggaacaaaagctggagctcggctcccgacatcttggaccattagctccacaggtatcttcttccctctagtggtcataacagcagcttcagctacctctcaatttcaatcgttgcgttacacacacaaaaaaccaacacacatccatcttcgatggatagcgattttattatctaactgctgatcgagtgtagccagatct

SEQ ID NO: 6 - DNA, 7758 bp, synthetic construct
  source 1..7758: mol_type = other DNA
tcattatcaatactcgccatttcaaagaatacgtaaataattaatagtagtgattttcctaactttatttagtcaaaaaattagccttttaattctgctgtaacccgtacatgcccaaaatagggggcgggttacacagaatatataacatcgtaggtgtctgggtgaacagtttattcctggcatccactaaatataatggagcccgctttttaagctggcatccagaaaaaaaaagaatcccagcaccaaaatattgttttcttcaccaaccatcagttcataggtccattctcttagcgcaactacagagaacaggggcacaaacaggcaaaaaacgggcacaacctcaatggagtgatgcaacctgcctggagtaaatgatgacacaaggcaattgacccacgcatgtatctatctcattttcttacaccttctattaccttctgctctctctgatttggaaaaagctgaaaaaaaaggttgaaaccagttccctgaaattattcccctacttgactaataagtatataaagacggtaggtattgattgtaattctgtaaatctatttcttaaacttctagatctttgtcgatcctaccatccactcgacacacccgccagcggccgctgccaagcttccgagctctcgaattcaaaggaggtaccaaccatgttgttacaggcattcttgtttctgttggccggttttgctgcgaagatttctgccggaagttcctatccctatgacgtgccagactacgcatccagcggaggaagttccgactcccctgatcgtccgtggaaccctccaaccttttccccggccttactagtagtaaccgagggtgacaacgcaacttttacctgttcattctctaacacaagcgagtcatttcacgtcgtatggcatagagaaagcccaagcggccaaaccgacacgctagcggccttccccgaagacaggtcacaaccaggacaagatgctagatttagagttacgcaactgcccaatggcagggatttccacatgtcagtggtgagagccaggagaaacgactcagggacgtatgtgtgcggcgtaatctctctggccccaaaaatccaaataaaagaatcattacgtgctgagttaagagttacagaacgtggcggcggaagtagcggtggcggaagcggtgggcagcccagggagcctcaagtctatacgctgcctccgtcaagagaagagatgactaagaaccaagtctctctaacttgtctagtaaaggggttttatccttcagatattgccgtcgagtgggagtcaaatgggcaaccggagaataattacaagacaactcctccggtcctagattccgatggcagtttcttcctttactcaaaactgactgttgataaaagccgttggcagcaaggaaatgttttttcttgtagcgtgatgcatgaagcgctgcataaccactatactcagaaatctttatccctgtcacctggcaaatgatctagaagttgtctcctcctgcactgactgactgatacaatcgatttctggatccgcaggcctctgctagcttgactgactgagatacagcgtaccttcagctcatcatgtaattagttatgtcacgcttacattcacgccctccccccacatccgctctaaccgaaaaggaaggagttagacaacctgaagtctaggtccctatttatttttttatagttatgttagtattaagaacgttatttatatttcaaatttttcttttttttctgtacagacgcgtgtacgcatgtaacattatactgaaaaccttgcttgagaaggttttgggacgctcgaaggctttaatttgcggccaaagcaagcaattcgccctatagtgagtcgtattacgcgcgctaaaacctctacaaatgtggtattggcccatctctatcggtatcgtagcataaccccttggggcctctaaacgggtcttgaggggttttttgtgcccctcgggccggattgctatctaccggcattggcgcagaaaaaaatgcctgatgcgacgctgcgcgtcttatactcccacatatgccagattcagcaacggatacggcttccccaacttgcccacttccatacgtgtcctccttaccagaaatttatccttaaggtcgtcagctatcctgcagggtgtttaaagattacggatatttaacttacttagaataatgccatttttttgagttataataatcctacgttagtgtgagcgggatttaaactgtgaggaccttaatacattcagacacttctgcggtatcaccctacttattcccttcgagattatatctaggaacccatcaggttggtggaagattacccgttctaagacttttcagcttcctctattgatgttacacctggacaccccttttctggcatccagtttttaatcttcagtggcatgtgagattctccgaaattaactaaagcaatcacacaattctctcggataccacctcggttgaaactgacaggtggtttgttacgcatgctaatgcaaaggagcctatatacctttggctcggctgctgtaacagggaatataaagggcagcataatttaggagtttagtgaacttgcaacatttactattttcccttcttacgtaaatatttttctttttaattctaaatcaatctttttcaattttttgtttgtattcttttcttgcttaaatctataactacaaaaaacacatacataaactaaaaatggccaaaaccttcaacatctctcagcaggatctggagctggtggaggtcgccactgagaagatcaccatgctctatgaggacaacaagcaccatgtcggggcggccatcaggaccaagactggggagatcatctctgctgtccacattgaagcctacattggcagggtcactgtctgtgctgaagccattgccattgggtctgctgtgagcaacgggcagaaggactttgacaccattgtggctgtcaggcacccctactctgatgaggtggacagatccatcagggtggtcagcccctgtggcatgtgtagagagctgatctctgactatgctcctgactgctttgtgctcattgagatgaatggcaagctggtcaaaaccaccattgaggaactcatccccctcaagtacaccaggaactaaggttaatataattatataaaaatattatcttcttttctttatatctagtgttatgtaaaataaattgatgactacggaaagcctttttatattgtttctttttcattctgagccacttaaatttcgtgaatgttcttgtaagggacggtagatttacaagtgatacaacaaaaagcaaggcgctttttctaataaaaagaagaaaagcatttaacaattgaacacctctatatcaacgaagaatattcctgcaggcgatctctcgatttcgatcaagacattcctttaatggtcttttctggacaccactaggggtcagaagtagttcatcaaactttcttccctccctaatctcattggttaccttgggctatcgaaacttaattaaccagtcaagtcagctacttggcgagatcgacttgtctgggtttcgactacgctcagaattgcgtcagtcaagttcgatctggtccttgctattgcacccgttctccgattacgagtttcatttaaatcatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcatttaaatttccgaactctccaaggccctcgtcggaaaatcttcaaacctttcgtccgatccatcttgcaggctacctctcgaacgaactatcgcaagtctcttggccggccttgcgccttggctattgcttggcagcgcctatcgccaggtattactccaatcccgaatatccgagatcgggatcacccgagagaagttcaacctacatcctcaatcccgatctatccgagatccgaggaatatcgaaatcggggcgcgccaacgaagcatctgtgcttcattttgtagaacaaaaatgcaacgcgagagcgctaatttttcaaacaaagaatctgagctgcatttttacagaacagaaatgcaacgcgaaagcgctattttaccaacgaagaatctgtgcttcatttttgtaaaacaaaaatgcaacgcgagagcgctaatttttcaaacaaagaatctgagctgcatttttacagaacagaaatgcaacgcgagagcgctattttaccaacaaagaatctatacttcttttttgttctacaaaaatgcatcccgagagcgctatttttctaacaaagcatcttagattactttttttctcctttgtgcgctctataatgcagtctcttgataactttttgcactgtaggtccgttaaggttagaagaaggctactttggtgtctattttctcttccataaaaaaagcctgactccacttcccgcgtttactgattactagcgaagctgcgggtgcattttttcaagataaaggcatccccgattatattctataccgatgtggattgcgcatactttgtgaacagaaagtgatagcgttgatgattcttcattggtcagaaaattatgaacggtttcttctattttgtctctatatactacgtataggaaatgtttacattttcgtattgttttcgattcactctatgaatagttcttactacaatttttttgtctaaagagtaatactagagataaacataaaaaatgtagaggtcgagtttagatgcaagttcaaggagcgaaaggtggatgggtaggttatatagggatatagcacagagatatatagcaaagagatacttttgagcaatgtttgtggaagcggtattcgcaatattttagtagctcgttacagtccggtgcgtttttggttttttgaaagtgcgtcttcagagcgcttttggttttcaaaagcgctctgaagttcctatacttcctagagaataggaacttcggaataggaacttcaaagcgtttccgaaaacgagcgcttccgaaaatgcaacgcgagctgcgcacatacagctcactgttcacgtcgcacctatatctgcgtgttgcctgtatatatatatacatgagaagaacggcatagtgcgtgtttatgcttaaatgcgtacttatatgcgtctatttatgtaggatgaaaggtagtctagtacctcctgtgatattatcccattccatgcggggtatcgtatgcttccttcagcactaccctttagctgttctatatgctgccactcctcaattggattagtctcatccttcaatgctatcatttcctttgatattggatcatacggcgcgcctggtgtaccgagaacgatcctctcagtgcgagtctcgacgatccatatcgttgcttggcagtcagccagtcggaatccagcttgggacccaggaagtccaatcgtcagatattgtactcaagcctggtcacggcagcgtaccgatctgtttaaacctagatattgatagtctgatcggtcaacgtataatcgagtcctagcttttgcaaacatctatcaagagacaggatcagcaggaggctttcgcatgattgaacaagatggattgcacgcaggttctccggcggcttgggtggagaggctattcggctatgactgggcacaacagacaatcggctgctctgatgccgccgtgttccggctgtcagcgcaggggcgtccggttctttttgtcaagaccgacctgtccggtgccctgaatgaactgcaagacgaggcagcgcggctatcgtggctggcgacgacgggcgttccttgcgcggctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgtctatgcccgacggcgaggatctcgtcgtgacccacggcgatgcctgcttgccgaatatcatggtggaaaatggccgcttttctggattcatcgactgtggccgtctgggtgtggcggaccgctatcaggacatagcgttggctacccgtgatattgctgaagagcttggcggcgaatgggctgaccgcttccttgtgctttacggtatcgccgcgcccgattcgcagcgcatcgccttctatcgccttcttgacgagttcttctgaccgattctaggtgcattggcgcagaaaaaaatgcctgatgcgacgctgcgcgtcttatactcccacatatgccagattcagcaacggatacggcttccccaacttgcccacttccatacgtgtcctccttaccagaaatttatccttaaggtcgtttaaactcgactctggctctatcgaatctccgtcgtttcgagcttacgcgaacagccgtggcgctcatttgctcgtcgggcatcgaatctcgtcagctatcgtcagcttacctttttggcagcgatcgccgcaattaaccctcactaaagggaacaaaagctggagctcggctcccgacatcttggaccattagctccacaggtatcttcttccctctagtggtcataacagcagcttcagctacctctcaatttcaatcgttgcgttacacacacaaaaaaccaacacacatccatcttcgatggatagcgattttattatctaactgctgatcgagtgtagccagatct

SEQ ID NO: 7 - DNA, 7935 bp, synthetic construct
  source 1..7935: mol_type = other DNA
tcattatcaatactcgccatttcaaagaatacgtaaataattaatagtagtgattttcctaactttatttagtcaaaaaattagccttttaattctgctgtaacccgtacatgcccaaaatagggggcgggttacacagaatatataacatcgtaggtgtctgggtgaacagtttattcctggcatccactaaatataatggagcccgctttttaagctggcatccagaaaaaaaaagaatcccagcaccaaaatattgttttcttcaccaaccatcagttcataggtccattctcttagcgcaactacagagaacaggggcacaaacaggcaaaaaacgggcacaacctcaatggagtgatgcaacctgcctggagtaaatgatgacacaaggcaattgacccacgcatgtatctatctcattttcttacaccttctattaccttctgctctctctgatttggaaaaagctgaaaaaaaaggttgaaaccagttccctgaaattattcccctacttgactaataagtatataaagacggtaggtattgattgtaattctgtaaatctatttcttaaacttctagatctttgtcgatcctaccatccactcgacacacccgccagcggccgctgccaagcttccgagctctcgaattcaaaggaggtaccaaccatggggagatttccttcaatttttactgcagttttattcgcagcatcctccgcattagctgctccagtcaacactacaacagaagatgaaacggcacaaattccggctgaagctgtcatcggttacttagatttagaaggggatttcgatgttgctgttttgccattttccaacagcacaaataacgggttattgtttataaatactactattgccagcattgctgctaaagaagaaggggtatctttggataaaagagagggaagttcctatccctatgacgtgccagactacgcatccagcggaggaagttccgactcccctgatcgtccgtggaaccctccaaccttttccccggccttactagtagtaaccgagggtgacaacgcaacttttacctgttcattctctaacacaagcgagtcatttcacgtcgtatggcatagagaaagcccaagcggccaaaccgacacgctagcggccttccccgaagacaggtcacaaccaggacaagatgctagatttagagttacgcaactgcccaatggcagggatttccacatgtcagtggtgagagccaggagaaacgactcagggacgtatgtgtgcggcgtaatctctctggccccaaaaatccaaataaaagaatcattacgtgctgagttaagagttacagaacgtggcggcggaagtagcggtggcggaagcggtgggcagcccagggagcctcaagtctatacgctgcctccgtcaagagaagagatgactaagaaccaagtctctctaacttgtctagtaaaggggttttatccttcagatattgccgtcgagtgggagtcaaatgggcaaccggagaataattacaagacaactcctccggtcctagattccgatggcagtttcttcctttactcaaaactgactgttgataaaagccgttggcagcaaggaaatgttttttcttgtagcgtgatgcatgaagcgctgcataaccactatactcagaaatctttatccctgtcacctggcaaatgatctagaagttgtctcctcctgcactgactgactgatacaatcgatttctggatccgcaggcctctgctagcttgactgactgagatacagcgtaccttcagctcaataaagcaatcttgatgaggataatgatttttttttgaatatacataaatactaccgtttttctgctagattttgtgatgacgtaaataagtacatattactttttaagccaagacaagattaagcattaactttacccttttctttctaagtttcaatattagttatcactgtttaaaagttatggcgagaacgtcggcggttaaaatatattaccctgaacgaaaagcaagcaattcgccctatagtgagtcgtattacgcgcgctaaaacctctacaaatgtggtattggcccatctctatcggtatcgtagcataaccccttggggcctctaaacgggtcttgaggggttttttgtgcccctcgggccggattgctatctaccggcattggcgcagaaaaaaatgcctgatgcgacgctgcgcgtcttatactcccacatatgccagattcagcaacggatacggcttccccaacttgcccacttccatacgtgtcctccttaccagaaatttatccttaaggtcgtcagctatcctgcagggtgtttaaagattacggatatttaacttacttagaataatgccatttttttgagttataataatcctacgttagtgtgagcgggatttaaactgtgaggaccttaatacattcagacacttctgcggtatcaccctacttattcccttcgagattatatctaggaacccatcaggttggtggaagattacccgttctaagacttttcagcttcctctattgatgttacacctggacaccccttttctggcatccagtttttaatcttcagtggcatgtgagattctccgaaattaactaaagcaatcacacaattctctcggataccacctcggttgaaactgacaggtggtttgttacgcatgctaatgcaaaggagcctatatacctttggctcggctgctgtaacagggaatataaagggcagcataatttaggagtttagtgaacttgcaacatttactattttcccttcttacgtaaatatttttctttttaattctaaatcaatctttttcaattttttgtttgtattcttttcttgcttaaatctataactacaaaaaacacatacataaactaaaaatggccaaaaccttcaacatctctcagcaggatctggagctggtggaggtcgccactgagaagatcaccatgctctatgaggacaacaagcaccatgtcggggcggccatcaggaccaagactggggagatcatctctgctgtccacattgaagcctacattggcagggtcactgtctgtgctgaagccattgccattgggtctgctgtgagcaacgggcagaaggactttgacaccattgtggctgtcaggcacccctactctgatgaggtggacagatccatcagggtggtcagcccctgtggcatgtgtagagagctgatctctgactatgctcctgactgctttgtgctcattgagatgaatggcaagctggtcaaaaccaccattgaggaactcatccccctcaagtacaccaggaactaaggttaatataattatataaaaatattatcttcttttctttatatctagtgttatgtaaaataaattgatgactacggaaagcctttttatattgtttctttttcattctgagccacttaaatttcgtgaatgttcttgtaagggacggtagatttacaagtgatacaacaaaaagcaaggcgctttttctaataaaaagaagaaaagcatttaacaattgaacacctctatatcaacgaagaatattcctgcaggcgatctctcgatttcgatcaagacattcctttaatggtcttttctggacaccactaggggtcagaagtagttcatcaaactttcttccctccctaatctcattggttaccttgggctatcgaaacttaattaaccagtcaagtcagctacttggcgagatcgacttgtctgggtttcgactacgctcagaattgcgtcagtcaagttcgatctggtccttgctattgcacccgttctccgattacgagtttcatttaaatcatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcatttaaatttccgaactctccaaggccctcgtcggaaaatcttcaaacctttcgtccgatccatcttgcaggctacctctcgaacgaactatcgcaagtctcttggccggccttgcgccttggctattgcttggcagcgcctatcgccaggtattactccaatcccgaatatccgagatcgggatcacccgagagaagttcaacctacatcctcaatcccgatctatccgagatccgaggaatatcgaaatcggggcgcgccaacgaagcatctgtgcttcattttgtagaacaaaaatgcaacgcgagagcgctaatttttcaaacaaagaatctgagctgcatttttacagaacagaaatgcaacgcgaaagcgctattttaccaacgaagaatctgtgcttcatttttgtaaaacaaaaatgcaacgcgagagcgctaatttttcaaacaaagaatctgagctgcatttttacagaacagaaatgcaacgcgagagcgctattttaccaacaaagaatctatacttcttttttgttctacaaaaatgcatcccgagagcgctatttttctaacaaagcatcttagattactttttttctcctttgtgcgctctataatgcagtctcttgataactttttgcactgtaggtccgttaaggttagaagaaggctactttggtgtctattttctcttccataaaaaaagcctgactccacttcccgcgtttactgattactagcgaagctgcgggtgcattttttcaagataaaggcatccccgattatattctataccgatgtggattgcgcatactttgtgaacagaaagtgatagcgttgatgattcttcattggtcagaaaattatgaacggtttcttctattttgtctctatatactacgtataggaaatgtttacattttcgtattgttttcgattcactctatgaatagttcttactacaatttttttgtctaaagagtaatactagagataaacataaaaaatgtagaggtcgagtttagatgcaagttcaaggagcgaaaggtggatgggtaggttatatagggatatagcacagagatatatagcaaagagatacttttgagcaatgtttgtggaagcggtattcgcaatattttagtagctcgttacagtccggtgcgtttttggttttttgaaagtgcgtcttcagagcgcttttggttttcaaaagcgctctgaagttcctatacttcctagagaataggaacttcggaataggaacttcaaagcgtttccgaaaacgagcgcttccgaaaatgcaacgcgagctgcgcacatacagctcactgttcacgtcgcacctatatctgcgtgttgcctgtatatatatatacatgagaagaacggcatagtgcgtgtttatgcttaaatgcgtacttatatgcgtctatttatgtaggatgaaaggtagtctagtacctcctgtgatattatcccattccatgcggggtatcgtatgcttccttcagcactaccctttagctgttctatatgctgccactcctcaattggattagtctcatccttcaatgctatcatttcctttgatattggatcatacggcgcgcctggtgtaccgagaacgatcctctcagtgcgagtctcgacgatccatatcgttgcttggcagtcagccagtcggaatccagcttgggacccaggaagtccaatcgtcagatattgtactcaagcctggtcacggcagcgtaccgatctgtttaaacctagatattgatagtctgatcggtcaacgtataatcgagtcctagcttttgcaaacatctatcaagagacaggatcagcaggaggctttcgcatgattgaacaagatggattgcacgcaggttctccggcggcttgggtggagaggctattcggctatgactgggcacaacagacaatcggctgctctgatgccgccgtgttccggctgtcagcgcaggggcgtccggttctttttgtcaagaccgacctgtccggtgccctgaatgaactgcaagacgaggcagcgcggctatcgtggctggcgacgacgggcgttccttgcgcggctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgtctatgcccgacggcgaggatctcgtcgtgacccacggcgatgcctgcttgccgaatatcatggtggaaaatggccgcttttctggattcatcgactgtggccgtctgggtgtggcggaccgctatcaggacatagcgttggctacccgtgatattgctgaagagcttggcggcgaatgggctgaccgcttccttgtgctttacggtatcgccgcgcccgattcgcagcgcatcgccttctatcgccttcttgacgagttcttctgaccgattctaggtgcattggcgcagaaaaaaatgcctgatgcgacgctgcgcgtcttatactcccacatatgccagattcagcaacggatacggcttccccaacttgcccacttccatacgtgtcctccttaccagaaatttatccttaaggtcgtttaaactcgactctggctctatcgaatctccgtcgtttcgagcttacgcgaacagccgtggcgctcatttgctcgtcgggcatcgaatctcgtcagctatcgtcagcttacctttttggcagcgatcgccgcaattaaccctcactaaagggaacaaaagctggagctcggctcccgacatcttggaccattagctccacaggtatcttcttccctctagtggtcataacagcagcttcagctacctctcaatttcaatcgttgcgttacacacacaaaaaaccaacacacatccatcttcgatggatagcgattttattatctaactgctgatcgagtgtagccagatct

SEQ ID NO: 8 - DNA, 7853 bp, synthetic construct
  source 1..7853: mol_type = other DNA
atccttttgttgtttccgggtgtacaatatggacttcctcttttctggcaaccaaacccatacatcgggattcctataataccttcgttggtctccctaacatgtaggtggcggaggggagatatacaatagaacagataccagacaagacataatgggctaaacaagactacaccaattacactgcctcattgatggtggtacataacgaactaatactgtagccctagacttgatagccatcatcatatcgaagtttcactaccctttttccatttgccatctattgaagtaataataggcgcatgcaacttcttttctttttttttcttttctctctcccccgttgttgtctcaccatatccgcaatgacaaaaaaatgatggaagacactaaaggaaaaaattaacgacaaagacagcaccaacagatgtcgttgttccagagctgatgaggggtatctcgaagcacacgaaactttttccttccttcattcacgcacactactctctaatgagcaacggtatacggccttccttccagttacttgaatttgaaataaaaaaaagtttgctgtcttgctatcaagtataaatagacctgcaattattaatcttttgtttcctcgtcattgttctcgttccctttcttccttgtttctttttctgcacaatatttcaagctataccaagcatacaatcaactagatctttgtcgatcctaccatccactcgacacacccgccagcggccgctgccaagcttccgagctctcgaattcaaaggaggtaccaaccatgtttaccttccttaagatcatattatggttattttcccttgccttggcctcagcaggaagttcctatccctatgacgtgccagactacgcatccagcggaggaagttccgactcccctgatcgtccgtggaaccctccaaccttttccccggccttactagtagtaaccgagggtgacaacgcaacttttacctgttcattctctaacacaagcgagtcatttcacgtcgtatggcatagagaaagcccaagcggccaaaccgacacgctagcggccttccccgaagacaggtcacaaccaggacaagatgctagatttagagttacgcaactgcccaatggcagggatttccacatgtcagtggtgagagccaggagaaacgactcagggacgtatgtgtgcggcgtaatctctctggccccaaaaatccaaataaaagaatcattacgtgctgagttaagagttacagaacgtggcggcggaagtagcggtggcggaagcggtgggcagcccagggagcctcaagtctatacgctgcctccgtcaagagaagagatgactaagaaccaagtctctctaacttgtctagtaaaggggttttatccttcagatattgccgtcgagtgggagtcaaatgggcaaccggagaataattacaagacaactcctccggtcctagattccgatggcagtttcttcctttactcaaaactgactgttgataaaagccgttggcagcaaggaaatgttttttcttgtagcgtgatgcatgaagcgctgcataaccactatactcagaaatctttatccctgtcacctggcaaatgatctagaagttgtctcctcctgcactgactgactgatacaatcgatttctggatccgcaggcctctgctagcttgactgactgagatacagcgtaccttcagctcaagtgcttttaactaagaattattagtcttttctgcttattttttcatcatagtttagaacactttatattaacgaatagtttatgaatctatttaggtttaaaaattgatacagttttataagttactttttcaaagactcgtgctgtctattgcataatgcactggaaggggaaaaaaaaggtgcacacgcgtggctttttcttgaatttgcagtttgaaaaataaagcaagcaattcgccctatagtgagtcgtattacgcgcgctaaaacctctacaaatgtggtattggcccatctctatcggtatcgtagcataaccccttggggcctctaaacgggtcttgaggggttttttgtgcccctcgggccggattgctatctaccggcattggcgcagaaaaaaatgcctgatgcgacgctgcgcgtcttatactcccacatatgccagattcagcaacggatacggcttccccaacttgcccacttccatacgtgtcctccttaccagaaatttatccttaaggtcgtcagctatcctgcagggtgtttaaagattacggatatttaacttacttagaataatgccatttttttgagttataataatcctacgttagtgtgagcgggatttaaactgtgaggaccttaatacattcagacacttctgcggtatcaccctacttattcccttcgagattatatctaggaacccatcaggttggtggaagattacccgttctaagacttttcagcttcctctattgatgttacacctggacaccccttttctggcatccagtttttaatcttcagtggcatgtgagattctccgaaattaactaaagcaatcacacaattctctcggataccacctcggttgaaactgacaggtggtttgttacgcatgctaatgcaaaggagcctatatacctttggctcggctgctgtaacagggaatataaagggcagcataatttaggagtttagtgaacttgcaacatttactattttcccttcttacgtaaatatttttctttttaattctaaatcaatctttttcaattttttgtttgtattcttttcttgcttaaatctataactacaaaaaacacatacataaactaaaaatggccaaaaccttcaacatctctcagcaggatctggagctggtggaggtcgccactgagaagatcaccatgctctatgaggacaacaagcaccatgtcggggcggccatcaggaccaagactggggagatcatctctgctgtccacattgaagcctacattggcagggtcactgtctgtgctgaagccattgccattgggtctgctgtgagcaacgggcagaaggactttgacaccattgtggctgtcaggcacccctactctgatgaggtggacagatccatcagggtggtcagcccctgtggcatgtgtagagagctgatctctgactatgctcctgactgctttgtgctcattgagatgaatggcaagctggtcaaaaccaccattgaggaactcatccccctcaagtacaccaggaactaaggttaatataattatataaaaatattatcttcttttctttatatctagtgttatgtaaaataaattgatgactacggaaagcctttttatattgtttctttttcattctgagccacttaaatttcgtgaatgttcttgtaagggacggtagatttacaagtgatacaacaaaaagcaaggcgctttttctaataaaaagaagaaaagcatttaacaattgaacacctctatatcaacgaagaatattcctgcaggcgatctctcgatttcgatcaagacattcctttaatggtcttttctggacaccactaggggtcagaagtagttcatcaaactttcttccctccctaatctcattggttaccttgggctatcgaaacttaattaaccagtcaagtcagctacttggcgagatcgacttgtctgggtttcgactacgctcagaattgcgtcagtcaagttcgatctggtccttgctattgcacccgttctccgattacgagtttcatttaaatcatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcatttaaatttccgaactctccaaggccctcgtcggaaaatcttcaaacctttcgtccgatccatcttgcaggctacctctcgaacgaactatcgcaagtctcttggccggccttgcgccttggctattgcttggcagcgcctatcgccaggtattactccaatcccgaatatccgagatcgggatcacccgagagaagttcaacctacatcctcaatcccgatctatccgagatccgaggaatatcgaaatcggggcgcgccaacgaagcatctgtgcttcattttgtagaacaaaaatgcaacgcgagagcgctaatttttcaaacaaagaatctgagctgcatttttacagaacagaaatgcaacgcgaaagcgctattttaccaacgaagaatctgtgcttcatttttgtaaaacaaaaatgcaacgcgagagcgctaatttttcaaacaaagaatctgagctgcatttttacagaacagaaatgcaacgcgagagcgctattttaccaacaaagaatctatacttcttttttgttctacaaaaatgcatcccgagagcgctatttttctaacaaagcatcttagattactttttttctcctttgtgcgctctataatgcagtctcttgataactttttgcactgtaggtccgttaaggttagaagaaggctactttggtgtctattttctcttccataaaaaaagcctgactccacttcccgcgtttactgattactagcgaagctgcgggtgcattttttcaagataaaggcatccccgattatattctataccgatgtggattgcgcatactttgtgaacagaaagtgatagcgttgatgattcttcattggtcagaaaattatgaacggtttcttctattttgtctctatatactacgtataggaaatgtttacattttcgtattgttttcgattcactctatgaatagttcttactacaatttttttgtctaaagagtaatactagagataaacataaaaaatgtagaggtcgagtttagatgcaagttcaaggagcgaaaggtggatgggtaggttatatagggatatagcacagagatatatagcaaagagatacttttgagcaatgtttgtggaagcggtattcgcaatattttagtagctcgttacagtccggtgcgtttttggttttttgaaagtgcgtcttcagagcgcttttggttttcaaaagcgctctgaagttcctatacttcctagagaataggaacttcggaataggaacttcaaagcgtttccgaaaacgagcgcttccgaaaatgcaacgcgagctgcgcacatacagctcactgttcacgtcgcacctatatctgcgtgttgcctgtatatatatatacatgagaagaacggcatagtgcgtgtttatgcttaaatgcgtacttatatgcgtctatttatgtaggatgaaaggtagtctagtacctcctgtgatattatcccattccatgcggggtatcgtatgcttccttcagcactaccctttagctgttctatatgctgccactcctcaattggattagtctcatccttcaatgctatcatttcctttgatattggatcatacggcgcgcctggtgtaccgagaacgatcctctcagtgcgagtctcgacgatccatatcgttgcttggcagtcagccagtcggaatccagcttgggacccaggaagtccaatcgtcagatattgtactcaagcctggtcacggcagcgtaccgatctgtttaaacctagatattgatagtctgatcggtcaacgtataatcgagtcctagcttttgcaaacatctatcaagagacaggatcagcaggaggctttcgcatgattgaacaagatggattgcacgcaggttctccggcggcttgggtggagaggctattcggctatgactgggcacaacagacaatcggctgctctgatgccgccgtgttccggctgtcagcgcaggggcgtccggttctttttgtcaagaccgacctgtccggtgccctgaatgaactgcaagacgaggcagcgcggctatcgtggctggcgacgacgggcgttccttgcgcggctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgtctatgcccgacggcgaggatctcgtcgtgacccacggcgatgcctgcttgccgaatatcatggtggaaaatggccgcttttctggattcatcgactgtggccgtctgggtgtggcggaccgctatcaggacatagcgttggctacccgtgatattgctgaagagcttggcggcgaatgggctgaccgcttccttgtgctttacggtatcgccgcgcccgattcgcagcgcatcgccttctatcgccttcttgacgagttcttctgaccgattctaggtgcattggcgcagaaaaaaatgcctgatgcgacgctgcgcgtcttatactcccacatatgccagattcagcaacggatacggcttccccaacttgcccacttccatacgtgtcctccttaccagaaatttatccttaaggtcgtttaaactcgactctggctctatcgaatctccgtcgtttcgagcttacgcgaacagccgtggcgctcatttgctcgtcgggcatcgaatctcgtcagctatcgtcagcttacctttttggcagcgatcgccgcaattaaccctcactaaagggaacaaaagctggagctcggctcccgacatcttggaccattagctccacaggtatcttcttccctctagtggtcataacagcagcttcagctacctctcaatttcaatcgttgcgttacacacacaaaaaaccaacacacatccatcttcgatggatagcgattttattatctaactgctgatcgagtgtagccagatct

SEQ ID NO: 9 - DNA, 7935 bp, synthetic construct
  source 1..7935: mol_type = other DNA
tcattatcaatactcgccatttcaaagaatacgtaaataattaatagtagtgattttcctaactttatttagtcaaaaaattagccttttaattctgctgtaacccgtacatgcccaaaatagggggcgggttacacagaatatataacatcgtaggtgtctgggtgaacagtttattcctggcatccactaaatataatggagcccgctttttaagctggcatccagaaaaaaaaagaatcccagcaccaaaatattgttttcttcaccaaccatcagttcataggtccattctcttagcgcaactacagagaacaggggcacaaacaggcaaaaaacgggcacaacctcaatggagtgatgcaacctgcctggagtaaatgatgacacaaggcaattgacccacgcatgtatctatctcattttcttacaccttctattaccttctgctctctctgatttggaaaaagctgaaaaaaaaggttgaaaccagttccctgaaattattcccctacttgactaataagtatataaagacggtaggtattgattgtaattctgtaaatctatttcttaaacttctagatctttgtcgatcctaccatccactcgacacacccgccagcggccgctgccaagcttccgagctctcgaattcaaaggaggtaccaaccatggggagatttccttcaatttttactgcagttttattcgcagcatcctccgcattagctgctccagtcaacactacaacagaagatgaaacggcacaaattccggctgaagctgtcatcggttacttagatttagaaggggatttcgatgttgctgttttgccattttccaacagcacaaataacgggttattgtttataaatactactattgccagcattgctgctaaagaagaaggggtatctttggataaaagagagggaagttcctatccctatgacgtgccagactacgcatccagcggaggaagttccgactcccctgatcgtccgtggaaccctccaaccttttccccggccttactagtagtaaccgagggtgacaacgcaacttttacctgttcattctctaacacaagcgagtcatttcacgtcgtatggcatagagaaagcccaagcggccaaaccgacacgctagcggccttccccgaagacaggtcacaaccaggacaagatgctagatttagagttacgcaactgcccaatggcagggatttccacatgtcagtggtgagagccaggagaaacgactcagggacgtatgtgtgcggcgtaatctctctggccccaaaaatccaaataaaagaatcattacgtgctgagttaagagttacagaacgtggcggcggaagtagcggtggcggaagcggtgggcagcccagggagcctcaagtctatacgctgcctccgtcaagagaagagatgactaagaaccaagtctctctaacttgtctagtaaaggggttttatccttcagatattgccgtcgagtgggagtcaaatgggcaaccggagaataattacaagacaactcctccggtcctagattccgatggcagtttcttcctttactcaaaactgactgttgataaaagccgttggcagcaaggaaatgttttttcttgtagcgtgatgcatgaagcgctgcataaccactatactcagaaatctttatccctgtcacctggcaaatgatctagaagttgtctcctcctgcactgactgactgatacaatcgatttctggatccgcaggcctctgctagcttgactgactgagatacagcgtaccttcagctcagccaattggtgcggcaattgataataacgaaaatgtcttttaatgatctgggtataatgaggaattttccgaacgtttttactttatatatatatatacatgtaacatatattctatacgctatagagaaaggaaatttttcaattaaaaaaaaaatagagaaagagtttcacttcttgattatcgctaacactaatggttgaagtactgctactttaattttataaagcaagcaattcgccctatagtgagtcgtattacgcgcgctaaaacctctacaaatgtggtattggcccatctctatcggtatcgtagcataaccccttggggcctctaaacgggtcttgaggggttttttgtgcccctcgggccggattgctatctaccggcattggcgcagaaaaaaatgcctgatgcgacgctgcgcgtcttatactcccacatatgccagattcagcaacggatacggcttccccaacttgcccacttccatacgtgtcctccttaccagaaatttatccttaaggtcgtcagctatcctgcagggtgtttaaagattacggatatttaacttacttagaataatgccatttttttgagttataataatcctacgttagtgtgagcgggatttaaactgtgaggaccttaatacattcagacacttctgcggtatcaccctacttattcccttcgagattatatctaggaacccatcaggttggtggaagattacccgttctaagacttttcagcttcctctattgatgttacacctggacaccccttttctggcatccagtttttaatcttcagtggcatgtgagattctccgaaattaactaaagcaatcacacaattctctcggataccacctcggttgaaactgacaggtggtttgttacgcatgctaatgcaaaggagcctatatacctttggctcggctgctgtaacagggaatataaagggcagcataatttaggagtttagtgaacttgcaacatttactattttcccttcttacgtaaatatttttctttttaattctaaatcaatctttttcaattttttgtttgtattcttttcttgcttaaatctataactacaaaaaacacatacataaactaaaaatggccaaaaccttcaacatctctcagcaggatctggagctggtggaggtcgccactgagaagatcaccatgctctatgaggacaacaagcaccatgtcggggcggccatcaggaccaagactggggagatcatctctgctgtccacattgaagcctacattggcagggtcactgtctgtgctgaagccattgccattgggtctgctgtgagcaacgggcagaaggactttgacaccattgtggctgtcaggcacccctactctgatgaggtggacagatccatcagggtggtcagcccctgtggcatgtgtagagagctgatctctgactatgctcctgactgctttgtgctcattgagatgaatggcaagctggtcaaaaccaccattgaggaactcatccccctcaagtacaccaggaactaaggttaatataattatataaaaatattatcttcttttctttatatctagtgttatgtaaaataaattgatgactacggaaagcctttttatattgtttctttttcattctgagccacttaaatttcgtgaatgttcttgtaagggacggtagatttacaagtgatacaacaaaaagcaaggcgctttttctaataaaaagaagaaaagcatttaacaattgaacacctctatatcaacgaagaatattcctgcaggcgatctctcgatttcgatcaagacattcctttaatggtcttttctggacaccactaggggtcagaagtagttcatcaaactttcttccctccctaatctcattggttaccttgggctatcgaaacttaattaaccagtcaagtcagctacttggcgagatcgacttgtctgggtttcgactacgctcagaattgcgtcagtcaagttcgatctggtccttgctattgcacccgttctccgattacgagtttcatttaaatcatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcatttaaatttccgaactctccaaggccctcgtcggaaaatcttcaaacctttcgtccgatccatcttgcaggctacctctcgaacgaactatcgcaagtctcttggccggccttgcgccttggctattgcttggcagcgcctatcgccaggtattactccaatcccgaatatccgagatcgggatcacccgagagaagttcaacctacatcctcaatcccgatctatccgagatccgaggaatatcgaaatcggggcgcgccaacgaagcatctgtgcttcattttgtagaacaaaaatgcaacgcgagagcgctaatttttcaaacaaagaatctgagctgcatttttacagaacagaaatgcaacgcgaaagcgctattttaccaacgaagaatctgtgcttcatttttgtaaaacaaaaatgcaacgcgagagcgctaatttttcaaacaaagaatctgagctgcatttttacagaacagaaatgcaacgcgagagcgctattttaccaacaaagaatctatacttcttttttgttctacaaaaatgcatcccgagagcgctatttttctaacaaagcatcttagattactttttttctcctttgtgcgctctataatgcagtctcttgataactttttgcactgtaggtccgttaaggttagaagaaggctactttggtgtctattttctcttccataaaaaaagcctgactccacttcccgcgtttactgattactagcgaagctgcgggtgcattttttcaagataaaggcatccccgattatattctataccgatgtggattgcgcatactttgtgaacagaaagtgatagcgttgatgattcttcattggtcagaaaattatgaacggtttcttctattttgtctctatatactacgtataggaaatgtttacattttcgtattgttttcgattcactctatgaatagttcttactacaatttttttgtctaaagagtaatactagagataaacataaaaaatgtagaggtcgagtttagatgcaagttcaaggagcgaaaggtggatgggtaggttatatagggatatagcacagagatatatagcaaagagatacttttgagcaatgtttgtggaagcggtattcgcaatattttagtagctcgttacagtccggtgcgtttttggttttttgaaagtgcgtcttcagagcgcttttggttttcaaaagcgctctgaagttcctatacttcctagagaataggaacttcggaataggaacttcaaagcgtttccgaaaacgagcgcttccgaaaatgcaacgcgagctgcgcacatacagctcactgttcacgtcgcacctatatctgcgtgttgcctgtatatatatatacatgagaagaacggcatagtgcgtgtttatgcttaaatgcgtacttatatgcgtctatttatgtaggatgaaaggtagtctagtacctcctgtgatattatcccattccatgcggggtatcgtatgcttccttcagcactaccctttagctgttctatatgctgccactcctcaattggattagtctcatccttcaatgctatcatttcctttgatattggatcatacggcgcgcctggtgtaccgagaacgatcctctcagtgcgagtctcgacgatccatatcgttgcttggcagtcagccagtcggaatccagcttgggacccaggaagtccaatcgtcagatattgtactcaagcctggtcacggcagcgtaccgatctgtttaaacctagatattgatagtctgatcggtcaacgtataatcgagtcctagcttttgcaaacatctatcaagagacaggatcagcaggaggctttcgcatgattgaacaagatggattgcacgcaggttctccggcggcttgggtggagaggctattcggctatgactgggcacaacagacaatcggctgctctgatgccgccgtgttccggctgtcagcgcaggggcgtccggttctttttgtcaagaccgacctgtccggtgccctgaatgaactgcaagacgaggcagcgcggctatcgtggctggcgacgacgggcgttccttgcgcggctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgtctatgcccgacggcgaggatctcgtcgtgacccacggcgatgcctgcttgccgaatatcatggtggaaaatggccgcttttctggattcatcgactgtggccgtctgggtgtggcggaccgctatcaggacatagcgttggctacccgtgatattgctgaagagcttggcggcgaatgggctgaccgcttccttgtgctttacggtatcgccgcgcccgattcgcagcgcatcgccttctatcgccttcttgacgagttcttctgaccgattctaggtgcattggcgcagaaaaaaatgcctgatgcgacgctgcgcgtcttatactcccacatatgccagattcagcaacggatacggcttccccaacttgcccacttccatacgtgtcctccttaccagaaatttatccttaaggtcgtttaaactcgactctggctctatcgaatctccgtcgtttcgagcttacgcgaacagccgtggcgctcatttgctcgtcgggcatcgaatctcgtcagctatcgtcagcttacctttttggcagcgatcgccgcaattaaccctcactaaagggaacaaaagctggagctcggctcccgacatcttggaccattagctccacaggtatcttcttccctctagtggtcataacagcagcttcagctacctctcaatttcaatcgttgcgttacacacacaaaaaaccaacacacatccatcttcgatggatagcgattttattatctaactgctgatcgagtgtagccagatct

SEQ ID NO: 10 - DNA, 7887 bp, synthetic construct
  source 1..7887: mol_type = other DNA
cgacacgcaaaagaaaaccttcgaggttgcgcacttcgcccacccatgaaccacacggttagtccaaaaggggcagttcagattccagatgcgggaattagcttgctgccaccctcacctcactaacgctgcggtgtgcggatacttcatgctatttatagacgcgcgtgtcggaatcagcacgcgcaagaaccaaatgggaaaatcggaatgggtccagaactgctttgagtgctggctattggcgtctgatttccgttttgggaatcctttgccgcgcgcccctctcaaaactccgcacaagtcccagaaagcgggaaagaaataaaacgccaccaaaaaaaaaaaaataaaagccaatcctcgaagcgtgggtggtaggccctggattatcccgtacaagtatttctcaggagtaaaaaaaccgtttgttttggaattccccatttcgcggccacctacgccgctatctttgcaacaactatctgcgataactcagcaaattttgcatattcgtgttgcagtattgcgataatgggagtcttacttccaacataacggcagaaagaaatgtgagaaaattttgcatcctttgcctccgttcaagtatataaagtcggcatgcttgataatctttctttccatcctacattgttctaattattcttattctcctttattctttcctaacataccaagaaattaatcttctgtcattcgcttaaacactatatcaataagatctttgtcgatcctaccatccactcgacacacccgccagcggccgctgccaagcttccgagctctcgaattcaaaggaggtaccaaccatgttgttacaggcattcttgtttctgttggccggttttgctgcgaagatttctgccggaagttcctatccctatgacgtgccagactacgcatccagcggaggaagttccgactcccctgatcgtccgtggaaccctccaaccttttccccggccttactagtagtaaccgagggtgacaacgcaacttttacctgttcattctctaacacaagcgagtcatttcacgtcgtatggcatagagaaagcccaagcggccaaaccgacacgctagcggccttccccgaagacaggtcacaaccaggacaagatgctagatttagagttacgcaactgcccaatggcagggatttccacatgtcagtggtgagagccaggagaaacgactcagggacgtatgtgtgcggcgtaatctctctggccccaaaaatccaaataaaagaatcattacgtgctgagttaagagttacagaacgtggcggcggaagtagcggtggcggaagcggtgggcagcccagggagcctcaagtctatacgctgcctccgtcaagagaagagatgactaagaaccaagtctctctaacttgtctagtaaaggggttttatccttcagatattgccgtcgagtgggagtcaaatgggcaaccggagaataattacaagacaactcctccggtcctagattccgatggcagtttcttcctttactcaaaactgactgttgataaaagccgttggcagcaaggaaatgttttttcttgtagcgtgatgcatgaagcgctgcataaccactatactcagaaatctttatccctgtcacctggcaaatgatctagaagttgtctcctcctgcactgactgactgatacaatcgatttctggatccgcaggcctctgctagcttgactgactgagatacagcgtaccttcagctcaagtgcttttaactaagaattattagtcttttctgcttattttttcatcatagtttagaacactttatattaacgaatagtttatgaatctatttaggtttaaaaattgatacagttttataagttactttttcaaagactcgtgctgtctattgcataatgcactggaaggggaaaaaaaaggtgcacacgcgtggctttttcttgaatttgcagtttgaaaaataaagcaagcaattcgccctatagtgagtcgtattacgcgcgctaaaacctctacaaatgtggtattggcccatctctatcggtatcgtagcataaccccttggggcctctaaacgggtcttgaggggttttttgtgcccctcgggccggattgctatctaccggcattggcgcagaaaaaaatgcctgatgcgacgctgcgcgtcttatactcccacatatgccagattcagcaacggatacggcttccccaacttgcccacttccatacgtgtcctccttaccagaaatttatccttaaggtcgtcagctatcctgcagggtgtttaaagattacggatatttaacttacttagaataatgccatttttttgagttataataatcctacgttagtgtgagcgggatttaaactgtgaggaccttaatacattcagacacttctgcggtatcaccctacttattcccttcgagattatatctaggaacccatcaggttggtggaagattacccgttctaagacttttcagcttcctctattgatgttacacctggacaccccttttctggcatccagtttttaatcttcagtggcatgtgagattctccgaaattaactaaagcaatcacacaattctctcggataccacctcggttgaaactgacaggtggtttgttacgcatgctaatgcaaaggagcctatatacctttggctcggctgctgtaacagggaatataaagggcagcataatttaggagtttagtgaacttgcaacatttactattttcccttcttacgtaaatatttttctttttaattctaaatcaatctttttcaattttttgtttgtattcttttcttgcttaaatctataactacaaaaaacacatacataaactaaaaatggccaaaaccttcaacatctctcagcaggatctggagctggtggaggtcgccactgagaagatcaccatgctctatgaggacaacaagcaccatgtcggggcggccatcaggaccaagactggggagatcatctctgctgtccacattgaagcctacattggcagggtcactgtctgtgctgaagccattgccattgggtctgctgtgagcaacgggcagaaggactttgacaccattgtggctgtcaggcacccctactctgatgaggtggacagatccatcagggtggtcagcccctgtggcatgtgtagagagctgatctctgactatgctcctgactgctttgtgctcattgagatgaatggcaagctggtcaaaaccaccattgaggaactcatccccctcaagtacaccaggaactaaggttaatataattatataaaaatattatcttcttttctttatatctagtgttatgtaaaataaattgatgactacggaaagcctttttatattgtttctttttcattctgagccacttaaatttcgtgaatgttcttgtaagggacggtagatttacaagtgatacaacaaaaagcaaggcgctttttctaataaaaagaagaaaagcatttaacaattgaacacctctatatcaacgaagaatattcctgcaggcgatctctcgatttcgatcaagacattcctttaatggtcttttctggacaccactaggggtcagaagtagttcatcaaactttcttccctccctaatctcattggttaccttgggctatcgaaacttaattaaccagtcaagtcagctacttggcgagatcgacttgtctgggtttcgactacgctcagaattgcgtcagtcaagttcgatctggtccttgctattgcacccgttctccgattacgagtttcatttaaatcatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcatttaaatttccgaactctccaaggccctcgtcggaaaatcttcaaacctttcgtccgatccatcttgcaggctacctctcgaacgaactatcgcaagtctcttggccggccttgcgccttggctattgcttggcagcgcctatcgccaggtattactccaatcccgaatatccgagatcgggatcacccgagagaagttcaacctacatcctcaatcccgatctatccgagatccgaggaatatcgaaatcggggcgcgccaacgaagcatctgtgcttcattttgtagaacaaaaatgcaacgcgagagcgctaatttttcaaacaaagaatctgagctgcatttttacagaacagaaatgcaacgcgaaagcgctattttaccaacgaagaatctgtgcttcatttttgtaaaacaaaaatgcaacgcgagagcgctaatttttcaaacaaagaatctgagctgcatttttacagaacagaaatgcaacgcgagagcgctattttaccaacaaagaatctatacttcttttttgttctacaaaaatgcatcccgagagcgctatttttctaacaaagcatcttagattactttttttctcctttgtgcgctctataatgcagtctcttgataactttttgcactgtaggtccgttaaggttagaagaaggctactttggtgtctattttctcttccataaaaaaagcctgactccacttcccgcgtttactgattactagcgaagctgcgggtgcattttttcaagataaaggcatccccgattatattctataccgatgtggattgcgcatactttgtgaacagaaagtgatagcgttgatgattcttcattggtcagaaaattatgaacggtttcttctattttgtctctatatactacgtataggaaatgtttacattttcgtattgttttcgattcactctatgaatagttcttactacaatttttttgtctaaagagtaatactagagataaacataaaaaatgtagaggtcgagtttagatgcaagttcaaggagcgaaaggtggatgggtaggttatatagggatatagcacagagatatatagcaaagagatacttttgagcaatgtttgtggaagcggtattcgcaatattttagtagctcgttacagtccggtgcgtttttggttttttgaaagtgcgtcttcagagcgcttttggttttcaaaagcgctctgaagttcctatacttcctagagaataggaacttcggaataggaacttcaaagcgtttccgaaaacgagcgcttccgaaaatgcaacgcgagctgcgcacatacagctcactgttcacgtcgcacctatatctgcgtgttgcctgtatatatatatacatgagaagaacggcatagtgcgtgtttatgcttaaatgcgtacttatatgcgtctatttatgtaggatgaaaggtagtctagtacctcctgtgatattatcccattccatgcggggtatcgtatgcttccttcagcactaccctttagctgttctatatgctgccactcctcaattggattagtctcatccttcaatgctatcatttcctttgatattggatcatacggcgcgcctggtgtaccgagaacgatcctctcagtgcgagtctcgacgatccatatcgttgcttggcagtcagccagtcggaatccagcttgggacccaggaagtccaatcgtcagatattgtactcaagcctggtcacggcagcgtaccgatctgtttaaacctagatattgatagtctgatcggtcaacgtataatcgagtcctagcttttgcaaacatctatcaagagacaggatcagcaggaggctttcgcatgattgaacaagatggattgcacgcaggttctccggcggcttgggtggagaggctattcggctatgactgggcacaacagacaatcggctgctctgatgccgccgtgttccggctgtcagcgcaggggcgtccggttctttttgtcaagaccgacctgtccggtgccctgaatgaactgcaagacgaggcagcgcggctatcgtggctggcgacgacgggcgttccttgcgcggctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgtctatgcccgacggcgaggatctcgtcgtgacccacggcgatgcctgcttgccgaatatcatggtggaaaatggccgcttttctggattcatcgactgtggccgtctgggtgtggcggaccgctatcaggacatagcgttggctacccgtgatattgctgaagagcttggcggcgaatgggctgaccgcttccttgtgctttacggtatcgccgcgcccgattcgcagcgcatcgccttctatcgccttcttgacgagttcttctgaccgattctaggtgcattggcgcagaaaaaaatgcctgatgcgacgctgcgcgtcttatactcccacatatgccagattcagcaacggatacggcttccccaacttgcccacttccatacgtgtcctccttaccagaaatttatccttaaggtcgtttaaactcgactctggctctatcgaatctccgtcgtttcgagcttacgcgaacagccgtggcgctcatttgctcgtcgggcatcgaatctcgtcagctatcgtcagcttacctttttggcagcgatcgccgcaattaaccctcactaaagggaacaaaagctggagctcggctcccgacatcttggaccattagctccacaggtatcttcttccctctagtggtcataacagcagcttcagctacctctcaatttcaatcgttgcgttacacacacaaaaaaccaacacacatccatcttcgatggatagcgattttattatctaactgctgatcgagtgtagccagatct

SEQ ID NO: 11 - DNA, 7887 bp, synthetic construct
  source 1..7887: mol_type = other DNA
cgacacgcaaaagaaaaccttcgaggttgcgcacttcgcccacccatgaaccacacggttagtccaaaaggggcagttcagattccagatgcgggaattagcttgctgccaccctcacctcactaacgctgcggtgtgcggatacttcatgctatttatagacgcgcgtgtcggaatcagcacgcgcaagaaccaaatgggaaaatcggaatgggtccagaactgctttgagtgctggctattggcgtctgatttccgttttgggaatcctttgccgcgcgcccctctcaaaactccgcacaagtcccagaaagcgggaaagaaataaaacgccaccaaaaaaaaaaaaataaaagccaatcctcgaagcgtgggtggtaggccctggattatcccgtacaagtatttctcaggagtaaaaaaaccgtttgttttggaattccccatttcgcggccacctacgccgctatctttgcaacaactatctgcgataactcagcaaattttgcatattcgtgttgcagtattgcgataatgggagtcttacttccaacataacggcagaaagaaatgtgagaaaattttgcatcctttgcctccgttcaagtatataaagtcggcatgcttgataatctttctttccatcctacattgttctaattattcttattctcctttattctttcctaacataccaagaaattaatcttctgtcattcgcttaaacactatatcaataagatctttgtcgatcctaccatccactcgacacacccgccagcggccgctgccaagcttccgagctctcgaattcaaaggaggtaccaaccatgttgttacaggcattcttgtttctgttggccggttttgctgcgaagatttctgccggaagttcctatccctatgacgtgccagactacgcatccagcggaggaagttccgactcccctgatcgtccgtggaaccctccaaccttttccccggccttactagtagtaaccgagggtgacaacgcaacttttacctgttcattctctaacacaagcgagtcatttcacgtcgtatggcatagagaaagcccaagcggccaaaccgacacgctagcggccttccccgaagacaggtcacaaccaggacaagatgctagatttagagttacgcaactgcccaatggcagggatttccacatgtcagtggtgagagccaggagaaacgactcagggacgtatgtgtgcggcgtaatctctctggccccaaaaatccaaataaaagaatcattacgtgctgagttaagagttacagaacgtggcggcggaagtagcggtggcggaagcggtgggcagcccagggagcctcaagtctatacgctgcctccgtcaagagaagagatgactaagaaccaagtctctctaacttgtctagtaaaggggttttatccttcagatattgccgtcgagtgggagtcaaatgggcaaccggagaataattacaagacaactcctccggtcctagattccgatggcagtttcttcctttactcaaaactgactgttgataaaagccgttggcagcaaggaaatgttttttcttgtagcgtgatgcatgaagcgctgcataaccactatactcagaaatctttatccctgtcacctggcaaatgatctagaagttgtctcctcctgcactgactgactgatacaatcgatttctggatccgcaggcctctgctagcttgactgactgagatacagcgtaccttcagctcagccaattggtgcggcaattgataataacgaaaatgtcttttaatgatctgggtataatgaggaattttccgaacgtttttactttatatatatatatacatgtaacatatattctatacgctatagagaaaggaaatttttcaattaaaaaaaaaatagagaaagagtttcacttcttgattatcgctaacactaatggttgaagtactgctactttaattttataaagcaagcaattcgccctatagtgagtcgtattacgcgcgctaaaacctctacaaatgtggtattggcccatctctatcggtatcgtagcataaccccttggggcctctaaacgggtcttgaggggttttttgtgcccctcgggccggattgctatctaccggcattggcgcagaaaaaaatgcctgatgcgacgctgcgcgtcttatactcccacatatgccagattcagcaacggatacggcttccccaacttgcccacttccatacgtgtcctccttaccagaaatttatccttaaggtcgtcagctatcctgcagggtgtttaaagattacggatatttaacttacttagaataatgccatttttttgagttataataatcctacgttagtgtgagcgggatttaaactgtgaggaccttaatacattcagacacttctgcggtatcaccctacttattcccttcgagattatatctaggaacccatcaggttggtggaagattacccgttctaagacttttcagcttcctctattgatgttacacctggacaccccttttctggcatccagtttttaatcttcagtggcatgtgagattctccgaaattaactaaagcaatcacacaattctctcggataccacctcggttgaaactgacaggtggtttgttacgcatgctaatgcaaaggagcctatatacctttggctcggctgctgtaacagggaatataaagggcagcataatttaggagtttagtgaacttgcaacatttactattttcccttcttacgtaaatatttttctttttaattctaaatcaatctttttcaattttttgtttgtattcttttcttgcttaaatctataactacaaaaaacacatacataaactaaaaatggccaaaaccttcaacatctctcagcaggatctggagctggtggaggtcgccactgagaagatcaccatgctctatgaggacaacaagcaccatgtcggggcggccatcaggaccaagactggggagatcatctctgctgtccacattgaagcctacattggcagggtcactgtctgtgctgaagccattgccattgggtctgctgtgagcaacgggcagaaggactttgacaccattgtggctgtcaggcacccctactctgatgaggtggacagatccatcagggtggtcagcccctgtggcatgtgtagagagctgatctctgactatgctcctgactgctttgtgctcattgagatgaatggcaagctggtcaaaaccaccattgaggaactcatccccctcaagtacaccaggaactaaggttaatataattatataaaaatattatcttcttttctttatatctagtgttatgtaaaataaattgatgactacggaaagcctttttatattgtttctttttcattctgagccacttaaatttcgtgaatgttcttgtaagggacggtagatttacaagtgatacaacaaaaagcaaggcgctttttctaataaaaagaagaaaagcatttaacaattgaacacctctatatcaacgaagaatattcctgcaggcgatctctcgatttcgatcaagacattcctttaatggtcttttctggacaccactaggggtcagaagtagttcatcaaactttcttccctccctaatctcattggttaccttgggctatcgaaacttaattaaccagtcaagtcagctacttggcgagatcgacttgtctgggtttcgactacgctcagaattgcgtcagtcaagttcgatctggtccttgctattgcacccgttctccgattacgagtttcatttaaatcatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcatttaaatttccgaactctccaaggccctcgtcggaaaatcttcaaacctttcgtccgatccatcttgcaggctacctctcgaacgaactatcgcaagtctcttggccggccttgcgccttggctattgcttggcagcgcctatcgccaggtattactccaatcccgaatatccgagatcgggatcacccgagagaagttcaacctacatcctcaatcccgatctatccgagatccgaggaatatcgaaatcggggcgcgccaacgaagcatctgtgcttcattttgtagaacaaaaatgcaacgcgagagcgctaatttttcaaacaaagaatctgagctgcatttttacagaacagaaatgcaacgcgaaagcgctattttaccaacgaagaatctgtgcttcatttttgtaaaacaaaaatgcaacgcgagagcgctaatttttcaaacaaagaatctgagctgcatttttacagaacagaaatgcaacgcgagagcgctattttaccaacaaagaatctatacttcttttttgttctacaaaaatgcatcccgagagcgctatttttctaacaaagcatcttagattactttttttctcctttgtgcgctctataatgcagtctcttgataactttttgcactgtaggtccgttaaggttagaagaaggctactttggtgtctattttctcttccataaaaaaagcctgactccacttcccgcgtttactgattactagcgaagctgcgggtgcattttttcaagataaaggcatccccgattatattctataccgatgtggattgcgcatactttgtgaacagaaagtgatagcgttgatgattcttcattggtcagaaaattatgaacggtttcttctattttgtctctatatactacgtataggaaatgtttacattttcgtattgttttcgattcactctatgaatagttcttactacaatttttttgtctaaagagtaatactagagataaacataaaaaatgtagaggtcgagtttagatgcaagttcaaggagcgaaaggtggatgggtaggttatatagggatatagcacagagatatatagcaaagagatacttttgagcaatgtttgtggaagcggtattcgcaatattttagtagctcgttacagtccggtgcgtttttggttttttgaaagtgcgtcttcagagcgcttttggttttcaaaagcgctctgaagttcctatacttcctagagaataggaacttcggaataggaacttcaaagcgtttccgaaaacgagcgcttccgaaaatgcaacgcgagctgcgcacatacagctcactgttcacgtcgcacctatatctgcgtgttgcctgtatatatatatacatgagaagaacggcatagtgcgtgtttatgcttaaatgcgtacttatatgcgtctatttatgtaggatgaaaggtagtctagtacctcctgtgatattatcccattccatgcggggtatcgtatgcttccttcagcactaccctttagctgttctatatgctgccactcctcaattggattagtctcatccttcaatgctatcatttcctttgatattggatcatacggcgcgcctggtgtaccgagaacgatcctctcagtgcgagtctcgacgatccatatcgttgcttggcagtcagccagtcggaatccagcttgggacccaggaagtccaatcgtcagatattgtactcaagcctggtcacggcagcgtaccgatctgtttaaacctagatattgatagtctgatcggtcaacgtataatcgagtcctagcttttgcaaacatctatcaagagacaggatcagcaggaggctttcgcatgattgaacaagatggattgcacgcaggttctccggcggcttgggtggagaggctattcggctatgactgggcacaacagacaatcggctgctctgatgccgccgtgttccggctgtcagcgcaggggcgtccggttctttttgtcaagaccgacctgtccggtgccctgaatgaactgcaagacgaggcagcgcggctatcgtggctggcgacgacgggcgttccttgcgcggctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgtctatgcccgacggcgaggatctcgtcgtgacccacggcgatgcctgcttgccgaatatcatggtggaaaatggccgcttttctggattcatcgactgtggccgtctgggtgtggcggaccgctatcaggacatagcgttggctacccgtgatattgctgaagagcttggcggcgaatgggctgaccgcttccttgtgctttacggtatcgccgcgcccgattcgcagcgcatcgccttctatcgccttcttgacgagttcttctgaccgattctaggtgcattggcgcagaaaaaaatgcctgatgcgacgctgcgcgtcttatactcccacatatgccagattcagcaacggatacggcttccccaacttgcccacttccatacgtgtcctccttaccagaaatttatccttaaggtcgtttaaactcgactctggctctatcgaatctccgtcgtttcgagcttacgcgaacagccgtggcgctcatttgctcgtcgggcatcgaatctcgtcagctatcgtcagcttacctttttggcagcgatcgccgcaattaaccctcactaaagggaacaaaagctggagctcggctcccgacatcttggaccattagctccacaggtatcttcttccctctagtggtcataacagcagcttcagctacctctcaatttcaatcgttgcgttacacacacaaaaaaccaacacacatccatcttcgatggatagcgattttattatctaactgctgatcgagtgtagccagatct

SEQ ID NO: 12 - DNA, 7876 bp, synthetic construct
  source 1..7876: mol_type = other DNA
tattgcttggcagcgcctatcgccaggtattactccaatcccgaatatccgagatcgggatcacccgagagaagttcaacctacatcctcaatcccgatctatccgagatccgaggaatatcgaaatcggggcgcgccaacgaagcatctgtgcttcattttgtagaacaaaaatgcaacgcgagagcgctaatttttcaaacaaagaatctgagctgcatttttacagaacagaaatgcaacgcgaaagcgctattttaccaacgaagaatctgtgcttcatttttgtaaaacaaaaatgcaacgcgagagcgctaatttttcaaacaaagaatctgagctgcatttttacagaacagaaatgcaacgcgagagcgctattttaccaacaaagaatctatacttcttttttgttctacaaaaatgcatcccgagagcgctatttttctaacaaagcatcttagattactttttttctcctttgtgcgctctataatgcagtctcttgataactttttgcactgtaggtccgttaaggttagaagaaggctactttggtgtctattttctcttccataaaaaaagcctgactccacttcccgcgtttactgattactagcgaagctgcgggtgcattttttcaagataaaggcatccccgattatattctataccgatgtggattgcgcatactttgtgaacagaaagtgatagcgttgatgattcttcattggtcagaaaattatgaacggtttcttctattttgtctctatatactacgtataggaaatgtttacattttcgtattgttttcgattcactctatgaatagttcttactacaatttttttgtctaaagagtaatactagagataaacataaaaaatgtagaggtcgagtttagatgcaagttcaaggagcgaaaggtggatgggtaggttatatagggatatagcacagagatatatagcaaagagatacttttgagcaatgtttgtggaagcggtattcgcaatattttagtagctcgttacagtccggtgcgtttttggttttttgaaagtgcgtcttcagagcgcttttggttttcaaaagcgctctgaagttcctatacttcctagagaataggaacttcggaataggaacttcaaagcgtttccgaaaacgagcgcttccgaaaatgcaacgcgagctgcgcacatacagctcactgttcacgtcgcacctatatctgcgtgttgcctgtatatatatatacatgagaagaacggcatagtgcgtgtttatgcttaaatgcgtacttatatgcgtctatttatgtaggatgaaaggtagtctagtacctcctgtgatattatcccattccatgcggggtatcgtatgcttccttcagcactaccctttagctgttctatatgctgccactcctcaattggattagtctcatccttcaatgctatcatttcctttgatattggatcatacggcgcgcctggtgtaccgagaacgatcctctcagtgcgagtctcgacgatccatatcgttgcttggcagtcagccagtcggaatccagcttgggacccaggaagtccaatcgtcagatattgtactcaagcctggtcacggcagcgtaccgatctgtttaaacctagatattgatagtctgatcggtcaacgtataatcgagtcctagcttttgcaaacatctatcaagagacaggatcagcaggaggctttcgcatgattgaacaagatggattgcacgcaggttctccggcggcttgggtggagaggctattcggctatgactgggcacaacagacaatcggctgctctgatgccgccgtgttccggctgtcagcgcaggggcgtccggttctttttgtcaagaccgacctgtccggtgccctgaatgaactgcaagacgaggcagcgcggctatcgtggctggcgacgacgggcgttccttgcgcggctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgtctatgcccgacggcgaggatctcgtcgtgacccacggcgatgcctgcttgccgaatatcatggtggaaaatggccgcttttctggattcatcgactgtggccgtctgggtgtggcggaccgctatcaggacatagcgttggctacccgtgatattgctgaagagcttggcggcgaatgggctgaccgcttccttgtgctttacggtatcgccgcgcccgattcgcagcgcatcgccttctatcgccttcttgacgagttcttctgaccgattctaggtgcattggcgcagaaaaaaatgcctgatgcgacgctgcgcgtcttatactcccacatatgccagattcagcaacggatacggcttccccaacttgcccacttccatacgtgtcctccttaccagaaatttatccttaaggtcgtttaaactcgactctggctctatcgaatctccgtcgtttcgagcttacgcgaacagccgtggcgctcatttgctcgtcgggcatcgaatctcgtcagctatcgtcagcttacctttttggcagcgatcgccgcaattaaccctcactaaagggaacaaaagctggagctcggctcccgacatcttggaccattagctccacaggtatcttcttccctctagtggtcataacagcagcttcagctacctctcaatttcaatcgttgcgttacacacacaaaaaaccaacacacatccatcttcgatggatagcgattttattatctaactgctgatcgagtgtagccagatcttgtattctgatagtatgtgtttgtgtatgttaaagatgtttcttgatgttttttcttctataaaacaagagaagtaaaagactgaacacttcgaattgaatattaccaaacttcttattacatttatatatgatatcaagaacttgttaaacacgccaggcttgacctcgaacggtgttttgatgtgttactcaaccgttaaatcgctgtttgagctgactaacaaggttgcttgtcttgaatccccctcaccccgaatttattacgaatttgcccacatggtcggtgctatatcgcattcgttgcactcaccgttcccaagaggagatcaatgttacatacgttcccgatgcccaaaacggtaaagttaaagcgatatagaagctggatgcatatctttttacacgtccgcggcgcgcccaccctcttttttctttctggttccttttcggtcgtttttcttttgtcggctgccttattcccgtggggatgaggcgagcgccgtttcggcttggatacgccatatgcaacaatgacgtcttggtgctggccatgcccctcatttatgtcgaaaaacaggccactgcccctcttttctgcggcccgggcaagaggagacttcatcagccacggcgacgtgctggttgcttctacaatggtatcttcaccttttacataatccaattctatcatgcccttacgtagatctttgtcgatcctaccatccactcgacacacccgccagcggccgctgccaagcttccgagctctcgaattcaaaggaggtaccaaccatgcgttttcccagcatctttaccgccgttttgttcgccgcctcatctgctctggctggaagttcctatccctatgacgtgccagactacgcatccagcggaggaagttccgactcccctgatcgtccgtggaaccctccaaccttttccccggccttactagtagtaaccgagggtgacaacgcaacttttacctgttcattctctaacacaagcgagtcatttcacgtcgtatggcatagagaaagcccaagcggccaaaccgacacgctagcggccttccccgaagacaggtcacaaccaggacaagatgctagatttagagttacgcaactgcccaatggcagggatttccacatgtcagtggtgagagccaggagaaacgactcagggacgtatgtgtgcggcgtaatctctctggccccaaaaatccaaataaaagaatcattacgtgctgagttaagagttacagaacgtggcggcggaagtagcggtggcggaagcggtgggcagcccagggagcctcaagtctatacgctgcctccgtcaagagaagagatgactaagaaccaagtctctctaacttgtctagtaaaggggttttatccttcagatattgccgtcgagtgggagtcaaatgggcaaccggagaataattacaagacaactcctccggtcctagattccgatggcagtttcttcctttactcaaaactgactgttgataaaagccgttggcagcaaggaaatgttttttcttgtagcgtgatgcatgaagcgctgcataaccactatactcagaaatctttatccctgtcacctggcaaatgatctagaagttgtctcctcctgcactgactgactgatacaatcgatttctggatccgcaggcctctgctagcttgactgactgagatacagcgtaccttcagctcatgaacccgatgcaaatgagacgatcgtctattcctggtccggttttctctgccctctcttctattcactttttttatactttatataaaattatataaatgacataactgaaacgccacacgtcctctcctattcgttaacgcctgtctgtagcgctgttactgaagctgcgcaagtagttttttcaccgtataggccctctttttctctctctttctttctctcccgcgctgatctcttcttcgaaacaaaagcaagcaattcgccctatagtgagtcgtattacgcgcgctaaaacctctacaaatgtggtattggcccatctctatcggtatcgtagcataaccccttggggcctctaaacgggtcttgaggggttttttgtgcccctcgggccggattgctatctaccggcattggcgcagaaaaaaatgcctgatgcgacgctgcgcgtcttatactcccacatatgccagattcagcaacggatacggcttccccaacttgcccacttccatacgtgtcctccttaccagaaatttatccttaaggtcgtcagctatcctgcagggtgtttaaagattacggatatttaacttacttagaataatgccatttttttgagttataataatcctacgttagtgtgagcgggatttaaactgtgaggaccttaatacattcagacacttctgcggtatcaccctacttattcccttcgagattatatctaggaacccatcaggttggtggaagattacccgttctaagacttttcagcttcctctattgatgttacacctggacaccccttttctggcatccagtttttaatcttcagtggcatgtgagattctccgaaattaactaaagcaatcacacaattctctcggataccacctcggttgaaactgacaggtggtttgttacgcatgctaatgcaaaggagcctatatacctttggctcggctgctgtaacagggaatataaagggcagcataatttaggagtttagtgaacttgcaacatttactattttcccttcttacgtaaatatttttctttttaattctaaatcaatctttttcaattttttgtttgtattcttttcttgcttaaatctataactacaaaaaacacatacataaactaaaaatggccaaaaccttcaacatctctcagcaggatctggagctggtggaggtcgccactgagaagatcaccatgctctatgaggacaacaagcaccatgtcggggcggccatcaggaccaagactggggagatcatctctgctgtccacattgaagcctacattggcagggtcactgtctgtgctgaagccattgccattgggtctgctgtgagcaacgggcagaaggactttgacaccattgtggctgtcaggcacccctactctgatgaggtggacagatccatcagggtggtcagcccctgtggcatgtgtagagagctgatctctgactatgctcctgactgctttgtgctcattgagatgaatggcaagctggtcaaaaccaccattgaggaactcatccccctcaagtacaccaggaactaaggttaatataattatataaaaatattatcttcttttctttatatctagtgttatgtaaaataaattgatgactacggaaagcctttttatattgtttctttttcattctgagccacttaaatttcgtgaatgttcttgtaagggacggtagatttacaagtgatacaacaaaaagcaaggcgctttttctaataaaaagaagaaaagcatttaacaattgaacacctctatatcaacgaagaatattcctgcaggcgatctctcgatttcgatcaagacattcctttaatggtcttttctggacaccactaggggtcagaagtagttcatcaaactttcttccctccctaatctcattggttaccttgggctatcgaaacttaattaaccagtcaagtcagctacttggcgagatcgacttgtctgggtttcgactacgctcagaattgcgtcagtcaagttcgatctggtccttgctattgcacccgttctccgattacgagtttcatttaaatcatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcatttaaatttccgaactctccaaggccctcgtcggaaaatcttcaaacctttcgtccgatccatcttgcaggctacctctcgaacgaactatcgcaagtctcttggccggccttgcgccttggc

SEQ ID NO: 13 - DNA, 7851 bp, synthetic construct
  source 1..7851: mol_type = other DNA
tgtattctgatagtatgtgtttgtgtatgttaaagatgtttcttgatgttttttcttctataaaacaagagaagtaaaagactgaacacttcgaattgaatattaccaaacttcttattacatttatatatgatatcaagaacttgttaaacacgccaggcttgacctcgaacggtgttttgatgtgttactcaaccgttaaatcgctgtttgagctgactaacaaggttgcttgtcttgaatccccctcaccccgaatttattacgaatttgcccacatggtcggtgctatatcgcattcgttgcactcaccgttcccaagaggagatcaatgttacatacgttcccgatgcccaaaacggtaaagttaaagcgatatagaagctggatgcatatctttttacacgtccgcggcgcgcccaccctcttttttctttctggttccttttcggtcgtttttcttttgtcggctgccttattcccgtggggatgaggcgagcgccgtttcggcttggatacgccatatgcaacaatgacgtcttggtgctggccatgcccctcatttatgtcgaaaaacaggccactgcccctcttttctgcggcccgggcaagaggagacttcatcagccacggcgacgtgctggttgcttctacaatggtatcttcaccttttacataatccaattctatcatgcccttacgtagatctttgtcgatcctaccatccactcgacacacccgccagcggccgctgccaagcttccgagctctcgaattcaaaggaggtaccaaccatgtttaccttccttaagatcatattatggttattttcccttgccttggcctcagcaggaagttcctatccctatgacgtgccagactacgcatccagcggaggaagttccgactcccctgatcgtccgtggaaccctccaaccttttccccggccttactagtagtaaccgagggtgacaacgcaacttttacctgttcattctctaacacaagcgagtcatttcacgtcgtatggcatagagaaagcccaagcggccaaaccgacacgctagcggccttccccgaagacaggtcacaaccaggacaagatgctagatttagagttacgcaactgcccaatggcagggatttccacatgtcagtggtgagagccaggagaaacgactcagggacgtatgtgtgcggcgtaatctctctggccccaaaaatccaaataaaagaatcattacgtgctgagttaagagttacagaacgtggcggcggaagtagcggtggcggaagcggtgggcagcccagggagcctcaagtctatacgctgcctccgtcaagagaagagatgactaagaaccaagtctctctaacttgtctagtaaaggggttttatccttcagatattgccgtcgagtgggagtcaaatgggcaaccggagaataattacaagacaactcctccggtcctagattccgatggcagtttcttcctttactcaaaactgactgttgataaaagccgttggcagcaaggaaatgttttttcttgtagcgtgatgcatgaagcgctgcataaccactatactcagaaatctttatccctgtcacctggcaaatgatctagaagttgtctcctcctgcactgactgactgatacaatcgatttctggatccgcaggcctctgctagcttgactgactgagatacagcgtaccttcagctcaataaagcaatcttgatgaggataatgatttttttttgaatatacataaatactaccgtttttctgctagattttgtgatgacgtaaataagtacatattactttttaagccaagacaagattaagcattaactttacccttttctttctaagtttcaatattagttatcactgtttaaaagttatggcgagaacgtcggcggttaaaatatattaccctgaacgaaaagcaagcaattcgccctatagtgagtcgtattacgcgcgctaaaacctctacaaatgtggtattggcccatctctatcggtatcgtagcataaccccttggggcctctaaacgggtcttgaggggttttttgtgcccctcgggccggattgctatctaccggcattggcgcagaaaaaaatgcctgatgcgacgctgcgcgtcttatactcccacatatgccagattcagcaacggatacggcttccccaacttgcccacttccatacgtgtcctccttaccagaaatttatccttaaggtcgtcagctatcctgcagggtgtttaaagattacggatatttaacttacttagaataatgccatttttttgagttataataatcctacgttagtgtgagcgggatttaaactgtgaggaccttaatacattcagacacttctgcggtatcaccctacttattcccttcgagattatatctaggaacccatcaggttggtggaagattacccgttctaagacttttcagcttcctctattgatgttacacctggacaccccttttctggcatccagtttttaatcttcagtggcatgtgagattctccgaaattaactaaagcaatcacacaattctctcggataccacctcggttgaaactgacaggtggtttgttacgcatgctaatgcaaaggagcctatatacctttggctcggctgctgtaacagggaatataaagggcagcataatttaggagtttagtgaacttgcaacatttactattttcccttcttacgtaaatatttttctttttaattctaaatcaatctttttcaattttttgtttgtattcttttcttgcttaaatctataactacaaaaaacacatacataaactaaaaatggccaaaaccttcaacatctctcagcaggatctggagctggtggaggtcgccactgagaagatcaccatgctctatgaggacaacaagcaccatgtcggggcggccatcaggaccaagactggggagatcatctctgctgtccacattgaagcctacattggcagggtcactgtctgtgctgaagccattgccattgggtctgctgtgagcaacgggcagaaggactttgacaccattgtggctgtcaggcacccctactctgatgaggtggacagatccatcagggtggtcagcccctgtggcatgtgtagagagctgatctctgactatgctcctgactgctttgtgctcattgagatgaatggcaagctggtcaaaaccaccattgaggaactcatccccctcaagtacaccaggaactaaggttaatataattatataaaaatattatcttcttttctttatatctagtgttatgtaaaataaattgatgactacggaaagcctttttatattgtttctttttcattctgagccacttaaatttcgtgaatgttcttgtaagggacggtagatttacaagtgatacaacaaaaagcaaggcgctttttctaataaaaagaagaaaagcatttaacaattgaacacctctatatcaacgaagaatattcctgcaggcgatctctcgatttcgatcaagacattcctttaatggtcttttctggacaccactaggggtcagaagtagttcatcaaactttcttccctccctaatctcattggttaccttgggctatcgaaacttaattaaccagtcaagtcagctacttggcgagatcgacttgtctgggtttcgactacgctcagaattgcgtcagtcaagttcgatctggtccttgctattgcacccgttctccgattacgagtttcatttaaatcatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcatttaaatttccgaactctccaaggccctcgtcggaaaatcttcaaacctttcgtccgatccatcttgcaggctacctctcgaacgaactatcgcaagtctcttggccggccttgcgccttggctattgcttggcagcgcctatcgccaggtattactccaatcccgaatatccgagatcgggatcacccgagagaagttcaacctacatcctcaatcccgatctatccgagatccgaggaatatcgaaatcggggcgcgccaacgaagcatctgtgcttcattttgtagaacaaaaatgcaacgcgagagcgctaatttttcaaacaaagaatctgagctgcatttttacagaacagaaatgcaacgcgaaagcgctattttaccaacgaagaatctgtgcttcatttttgtaaaacaaaaatgcaacgcgagagcgctaatttttcaaacaaagaatctgagctgcatttttacagaacagaaatgcaacgcgagagcgctattttaccaacaaagaatctatacttcttttttgttctacaaaaatgcatcccgagagcgctatttttctaacaaagcatcttagattactttttttctcctttgtgcgctctataatgcagtctcttgataactttttgcactgtaggtccgttaaggttagaagaaggctactttggtgtctattttctcttccataaaaaaagcctgactccacttcccgcgtttactgattactagcgaagctgcgggtgcattttttcaagataaaggcatccccgattatattctataccgatgtggattgcgcatactttgtgaacagaaagtgatagcgttgatgattcttcattggtcagaaaattatgaacggtttcttctattttgtctctatatactacgtataggaaatgtttacattttcgtattgttttcgattcactctatgaatagttcttactacaatttttttgtctaaagagtaatactagagataaacataaaaaatgtagaggtcgagtttagatgcaagttcaaggagcgaaaggtggatgggtaggttatatagggatatagcacagagatatatagcaaagagatacttttgagcaatgtttgtggaagcggtattcgcaatattttagtagctcgttacagtccggtgcgtttttggttttttgaaagtgcgtcttcagagcgcttttggttttcaaaagcgctctgaagttcctatacttcctagagaataggaacttcggaataggaacttcaaagcgtttccgaaaacgagcgcttccgaaaatgcaacgcgagctgcgcacatacagctcactgttcacgtcgcacctatatctgcgtgttgcctgtatatatatatacatgagaagaacggcatagtgcgtgtttatgcttaaatgcgtacttatatgcgtctatttatgtaggatgaaaggtagtctagtacctcctgtgatattatcccattccatgcggggtatcgtatgcttccttcagcactaccctttagctgttctatatgctgccactcctcaattggattagtctcatccttcaatgctatcatttcctttgatattggatcatacggcgcgcctggtgtaccgagaacgatcctctcagtgcgagtctcgacgatccatatcgttgcttggcagtcagccagtcggaatccagcttgggacccaggaagtccaatcgtcagatattgtactcaagcctggtcacggcagcgtaccgatctgtttaaacctagatattgatagtctgatcggtcaacgtataatcgagtcctagcttttgcaaacatctatcaagagacaggatcagcaggaggctttcgcatgattgaacaagatggattgcacgcaggttctccggcggcttgggtggagaggctattcggctatgactgggcacaacagacaatcggctgctctgatgccgccgtgttccggctgtcagcgcaggggcgtccggttctttttgtcaagaccgacctgtccggtgccctgaatgaactgcaagacgaggcagcgcggctatcgtggctggcgacgacgggcgttccttgcgcggctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgtctatgcccgacggcgaggatctcgtcgtgacccacggcgatgcctgcttgccgaatatcatggtggaaaatggccgcttttctggattcatcgactgtggccgtctgggtgtggcggaccgctatcaggacatagcgttggctacccgtgatattgctgaagagcttggcggcgaatgggctgaccgcttccttgtgctttacggtatcgccgcgcccgattcgcagcgcatcgccttctatcgccttcttgacgagttcttctgaccgattctaggtgcattggcgcagaaaaaaatgcctgatgcgacgctgcgcgtcttatactcccacatatgccagattcagcaacggatacggcttccccaacttgcccacttccatacgtgtcctccttaccagaaatttatccttaaggtcgtttaaactcgactctggctctatcgaatctccgtcgtttcgagcttacgcgaacagccgtggcgctcatttgctcgtcgggcatcgaatctcgtcagctatcgtcagcttacctttttggcagcgatcgccgcaattaaccctcactaaagggaacaaaagctggagctcggctcccgacatcttggaccattagctccacaggtatcttcttccctctagtggtcataacagcagcttcagctacctctcaatttcaatcgttgcgttacacacacaaaaaaccaacacacatccatcttcgatggatagcgattttattatctaactgctgatcgagtgtagccagatct

SEQ ID NO: 14 - DNA, 7851 bp, synthetic construct
  source 1..7851: mol_type = other DNA
tgtattctgatagtatgtgtttgtgtatgttaaagatgtttcttgatgttttttcttctataaaacaagagaagtaaaagactgaacacttcgaattgaatattaccaaacttcttattacatttatatatgatatcaagaacttgttaaacacgccaggcttgacctcgaacggtgttttgatgtgttactcaaccgttaaatcgctgtttgagctgactaacaaggttgcttgtcttgaatccccctcaccccgaatttattacgaatttgcccacatggtcggtgctatatcgcattcgttgcactcaccgttcccaagaggagatcaatgttacatacgttcccgatgcccaaaacggtaaagttaaagcgatatagaagctggatgcatatctttttacacgtccgcggcgcgcccaccctcttttttctttctggttccttttcggtcgtttttcttttgtcggctgccttattcccgtggggatgaggcgagcgccgtttcggcttggatacgccatatgcaacaatgacgtcttggtgctggccatgcccctcatttatgtcgaaaaacaggccactgcccctcttttctgcggcccgggcaagaggagacttcatcagccacggcgacgtgctggttgcttctacaatggtatcttcaccttttacataatccaattctatcatgcccttacgtagatctttgtcgatcctaccatccactcgacacacccgccagcggccgctgccaagcttccgagctctcgaattcaaaggaggtaccaaccatgtttaccttccttaagatcatattatggttattttcccttgccttggcctcagcaggaagttcctatccctatgacgtgccagactacgcatccagcggaggaagttccgactcccctgatcgtccgtggaaccctccaaccttttccccggccttactagtagtaaccgagggtgacaacgcaacttttacctgttcattctctaacacaagcgagtcatttcacgtcgtatggcatagagaaagcccaagcggccaaaccgacacgctagcggccttccccgaagacaggtcacaaccaggacaagatgctagatttagagttacgcaactgcccaatggcagggatttccacatgtcagtggtgagagccaggagaaacgactcagggacgtatgtgtgcggcgtaatctctctggccccaaaaatccaaataaaagaatcattacgtgctgagttaagagttacagaacgtggcggcggaagtagcggtggcggaagcggtgggcagcccagggagcctcaagtctatacgctgcctccgtcaagagaagagatgactaagaaccaagtctctctaacttgtctagtaaaggggttttatccttcagatattgccgtcgagtgggagtcaaatgggcaaccggagaataattacaagacaactcctccggtcctagattccgatggcagtttcttcctttactcaaaactgactgttgataaaagccgttggcagcaaggaaatgttttttcttgtagcgtgatgcatgaagcgctgcataaccactatactcagaaatctttatccctgtcacctggcaaatgatctagaagttgtctcctcctgcactgactgactgatacaatcgatttctggatccgcaggcctctgctagcttgactgactgagatacagcgtaccttcagctcaagtgcttttaactaagaattattagtcttttctgcttattttttcatcatagtttagaacactttatattaacgaatagtttatgaatctatttaggtttaaaaattgatacagttttataagttactttttcaaagactcgtgctgtctattgcataatgcactggaaggggaaaaaaaaggtgcacacgcgtggctttttcttgaatttgcagtttgaaaaataaagcaagcaattcgccctatagtgagtcgtattacgcgcgctaaaacctctacaaatgtggtattggcccatctctatcggtatcgtagcataaccccttggggcctctaaacgggtcttgaggggttttttgtgcccctcgggccggattgctatctaccggcattggcgcagaaaaaaatgcctgatgcgacgctgcgcgtcttatactcccacatatgccagattcagcaacggatacggcttccccaacttgcccacttccatacgtgtcctccttaccagaaatttatccttaaggtcgtcagctatcctgcagggtgtttaaagattacggatatttaacttacttagaataatgccatttttttgagttataataatcctacgttagtgtgagcgggatttaaactgtgaggaccttaatacattcagacacttctgcggtatcaccctacttattcccttcgagattatatctaggaacccatcaggttggtggaagattacccgttctaagacttttcagcttcctctattgatgttacacctggacaccccttttctggcatccagtttttaatcttcagtggcatgtgagattctccgaaattaactaaagcaatcacacaattctctcggataccacctcggttgaaactgacaggtggtttgttacgcatgctaatgcaaaggagcctatatacctttggctcggctgctgtaacagggaatataaagggcagcataatttaggagtttagtgaacttgcaacatttactattttcccttcttacgtaaatatttttctttttaattctaaatcaatctttttcaattttttgtttgtattcttttcttgcttaaatctataactacaaaaaacacatacataaactaaaaatggccaaaaccttcaacatctctcagcaggatctggagctggtggaggtcgccactgagaagatcaccatgctctatgaggacaacaagcaccatgtcggggcggccatcaggaccaagactggggagatcatctctgctgtccacattgaagcctacattggcagggtcactgtctgtgctgaagccattgccattgggtctgctgtgagcaacgggcagaaggactttgacaccattgtggctgtcaggcacccctactctgatgaggtggacagatccatcagggtggtcagcccctgtggcatgtgtagagagctgatctctgactatgctcctgactgctttgtgctcattgagatgaatggcaagctggtcaaaaccaccattgaggaactcatccccctcaagtacaccaggaactaaggttaatataattatataaaaatattatcttcttttctttatatctagtgttatgtaaaataaattgatgactacggaaagcctttttatattgtttctttttcattctgagccacttaaatttcgtgaatgttcttgtaagggacggtagatttacaagtgatacaacaaaaagcaaggcgctttttctaataaaaagaagaaaagcatttaacaattgaacacctctatatcaacgaagaatattcctgcaggcgatctctcgatttcgatcaagacattcctttaatggtcttttctggacaccactaggggtcagaagtagttcatcaaactttcttccctccctaatctcattggttaccttgggctatcgaaacttaattaaccagtcaagtcagctacttggcgagatcgacttgtctgggtttcgactacgctcagaattgcgtcagtcaagttcgatctggtccttgctattgcacccgttctccgattacgagtttcatttaaatcatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcatttaaatttccgaactctccaaggccctcgtcggaaaatcttcaaacctttcgtccgatccatcttgcaggctacctctcgaacgaactatcgcaagtctcttggccggccttgcgccttggctattgcttggcagcgcctatcgccaggtattactccaatcccgaatatccgagatcgggatcacccgagagaagttcaacctacatcctcaatcccgatctatccgagatccgaggaatatcgaaatcggggcgcgccaacgaagcatctgtgcttcattttgtagaacaaaaatgcaacgcgagagcgctaatttttcaaacaaagaatctgagctgcatttttacagaacagaaatgcaacgcgaaagcgctattttaccaacgaagaatctgtgcttcatttttgtaaaacaaaaatgcaacgcgagagcgctaatttttcaaacaaagaatctgagctgcatttttacagaacagaaatgcaacgcgagagcgctattttaccaacaaagaatctatacttcttttttgttctacaaaaatgcatcccgagagcgctatttttctaacaaagcatcttagattactttttttctcctttgtgcgctctataatgcagtctcttgataactttttgcactgtaggtccgttaaggttagaagaaggctactttggtgtctattttctcttccataaaaaaagcctgactccacttcccgcgtttactgattactagcgaagctgcgggtgcattttttcaagataaaggcatccccgattatattctataccgatgtggattgcgcatactttgtgaacagaaagtgatagcgttgatgattcttcattggtcagaaaattatgaacggtttcttctattttgtctctatatactacgtataggaaatgtttacattttcgtattgttttcgattcactctatgaatagttcttactacaatttttttgtctaaagagtaatactagagataaacataaaaaatgtagaggtcgagtttagatgcaagttcaaggagcgaaaggtggatgggtaggttatatagggatatagcacagagatatatagcaaagagatacttttgagcaatgtttgtggaagcggtattcgcaatattttagtagctcgttacagtccggtgcgtttttggttttttgaaagtgcgtcttcagagcgcttttggttttcaaaagcgctctgaagttcctatacttcctagagaataggaacttcggaataggaacttcaaagcgtttccgaaaacgagcgcttccgaaaatgcaacgcgagctgcgcacatacagctcactgttcacgtcgcacctatatctgcgtgttgcctgtatatatatatacatgagaagaacggcatagtgcgtgtttatgcttaaatgcgtacttatatgcgtctatttatgtaggatgaaaggtagtctagtacctcctgtgatattatcccattccatgcggggtatcgtatgcttccttcagcactaccctttagctgttctatatgctgccactcctcaattggattagtctcatccttcaatgctatcatttcctttgatattggatcatacggcgcgcctggtgtaccgagaacgatcctctcagtgcgagtctcgacgatccatatcgttgcttggcagtcagccagtcggaatccagcttgggacccaggaagtccaatcgtcagatattgtactcaagcctggtcacggcagcgtaccgatctgtttaaacctagatattgatagtctgatcggtcaacgtataatcgagtcctagcttttgcaaacatctatcaagagacaggatcagcaggaggctttcgcatgattgaacaagatggattgcacgcaggttctccggcggcttgggtggagaggctattcggctatgactgggcacaacagacaatcggctgctctgatgccgccgtgttccggctgtcagcgcaggggcgtccggttctttttgtcaagaccgacctgtccggtgccctgaatgaactgcaagacgaggcagcgcggctatcgtggctggcgacgacgggcgttccttgcgcggctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgtctatgcccgacggcgaggatctcgtcgtgacccacggcgatgcctgcttgccgaatatcatggtggaaaatggccgcttttctggattcatcgactgtggccgtctgggtgtggcggaccgctatcaggacatagcgttggctacccgtgatattgctgaagagcttggcggcgaatgggctgaccgcttccttgtgctttacggtatcgccgcgcccgattcgcagcgcatcgccttctatcgccttcttgacgagttcttctgaccgattctaggtgcattggcgcagaaaaaaatgcctgatgcgacgctgcgcgtcttatactcccacatatgccagattcagcaacggatacggcttccccaacttgcccacttccatacgtgtcctccttaccagaaatttatccttaaggtcgtttaaactcgactctggctctatcgaatctccgtcgtttcgagcttacgcgaacagccgtggcgctcatttgctcgtcgggcatcgaatctcgtcagctatcgtcagcttacctttttggcagcgatcgccgcaattaaccctcactaaagggaacaaaagctggagctcggctcccgacatcttggaccattagctccacaggtatcttcttccctctagtggtcataacagcagcttcagctacctctcaatttcaatcgttgcgttacacacacaaaaaaccaacacacatccatcttcgatggatagcgattttattatctaactgctgatcgagtgtagccagatct

SEQ ID NO: 15 - DNA, 7851 bp, synthetic construct
  source 1..7851: mol_type = other DNA
tgtattctgatagtatgtgtttgtgtatgttaaagatgtttcttgatgttttttcttctataaaacaagagaagtaaaagactgaacacttcgaattgaatattaccaaacttcttattacatttatatatgatatcaagaacttgttaaacacgccaggcttgacctcgaacggtgttttgatgtgttactcaaccgttaaatcgctgtttgagctgactaacaaggttgcttgtcttgaatccccctcaccccgaatttattacgaatttgcccacatggtcggtgctatatcgcattcgttgcactcaccgttcccaagaggagatcaatgttacatacgttcccgatgcccaaaacggtaaagttaaagcgatatagaagctggatgcatatctttttacacgtccgcggcgcgcccaccctcttttttctttctggttccttttcggtcgtttttcttttgtcggctgccttattcccgtggggatgaggcgagcgccgtttcggcttggatacgccatatgcaacaatgacgtcttggtgctggccatgcccctcatttatgtcgaaaaacaggccactgcccctcttttctgcggcccgggcaagaggagacttcatcagccacggcgacgtgctggttgcttctacaatggtatcttcaccttttacataatccaattctatcatgcccttacgtagatctttgtcgatcctaccatccactcgacacacccgccagcggccgctgccaagcttccgagctctcgaattcaaaggaggtaccaaccatgtttaccttccttaagatcatattatggttattttcccttgccttggcctcagcaggaagttcctatccctatgacgtgccagactacgcatccagcggaggaagttccgactcccctgatcgtccgtggaaccctccaaccttttccccggccttactagtagtaaccgagggtgacaacgcaacttttacctgttcattctctaacacaagcgagtcatttcacgtcgtatggcatagagaaagcccaagcggccaaaccgacacgctagcggccttccccgaagacaggtcacaaccaggacaagatgctagatttagagttacgcaactgcccaatggcagggatttccacatgtcagtggtgagagccaggagaaacgactcagggacgtatgtgtgcggcgtaatctctctggccccaaaaatccaaataaaagaatcattacgtgctgagttaagagttacagaacgtggcggcggaagtagcggtggcggaagcggtgggcagcccagggagcctcaagtctatacgctgcctccgtcaagagaagagatgactaagaaccaagtctctctaacttgtctagtaaaggggttttatccttcagatattgccgtcgagtgggagtcaaatgggcaaccggagaataattacaagacaactcctccggtcctagattccgatggcagtttcttcctttactcaaaactgactgttgataaaagccgttggcagcaaggaaatgttttttcttgtagcgtgatgcatgaagcgctgcataaccactatactcagaaatctttatccctgtcacctggcaaatgatctagaagttgtctcctcctgcactgactgactgatacaatcgatttctggatccgcaggcctctgctagcttgactgactgagatacagcgtaccttcagctcagccaattggtgcggcaattgataataacgaaaatgtcttttaatgatctgggtataatgaggaattttccgaacgtttttactttatatatatatatacatgtaacatatattctatacgctatagagaaaggaaatttttcaattaaaaaaaaaatagagaaagagtttcacttcttgattatcgctaacactaatggttgaagtactgctactttaattttataaagcaagcaattcgccctatagtgagtcgtattacgcgcgctaaaacctctacaaatgtggtattggcccatctctatcggtatcgtagcataaccccttggggcctctaaacgggtcttgaggggttttttgtgcccctcgggccggattgctatctaccggcattggcgcagaaaaaaatgcctgatgcgacgctgcgcgtcttatactcccacatatgccagattcagcaacggatacggcttccccaacttgcccacttccatacgtgtcctccttaccagaaatttatccttaaggtcgtcagctatcctgcagggtgtttaaagattacggatatttaacttacttagaataatgccatttttttgagttataataatcctacgttagtgtgagcgggatttaaactgtgaggaccttaatacattcagacacttctgcggtatcaccctacttattcccttcgagattatatctaggaacccatcaggttggtggaagattacccgttctaagacttttcagcttcctctattgatgttacacctggacaccccttttctggcatccagtttttaatcttcagtggcatgtgagattctccgaaattaactaaagcaatcacacaattctctcggataccacctcggttgaaactgacaggtggtttgttacgcatgctaatgcaaaggagcctatatacctttggctcggctgctgtaacagggaatataaagggcagcataatttaggagtttagtgaacttgcaacatttactattttcccttcttacgtaaatatttttctttttaattctaaatcaatctttttcaattttttgtttgtattcttttcttgcttaaatctataactacaaaaaacacatacataaactaaaaatggccaaaaccttcaacatctctcagcaggatctggagctggtggaggtcgccactgagaagatcaccatgctctatgaggacaacaagcaccatgtcggggcggccatcaggaccaagactggggagatcatctctgctgtccacattgaagcctacattggcagggtcactgtctgtgctgaagccattgccattgggtctgctgtgagcaacgggcagaaggactttgacaccattgtggctgtcaggcacccctactctgatgaggtggacagatccatcagggtggtcagcccctgtggcatgtgtagagagctgatctctgactatgctcctgactgctttgtgctcattgagatgaatggcaagctggtcaaaaccaccattgaggaactcatccccctcaagtacaccaggaactaaggttaatataattatataaaaatattatcttcttttctttatatctagtgttatgtaaaataaattgatgactacggaaagcctttttatattgtttctttttcattctgagccacttaaatttcgtgaatgttcttgtaagggacggtagatttacaagtgatacaacaaaaagcaaggcgctttttctaataaaaagaagaaaagcatttaacaattgaacacctctatatcaacgaagaatattcctgcaggcgatctctcgatttcgatcaagacattcctttaatggtcttttctggacaccactaggggtcagaagtagttcatcaaactttcttccctccctaatctcattggttaccttgggctatcgaaacttaattaaccagtcaagtcagctacttggcgagatcgacttgtctgggtttcgactacgctcagaattgcgtcagtcaagttcgatctggtccttgctattgcacccgttctccgattacgagtttcatttaaatcatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcatttaaatttccgaactctccaaggccctcgtcggaaaatcttcaaacctttcgtccgatccatcttgcaggctacctctcgaacgaactatcgcaagtctcttggccggccttgcgccttggctattgcttggcagcgcctatcgccaggtattactccaatcccgaatatccgagatcgggatcacccgagagaagttcaacctacatcctcaatcccgatctatccgagatccgaggaatatcgaaatcggggcgcgccaacgaagcatctgtgcttcattttgtagaacaaaaatgcaacgcgagagcgctaatttttcaaacaaagaatctgagctgcatttttacagaacagaaatgcaacgcgaaagcgctattttaccaacgaagaatctgtgcttcatttttgtaaaacaaaaatgcaacgcgagagcgctaatttttcaaacaaagaatctgagctgcatttttacagaacagaaatgcaacgcgagagcgctattttaccaacaaagaatctatacttcttttttgttctacaaaaatgcatcccgagagcgctatttttctaacaaagcatcttagattactttttttctcctttgtgcgctctataatgcagtctcttgataactttttgcactgtaggtccgttaaggttagaagaaggctactttggtgtctattttctcttccataaaaaaagcctgactccacttcccgcgtttactgattactagcgaagctgcgggtgcattttttcaagataaaggcatccccgattatattctataccgatgtggattgcgcatactttgtgaacagaaagtgatagcgttgatgattcttcattggtcagaaaattatgaacggtttcttctattttgtctctatatactacgtataggaaatgtttacattttcgtattgttttcgattcactctatgaatagttcttactacaatttttttgtctaaagagtaatactagagataaacataaaaaatgtagaggtcgagtttagatgcaagttcaaggagcgaaaggtggatgggtaggttatatagggatatagcacagagatatatagcaaagagatacttttgagcaatgtttgtggaagcggtattcgcaatattttagtagctcgttacagtccggtgcgtttttggttttttgaaagtgcgtcttcagagcgcttttggttttcaaaagcgctctgaagttcctatacttcctagagaataggaacttcggaataggaacttcaaagcgtttccgaaaacgagcgcttccgaaaatgcaacgcgagctgcgcacatacagctcactgttcacgtcgcacctatatctgcgtgttgcctgtatatatatatacatgagaagaacggcatagtgcgtgtttatgcttaaatgcgtacttatatgcgtctatttatgtaggatgaaaggtagtctagtacctcctgtgatattatcccattccatgcggggtatcgtatgcttccttcagcactaccctttagctgttctatatgctgccactcctcaattggattagtctcatccttcaatgctatcatttcctttgatattggatcatacggcgcgcctggtgtaccgagaacgatcctctcagtgcgagtctcgacgatccatatcgttgcttggcagtcagccagtcggaatccagcttgggacccaggaagtccaatcgtcagatattgtactcaagcctggtcacggcagcgtaccgatctgtttaaacctagatattgatagtctgatcggtcaacgtataatcgagtcctagcttttgcaaacatctatcaagagacaggatcagcaggaggctttcgcatgattgaacaagatggattgcacgcaggttctccggcggcttgggtggagaggctattcggctatgactgggcacaacagacaatcggctgctctgatgccgccgtgttccggctgtcagcgcaggggcgtccggttctttttgtcaagaccgacctgtccggtgccctgaatgaactgcaagacgaggcagcgcggctatcgtggctggcgacgacgggcgttccttgcgcggctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgtctatgcccgacggcgaggatctcgtcgtgacccacggcgatgcctgcttgccgaatatcatggtggaaaatggccgcttttctggattcatcgactgtggccgtctgggtgtggcggaccgctatcaggacatagcgttggctacccgtgatattgctgaagagcttggcggcgaatgggctgaccgcttccttgtgctttacggtatcgccgcgcccgattcgcagcgcatcgccttctatcgccttcttgacgagttcttctgaccgattctaggtgcattggcgcagaaaaaaatgcctgatgcgacgctgcgcgtcttatactcccacatatgccagattcagcaacggatacggcttccccaacttgcccacttccatacgtgtcctccttaccagaaatttatccttaaggtcgtttaaactcgactctggctctatcgaatctccgtcgtttcgagcttacgcgaacagccgtggcgctcatttgctcgtcgggcatcgaatctcgtcagctatcgtcagcttacctttttggcagcgatcgccgcaattaaccctcactaaagggaacaaaagctggagctcggctcccgacatcttggaccattagctccacaggtatcttcttccctctagtggtcataacagcagcttcagctacctctcaatttcaatcgttgcgttacacacacaaaaaaccaacacacatccatcttcgatggatagcgattttattatctaactgctgatcgagtgtagccagatct

SEQ ID NO: 16 - DNA, 7574 bp, synthetic construct
  source 1..7574: mol_type = other DNA
catagcttcaaaatgtttctactccttttttactcttccagattttctcggactccgcgcatcgccgtaccacttcaaaacacccaagcacagcatactaaatttcccctctttcttcctctagggtgtcgttaattacccgtactaaaggtttggaaaagaaaaaagagaccgcctcgtttctttttcttcgtcgaaaaaggcaataaaaatttttatcacgtttctttttcttgaaaattttttttttgatttttttctctttcgatgacctcccattgatatttaagttaataaacggtcttcaatttctcaagtttcagtttcatttttcttgttctattacaactttttttacttcttgctcattagaaagaaagcatagcaatctaatctaagagatctttgtcgatcctaccatccactcgacacacccgccagcggccgctgccaagcttccgagctctcgaattcaaaggaggtaccaaccatgcgttttcccagcatctttaccgccgttttgttcgccgcctcatctgctctggctggaagttcctatccctatgacgtgccagactacgcatccagcggaggaagttccgactcccctgatcgtccgtggaaccctccaaccttttccccggccttactagtagtaaccgagggtgacaacgcaacttttacctgttcattctctaacacaagcgagtcatttcacgtcgtatggcatagagaaagcccaagcggccaaaccgacacgctagcggccttccccgaagacaggtcacaaccaggacaagatgctagatttagagttacgcaactgcccaatggcagggatttccacatgtcagtggtgagagccaggagaaacgactcagggacgtatgtgtgcggcgtaatctctctggccccaaaaatccaaataaaagaatcattacgtgctgagttaagagttacagaacgtggcggcggaagtagcggtggcggaagcggtgggcagcccagggagcctcaagtctatacgctgcctccgtcaagagaagagatgactaagaaccaagtctctctaacttgtctagtaaaggggttttatccttcagatattgccgtcgagtgggagtcaaatgggcaaccggagaataattacaagacaactcctccggtcctagattccgatggcagtttcttcctttactcaaaactgactgttgataaaagccgttggcagcaaggaaatgttttttcttgtagcgtgatgcatgaagcgctgcataaccactatactcagaaatctttatccctgtcacctggcaaatgatctagaagttgtctcctcctgcactgactgactgatacaatcgatttctggatccgcaggcctctgctagcttgactgactgagatacagcgtaccttcagctcatcatgtaattagttatgtcacgcttacattcacgccctccccccacatccgctctaaccgaaaaggaaggagttagacaacctgaagtctaggtccctatttatttttttatagttatgttagtattaagaacgttatttatatttcaaatttttcttttttttctgtacagacgcgtgtacgcatgtaacattatactgaaaaccttgcttgagaaggttttgggacgctcgaaggctttaatttgcggccaaagcaagcaattcgccctatagtgagtcgtattacgcgcgctaaaacctctacaaatgtggtattggcccatctctatcggtatcgtagcataaccccttggggcctctaaacgggtcttgaggggttttttgtgcccctcgggccggattgctatctaccggcattggcgcagaaaaaaatgcctgatgcgacgctgcgcgtcttatactcccacatatgccagattcagcaacggatacggcttccccaacttgcccacttccatacgtgtcctccttaccagaaatttatccttaaggtcgtcagctatcctgcagggtgtttaaagattacggatatttaacttacttagaataatgccatttttttgagttataataatcctacgttagtgtgagcgggatttaaactgtgaggaccttaatacattcagacacttctgcggtatcaccctacttattcccttcgagattatatctaggaacccatcaggttggtggaagattacccgttctaagacttttcagcttcctctattgatgttacacctggacaccccttttctggcatccagtttttaatcttcagtggcatgtgagattctccgaaattaactaaagcaatcacacaattctctcggataccacctcggttgaaactgacaggtggtttgttacgcatgctaatgcaaaggagcctatatacctttggctcggctgctgtaacagggaatataaagggcagcataatttaggagtttagtgaacttgcaacatttactattttcccttcttacgtaaatatttttctttttaattctaaatcaatctttttcaattttttgtttgtattcttttcttgcttaaatctataactacaaaaaacacatacataaactaaaaatggccaaaaccttcaacatctctcagcaggatctggagctggtggaggtcgccactgagaagatcaccatgctctatgaggacaacaagcaccatgtcggggcggccatcaggaccaagactggggagatcatctctgctgtccacattgaagcctacattggcagggtcactgtctgtgctgaagccattgccattgggtctgctgtgagcaacgggcagaaggactttgacaccattgtggctgtcaggcacccctactctgatgaggtggacagatccatcagggtggtcagcccctgtggcatgtgtagagagctgatctctgactatgctcctgactgctttgtgctcattgagatgaatggcaagctggtcaaaaccaccattgaggaactcatccccctcaagtacaccaggaactaaggttaatataattatataaaaatattatcttcttttctttatatctagtgttatgtaaaataaattgatgactacggaaagcctttttatattgtttctttttcattctgagccacttaaatttcgtgaatgttcttgtaagggacggtagatttacaagtgatacaacaaaaagcaaggcgctttttctaataaaaagaagaaaagcatttaacaattgaacacctctatatcaacgaagaatattcctgcaggcgatctctcgatttcgatcaagacattcctttaatggtcttttctggacaccactaggggtcagaagtagttcatcaaactttcttccctccctaatctcattggttaccttgggctatcgaaacttaattaaccagtcaagtcagctacttggcgagatcgacttgtctgggtttcgactacgctcagaattgcgtcagtcaagttcgatctggtccttgctattgcacccgttctccgattacgagtttcatttaaatcatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcatttaaatttccgaactctccaaggccctcgtcggaaaatcttcaaacctttcgtccgatccatcttgcaggctacctctcgaacgaactatcgcaagtctcttggccggccttgcgccttggctattgcttggcagcgcctatcgccaggtattactccaatcccgaatatccgagatcgggatcacccgagagaagttcaacctacatcctcaatcccgatctatccgagatccgaggaatatcgaaatcggggcgcgccaacgaagcatctgtgcttcattttgtagaacaaaaatgcaacgcgagagcgctaatttttcaaacaaagaatctgagctgcatttttacagaacagaaatgcaacgcgaaagcgctattttaccaacgaagaatctgtgcttcatttttgtaaaacaaaaatgcaacgcgagagcgctaatttttcaaacaaagaatctgagctgcatttttacagaacagaaatgcaacgcgagagcgctattttaccaacaaagaatctatacttcttttttgttctacaaaaatgcatcccgagagcgctatttttctaacaaagcatcttagattactttttttctcctttgtgcgctctataatgcagtctcttgataactttttgcactgtaggtccgttaaggttagaagaaggctactttggtgtctattttctcttccataaaaaaagcctgactccacttcccgcgtttactgattactagcgaagctgcgggtgcattttttcaagataaaggcatccccgattatattctataccgatgtggattgcgcatactttgtgaacagaaagtgatagcgttgatgattcttcattggtcagaaaattatgaacggtttcttctattttgtctctatatactacgtataggaaatgtttacattttcgtattgttttcgattcactctatgaatagttcttactacaatttttttgtctaaagagtaatactagagataaacataaaaaatgtagaggtcgagtttagatgcaagttcaaggagcgaaaggtggatgggtaggttatatagggatatagcacagagatatatagcaaagagatacttttgagcaatgtttgtggaagcggtattcgcaatattttagtagctcgttacagtccggtgcgtttttggttttttgaaagtgcgtcttcagagcgcttttggttttcaaaagcgctctgaagttcctatacttcctagagaataggaacttcggaataggaacttcaaagcgtttccgaaaacgagcgcttccgaaaatgcaacgcgagctgcgcacatacagctcactgttcacgtcgcacctatatctgcgtgttgcctgtatatatatatacatgagaagaacggcatagtgcgtgtttatgcttaaatgcgtacttatatgcgtctatttatgtaggatgaaaggtagtctagtacctcctgtgatattatcccattccatgcggggtatcgtatgcttccttcagcactaccctttagctgttctatatgctgccactcctcaattggattagtctcatccttcaatgctatcatttcctttgatattggatcatacggcgcgcctggtgtaccgagaacgatcctctcagtgcgagtctcgacgatccatatcgttgcttggcagtcagccagtcggaatccagcttgggacccaggaagtccaatcgtcagatattgtactcaagcctggtcacggcagcgtaccgatctgtttaaacctagatattgatagtctgatcggtcaacgtataatcgagtcctagcttttgcaaacatctatcaagagacaggatcagcaggaggctttcgcatgattgaacaagatggattgcacgcaggttctccggcggcttgggtggagaggctattcggctatgactgggcacaacagacaatcggctgctctgatgccgccgtgttccggctgtcagcgcaggggcgtccggttctttttgtcaagaccgacctgtccggtgccctgaatgaactgcaagacgaggcagcgcggctatcgtggctggcgacgacgggcgttccttgcgcggctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgtctatgcccgacggcgaggatctcgtcgtgacccacggcgatgcctgcttgccgaatatcatggtggaaaatggccgcttttctggattcatcgactgtggccgtctgggtgtggcggaccgctatcaggacatagcgttggctacccgtgatattgctgaagagcttggcggcgaatgggctgaccgcttccttgtgctttacggtatcgccgcgcccgattcgcagcgcatcgccttctatcgccttcttgacgagttcttctgaccgattctaggtgcattggcgcagaaaaaaatgcctgatgcgacgctgcgcgtcttatactcccacatatgccagattcagcaacggatacggcttccccaacttgcccacttccatacgtgtcctccttaccagaaatttatccttaaggtcgtttaaactcgactctggctctatcgaatctccgtcgtttcgagcttacgcgaacagccgtggcgctcatttgctcgtcgggcatcgaatctcgtcagctatcgtcagcttacctttttggcagcgatcgccgcaattaaccctcactaaagggaacaaaagctggagctcggctcccgacatcttggaccattagctccacaggtatcttcttccctctagtggtcataacagcagcttcagctacctctcaatttcaatcgttgcgttacacacacaaaaaaccaacacacatccatcttcgatggatagcgattttattatctaactgctgatcgagtgtagccagatct

SEQ ID NO: 17 - DNA, 7574 bp, synthetic construct
  source 1..7574: mol_type = other DNA
tattgcttggcagcgcctatcgccaggtattactccaatcccgaatatccgagatcgggatcacccgagagaagttcaacctacatcctcaatcccgatctatccgagatccgaggaatatcgaaatcggggcgcgccaacgaagcatctgtgcttcattttgtagaacaaaaatgcaacgcgagagcgctaatttttcaaacaaagaatctgagctgcatttttacagaacagaaatgcaacgcgaaagcgctattttaccaacgaagaatctgtgcttcatttttgtaaaacaaaaatgcaacgcgagagcgctaatttttcaaacaaagaatctgagctgcatttttacagaacagaaatgcaacgcgagagcgctattttaccaacaaagaatctatacttcttttttgttctacaaaaatgcatcccgagagcgctatttttctaacaaagcatcttagattactttttttctcctttgtgcgctctataatgcagtctcttgataactttttgcactgtaggtccgttaaggttagaagaaggctactttggtgtctattttctcttccataaaaaaagcctgactccacttcccgcgtttactgattactagcgaagctgcgggtgcattttttcaagataaaggcatccccgattatattctataccgatgtggattgcgcatactttgtgaacagaaagtgatagcgttgatgattcttcattggtcagaaaattatgaacggtttcttctattttgtctctatatactacgtataggaaatgtttacattttcgtattgttttcgattcactctatgaatagttcttactacaatttttttgtctaaagagtaatactagagataaacataaaaaatgtagaggtcgagtttagatgcaagttcaaggagcgaaaggtggatgggtaggttatatagggatatagcacagagatatatagcaaagagatacttttgagcaatgtttgtggaagcggtattcgcaatattttagtagctcgttacagtccggtgcgtttttggttttttgaaagtgcgtcttcagagcgcttttggttttcaaaagcgctctgaagttcctatacttcctagagaataggaacttcggaataggaacttcaaagcgtttccgaaaacgagcgcttccgaaaatgcaacgcgagctgcgcacatacagctcactgttcacgtcgcacctatatctgcgtgttgcctgtatatatatatacatgagaagaacggcatagtgcgtgtttatgcttaaatgcgtacttatatgcgtctatttatgtaggatgaaaggtagtctagtacctcctgtgatattatcccattccatgcggggtatcgtatgcttccttcagcactaccctttagctgttctatatgctgccactcctcaattggattagtctcatccttcaatgctatcatttcctttgatattggatcatacggcgcgcctggtgtaccgagaacgatcctctcagtgcgagtctcgacgatccatatcgttgcttggcagtcagccagtcggaatccagcttgggacccaggaagtccaatcgtcagatattgtactcaagcctggtcacggcagcgtaccgatctgtttaaacctagatattgatagtctgatcggtcaacgtataatcgagtcctagcttttgcaaacatctatcaagagacaggatcagcaggaggctttcgcatgattgaacaagatggattgcacgcaggttctccggcggcttgggtggagaggctattcggctatgactgggcacaacagacaatcggctgctctgatgccgccgtgttccggctgtcagcgcaggggcgtccggttctttttgtcaagaccgacctgtccggtgccctgaatgaactgcaagacgaggcagcgcggctatcgtggctggcgacgacgggcgttccttgcgcggctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgtctatgcccgacggcgaggatctcgtcgtgacccacggcgatgcctgcttgccgaatatcatggtggaaaatggccgcttttctggattcatcgactgtggccgtctgggtgtggcggaccgctatcaggacatagcgttggctacccgtgatattgctgaagagcttggcggcgaatgggctgaccgcttccttgtgctttacggtatcgccgcgcccgattcgcagcgcatcgccttctatcgccttcttgacgagttcttctgaccgattctaggtgcattggcgcagaaaaaaatgcctgatgcgacgctgcgcgtcttatactcccacatatgccagattcagcaacggatacggcttccccaacttgcccacttccatacgtgtcctccttaccagaaatttatccttaaggtcgtttaaactcgactctggctctatcgaatctccgtcgtttcgagcttacgcgaacagccgtggcgctcatttgctcgtcgggcatcgaatctcgtcagctatcgtcagcttacctttttggcagcgatcgccgcaattaaccctcactaaagggaacaaaagctggagctcggctcccgacatcttggaccattagctccacaggtatcttcttccctctagtggtcataacagcagcttcagctacctctcaatttcaatcgttgcgttacacacacaaaaaaccaacacacatccatcttcgatggatagcgattttattatctaactgctgatcgagtgtagccagatctcatagcttcaaaatgtttctactccttttttactcttccagattttctcggactccgcgcatcgccgtaccacttcaaaacacccaagcacagcatactaaatttcccctctttcttcctctagggtgtcgttaattacccgtactaaaggtttggaaaagaaaaaagagaccgcctcgtttctttttcttcgtcgaaaaaggcaataaaaatttttatcacgtttctttttcttgaaaattttttttttgatttttttctctttcgatgacctcccattgatatttaagttaataaacggtcttcaatttctcaagtttcagtttcatttttcttgttctattacaactttttttacttcttgctcattagaaagaaagcatagcaatctaatctaagagatctttgtcgatcctaccatccactcgacacacccgccagcggccgctgccaagcttccgagctctcgaattcaaaggaggtaccaaccatgttgttacaggcattcttgtttctgttggccggttttgctgcgaagatttctgccggaagttcctatccctatgacgtgccagactacgcatccagcggaggaagttccgactcccctgatcgtccgtggaaccctccaaccttttccccggccttactagtagtaaccgagggtgacaacgcaacttttacctgttcattctctaacacaagcgagtcatttcacgtcgtatggcatagagaaagcccaagcggccaaaccgacacgctagcggccttccccgaagacaggtcacaaccaggacaagatgctagatttagagttacgcaactgcccaatggcagggatttccacatgtcagtggtgagagccaggagaaacgactcagggacgtatgtgtgcggcgtaatctctctggccccaaaaatccaaataaaagaatcattacgtgctgagttaagagttacagaacgtggcggcggaagtagcggtggcggaagcggtgggcagcccagggagcctcaagtctatacgctgcctccgtcaagagaagagatgactaagaaccaagtctctctaacttgtctagtaaaggggttttatccttcagatattgccgtcgagtgggagtcaaatgggcaaccggagaataattacaagacaactcctccggtcctagattccgatggcagtttcttcctttactcaaaactgactgttgataaaagccgttggcagcaaggaaatgttttttcttgtagcgtgatgcatgaagcgctgcataaccactatactcagaaatctttatccctgtcacctggcaaatgatctagaagttgtctcctcctgcactgactgactgatacaatcgatttctggatccgcaggcctctgctagcttgactgactgagatacagcgtaccttcagctcatcatgtaattagttatgtcacgcttacattcacgccctccccccacatccgctctaaccgaaaaggaaggagttagacaacctgaagtctaggtccctatttatttttttatagttatgttagtattaagaacgttatttatatttcaaatttttcttttttttctgtacagacgcgtgtacgcatgtaacattatactgaaaaccttgcttgagaaggttttgggacgctcgaaggctttaatttgcggccaaagcaagcaattcgccctatagtgagtcgtattacgcgcgctaaaacctctacaaatgtggtattggcccatctctatcggtatcgtagcataaccccttggggcctctaaacgggtcttgaggggttttttgtgcccctcgggccggattgctatctaccggcattggcgcagaaaaaaatgcctgatgcgacgctgcgcgtcttatactcccacatatgccagattcagcaacggatacggcttccccaacttgcccacttccatacgtgtcctccttaccagaaatttatccttaaggtcgtcagctatcctgcagggtgtttaaagattacggatatttaacttacttagaataatgccatttttttgagttataataatcctacgttagtgtgagcgggatttaaactgtgaggaccttaatacattcagacacttctgcggtatcaccctacttattcccttcgagattatatctaggaacccatcaggttggtggaagattacccgttctaagacttttcagcttcctctattgatgttacacctggacaccccttttctggcatccagtttttaatcttcagtggcatgtgagattctccgaaattaactaaagcaatcacacaattctctcggataccacctcggttgaaactgacaggtggtttgttacgcatgctaatgcaaaggagcctatatacctttggctcggctgctgtaacagggaatataaagggcagcataatttaggagtttagtgaacttgcaacatttactattttcccttcttacgtaaatatttttctttttaattctaaatcaatctttttcaattttttgtttgtattcttttcttgcttaaatctataactacaaaaaacacatacataaactaaaaatggccaaaaccttcaacatctctcagcaggatctggagctggtggaggtcgccactgagaagatcaccatgctctatgaggacaacaagcaccatgtcggggcggccatcaggaccaagactggggagatcatctctgctgtccacattgaagcctacattggcagggtcactgtctgtgctgaagccattgccattgggtctgctgtgagcaacgggcagaaggactttgacaccattgtggctgtcaggcacccctactctgatgaggtggacagatccatcagggtggtcagcccctgtggcatgtgtagagagctgatctctgactatgctcctgactgctttgtgctcattgagatgaatggcaagctggtcaaaaccaccattgaggaactcatccccctcaagtacaccaggaactaaggttaatataattatataaaaatattatcttcttttctttatatctagtgttatgtaaaataaattgatgactacggaaagcctttttatattgtttctttttcattctgagccacttaaatttcgtgaatgttcttgtaagggacggtagatttacaagtgatacaacaaaaagcaaggcgctttttctaataaaaagaagaaaagcatttaacaattgaacacctctatatcaacgaagaatattcctgcaggcgatctctcgatttcgatcaagacattcctttaatggtcttttctggacaccactaggggtcagaagtagttcatcaaactttcttccctccctaatctcattggttaccttgggctatcgaaacttaattaaccagtcaagtcagctacttggcgagatcgacttgtctgggtttcgactacgctcagaattgcgtcagtcaagttcgatctggtccttgctattgcacccgttctccgattacgagtttcatttaaatcatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcatttaaatttccgaactctccaaggccctcgtcggaaaatcttcaaacctttcgtccgatccatcttgcaggctacctctcgaacgaactatcgcaagtctcttggccggccttgcgccttggc

SEQ ID NO: 18 - DNA, 7778 bp, synthetic construct
  source 1..7778: mol_type = other DNA
catagcttcaaaatgtttctactccttttttactcttccagattttctcggactccgcgcatcgccgtaccacttcaaaacacccaagcacagcatactaaatttcccctctttcttcctctagggtgtcgttaattacccgtactaaaggtttggaaaagaaaaaagagaccgcctcgtttctttttcttcgtcgaaaaaggcaataaaaatttttatcacgtttctttttcttgaaaattttttttttgatttttttctctttcgatgacctcccattgatatttaagttaataaacggtcttcaatttctcaagtttcagtttcatttttcttgttctattacaactttttttacttcttgctcattagaaagaaagcatagcaatctaatctaagagatctttgtcgatcctaccatccactcgacacacccgccagcggccgctgccaagcttccgagctctcgaattcaaaggaggtaccaaccatggggagatttccttcaatttttactgcagttttattcgcagcatcctccgcattagctgctccagtcaacactacaacagaagatgaaacggcacaaattccggctgaagctgtcatcggttacttagatttagaaggggatttcgatgttgctgttttgccattttccaacagcacaaataacgggttattgtttataaatactactattgccagcattgctgctaaagaagaaggggtatctttggataaaagagagggaagttcctatccctatgacgtgccagactacgcatccagcggaggaagttccgactcccctgatcgtccgtggaaccctccaaccttttccccggccttactagtagtaaccgagggtgacaacgcaacttttacctgttcattctctaacacaagcgagtcatttcacgtcgtatggcatagagaaagcccaagcggccaaaccgacacgctagcggccttccccgaagacaggtcacaaccaggacaagatgctagatttagagttacgcaactgcccaatggcagggatttccacatgtcagtggtgagagccaggagaaacgactcagggacgtatgtgtgcggcgtaatctctctggccccaaaaatccaaataaaagaatcattacgtgctgagttaagagttacagaacgtggcggcggaagtagcggtggcggaagcggtgggcagcccagggagcctcaagtctatacgctgcctccgtcaagagaagagatgactaagaaccaagtctctctaacttgtctagtaaaggggttttatccttcagatattgccgtcgagtgggagtcaaatgggcaaccggagaataattacaagacaactcctccggtcctagattccgatggcagtttcttcctttactcaaaactgactgttgataaaagccgttggcagcaaggaaatgttttttcttgtagcgtgatgcatgaagcgctgcataaccactatactcagaaatctttatccctgtcacctggcaaatgatctagaagttgtctcctcctgcactgactgactgatacaatcgatttctggatccgcaggcctctgctagcttgactgactgagatacagcgtaccttcagctcatcatgtaattagttatgtcacgcttacattcacgccctccccccacatccgctctaaccgaaaaggaaggagttagacaacctgaagtctaggtccctatttatttttttatagttatgttagtattaagaacgttatttatatttcaaatttttcttttttttctgtacagacgcgtgtacgcatgtaacattatactgaaaaccttgcttgagaaggttttgggacgctcgaaggctttaatttgcggccaaagcaagcaattcgccctatagtgagtcgtattacgcgcgctaaaacctctacaaatgtggtattggcccatctctatcggtatcgtagcataaccccttggggcctctaaacgggtcttgaggggttttttgtgcccctcgggccggattgctatctaccggcattggcgcagaaaaaaatgcctgatgcgacgctgcgcgtcttatactcccacatatgccagattcagcaacggatacggcttccccaacttgcccacttccatacgtgtcctccttaccagaaatttatccttaaggtcgtcagctatcctgcagggtgtttaaagattacggatatttaacttacttagaataatgccatttttttgagttataataatcctacgttagtgtgagcgggatttaaactgtgaggaccttaatacattcagacacttctgcggtatcaccctacttattcccttcgagattatatctaggaacccatcaggttggtggaagattacccgttctaagacttttcagcttcctctattgatgttacacctggacaccccttttctggcatccagtttttaatcttcagtggcatgtgagattctccgaaattaactaaagcaatcacacaattctctcggataccacctcggttgaaactgacaggtggtttgttacgcatgctaatgcaaaggagcctatatacctttggctcggctgctgtaacagggaatataaagggcagcataatttaggagtttagtgaacttgcaacatttactattttcccttcttacgtaaatatttttctttttaattctaaatcaatctttttcaattttttgtttgtattcttttcttgcttaaatctataactacaaaaaacacatacataaactaaaaatggccaaaaccttcaacatctctcagcaggatctggagctggtggaggtcgccactgagaagatcaccatgctctatgaggacaacaagcaccatgtcggggcggccatcaggaccaagactggggagatcatctctgctgtccacattgaagcctacattggcagggtcactgtctgtgctgaagccattgccattgggtctgctgtgagcaacgggcagaaggactttgacaccattgtggctgtcaggcacccctactctgatgaggtggacagatccatcagggtggtcagcccctgtggcatgtgtagagagctgatctctgactatgctcctgactgctttgtgctcattgagatgaatggcaagctggtcaaaaccaccattgaggaactcatccccctcaagtacaccaggaactaaggttaatataattatataaaaatattatcttcttttctttatatctagtgttatgtaaaataaattgatgactacggaaagcctttttatattgtttctttttcattctgagccacttaaatttcgtgaatgttcttgtaagggacggtagatttacaagtgatacaacaaaaagcaaggcgctttttctaataaaaagaagaaaagcatttaacaattgaacacctctatatcaacgaagaatattcctgcaggcgatctctcgatttcgatcaagacattcctttaatggtcttttctggacaccactaggggtcagaagtagttcatcaaactttcttccctccctaatctcattggttaccttgggctatcgaaacttaattaaccagtcaagtcagctacttggcgagatcgacttgtctgggtttcgactacgctcagaattgcgtcagtcaagttcgatctggtccttgctattgcacccgttctccgattacgagtttcatttaaatcatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcatttaaatttccgaactctccaaggccctcgtcggaaaatcttcaaacctttcgtccgatccatcttgcaggctacctctcgaacgaactatcgcaagtctcttggccggccttgcgccttggctattgcttggcagcgcctatcgccaggtattactccaatcccgaatatccgagatcgggatcacccgagagaagttcaacctacatcctcaatcccgatctatccgagatccgaggaatatcgaaatcggggcgcgccaacgaagcatctgtgcttcattttgtagaacaaaaatgcaacgcgagagcgctaatttttcaaacaaagaatctgagctgcatttttacagaacagaaatgcaacgcgaaagcgctattttaccaacgaagaatctgtgcttcatttttgtaaaacaaaaatgcaacgcgagagcgctaatttttcaaacaaagaatctgagctgcatttttacagaacagaaatgcaacgcgagagcgctattttaccaacaaagaatctatacttcttttttgttctacaaaaatgcatcccgagagcgctatttttctaacaaagcatcttagattactttttttctcctttgtgcgctctataatgcagtctcttgataactttttgcactgtaggtccgttaaggttagaagaaggctactttggtgtctattttctcttccataaaaaaagcctgactccacttcccgcgtttactgattactagcgaagctgcgggtgcattttttcaagataaaggcatccccgattatattctataccgatgtggattgcgcatactttgtgaacagaaagtgatagcgttgatgattcttcattggtcagaaaattatgaacggtttcttctattttgtctctatatactacgtataggaaatgtttacattttcgtattgttttcgattcactctatgaatagttcttactacaatttttttgtctaaagagtaatactagagataaacataaaaaatgtagaggtcgagtttagatgcaagttcaaggagcgaaaggtggatgggtaggttatatagggatatagcacagagatatatagcaaagagatacttttgagcaatgtttgtggaagcggtattcgcaatattttagtagctcgttacagtccggtgcgtttttggttttttgaaagtgcgtcttcagagcgcttttggttttcaaaagcgctctgaagttcctatacttcctagagaataggaacttcggaataggaacttcaaagcgtttccgaaaacgagcgcttccgaaaatgcaacgcgagctgcgcacatacagctcactgttcacgtcgcacctatatctgcgtgttgcctgtatatatatatacatgagaagaacggcatagtgcgtgtttatgcttaaatgcgtacttatatgcgtctatttatgtaggatgaaaggtagtctagtacctcctgtgatattatcccattccatgcggggtatcgtatgcttccttcagcactaccctttagctgttctatatgctgccactcctcaattggattagtctcatccttcaatgctatcatttcctttgatattggatcatacggcgcgcctggtgtaccgagaacgatcctctcagtgcgagtctcgacgatccatatcgttgcttggcagtcagccagtcggaatccagcttgggacccaggaagtccaatcgtcagatattgtactcaagcctggtcacggcagcgtaccgatctgtttaaacctagatattgatagtctgatcggtcaacgtataatcgagtcctagcttttgcaaacatctatcaagagacaggatcagcaggaggctttcgcatgattgaacaagatggattgcacgcaggttctccggcggcttgggtggagaggctattcggctatgactgggcacaacagacaatcggctgctctgatgccgccgtgttccggctgtcagcgcaggggcgtccggttctttttgtcaagaccgacctgtccggtgccctgaatgaactgcaagacgaggcagcgcggctatcgtggctggcgacgacgggcgttccttgcgcggctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgtctatgcccgacggcgaggatctcgtcgtgacccacggcgatgcctgcttgccgaatatcatggtggaaaatggccgcttttctggattcatcgactgtggccgtctgggtgtggcggaccgctatcaggacatagcgttggctacccgtgatattgctgaagagcttggcggcgaatgggctgaccgcttccttgtgctttacggtatcgccgcgcccgattcgcagcgcatcgccttctatcgccttcttgacgagttcttctgaccgattctaggtgcattggcgcagaaaaaaatgcctgatgcgacgctgcgcgtcttatactcccacatatgccagattcagcaacggatacggcttccccaacttgcccacttccatacgtgtcctccttaccagaaatttatccttaaggtcgtttaaactcgactctggctctatcgaatctccgtcgtttcgagcttacgcgaacagccgtggcgctcatttgctcgtcgggcatcgaatctcgtcagctatcgtcagcttacctttttggcagcgatcgccgcaattaaccctcactaaagggaacaaaagctggagctcggctcccgacatcttggaccattagctccacaggtatcttcttccctctagtggtcataacagcagcttcagctacctctcaatttcaatcgttgcgttacacacacaaaaaaccaacacacatccatcttcgatggatagcgattttattatctaactgctgatcgagtgtagccagatct

SEQ ID NO: 19 - DNA, 7574 bp, synthetic construct
  source 1..7574: mol_type = other DNA
tattgcttggcagcgcctatcgccaggtattactccaatcccgaatatccgagatcgggatcacccgagagaagttcaacctacatcctcaatcccgatctatccgagatccgaggaatatcgaaatcggggcgcgccaacgaagcatctgtgcttcattttgtagaacaaaaatgcaacgcgagagcgctaatttttcaaacaaagaatctgagctgcatttttacagaacagaaatgcaacgcgaaagcgctattttaccaacgaagaatctgtgcttcatttttgtaaaacaaaaatgcaacgcgagagcgctaatttttcaaacaaagaatctgagctgcatttttacagaacagaaatgcaacgcgagagcgctattttaccaacaaagaatctatacttcttttttgttctacaaaaatgcatcccgagagcgctatttttctaacaaagcatcttagattactttttttctcctttgtgcgctctataatgcagtctcttgataactttttgcactgtaggtccgttaaggttagaagaaggctactttggtgtctattttctcttccataaaaaaagcctgactccacttcccgcgtttactgattactagcgaagctgcgggtgcattttttcaagataaaggcatccccgattatattctataccgatgtggattgcgcatactttgtgaacagaaagtgatagcgttgatgattcttcattggtcagaaaattatgaacggtttcttctattttgtctctatatactacgtataggaaatgtttacattttcgtattgttttcgattcactctatgaatagttcttactacaatttttttgtctaaagagtaatactagagataaacataaaaaatgtagaggtcgagtttagatgcaagttcaaggagcgaaaggtggatgggtaggttatatagggatatagcacagagatatatagcaaagagatacttttgagcaatgtttgtggaagcggtattcgcaatattttagtagctcgttacagtccggtgcgtttttggttttttgaaagtgcgtcttcagagcgcttttggttttcaaaagcgctctgaagttcctatacttcctagagaataggaacttcggaataggaacttcaaagcgtttccgaaaacgagcgcttccgaaaatgcaacgcgagctgcgcacatacagctcactgttcacgtcgcacctatatctgcgtgttgcctgtatatatatatacatgagaagaacggcatagtgcgtgtttatgcttaaatgcgtacttatatgcgtctatttatgtaggatgaaaggtagtctagtacctcctgtgatattatcccattccatgcggggtatcgtatgcttccttcagcactaccctttagctgttctatatgctgccactcctcaattggattagtctcatccttcaatgctatcatttcctttgatattggatcatacggcgcgcctggtgtaccgagaacgatcctctcagtgcgagtctcgacgatccatatcgttgcttggcagtcagccagtcggaatccagcttgggacccaggaagtccaatcgtcagatattgtactcaagcctggtcacggcagcgtaccgatctgtttaaacctagatattgatagtctgatcggtcaacgtataatcgagtcctagcttttgcaaacatctatcaagagacaggatcagcaggaggctttcgcatgattgaacaagatggattgcacgcaggttctccggcggcttgggtggagaggctattcggctatgactgggcacaacagacaatcggctgctctgatgccgccgtgttccggctgtcagcgcaggggcgtccggttctttttgtcaagaccgacctgtccggtgccctgaatgaactgcaagacgaggcagcgcggctatcgtggctggcgacgacgggcgttccttgcgcggctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgtctatgcccgacggcgaggatctcgtcgtgacccacggcgatgcctgcttgccgaatatcatggtggaaaatggccgcttttctggattcatcgactgtggccgtctgggtgtggcggaccgctatcaggacatagcgttggctacccgtgatattgctgaagagcttggcggcgaatgggctgaccgcttccttgtgctttacggtatcgccgcgcccgattcgcagcgcatcgccttctatcgccttcttgacgagttcttctgaccgattctaggtgcattggcgcagaaaaaaatgcctgatgcgacgctgcgcgtcttatactcccacatatgccagattcagcaacggatacggcttccccaacttgcccacttccatacgtgtcctccttaccagaaatttatccttaaggtcgtttaaactcgactctggctctatcgaatctccgtcgtttcgagcttacgcgaacagccgtggcgctcatttgctcgtcgggcatcgaatctcgtcagctatcgtcagcttacctttttggcagcgatcgccgcaattaaccctcactaaagggaacaaaagctggagctcggctcccgacatcttggaccattagctccacaggtatcttcttccctctagtggtcataacagcagcttcagctacctctcaatttcaatcgttgcgttacacacacaaaaaaccaacacacatccatcttcgatggatagcgattttattatctaactgctgatcgagtgtagccagatctcatagcttcaaaatgtttctactccttttttactcttccagattttctcggactccgcgcatcgccgtaccacttcaaaacacccaagcacagcatactaaatttcccctctttcttcctctagggtgtcgttaattacccgtactaaaggtttggaaaagaaaaaagagaccgcctcgtttctttttcttcgtcgaaaaaggcaataaaaatttttatcacgtttctttttcttgaaaattttttttttgatttttttctctttcgatgacctcccattgatatttaagttaataaacggtcttcaatttctcaagtttcagtttcatttttcttgttctattacaactttttttacttcttgctcattagaaagaaagcatagcaatctaatctaagagatctttgtcgatcctaccatccactcgacacacccgccagcggccgctgccaagcttccgagctctcgaattcaaaggaggtaccaaccatgtttaccttccttaagatcatattatggttattttcccttgccttggcctcagcaggaagttcctatccctatgacgtgccagactacgcatccagcggaggaagttccgactcccctgatcgtccgtggaaccctccaaccttttccccggccttactagtagtaaccgagggtgacaacgcaacttttacctgttcattctctaacacaagcgagtcatttcacgtcgtatggcatagagaaagcccaagcggccaaaccgacacgctagcggccttccccgaagacaggtcacaaccaggacaagatgctagatttagagttacgcaactgcccaatggcagggatttccacatgtcagtggtgagagccaggagaaacgactcagggacgtatgtgtgcggcgtaatctctctggccccaaaaatccaaataaaagaatcattacgtgctgagttaagagttacagaacgtggcggcggaagtagcggtggcggaagcggtgggcagcccagggagcctcaagtctatacgctgcctccgtcaagagaagagatgactaagaaccaagtctctctaacttgtctagtaaaggggttttatccttcagatattgccgtcgagtgggagtcaaatgggcaaccggagaataattacaagacaactcctccggtcctagattccgatggcagtttcttcctttactcaaaactgactgttgataaaagccgttggcagcaaggaaatgttttttcttgtagcgtgatgcatgaagcgctgcataaccactatactcagaaatctttatccctgtcacctggcaaatgatctagaagttgtctcctcctgcactgactgactgatacaatcgatttctggatccgcaggcctctgctagcttgactgactgagatacagcgtaccttcagctcatcatgtaattagttatgtcacgcttacattcacgccctccccccacatccgctctaaccgaaaaggaaggagttagacaacctgaagtctaggtccctatttatttttttatagttatgttagtattaagaacgttatttatatttcaaatttttcttttttttctgtacagacgcgtgtacgcatgtaacattatactgaaaaccttgcttgagaaggttttgggacgctcgaaggctttaatttgcggccaaagcaagcaattcgccctatagtgagtcgtattacgcgcgctaaaacctctacaaatgtggtattggcccatctctatcggtatcgtagcataaccccttggggcctctaaacgggtcttgaggggttttttgtgcccctcgggccggattgctatctaccggcattggcgcagaaaaaaatgcctgatgcgacgctgcgcgtcttatactcccacatatgccagattcagcaacggatacggcttccccaacttgcccacttccatacgtgtcctccttaccagaaatttatccttaaggtcgtcagctatcctgcagggtgtttaaagattacggatatttaacttacttagaataatgccatttttttgagttataataatcctacgttagtgtgagcgggatttaaactgtgaggaccttaatacattcagacacttctgcggtatcaccctacttattcccttcgagattatatctaggaacccatcaggttggtggaagattacccgttctaagacttttcagcttcctctattgatgttacacctggacaccccttttctggcatccagtttttaatcttcagtggcatgtgagattctccgaaattaactaaagcaatcacacaattctctcggataccacctcggttgaaactgacaggtggtttgttacgcatgctaatgcaaaggagcctatatacctttggctcggctgctgtaacagggaatataaagggcagcataatttaggagtttagtgaacttgcaacatttactattttcccttcttacgtaaatatttttctttttaattctaaatcaatctttttcaattttttgtttgtattcttttcttgcttaaatctataactacaaaaaacacatacataaactaaaaatggccaaaaccttcaacatctctcagcaggatctggagctggtggaggtcgccactgagaagatcaccatgctctatgaggacaacaagcaccatgtcggggcggccatcaggaccaagactggggagatcatctctgctgtccacattgaagcctacattggcagggtcactgtctgtgctgaagccattgccattgggtctgctgtgagcaacgggcagaaggactttgacaccattgtggctgtcaggcacccctactctgatgaggtggacagatccatcagggtggtcagcccctgtggcatgtgtagagagctgatctctgactatgctcctgactgctttgtgctcattgagatgaatggcaagctggtcaaaaccaccattgaggaactcatccccctcaagtacaccaggaactaaggttaatataattatataaaaatattatcttcttttctttatatctagtgttatgtaaaataaattgatgactacggaaagcctttttatattgtttctttttcattctgagccacttaaatttcgtgaatgttcttgtaagggacggtagatttacaagtgatacaacaaaaagcaaggcgctttttctaataaaaagaagaaaagcatttaacaattgaacacctctatatcaacgaagaatattcctgcaggcgatctctcgatttcgatcaagacattcctttaatggtcttttctggacaccactaggggtcagaagtagttcatcaaactttcttccctccctaatctcattggttaccttgggctatcgaaacttaattaaccagtcaagtcagctacttggcgagatcgacttgtctgggtttcgactacgctcagaattgcgtcagtcaagttcgatctggtccttgctattgcacccgttctccgattacgagtttcatttaaatcatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcatttaaatttccgaactctccaaggccctcgtcggaaaatcttcaaacctttcgtccgatccatcttgcaggctacctctcgaacgaactatcgcaagtctcttggccggccttgcgccttggc

SEQ ID NO: 20 - DNA, 8084 bp, synthetic construct
  source 1..8084: mol_type = other DNA
atccttttgttgtttccgggtgtacaatatggacttcctcttttctggcaaccaaacccatacatcgggattcctataataccttcgttggtctccctaacatgtaggtggcggaggggagatatacaatagaacagataccagacaagacataatgggctaaacaagactacaccaattacactgcctcattgatggtggtacataacgaactaatactgtagccctagacttgatagccatcatcatatcgaagtttcactaccctttttccatttgccatctattgaagtaataataggcgcatgcaacttcttttctttttttttcttttctctctcccccgttgttgtctcaccatatccgcaatgacaaaaaaatgatggaagacactaaaggaaaaaattaacgacaaagacagcaccaacagatgtcgttgttccagagctgatgaggggtatctcgaagcacacgaaactttttccttccttcattcacgcacactactctctaatgagcaacggtatacggccttccttccagttacttgaatttgaaataaaaaaaagtttgctgtcttgctatcaagtataaatagacctgcaattattaatcttttgtttcctcgtcattgttctcgttccctttcttccttgtttctttttctgcacaatatttcaagctataccaagcatacaatcaactagatctttgtcgatcctaccatccactcgacacacccgccagcggccgctgccaagcttccgagctctcgaattcaaaggaggtaccaaccatggggagatttccttcaatttttactgcagttttattcgcagcatcctccgcattagctgctccagtcaacactacaacagaagatgaaacggcacaaattccggctgaagctgtcatcggttacttagatttagaaggggatttcgatgttgctgttttgccattttccaacagcacaaataacgggttattgtttataaatactactattgccagcattgctgctaaagaagaaggggtatctttggataaaagagagggaagttcctatccctatgacgtgccagactacgcatccagcggaggaagttccgactcccctgatcgtccgtggaaccctccaaccttttccccggccttactagtagtaaccgagggtgacaacgcaacttttacctgttcattctctaacacaagcgagtcatttcacgtcgtatggcatagagaaagcccaagcggccaaaccgacacgctagcggccttccccgaagacaggtcacaaccaggacaagatgctagatttagagttacgcaactgcccaatggcagggatttccacatgtcagtggtgagagccaggagaaacgactcagggacgtatgtgtgcggcgtaatctctctggccccaaaaatccaaataaaagaatcattacgtgctgagttaagagttacagaacgtggcggcggaagtagcggtggcggaagcggtgggcagcccagggagcctcaagtctatacgctgcctccgtcaagagaagagatgactaagaaccaagtctctctaacttgtctagtaaaggggttttatccttcagatattgccgtcgagtgggagtcaaatgggcaaccggagaataattacaagacaactcctccggtcctagattccgatggcagtttcttcctttactcaaaactgactgttgataaaagccgttggcagcaaggaaatgttttttcttgtagcgtgatgcatgaagcgctgcataaccactatactcagaaatctttatccctgtcacctggcaaatgatctagaagttgtctcctcctgcactgactgactgatacaatcgatttctggatccgcaggcctctgctagcttgactgactgagatacagcgtaccttcagctcatcatgtaattagttatgtcacgcttacattcacgccctccccccacatccgctctaaccgaaaaggaaggagttagacaacctgaagtctaggtccctatttatttttttatagttatgttagtattaagaacgttatttatatttcaaatttttcttttttttctgtacagacgcgtgtacgcatgtaacattatactgaaaaccttgcttgagaaggttttgggacgctcgaaggctttaatttgcggccaaagcaagcaattcgccctatagtgagtcgtattacgcgcgctaaaacctctacaaatgtggtattggcccatctctatcggtatcgtagcataaccccttggggcctctaaacgggtcttgaggggttttttgtgcccctcgggccggattgctatctaccggcattggcgcagaaaaaaatgcctgatgcgacgctgcgcgtcttatactcccacatatgccagattcagcaacggatacggcttccccaacttgcccacttccatacgtgtcctccttaccagaaatttatccttaaggtcgtcagctatcctgcagggtgtttaaagattacggatatttaacttacttagaataatgccatttttttgagttataataatcctacgttagtgtgagcgggatttaaactgtgaggaccttaatacattcagacacttctgcggtatcaccctacttattcccttcgagattatatctaggaacccatcaggttggtggaagattacccgttctaagacttttcagcttcctctattgatgttacacctggacaccccttttctggcatccagtttttaatcttcagtggcatgtgagattctccgaaattaactaaagcaatcacacaattctctcggataccacctcggttgaaactgacaggtggtttgttacgcatgctaatgcaaaggagcctatatacctttggctcggctgctgtaacagggaatataaagggcagcataatttaggagtttagtgaacttgcaacatttactattttcccttcttacgtaaatatttttctttttaattctaaatcaatctttttcaattttttgtttgtattcttttcttgcttaaatctataactacaaaaaacacatacataaactaaaaatggccaaaaccttcaacatctctcagcaggatctggagctggtggaggtcgccactgagaagatcaccatgctctatgaggacaacaagcaccatgtcggggcggccatcaggaccaagactggggagatcatctctgctgtccacattgaagcctacattggcagggtcactgtctgtgctgaagccattgccattgggtctgctgtgagcaacgggcagaaggactttgacaccattgtggctgtcaggcacccctactctgatgaggtggacagatccatcagggtggtcagcccctgtggcatgtgtagagagctgatctctgactatgctcctgactgctttgtgctcattgagatgaatggcaagctggtcaaaaccaccattgaggaactcatccccctcaagtacaccaggaactaaggttaatataattatataaaaatattatcttcttttctttatatctagtgttatgtaaaataaattgatgactacggaaagcctttttatattgtttctttttcattctgagccacttaaatttcgtgaatgttcttgtaagggacggtagatttacaagtgatacaacaaaaagcaaggcgctttttctaataaaaagaagaaaagcatttaacaattgaacacctctatatcaacgaagaatattcctgcaggcgatctctcgatttcgatcaagacattcctttaatggtcttttctggacaccactaggggtcagaagtagttcatcaaactttcttccctccctaatctcattggttaccttgggctatcgaaacttaattaaccagtcaagtcagctacttggcgagatcgacttgtctgggtttcgactacgctcagaattgcgtcagtcaagttcgatctggtccttgctattgcacccgttctccgattacgagtttcatttaaatcatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcatttaaatttccgaactctccaaggccctcgtcggaaaatcttcaaacctttcgtccgatccatcttgcaggctacctctcgaacgaactatcgcaagtctcttggccggccttgcgccttggctattgcttggcagcgcctatcgccaggtattactccaatcccgaatatccgagatcgggatcacccgagagaagttcaacctacatcctcaatcccgatctatccgagatccgaggaatatcgaaatcggggcgcgccaacgaagcatctgtgcttcattttgtagaacaaaaatgcaacgcgagagcgctaatttttcaaacaaagaatctgagctgcatttttacagaacagaaatgcaacgcgaaagcgctattttaccaacgaagaatctgtgcttcatttttgtaaaacaaaaatgcaacgcgagagcgctaatttttcaaacaaagaatctgagctgcatttttacagaacagaaatgcaacgcgagagcgctattttaccaacaaagaatctatacttcttttttgttctacaaaaatgcatcccgagagcgctatttttctaacaaagcatcttagattactttttttctcctttgtgcgctctataatgcagtctcttgataactttttgcactgtaggtccgttaaggttagaagaaggctactttggtgtctattttctcttccataaaaaaagcctgactccacttcccgcgtttactgattactagcgaagctgcgggtgcattttttcaagataaaggcatccccgattatattctataccgatgtggattgcgcatactttgtgaacagaaagtgatagcgttgatgattcttcattggtcagaaaattatgaacggtttcttctattttgtctctatatactacgtataggaaatgtttacattttcgtattgttttcgattcactctatgaatagttcttactacaatttttttgtctaaagagtaatactagagataaacataaaaaatgtagaggtcgagtttagatgcaagttcaaggagcgaaaggtggatgggtaggttatatagggatatagcacagagatatatagcaaagagatacttttgagcaatgtttgtggaagcggtattcgcaatattttagtagctcgttacagtccggtgcgtttttggttttttgaaagtgcgtcttcagagcgcttttggttttcaaaagcgctctgaagttcctatacttcctagagaataggaacttcggaataggaacttcaaagcgtttccgaaaacgagcgcttccgaaaatgcaacgcgagctgcgcacatacagctcactgttcacgtcgcacctatatctgcgtgttgcctgtatatatatatacatgagaagaacggcatagtgcgtgtttatgcttaaatgcgtacttatatgcgtctatttatgtaggatgaaaggtagtctagtacctcctgtgatattatcccattccatgcggggtatcgtatgcttccttcagcactaccctttagctgttctatatgctgccactcctcaattggattagtctcatccttcaatgctatcatttcctttgatattggatcatacggcgcgcctggtgtaccgagaacgatcctctcagtgcgagtctcgacgatccatatcgttgcttggcagtcagccagtcggaatccagcttgggacccaggaagtccaatcgtcagatattgtactcaagcctggtcacggcagcgtaccgatctgtttaaacctagatattgatagtctgatcggtcaacgtataatcgagtcctagcttttgcaaacatctatcaagagacaggatcagcaggaggctttcgcatgattgaacaagatggattgcacgcaggttctccggcggcttgggtggagaggctattcggctatgactgggcacaacagacaatcggctgctctgatgccgccgtgttccggctgtcagcgcaggggcgtccggttctttttgtcaagaccgacctgtccggtgccctgaatgaactgcaagacgaggcagcgcggctatcgtggctggcgacgacgggcgttccttgcgcggctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgtctatgcccgacggcgaggatctcgtcgtgacccacggcgatgcctgcttgccgaatatcatggtggaaaatggccgcttttctggattcatcgactgtggccgtctgggtgtggcggaccgctatcaggacatagcgttggctacccgtgatattgctgaagagcttggcggcgaatgggctgaccgcttccttgtgctttacggtatcgccgcgcccgattcgcagcgcatcgccttctatcgccttcttgacgagttcttctgaccgattctaggtgcattggcgcagaaaaaaatgcctgatgcgacgctgcgcgtcttatactcccacatatgccagattcagcaacggatacggcttccccaacttgcccacttccatacgtgtcctccttaccagaaatttatccttaaggtcgtttaaactcgactctggctctatcgaatctccgtcgtttcgagcttacgcgaacagccgtggcgctcatttgctcgtcgggcatcgaatctcgtcagctatcgtcagcttacctttttggcagcgatcgccgcaattaaccctcactaaagggaacaaaagctggagctcggctcccgacatcttggaccattagctccacaggtatcttcttccctctagtggtcataacagcagcttcagctacctctcaatttcaatcgttgcgttacacacacaaaaaaccaacacacatccatcttcgatggatagcgattttattatctaactgctgatcgagtgtagccagatct

SEQ ID NO: 21 - DNA, 8118 bp, synthetic construct
  source 1..8118: mol_type = other DNA
cgacacgcaaaagaaaaccttcgaggttgcgcacttcgcccacccatgaaccacacggttagtccaaaaggggcagttcagattccagatgcgggaattagcttgctgccaccctcacctcactaacgctgcggtgtgcggatacttcatgctatttatagacgcgcgtgtcggaatcagcacgcgcaagaaccaaatgggaaaatcggaatgggtccagaactgctttgagtgctggctattggcgtctgatttccgttttgggaatcctttgccgcgcgcccctctcaaaactccgcacaagtcccagaaagcgggaaagaaataaaacgccaccaaaaaaaaaaaaataaaagccaatcctcgaagcgtgggtggtaggccctggattatcccgtacaagtatttctcaggagtaaaaaaaccgtttgttttggaattccccatttcgcggccacctacgccgctatctttgcaacaactatctgcgataactcagcaaattttgcatattcgtgttgcagtattgcgataatgggagtcttacttccaacataacggcagaaagaaatgtgagaaaattttgcatcctttgcctccgttcaagtatataaagtcggcatgcttgataatctttctttccatcctacattgttctaattattcttattctcctttattctttcctaacataccaagaaattaatcttctgtcattcgcttaaacactatatcaataagatctttgtcgatcctaccatccactcgacacacccgccagcggccgctgccaagcttccgagctctcgaattcaaaggaggtaccaaccatggggagatttccttcaatttttactgcagttttattcgcagcatcctccgcattagctgctccagtcaacactacaacagaagatgaaacggcacaaattccggctgaagctgtcatcggttacttagatttagaaggggatttcgatgttgctgttttgccattttccaacagcacaaataacgggttattgtttataaatactactattgccagcattgctgctaaagaagaaggggtatctttggataaaagagagggaagttcctatccctatgacgtgccagactacgcatccagcggaggaagttccgactcccctgatcgtccgtggaaccctccaaccttttccccggccttactagtagtaaccgagggtgacaacgcaacttttacctgttcattctctaacacaagcgagtcatttcacgtcgtatggcatagagaaagcccaagcggccaaaccgacacgctagcggccttccccgaagacaggtcacaaccaggacaagatgctagatttagagttacgcaactgcccaatggcagggatttccacatgtcagtggtgagagccaggagaaacgactcagggacgtatgtgtgcggcgtaatctctctggccccaaaaatccaaataaaagaatcattacgtgctgagttaagagttacagaacgtggcggcggaagtagcggtggcggaagcggtgggcagcccagggagcctcaagtctatacgctgcctccgtcaagagaagagatgactaagaaccaagtctctctaacttgtctagtaaaggggttttatccttcagatattgccgtcgagtgggagtcaaatgggcaaccggagaataattacaagacaactcctccggtcctagattccgatggcagtttcttcctttactcaaaactgactgttgataaaagccgttggcagcaaggaaatgttttttcttgtagcgtgatgcatgaagcgctgcataaccactatactcagaaatctttatccctgtcacctggcaaatgatctagaagttgtctcctcctgcactgactgactgatacaatcgatttctggatccgcaggcctctgctagcttgactgactgagatacagcgtaccttcagctcatcatgtaattagttatgtcacgcttacattcacgccctccccccacatccgctctaaccgaaaaggaaggagttagacaacctgaagtctaggtccctatttatttttttatagttatgttagtattaagaacgttatttatatttcaaatttttcttttttttctgtacagacgcgtgtacgcatgtaacattatactgaaaaccttgcttgagaaggttttgggacgctcgaaggctttaatttgcggccaaagcaagcaattcgccctatagtgagtcgtattacgcgcgctaaaacctctacaaatgtggtattggcccatctctatcggtatcgtagcataaccccttggggcctctaaacgggtcttgaggggttttttgtgcccctcgggccggattgctatctaccggcattggcgcagaaaaaaatgcctgatgcgacgctgcgcgtcttatactcccacatatgccagattcagcaacggatacggcttccccaacttgcccacttccatacgtgtcctccttaccagaaatttatccttaaggtcgtcagctatcctgcagggtgtttaaagattacggatatttaacttacttagaataatgccatttttttgagttataataatcctacgttagtgtgagcgggatttaaactgtgaggaccttaatacattcagacacttctgcggtatcaccctacttattcccttcgagattatatctaggaacccatcaggttggtggaagattacccgttctaagacttttcagcttcctctattgatgttacacctggacaccccttttctggcatccagtttttaatcttcagtggcatgtgagattctccgaaattaactaaagcaatcacacaattctctcggataccacctcggttgaaactgacaggtggtttgttacgcatgctaatgcaaaggagcctatatacctttggctcggctgctgtaacagggaatataaagggcagcataatttaggagtttagtgaacttgcaacatttactattttcccttcttacgtaaatatttttctttttaattctaaatcaatctttttcaattttttgtttgtattcttttcttgcttaaatctataactacaaaaaacacatacataaactaaaaatggccaaaaccttcaacatctctcagcaggatctggagctggtggaggtcgccactgagaagatcaccatgctctatgaggacaacaagcaccatgtcggggcggccatcaggaccaagactggggagatcatctctgctgtccacattgaagcctacattggcagggtcactgtctgtgctgaagccattgccattgggtctgctgtgagcaacgggcagaaggactttgacaccattgtggctgtcaggcacccctactctgatgaggtggacagatccatcagggtggtcagcccctgtggcatgtgtagagagctgatctctgactatgctcctgactgctttgtgctcattgagatgaatggcaagctggtcaaaaccaccattgaggaactcatccccctcaagtacaccaggaactaaggttaatataattatataaaaatattatcttcttttctttatatctagtgttatgtaaaataaattgatgactacggaaagcctttttatattgtttctttttcattctgagccacttaaatttcgtgaatgttcttgtaagggacggtagatttacaagtgatacaacaaaaagcaaggcgctttttctaataaaaagaagaaaagcatttaacaattgaacacctctatatcaacgaagaatattcctgcaggcgatctctcgatttcgatcaagacattcctttaatggtcttttctggacaccactaggggtcagaagtagttcatcaaactttcttccctccctaatctcattggttaccttgggctatcgaaacttaattaaccagtcaagtcagctacttggcgagatcgacttgtctgggtttcgactacgctcagaattgcgtcagtcaagttcgatctggtccttgctattgcacccgttctccgattacgagtttcatttaaatcatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcatttaaatttccgaactctccaaggccctcgtcggaaaatcttcaaacctttcgtccgatccatcttgcaggctacctctcgaacgaactatcgcaagtctcttggccggccttgcgccttggctattgcttggcagcgcctatcgccaggtattactccaatcccgaatatccgagatcgggatcacccgagagaagttcaacctacatcctcaatcccgatctatccgagatccgaggaatatcgaaatcggggcgcgccaacgaagcatctgtgcttcattttgtagaacaaaaatgcaacgcgagagcgctaatttttcaaacaaagaatctgagctgcatttttacagaacagaaatgcaacgcgaaagcgctattttaccaacgaagaatctgtgcttcatttttgtaaaacaaaaatgcaacgcgagagcgctaatttttcaaacaaagaatctgagctgcatttttacagaacagaaatgcaacgcgagagcgctattttaccaacaaagaatctatacttcttttttgttctacaaaaatgcatcccgagagcgctatttttctaacaaagcatcttagattactttttttctcctttgtgcgctctataatgcagtctcttgataactttttgcactgtaggtccgttaaggttagaagaaggctactttggtgtctattttctcttccataaaaaaagcctgactccacttcccgcgtttactgattactagcgaagctgcgggtgcattttttcaagataaaggcatccccgattatattctataccgatgtggattgcgcatactttgtgaacagaaagtgatagcgttgatgattcttcattggtcagaaaattatgaacggtttcttctattttgtctctatatactacgtataggaaatgtttacattttcgtattgttttcgattcactctatgaatagttcttactacaatttttttgtctaaagagtaatactagagataaacataaaaaatgtagaggtcgagtttagatgcaagttcaaggagcgaaaggtggatgggtaggttatatagggatatagcacagagatatatagcaaagagatacttttgagcaatgtttgtggaagcggtattcgcaatattttagtagctcgttacagtccggtgcgtttttggttttttgaaagtgcgtcttcagagcgcttttggttttcaaaagcgctctgaagttcctatacttcctagagaataggaacttcggaataggaacttcaaagcgtttccgaaaacgagcgcttccgaaaatgcaacgcgagctgcgcacatacagctcactgttcacgtcgcacctatatctgcgtgttgcctgtatatatatatacatgagaagaacggcatagtgcgtgtttatgcttaaatgcgtacttatatgcgtctatttatgtaggatgaaaggtagtctagtacctcctgtgatattatcccattccatgcggggtatcgtatgcttccttcagcactaccctttagctgttctatatgctgccactcctcaattggattagtctcatccttcaatgctatcatttcctttgatattggatcatacggcgcgcctggtgtaccgagaacgatcctctcagtgcgagtctcgacgatccatatcgttgcttggcagtcagccagtcggaatccagcttgggacccaggaagtccaatcgtcagatattgtactcaagcctggtcacggcagcgtaccgatctgtttaaacctagatattgatagtctgatcggtcaacgtataatcgagtcctagcttttgcaaacatctatcaagagacaggatcagcaggaggctttcgcatgattgaacaagatggattgcacgcaggttctccggcggcttgggtggagaggctattcggctatgactgggcacaacagacaatcggctgctctgatgccgccgtgttccggctgtcagcgcaggggcgtccggttctttttgtcaagaccgacctgtccggtgccctgaatgaactgcaagacgaggcagcgcggctatcgtggctggcgacgacgggcgttccttgcgcggctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgtctatgcccgacggcgaggatctcgtcgtgacccacggcgatgcctgcttgccgaatatcatggtggaaaatggccgcttttctggattcatcgactgtggccgtctgggtgtggcggaccgctatcaggacatagcgttggctacccgtgatattgctgaagagcttggcggcgaatgggctgaccgcttccttgtgctttacggtatcgccgcgcccgattcgcagcgcatcgccttctatcgccttcttgacgagttcttctgaccgattctaggtgcattggcgcagaaaaaaatgcctgatgcgacgctgcgcgtcttatactcccacatatgccagattcagcaacggatacggcttccccaacttgcccacttccatacgtgtcctccttaccagaaatttatccttaaggtcgtttaaactcgactctggctctatcgaatctccgtcgtttcgagcttacgcgaacagccgtggcgctcatttgctcgtcgggcatcgaatctcgtcagctatcgtcagcttacctttttggcagcgatcgccgcaattaaccctcactaaagggaacaaaagctggagctcggctcccgacatcttggaccattagctccacaggtatcttcttccctctagtggtcataacagcagcttcagctacctctcaatttcaatcgttgcgttacacacacaaaaaaccaacacacatccatcttcgatggatagcgattttattatctaactgctgatcgagtgtagccagatct

SEQ ID NO: 22 - DNA, 7962 bp, synthetic construct
  source 1..7962: mol_type = other DNA
tcattatcaatactcgccatttcaaagaatacgtaaataattaatagtagtgattttcctaactttatttagtcaaaaaattagccttttaattctgctgtaacccgtacatgcccaaaatagggggcgggttacacagaatatataacatcgtaggtgtctgggtgaacagtttattcctggcatccactaaatataatggagcccgctttttaagctggcatccagaaaaaaaaagaatcccagcaccaaaatattgttttcttcaccaaccatcagttcataggtccattctcttagcgcaactacagagaacaggggcacaaacaggcaaaaaacgggcacaacctcaatggagtgatgcaacctgcctggagtaaatgatgacacaaggcaattgacccacgcatgtatctatctcattttcttacaccttctattaccttctgctctctctgatttggaaaaagctgaaaaaaaaggttgaaaccagttccctgaaattattcccctacttgactaataagtatataaagacggtaggtattgattgtaattctgtaaatctatttcttaaacttctagatctttgtcgatcctaccatccactcgacacacccgccagcggccgctgccaagcttccgagctctcgaattcaaaggaggtaccaaccatggggagatttccttcaatttttactgcagttttattcgcagcatcctccgcattagctgctccagtcaacactacaacagaagatgaaacggcacaaattccggctgaagctgtcatcggttacttagatttagaaggggatttcgatgttgctgttttgccattttccaacagcacaaataacgggttattgtttataaatactactattgccagcattgctgctaaagaagaaggggtatctttggataaaagagagggaagttcctatccctatgacgtgccagactacgcatccagcggaggaagttccgactcccctgatcgtccgtggaaccctccaaccttttccccggccttactagtagtaaccgagggtgacaacgcaacttttacctgttcattctctaacacaagcgagtcatttcacgtcgtatggcatagagaaagcccaagcggccaaaccgacacgctagcggccttccccgaagacaggtcacaaccaggacaagatgctagatttagagttacgcaactgcccaatggcagggatttccacatgtcagtggtgagagccaggagaaacgactcagggacgtatgtgtgcggcgtaatctctctggccccaaaaatccaaataaaagaatcattacgtgctgagttaagagttacagaacgtggcggcggaagtagcggtggcggaagcggtgggcagcccagggagcctcaagtctatacgctgcctccgtcaagagaagagatgactaagaaccaagtctctctaacttgtctagtaaaggggttttatccttcagatattgccgtcgagtgggagtcaaatgggcaaccggagaataattacaagacaactcctccggtcctagattccgatggcagtttcttcctttactcaaaactgactgttgataaaagccgttggcagcaaggaaatgttttttcttgtagcgtgatgcatgaagcgctgcataaccactatactcagaaatctttatccctgtcacctggcaaatgatctagaagttgtctcctcctgcactgactgactgatacaatcgatttctggatccgcaggcctctgctagcttgactgactgagatacagcgtaccttcagctcatcatgtaattagttatgtcacgcttacattcacgccctccccccacatccgctctaaccgaaaaggaaggagttagacaacctgaagtctaggtccctatttatttttttatagttatgttagtattaagaacgttatttatatttcaaatttttcttttttttctgtacagacgcgtgtacgcatgtaacattatactgaaaaccttgcttgagaaggttttgggacgctcgaaggctttaatttgcggccaaagcaagcaattcgccctatagtgagtcgtattacgcgcgctaaaacctctacaaatgtggtattggcccatctctatcggtatcgtagcataaccccttggggcctctaaacgggtcttgaggggttttttgtgcccctcgggccggattgctatctaccggcattggcgcagaaaaaaatgcctgatgcgacgctgcgcgtcttatactcccacatatgccagattcagcaacggatacggcttccccaacttgcccacttccatacgtgtcctccttaccagaaatttatccttaaggtcgtcagctatcctgcagggtgtttaaagattacggatatttaacttacttagaataatgccatttttttgagttataataatcctacgttagtgtgagcgggatttaaactgtgaggaccttaatacattcagacacttctgcggtatcaccctacttattcccttcgagattatatctaggaacccatcaggttggtggaagattacccgttctaagacttttcagcttcctctattgatgttacacctggacaccccttttctggcatccagtttttaatcttcagtggcatgtgagattctccgaaattaactaaagcaatcacacaattctctcggataccacctcggttgaaactgacaggtggtttgttacgcatgctaatgcaaaggagcctatatacctttggctcggctgctgtaacagggaatataaagggcagcataatttaggagtttagtgaacttgcaacatttactattttcccttcttacgtaaatatttttctttttaattctaaatcaatctttttcaattttttgtttgtattcttttcttgcttaaatctataactacaaaaaacacatacataaactaaaaatggccaaaaccttcaacatctctcagcaggatctggagctggtggaggtcgccactgagaagatcaccatgctctatgaggacaacaagcaccatgtcggggcggccatcaggaccaagactggggagatcatctctgctgtccacattgaagcctacattggcagggtcactgtctgtgctgaagccattgccattgggtctgctgtgagcaacgggcagaaggactttgacaccattgtggctgtcaggcacccctactctgatgaggtggacagatccatcagggtggtcagcccctgtggcatgtgtagagagctgatctctgactatgctcctgactgctttgtgctcattgagatgaatggcaagctggtcaaaaccaccattgaggaactcatccccctcaagtacaccaggaactaaggttaatataattatataaaaatattatcttcttttctttatatctagtgttatgtaaaataaattgatgactacggaaagcctttttatattgtttctttttcattctgagccacttaaatttcgtgaatgttcttgtaagggacggtagatttacaagtgatacaacaaaaagcaaggcgctttttctaataaaaagaagaaaagcatttaacaattgaacacctctatatcaacgaagaatattcctgcaggcgatctctcgatttcgatcaagacattcctttaatggtcttttctggacaccactaggggtcagaagtagttcatcaaactttcttccctccctaatctcattggttaccttgggctatcgaaacttaattaaccagtcaagtcagctacttggcgagatcgacttgtctgggtttcgactacgctcagaattgcgtcagtcaagttcgatctggtccttgctattgcacccgttctccgattacgagtttcatttaaatcatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcatttaaatttccgaactctccaaggccctcgtcggaaaatcttcaaacctttcgtccgatccatcttgcaggctacctctcgaacgaactatcgcaagtctcttggccggccttgcgccttggctattgcttggcagcgcctatcgccaggtattactccaatcccgaatatccgagatcgggatcacccgagagaagttcaacctacatcctcaatcccgatctatccgagatccgaggaatatcgaaatcggggcgcgccaacgaagcatctgtgcttcattttgtagaacaaaaatgcaacgcgagagcgctaatttttcaaacaaagaatctgagctgcatttttacagaacagaaatgcaacgcgaaagcgctattttaccaacgaagaatctgtgcttcatttttgtaaaacaaaaatgcaacgcgagagcgctaatttttcaaacaaagaatctgagctgcatttttacagaacagaaatgcaacgcgagagcgctattttaccaacaaagaatctatacttcttttttgttctacaaaaatgcatcccgagagcgctatttttctaacaaagcatcttagattactttttttctcctttgtgcgctctataatgcagtctcttgataactttttgcactgtaggtccgttaaggttagaagaaggctactttggtgtctattttctcttccataaaaaaagcctgactccacttcccgcgtttactgattactagcgaagctgcgggtgcattttttcaagataaaggcatccccgattatattctataccgatgtggattgcgcatactttgtgaacagaaagtgatagcgttgatgattcttcattggtcagaaaattatgaacggtttcttctattttgtctctatatactacgtataggaaatgtttacattttcgtattgttttcgattcactctatgaatagttcttactacaatttttttgtctaaagagtaatactagagataaacataaaaaatgtagaggtcgagtttagatgcaagttcaaggagcgaaaggtggatgggtaggttatatagggatatagcacagagatatatagcaaagagatacttttgagcaatgtttgtggaagcggtattcgcaatattttagtagctcgttacagtccggtgcgtttttggttttttgaaagtgcgtcttcagagcgcttttggttttcaaaagcgctctgaagttcctatacttcctagagaataggaacttcggaataggaacttcaaagcgtttccgaaaacgagcgcttccgaaaatgcaacgcgagctgcgcacatacagctcactgttcacgtcgcacctatatctgcgtgttgcctgtatatatatatacatgagaagaacggcatagtgcgtgtttatgcttaaatgcgtacttatatgcgtctatttatgtaggatgaaaggtagtctagtacctcctgtgatattatcccattccatgcggggtatcgtatgcttccttcagcactaccctttagctgttctatatgctgccactcctcaattggattagtctcatccttcaatgctatcatttcctttgatattggatcatacggcgcgcctggtgtaccgagaacgatcctctcagtgcgagtctcgacgatccatatcgttgcttggcagtcagccagtcggaatccagcttgggacccaggaagtccaatcgtcagatattgtactcaagcctggtcacggcagcgtaccgatctgtttaaacctagatattgatagtctgatcggtcaacgtataatcgagtcctagcttttgcaaacatctatcaagagacaggatcagcaggaggctttcgcatgattgaacaagatggattgcacgcaggttctccggcggcttgggtggagaggctattcggctatgactgggcacaacagacaatcggctgctctgatgccgccgtgttccggctgtcagcgcaggggcgtccggttctttttgtcaagaccgacctgtccggtgccctgaatgaactgcaagacgaggcagcgcggctatcgtggctggcgacgacgggcgttccttgcgcggctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgtctatgcccgacggcgaggatctcgtcgtgacccacggcgatgcctgcttgccgaatatcatggtggaaaatggccgcttttctggattcatcgactgtggccgtctgggtgtggcggaccgctatcaggacatagcgttggctacccgtgatattgctgaagagcttggcggcgaatgggctgaccgcttccttgtgctttacggtatcgccgcgcccgattcgcagcgcatcgccttctatcgccttcttgacgagttcttctgaccgattctaggtgcattggcgcagaaaaaaatgcctgatgcgacgctgcgcgtcttatactcccacatatgccagattcagcaacggatacggcttccccaacttgcccacttccatacgtgtcctccttaccagaaatttatccttaaggtcgtttaaactcgactctggctctatcgaatctccgtcgtttcgagcttacgcgaacagccgtggcgctcatttgctcgtcgggcatcgaatctcgtcagctatcgtcagcttacctttttggcagcgatcgccgcaattaaccctcactaaagggaacaaaagctggagctcggctcccgacatcttggaccattagctccacaggtatcttcttccctctagtggtcataacagcagcttcagctacctctcaatttcaatcgttgcgttacacacacaaaaaaccaacacacatccatcttcgatggatagcgattttattatctaactgctgatcgagtgtagccagatct

SEQ ID NO: 23 - DNA, 8082 bp, synthetic construct
  source 1..8082: mol_type = other DNA
tgtattctgatagtatgtgtttgtgtatgttaaagatgtttcttgatgttttttcttctataaaacaagagaagtaaaagactgaacacttcgaattgaatattaccaaacttcttattacatttatatatgatatcaagaacttgttaaacacgccaggcttgacctcgaacggtgttttgatgtgttactcaaccgttaaatcgctgtttgagctgactaacaaggttgcttgtcttgaatccccctcaccccgaatttattacgaatttgcccacatggtcggtgctatatcgcattcgttgcactcaccgttcccaagaggagatcaatgttacatacgttcccgatgcccaaaacggtaaagttaaagcgatatagaagctggatgcatatctttttacacgtccgcggcgcgcccaccctcttttttctttctggttccttttcggtcgtttttcttttgtcggctgccttattcccgtggggatgaggcgagcgccgtttcggcttggatacgccatatgcaacaatgacgtcttggtgctggccatgcccctcatttatgtcgaaaaacaggccactgcccctcttttctgcggcccgggcaagaggagacttcatcagccacggcgacgtgctggttgcttctacaatggtatcttcaccttttacataatccaattctatcatgcccttacgtagatctttgtcgatcctaccatccactcgacacacccgccagcggccgctgccaagcttccgagctctcgaattcaaaggaggtaccaaccatggggagatttccttcaatttttactgcagttttattcgcagcatcctccgcattagctgctccagtcaacactacaacagaagatgaaacggcacaaattccggctgaagctgtcatcggttacttagatttagaaggggatttcgatgttgctgttttgccattttccaacagcacaaataacgggttattgtttataaatactactattgccagcattgctgctaaagaagaaggggtatctttggataaaagagagggaagttcctatccctatgacgtgccagactacgcatccagcggaggaagttccgactcccctgatcgtccgtggaaccctccaaccttttccccggccttactagtagtaaccgagggtgacaacgcaacttttacctgttcattctctaacacaagcgagtcatttcacgtcgtatggcatagagaaagcccaagcggccaaaccgacacgctagcggccttccccgaagacaggtcacaaccaggacaagatgctagatttagagttacgcaactgcccaatggcagggatttccacatgtcagtggtgagagccaggagaaacgactcagggacgtatgtgtgcggcgtaatctctctggccccaaaaatccaaataaaagaatcattacgtgctgagttaagagttacagaacgtggcggcggaagtagcggtggcggaagcggtgggcagcccagggagcctcaagtctatacgctgcctccgtcaagagaagagatgactaagaaccaagtctctctaacttgtctagtaaaggggttttatccttcagatattgccgtcgagtgggagtcaaatgggcaaccggagaataattacaagacaactcctccggtcctagattccgatggcagtttcttcctttactcaaaactgactgttgataaaagccgttggcagcaaggaaatgttttttcttgtagcgtgatgcatgaagcgctgcataaccactatactcagaaatctttatccctgtcacctggcaaatgatctagaagttgtctcctcctgcactgactgactgatacaatcgatttctggatccgcaggcctctgctagcttgactgactgagatacagcgtaccttcagctcatcatgtaattagttatgtcacgcttacattcacgccctccccccacatccgctctaaccgaaaaggaaggagttagacaacctgaagtctaggtccctatttatttttttatagttatgttagtattaagaacgttatttatatttcaaatttttcttttttttctgtacagacgcgtgtacgcatgtaacattatactgaaaaccttgcttgagaaggttttgggacgctcgaaggctttaatttgcggccaaagcaagcaattcgccctatagtgagtcgtattacgcgcgctaaaacctctacaaatgtggtattggcccatctctatcggtatcgtagcataaccccttggggcctctaaacgggtcttgaggggttttttgtgcccctcgggccggattgctatctaccggcattggcgcagaaaaaaatgcctgatgcgacgctgcgcgtcttatactcccacatatgccagattcagcaacggatacggcttccccaacttgcccacttccatacgtgtcctccttaccagaaatttatccttaaggtcgtcagctatcctgcagggtgtttaaagattacggatatttaacttacttagaataatgccatttttttgagttataataatcctacgttagtgtgagcgggatttaaactgtgaggaccttaatacattcagacacttctgcggtatcaccctacttattcccttcgagattatatctaggaacccatcaggttggtggaagattacccgttctaagacttttcagcttcctctattgatgttacacctggacaccccttttctggcatccagtttttaatcttcagtggcatgtgagattctccgaaattaactaaagcaatcacacaattctctcggataccacctcggttgaaactgacaggtggtttgttacgcatgctaatgcaaaggagcctatatacctttggctcggctgctgtaacagggaatataaagggcagcataatttaggagtttagtgaacttgcaacatttactattttcccttcttacgtaaatatttttctttttaattctaaatcaatctttttcaattttttgtttgtattcttttcttgcttaaatctataactacaaaaaacacatacataaactaaaaatggccaaaaccttcaacatctctcagcaggatctggagctggtggaggtcgccactgagaagatcaccatgctctatgaggacaacaagcaccatgtcggggcggccatcaggaccaagactggggagatcatctctgctgtccacattgaagcctacattggcagggtcactgtctgtgctgaagccattgccattgggtctgctgtgagcaacgggcagaaggactttgacaccattgtggctgtcaggcacccctactctgatgaggtggacagatccatcagggtggtcagcccctgtggcatgtgtagagagctgatctctgactatgctcctgactgctttgtgctcattgagatgaatggcaagctggtcaaaaccaccattgaggaactcatccccctcaagtacaccaggaactaaggttaatataattatataaaaatattatcttcttttctttatatctagtgttatgtaaaataaattgatgactacggaaagcctttttatattgtttctttttcattctgagccacttaaatttcgtgaatgttcttgtaagggacggtagatttacaagtgatacaacaaaaagcaaggcgctttttctaataaaaagaagaaaagcatttaacaattgaacacctctatatcaacgaagaatattcctgcaggcgatctctcgatttcgatcaagacattcctttaatggtcttttctggacaccactaggggtcagaagtagttcatcaaactttcttccctccctaatctcattggttaccttgggctatcgaaacttaattaaccagtcaagtcagctacttggcgagatcgacttgtctgggtttcgactacgctcagaattgcgtcagtcaagttcgatctggtccttgctattgcacccgttctccgattacgagtttcatttaaatcatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcatttaaatttccgaactctccaaggccctcgtcggaaaatcttcaaacctttcgtccgatccatcttgcaggctacctctcgaacgaactatcgcaagtctcttggccggccttgcgccttggctattgcttggcagcgcctatcgccaggtattactccaatcccgaatatccgagatcgggatcacccgagagaagttcaacctacatcctcaatcccgatctatccgagatccgaggaatatcgaaatcggggcgcgccaacgaagcatctgtgcttcattttgtagaacaaaaatgcaacgcgagagcgctaatttttcaaacaaagaatctgagctgcatttttacagaacagaaatgcaacgcgaaagcgctattttaccaacgaagaatctgtgcttcatttttgtaaaacaaaaatgcaacgcgagagcgctaatttttcaaacaaagaatctgagctgcatttttacagaacagaaatgcaacgcgagagcgctattttaccaacaaagaatctatacttcttttttgttctacaaaaatgcatcccgagagcgctatttttctaacaaagcatcttagattactttttttctcctttgtgcgctctataatgcagtctcttgataactttttgcactgtaggtccgttaaggttagaagaaggctactttggtgtctattttctcttccataaaaaaagcctgactccacttcccgcgtttactgattactagcgaagctgcgggtgcattttttcaagataaaggcatccccgattatattctataccgatgtggattgcgcatactttgtgaacagaaagtgatagcgttgatgattcttcattggtcagaaaattatgaacggtttcttctattttgtctctatatactacgtataggaaatgtttacattttcgtattgttttcgattcactctatgaatagttcttactacaatttttttgtctaaagagtaatactagagataaacataaaaaatgtagaggtcgagtttagatgcaagttcaaggagcgaaaggtggatgggtaggttatatagggatatagcacagagatatatagcaaagagatacttttgagcaatgtttgtggaagcggtattcgcaatattttagtagctcgttacagtccggtgcgtttttggttttttgaaagtgcgtcttcagagcgcttttggttttcaaaagcgctctgaagttcctatacttcctagagaataggaacttcggaataggaacttcaaagcgtttccgaaaacgagcgcttccgaaaatgcaacgcgagctgcgcacatacagctcactgttcacgtcgcacctatatctgcgtgttgcctgtatatatatatacatgagaagaacggcatagtgcgtgtttatgcttaaatgcgtacttatatgcgtctatttatgtaggatgaaaggtagtctagtacctcctgtgatattatcccattccatgcggggtatcgtatgcttccttcagcactaccctttagctgttctatatgctgccactcctcaattggattagtctcatccttcaatgctatcatttcctttgatattggatcatacggcgcgcctggtgtaccgagaacgatcctctcagtgcgagtctcgacgatccatatcgttgcttggcagtcagccagtcggaatccagcttgggacccaggaagtccaatcgtcagatattgtactcaagcctggtcacggcagcgtaccgatctgtttaaacctagatattgatagtctgatcggtcaacgtataatcgagtcctagcttttgcaaacatctatcaagagacaggatcagcaggaggctttcgcatgattgaacaagatggattgcacgcaggttctccggcggcttgggtggagaggctattcggctatgactgggcacaacagacaatcggctgctctgatgccgccgtgttccggctgtcagcgcaggggcgtccggttctttttgtcaagaccgacctgtccggtgccctgaatgaactgcaagacgaggcagcgcggctatcgtggctggcgacgacgggcgttccttgcgcggctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgtctatgcccgacggcgaggatctcgtcgtgacccacggcgatgcctgcttgccgaatatcatggtggaaaatggccgcttttctggattcatcgactgtggccgtctgggtgtggcggaccgctatcaggacatagcgttggctacccgtgatattgctgaagagcttggcggcgaatgggctgaccgcttccttgtgctttacggtatcgccgcgcccgattcgcagcgcatcgccttctatcgccttcttgacgagttcttctgaccgattctaggtgcattggcgcagaaaaaaatgcctgatgcgacgctgcgcgtcttatactcccacatatgccagattcagcaacggatacggcttccccaacttgcccacttccatacgtgtcctccttaccagaaatttatccttaaggtcgtttaaactcgactctggctctatcgaatctccgtcgtttcgagcttacgcgaacagccgtggcgctcatttgctcgtcgggcatcgaatctcgtcagctatcgtcagcttacctttttggcagcgatcgccgcaattaaccctcactaaagggaacaaaagctggagctcggctcccgacatcttggaccattagctccacaggtatcttcttccctctagtggtcataacagcagcttcagctacctctcaatttcaatcgttgcgttacacacacaaaaaaccaacacacatccatcttcgatggatagcgattttattatctaactgctgatcgagtgtagccagatct

SEQ ID NO: 24 - DNA, 22 bp, synthetic construct
  source 1..22: mol_type = other DNA
catatcacataggaagcaacag

SEQ ID NO: 25 - DNA, 21 bp, synthetic construct
  source 1..21: mol_type = other DNA
ctacgataccgatagagatgg

(19) United States
(12) Patent Application Publication
(10) Pub. No.: US 2026/0124260 A1
(43) Pub. Date: May 7, 2026

(54) ENGINEERED YEAST FOR DELIVERY OF IMMUNE CHECKPOINT INHIBITOR PROTEINS TO GASTROINTESTINAL TUMORS

(71) Applicant: Washington University, St. Louis, MO (US)

(72) Inventors: Miranda Wallace, St. Louis, MO (US); Beth Helmink, Houston, TX (US); Olivia Gorushi, St. Louis, MO (US); Gautam Dantas, St. Louis, MO (US); Jerome Prusa, St. Louis, MO (US); Suryang Kwak, Seoul (KR)

(73) Assignee: Washington University, St. Louis, MO (US)

(21) Appl. No.: 19/336,492

(22) Filed: Sep. 22, 2025

Related U.S. Application Data
(60) Provisional application No. 63/697,214, filed on Sep. 20, 2024.

(51) Int. Cl. A61K 36/064; A61K 38/17; A61P 35/00

(52) U.S. Cl. CPC A61K 36/064; A61K 38/1774; A61P 35/00

(57) ABSTRACT

Among the various aspects of the present disclosure is the provision of an engineered yeast for delivery of immune checkpoint inhibitor (ICI) proteins to gastrointestinal tumors. The present teachings include methods for suppressing or reducing tumors through the administration of an engineered Sb that can deliver immune checkpoint inhibitors (ICIs) into the gastrointestinal tract. The present teachings also include a composition of a genetically engineered Sb consisting of Sb capable of expressing and secreting ICIs.

### CROSS-REFERENCE TO RELATED APPLICATIONS

This application claims the benefit of priority to U.S. Provisional Application Ser. No. 63/697,214 filed on Sep. 20, 2024, which is incorporated herein by reference in its entirety.

### STATEMENT REGARDING FEDERALLY SPONSORED RESEARCH OR DEVELOPMENT

This invention was made with government support under A1155893 and AT009741 awarded by the National Institutes of Health. The government has certain rights in the invention.

### MATERIAL INCORPORATED-BY-REFERENCE

The Sequence Listing, which is a part of the present disclosure, includes a computer readable document entitled “020850-US-NP_SEQ_LISTING.XML” (202,665 bytes) created Sep. 22, 2025 comprising nucleotide and/or amino acid sequences of the present invention. The subject matter of the Sequence Listing is incorporated herein by reference in its entirety.

### FIELD OF THE INVENTION

The present disclosure generally relates to engineered yeast that produce therapeutic proteins to treat GI tumors.

### BACKGROUND OF THE INVENTION

Microbes have an increasingly appreciated role in the development and treatment of a myriad of diseases. Although beneficial microbes in their native form have demonstrated clinical utility, such as probiotic formulations and fecal microbiota transplantation, engineered probiotics have been developed in recent years as living carriers of therapeutics that treat a variety of diseases. As many probiotics are capable of heterologous production of various functional therapies, are genetically tractable, and are safe and viable following oral administration, diseases manifesting in the mammalian gastrointestinal (GI) tract are an attractive target for engineered probiotics. Protein-based therapies, or ‘biologics’, represent a recently growing market as exemplified by monoclonal antibodies used for anticancer immunotherapy, GI infections, and inflammatory bowel disease. Engineered probiotics are a promising solution to achieve delivery of protein-based therapies to disease sites in the GI tract following oral administration, as protein-based therapies typically have poor oral bioavailability in the lower GI tract.

The probiotic yeast Saccharomyces cerevisiae var. boulardii (Sb) is a strain of Saccharomyces cerevisiae, a model yeast with a wide array of available genetic tools. Sb has a favorable safety profile in humans and can be used to treat diarrhea associated with antibiotic use or infectious disease. Like bacterial probiotics, Sb is capable of robust heterologous protein expression and can survive the harsh environment of the mammalian GI tract. Sb can readily secrete therapeutic payloads into the extracellular space in appreciable quantities, has a short gut transit time, and does not innately colonize the mammalian GI tract, allowing for precise therapeutic regimens without the need for antibiotics or synthetic kill-switches upon completion of treatment. As a yeast, Sb has decreased risk of horizontal gene transfer with the host microbiome. As such, it is less likely to acquire antibiotic resistance genes or virulence factors that could endanger the host in comparison to bacterial probiotics. Lastly, Sb possesses innate therapeutic activity against GI cancers and other GI diseases. Together, these qualities make Sb exceptionally well-suited as a therapeutic delivery chassis to treat diseases of the GI tract.

GI cancers are a leading cause of cancer-related mortality worldwide, with colorectal cancer (CRC) accounting for almost two million new cases and nearly one million deaths annually. Immune checkpoint inhibitors (ICIs) have revolutionized the cancer therapy landscape and are highly efficacious in a subset of CRC cases. They work by blocking immunosuppressive pathways exploited by tumors, targeting the programmed cell death protein 1 (PD-1)/programmed cell death ligand 1 (PD-L1) axis or cytotoxic T-lymphocyte-associated protein 4 (CTLA-4). In recent years, “miniature” antibody variants (termed microbodies and nanobodies) have been developed that maintain the functional components required to bind to their target and perform immune checkpoint blockade with improved tissue diffusion and tumor penetration. Alternative delivery strategies utilizing microbes as delivery vessels for ICI variants could further improve ICI efficacy and may expand the subset of patients that benefit from immunotherapies.

Current pre-clinical efforts to develop engineered anticancer probiotics have focused largely on engineering bacterial chassis that are administered systemically or intratumorally. Although previous studies have established the utility of engineered Saccharomyces yeasts for GI-directed therapies to treat diseases such as Clostridioides difficile infection, inflammatory bowel disease, and obesity, the potential for probiotic yeasts as a carrier for gut-directed, anticancer therapies is yet to be reported. Furthermore, although the growth and viability of Sb in the harsh and distinct conditions of the GI tract have been extensively characterized, a robust analysis of the performance of secretion circuits in Sb in the context of gut-relevant conditions would benefit efforts to develop GI-directed therapeutic delivery circuits in yeast.

### SUMMARY OF THE INVENTION

Among the various aspects of the present disclosure is the provision of an engineered yeast used to produce and deliver engineered proteins that function as immune checkpoint inhibitor (ICI) proteins for the treatment of gastrointestinal (GI) tumors.

Briefly, therefore, the present disclosure is directed to methods and compositions related to treating a GI disorder by delivery of ICIs from engineered yeast.

The present teachings include a composition to suppress or reduce tumors in a patient. The composition includes an engineered yeast containing a nucleotide sequence encoding a high-affinity programmed cell death 1 ectodomain protein (haPD-1). The engineered yeast is configured to secrete the haPD-1. In some aspects, the engineered yeast is a yeast species consisting of Saccharomyces cerevisiae var. boulardii (Sb). In some aspects, the composition is further configured for oral administration and for release of the haPD-1 into the patient's stomach. In some aspects, the haPD-1 includes a miniature antibody variant configured to bind the patient's programmed death ligand 1 (PD-L1) with high affinity. In some aspects, the composition acts as an immune checkpoint inhibitor (ICI) for gastrointestinal tumors.

In another aspect, a method of suppressing or reducing gastrointestinal tumors in a patient is disclosed that includes administering a therapeutically effect amount of any of the compositions described above.

Other objects and features will be in part apparent and in part pointed out hereinafter.

### DESCRIPTION OF THE DRAWINGS

The patent or application file contains at least one drawing executed in color. Copies of this patent or patent application publication with color drawing(s) will be provided by the Office upon request and payment of the necessary fee.

FIG. 1(A-F): Recombinant haPD-1 binds PD-L1 and performs functional immune checkpoint blockade. haPD-1 produced via an E. coli BL21(DE3) expression vector binds to hPD-L1 in a dose-dependent manner (K_{D}=7.299 nM, Specific binding with Hill Slope equation, GraphPad Prism v10.1.0) with comparable affinity to full-sized IgG antibodies (FIG. 1A). E. coli-derived haPD-1 performs functional hPD-1/hPD-L1 blockade (IC₅₀=33.15 nM, data fitted to a 4-parameter logistic curve using Prism v10.1.0) (FIG. 1B). Sb secretion system design and application (FIG. 1C). haPD-1 secretion is higher than rPD-L1 nb secretion, despite a slightly slower growth dynamic in Sb_haPD-1 compared to Sb_rPD-L1nb (FIG. 1D). Sb_haPD-1 supernatant has similar hPD-L1 binding (FIG. 1E) and hPD-1/hPD-L1 blockade (FIG. 1F) activity to purified, E. coli-derived haPD-1. Data are represented as mean±SD collected from at least two technical replicates.

FIG. 2(A-E): Sb_haPD-1 transiently traverses the gut without significant plasmid loss. Sb_haPD-1 titers peak in distal SI and colon regions at 1- and 2-hrs post-gavage, respectively (FIG. 2A), with total GI titers returning to baseline levels after 12 hrs (FIG. 2B). Episomal secretion cassette is maintained with minimal loss when grown in the absence of blasticidin selection in vitro (FIG. 2C). Sb_haPD-1 colonies harvested from mouse stool 24 hrs post-gavage maintain blasticidin resistance comparable to Sb_Vector control (p<0.2162, Mann-Whitney test) (FIG. 2D). These same colonies maintain enhanced haPD-1 secretion capacity 24 hrs relative to Sb_Vector control (p<0.0001, Mann-Whitney test) (FIG. 2E). Data in FIG. 2A and FIG. 2B are represented as mean±SEM collected from at least 3 biological replicates. Data in FIG. 2C, FIG. 2D, and FIG. 2E are represented as mean±SD collected from at least 3 technical replicates.

FIG. 3(A-E): Sb_haPD-1 is capable of secreting heterologous proteins in simulated GI conditions. Sb_haPD-1 growth (solid lines) and secretion (dashed lines) in various growth conditions (black, standard lab conditions; mauve, 37° C.; blue, anaerobic; pink, 0.1% glucose; red, physiological conditions). Significant differences in growth are noted in anaerobic (p<0.0424) and physiological (p<0.0004) conditions (extra sum-of-squares test with Bonferroni correction). Significant differences in secretion are noted in 0.1% glucose (p<0.0148) and physiological (p<0.005112) conditions (multiple t-tests with Bonferroni correction). (FIG. 3A, FIG. 3B, FIG. 3C, and FIG. 3D). Changes in relative secretion (i.e., [haPD-1]/cell density, or μg/CFU) in each condition normalized to standard lab condition control are quantified in (FIG. 3E). Significant differences compared to timepoint-matched lab conditions are denoted with asterisks (mixed effect analysis with Dunnett's multiple comparisons test). *p<0.05; **p<0.005; ***p<0.0005. Data are represented as mean±SD collected from at least four technical replicates.

FIG. 4(A-E): Sb_haPD-1 is well tolerated and reduces intestinal tumor burden. Overview of induction, treatment, and post-treatment schedule for experiments treating APC^{min} mice treated with PBS (black), Sb_Vector (orange), Sb_haPD-1 (blue), or α-mPD-L1 (purple) (FIG. 4A). Sb_haPD-1 treatment results in significantly fewer tumors (24.95% reduction, p<0.0239, ordinary one-way ANOVA with Dunnett's multiple comparisons test) in the distal SI relative to PBS control (FIG. 4B). Sb_haPD-1-treated mice have comparable survival (FIG. 4C) and weight loss (FIG. 4D) to PBS treatment group (n.s., Mantel-Cox test for survival, n.s., multiple Mann-Whitney tests with Bonferroni correction for weight). (FIG. 4C). Systemic levels of inflammatory cytokines IFN-γ, TNF-α, and IL-17 remain stable during Sb_haPD-1 treatment (n.s., ordinary one-way ANOVA with Dunnett's multiple comparisons test for IFN-γ and TNF-α and Kruskal-Wallis ANOVA with Dunn's multiple comparison test for IL-17) €. Data are represented as mean±SD. FIG. 4B, FIG. 4C, and FIG. 4D represent data combined from three independent mouse experiments (with n≥4 mice per treatment group except α-mPD-L1, which was only included in one experiment (n=10)). FIG. 4E represents data from a single mouse experiment (with n≥10 mice per treatment group). See FIG. 9(A-C) for detailed breakdown of independent mouse experiments.

FIG. 5(A-G): Sb_haPD-1 treatment results in shifts in immune cell and bacterial populations. Sb_haPD-1-treated mice have decreased Tregs (p<0.0490) (FIG. 5A), increased CD4⁺TNFα⁺ T cells (p<0.0902) (FIG. 5B), and decreased CD4⁺IFNγ⁺ T cells (p<0.0454) (FIG. 5C) compared to PBS control groups (ordinary one-way ANOVA with Dunnett's multiple comparisons). The top 15 prevalent genera at sacrifice across treatment groups are shown in (FIG. 5D). Statistically significant (MaAsLin2 linear modeling, Benjamini-Hochberg corrected) enrichments/depletion coefficients of taxa in mice posttreatment (d50) relative to pretreatment (d36) according to treatment (FIG. 5E). No statistically significant shifts were detected in α-mPD-L1-treated mice €. Bray-Curtis dissimilarity between pre- and post-treatment samples within each group is shown in (FIG. 5F). This dissimilarity metric is significantly higher in Sb_haPD-1-treated mice compared to PBS-treated mice (p<0.0001, Kruskal-Wallis with Dunn's multiple comparisons). Principal coordinate analysis of Bray-Curtis dissimilarity vectors shows significant clustering of post-treatment samples by treatment group (PERMANOVA, Benjamini-Hochberg corrected) (FIG. 5G). Data in FIG. 5A, FIG. 5B, and FIG. 5C are represented as mean±SD and data in FIG. 5F are represented as mean (red line) with upper and lower quartiles of the mean (white lines). Data was collected from a single mouse experiment (n≥10 mice per treatment group).

FIG. 6(A-F) (S1): Characterization of purified ICI variants. 0.5 μg (Lanes 1 and 3) and 5 μg (Lanes 2 and 4) of recombinant haPD-1 (Lanes 1 and 2) and rPD-L1 nb (Lanes 3 and 4) were loaded into a total protein stain (FIG. 6A), αHA Western blot (FIG. 6B), and αHis Western blot (FIG. 6C) to verify protein sizes (30.1 kDa for haPD-1 and 16 kDa for rPD-L1 nb) and purity. Binding curves for purified ICI variants and full-sized αPD-L1 antibodies generated from an ELISA-based mouse PD-L1 binding assay (FIG. 6D). Mouse PD-1/PD-L1 blockade assay induction curves for ICI variants and full-sized αPD-L1 antibodies (FIG. 6E). Following intraperitoneal injection of 100 ug purified haPD-1 or rPD-L1 nb into mice, ICI variants were quantified in the blood via ELISA. Both ICI variants are rapidly degraded and undetectable within 24 hrs post-injection (FIG. 6F). Data in FIG. 6D and FIG. 6E are represented as mean±SD collected across two technical replicates. Data in FIG. 6F are represented as mean±SEM collected across three biological replicates.

FIG. 7(A-D) (S2): Design of various ELISA formats used to quantify ICIs and measure ICI binding affinities. The ICI ELISA (FIG. 7A) is binding independent and used to directly quantify ICI concentrations unbiased by binding affinity. The sandwich ELISAs (FIG. 7B, FIG. 7C, and FIG. 7D) are binding dependent and used to quantify ICIs with functional PD-L1 binding activity. The ICI sandwich ELISA (FIG. 7B) relies on the presence of the HA epitope for detection and quantification of PD-L1-bound ICI variants. The PD-L1 sandwich ELISAs (FIG. 7C and FIG. 7D) instead use epitope-tagged human (FIG. 7C) or mouse (FIG. 7D) PD-L1 as the capture antigen to enable binding affinity comparison across different ICIs.

FIG. 8(A-D) (S3): Characterization of secretion system using model and ICI payloads. αHA ELISA-based detection of 48-hr culture broths collected from Sb strains expressing secreted and non-secreted HA-tagged Gaussia luciferase (Sb_GLuc and Sb_GLuc-ns, respectively) (FIG. 8A). αHA ELISA-based detection of secreted rPD-L1 nb and haPD-1 in 48-hr Sb culture broths (FIG. 8B). αHA western blot of 48-hr Sb culture broths from strains expressing secreted and non-secreted GLuc, secreted rPD-L1nb, and secreted haPD-1 (FIG. 8C). Stool collected at 4 hrs post-gavage in Sb_GLuc-treated mice have significantly higher (p<6E-06, multiple t-tests, Benjamini-Hochberg corrected) luminescence compared to empty vector control (FIG. 8D). Data in FIG. 8A and FIG. 8B are represented as mean±SD collected across three technical replicates. Data in FIG. 8D are represented as mean±SEM collected across four biological replicates.

FIG. 9(A-C) (S4): Tumor reduction by Sb_haPD-1. Relative tumor reductions in the distal SI observed within each independent mouse experiment (FIG. 9A). Raw tumor counts in the distal SI observed across all three mouse experiments (FIG. 9B). Relative tumor reductions in the colon observed across all three mouse experiments (FIG. 9C). Data for all panels were collected across three independent experiments (n≥4 mice per treatment group). Data in FIG. 9C are represented as mean±SEM.

FIG. 10(A-E) (S5): Treatment-dependent effects in the gut microbiome. Relative abundance at d50 in mice according to treatment group at the phylum (FIG. 10A) and family (FIG. 10B) levels. There are no significant differences in d50 Shannon diversity across treatment groups (n.s., ordinary one-way ANOVA with Dunnett's multiple comparisons test) (FIG. 10C). There are also no significant differences when comparing relative Shannon diversity change (d50/d36) across treatment groups (n.s., Kruskal-Wallis ANOVA with Dunn's multiple comparisons test) (FIG. 10D). Principle coordinate analysis shows statistically significant clustering within several treatment groups when comparing d36 samples to d50 samples (PERMANOVA with Benjamini-Hochberg correction) (FIG. 10E). Data were collected from a single mouse experiment (n≥10 mice per treatment group).

FIG. 11A Panel A: Sb_haPD-1 strains have a wide range of expression as determined by PD-L1 binding ELISA, which is positively correlated between two timepoints tested in vitro. Data shown are ELISA OD450 values adjusted by cell density (OD600).

FIG. 11B Panel B: Secretion signal identity has a significant impact on haPD-1 expression. The IV secretion signal has the highest signal.

FIG. 11C Panel C: Sb_haPD-1max (MJW126) has a 28-fold improvement in EC50 in PD-L1 binding ELISA assay (4PL logistic curve)

FIG. 11D Panel D: Sb_haPD-1 max culture broths demonstrate elevated activity in PD-1/PD-L1 blockade assay (Promega J1250)

FIG. 12A Sb_haPD-1max is now detectable in stools. Sb_haPD-1proto continues to not be detectable in stools

FIG. 12B Sb_haPD-1max is now detectable in stools. Sb_haPD-1proto continues to not be detectable in stools

Those of skill in the art will understand that the drawings, described below, are for illustrative purposes only. The drawings are not intended to limit the scope of the present teachings in any way.

### DETAILED DESCRIPTION OF THE INVENTION

Engineered probiotics are an emerging platform for in situ delivery of therapeutics to relevant body sites. In various aspects, a composition comprising an orally administered, yeast-based therapeutic delivery system is disclosed that is configured to deliver next-generation immune checkpoint inhibitor (ICI) proteins directly to gastrointestinal tumors. To achieve this, Saccharomyces cerevisiae var. boulardii (Sb), a probiotic yeast with high genetic tractability and innate anticancer activity, was engineered to secrete “miniature” antibody variants that target programmed death ligand 1 (Sb_haPD-1). As described in the examples below, when tested in an ICI-refractory CRC mouse model, Sb_haPD-1 significantly reduced intestinal tumor burden and resulted in significant shifts to the immune cell profile and microbiome composition. In various aspects, this oral therapeutic platform is modular and highly customizable, opening new avenues of targeted drug delivery that can be applied to treat a myriad of gastrointestinal malignancies.

The present disclosure is based, at least in part, on a genetically engineered strain of Saccharomyces cerevisiae var. boulardii (Sb). This Sb strain has been transformed with a modified plasmid OGS539 that enables Sb to secrete a high-affinity programmed cell death 1 ectodomain protein (haPD-1) extracellularly (this engineered yeast strain is hereafter termed Sb_haPD-1). The mechanism of action of haPD-1 is blockade of the immune checkpoint axis between PD-1 and programmed cell death ligand 1 (PD-L1) by binding PD-L1 with high affinity.

haPD-1 is a promising alternative to conventional immune checkpoint inhibitors (ICIs) that are larger IgG antibody proteins (150 kDa) used clinically to treat various cancers. Conventional PD-1/PD-L1 therapies are administered systemically, are frequently associated with off-target toxicities, are costly and labor-intensive to produce, and have limited efficacy in treating GI cancers. As an alternative to conventional therapy, Sb_haPD-1 can be administered orally and survives the gastrointestinal tract while maintaining the ability to secrete haPD-1. This may increase exposure and specificity of ICI therapy to tumors of the gastrointestinal tract, while limiting off-target exposures that provoke toxicity.

As demonstrated in the examples herein, Sb_haPD-1 produces and secretes a functional haPD-1 product that can bind mouse and human PD-L1 and block the PD-1/PD-L1 axis. Reduction of intestinal tumor burdens is demonstrated in a mouse model following a multi-day oral gavage regimen of Sb_haPD-1 with associated shifts to the gut microbiome and immune cell landscape. This same mouse model was not responsive to conventional therapy comprised of systemic injection of an anti-PD-L1 IgG antibody.

AS further described in the examples herein, the first development and validation of an engineered ICI secretion system in Sb to treat intestinal tumors via oral administration is shown. As a promising alternative to conventional ICIs in IgG antibody formats, functional ICI variants in smaller protein formats are integrated into Sb secretion circuits to allow efficient expression and secretion. It is demonstrated that Sb probiotics engineered to secrete ICI variants successfully engage the target PD-1/PD-L1 pathway and reduce intestinal tumor burden in a murine model of CRC that is recalcitrant to conventional ICI therapy. The individual components of this construct and its performance in GI conditions, both in vitro and in vivo, are also extensively characterized. This yeast-based platform for the delivery of ICIs within the GI tract is presented as a modular prototype that can be customized to treat a myriad of GI diseases.

In some aspects, the technology described herein can enable non-invasive and direct treatment of gastrointestinal tumors with ICI therapy, which can result in less treatment-associated toxicity in comparison to conventional ICIs. This allows access of ICI treatment to an expanded patient population and reduce incidence of treatment cessation due to off-target toxicities.

In various aspects, the engineered yeast comprises a nucleotide sequence encoding a high-affinity programmed cell death 1 ectodomain protein (haPD-1) comprising a PD-1 ectodomain fused to a IgG1 CH3 domain. In some aspects, the nucleotide sequence encoding the haPD-1 protein is selected from one of the sequences of TABLE 1 below:

TABLE 1
haPD-1 ENCODING SEQUENCES

| SEQ ID NO: | Name | Promoter | Secretion Signal | Terminator |
|---|---|---|---|---|
| 1 | MJW110 | pTEF1 | Sed1p | tENO2 |
| 2 | MJW111 | pTEF1 | Sed1p | tCYCE1 |
| 3 | MJW112 | pTEF1 | Sed1p | tTDH1 |
| 4 | MJW113 | pADH1 | MFa | tTPS1 |
| 5 | MJW114 | pTDH3 | IV | tTDH1 |
| 6 | MJW115 | pTDH3 | IV | tCYCE1 |
| 7 | MJW116 | pTDH3 | MFa | tTDH1 |
| 8 | MJW117 | pADH1 | SAG1 | tENO2 |
| 9 | MJW118 | pTDH3 | MFa | tSSA1 |
| 10 | MJW119 | pCCW12 | IV | tENO2 |
| 11 | MJW120 | pCCW12 | IV | tSSA1 |
| 12 | MJW121 | pALD6 | AT | tTPS1 |
| 13 | MJW122 | pALD6 | SAG1 | tTDH1 |
| 14 | MJW123 | pALD6 | SAG1 | tENO2 |
| 15 | MJW124 | pALD6 | SAG1 | tSSA1 |
| 16 | MJW125 | pTEF1 | AT | tCYCE1 |
| 17 | MJW126 | pTEF1 | IV | tCYCE1 |
| 18 | MJW127 | pTEF1 | MFa | tCYCE1 |
| 19 | MJW128 | pTEF1 | SAG1 | tCYCE1 |
| 20 | MJW129 | pALD6 | MFa | tCYCE1 |
| 23 | MJW130 | pADH1 | MFa | tCYCE1 |
| 21 | MJW131 | pCCW12 | MFa | tCYCE1 |
| 22 | MJW132 | pTDH3 | MFa | tCYCE1 |

### Immune Checkpoint Modulation Agents

As described herein, PD-1/PDL-1 signaling has been implicated in various diseases, disorders, and conditions. As such, modulation of PD-1/PDL-1 (e.g., modulation of PD-1 and/or PDL-1 expression or binding in cancer cells) can be used for treatment of such conditions. A PD-1/PDL-1 modulation agent can modulate immune response or induce or inhibit programmed cell death. Immune modulation can comprise modulating the expression of PD-1 on cells, modulating the quantity of cells that express PD-1, or modulating the quality of the PD-1 expressing cells.

Immune checkpoint modulation agents can be any composition or method that can modulate immune checkpoint factor expression on cells (e.g., PD-1 and PDL-1). For example, an immune checkpoint modulation agent can be an activator, an inhibitor, an agonist, or an antagonist. As another example, the immune checkpoint modulation can be the result of gene editing.

An immune checkpoint modulation agent can be an immune checkpoint inhibitor (ICI) antibody, associated fragment proteins, variants, and any combination thereof (e.g., a monoclonal antibody and protein variants to PD-1/PDL-1).

An immune checkpoint modulating agent can be an agent that induces or inhibits progenitor cell differentiation into PD-1 expressing cells (e.g., through a PDL-1 antibody fragment). For example, an ICI variant can be used to engage the PD-1/PDL-1 pathway.

Immune Checkpoint Signal Reduction, Elimination, or Inhibition by Small Molecule Inhibitors, shRNA, siRNA, or ASOs

As described herein, an immune checkpoint modulation agent can be used for use in GI disorder therapy, including but not limited GI cancer therapy. An immune checkpoint modulation agent can be used to reduce/eliminate or enhance/increase immune signals. For example, an immune checkpoint modulation agent can be a small molecule inhibitor of immune checkpoint factors. As another example, an immune checkpoint modulation agent can be a short hairpin RNA (shRNA). As another example, an immune checkpoint modulation agent can be a short interfering RNA (siRNA).

As another example, RNA (e.g., long noncoding RNA (lncRNA)) can be targeted with antisense oligonucleotides (ASOs) as a therapeutic. Processes for making ASOs targeted to RNAs are well known, see e.g. Zhou et al. 2016 Methods Mol Biol. 1402:199-213. Except as otherwise noted herein, therefore, the process of the present disclosure can be carried out in accordance with such processes.

### Immune Checkpoint Inhibiting Agent

One aspect of the present disclosure provides for targeting of PDL-1, its receptor, or its downstream signaling. The present disclosure provides methods of treating or preventing a GI disorder based on the discovery that ICI antibody variants successfully engage the PD-1/PDL-1 pathway.

As described herein, inhibitors of immune checkpoint factors (e.g., antibodies, fusion proteins, small molecules) can reduce or prevent a GI disorder. An immune checkpoint inhibiting agent can be any agent that can engage the PD-1/PDL-1 pathway, upregulate the PD-1/PDL-1 pathway, or knock-in the PD-1/PDL-1 pathway.

As an example, an immune checkpoint inhibiting agent can modulate PD-1/PDL-1 signaling.

For example, the immune checkpoint inhibiting agent can be an anti-PD-1 antibody. As an example, the anti-PD-1 antibody can be anti-PDL-1 antibody, Furthermore, the anti-PD-1 antibody can be a murine antibody, a humanized murine antibody, a human antibody, and any variant fragments thereof.

As another example, the immune checkpoint inhibiting agent can be an anti-PDL1 antibody, wherein the anti-PDL1 antibody facilitates binding of PDL-1 to its receptor, PD-1, or engages activation of PD-1/PDL-1 and downstream signaling.

As another example, the immune checkpoint inhibiting agent can be a fusion protein. For example, the fusion protein can be a decoy receptor for PD-1. Furthermore, the fusion protein can comprise a mouse or human Fc antibody domain fused to the ectodomain of PDL-1.

As another example, an immune checkpoint inhibiting agent can be Sb_haPD-1, which has been shown to be a potent and specific engager PD-1/PDL-1 signaling.

As another example, an immune checkpoint inhibiting agent can be a protein that targets PDL-1. For example, the immune checkpoint inhibiting agent can be a viral protein, which has been shown to target PDL-1.

As another example, an immune checkpoint inhibiting agent can be a short hairpin RNA (shRNA) or a short interfering RNA (siRNA) targeting PDL-1 or PD-1.

As another example, an immune checkpoint inhibiting agent can be an sgRNA targeting PDL-1 or PD-1.

Methods for preparing an immune checkpoint inhibiting agent (e.g., an agent capable of modulating PD-1/PDL-1 signaling) can comprise construction of a protein/Ab scaffold containing the natural PD-1 receptor as an immune checkpoint neutralizing agent; developing activators of the PD-1 receptor “down-stream”; or developing activators of PDL-1 production “up-stream”.

Inhibiting immune checkpoints can be performed by genetically modifying PD-1/PDL-1 in a subject or genetically modifying a subject to reduce or prevent expression of the PD-1 or PDL-1 gene, such as through the use of CRISPR-Cas9 or analogous technologies, wherein, such modification reduces or prevents a GI disorder.

### Chemical Agent:

Examples of immune checkpoint modulation agents are described herein. Immune checkpoint modulation agents can be Sb_haPD-1.

R groups can be optionally substituted with one or more groups independently selected from the group consisting of hydroxyl; C₁₋₁₀alkyl hydroxyl; amine; C₁₋₁₀carboxylic acid; C₁₋₁₀carboxyl; straight chain or branched C₁₋₁₀alkyl, optionally containing unsaturation; a C₂₋₁₀cycloalkyl optionally containing unsaturation or one oxygen or nitrogen atom; straight chain or branched C₁₋₁₀alkyl amine; heterocyclyl; heterocyclic amine; and aryl comprising a phenyl; heteroaryl containing from 1 to 4 N, O, or S atoms; unsubstituted phenyl ring; substituted phenyl ring; unsubstituted heterocyclyl; and substituted heterocyclyl, wherein the unsubstituted phenyl ring or substituted phenyl ring can be optionally substituted with one or more groups independently selected from the group consisting of hydroxyl; C₁₋₁₀alkyl hydroxyl; amine; C₁₋₁₀carboxylic acid; C₁₋₁₀carboxyl; straight chain or branched C₁₋₁₀alkyl, optionally containing unsaturation; straight chain or branched C₁₋₁₀alkyl amine, optionally containing unsaturation; a C₂₋₁₀cycloalkyl optionally containing unsaturation or one oxygen or nitrogen atom; straight chain or branched C₁₋₁₀alkyl amine; heterocyclyl; heterocyclic amine; aryl comprising a phenyl; and heteroaryl containing from 1 to 4 N, O, or S atoms; and the unsubstituted heterocyclyl or substituted heterocyclyl can be optionally substituted with one or more groups independently selected from the group consisting of hydroxyl; C₁₋₁₀alkyl hydroxyl; amine; C₁₋₁₀carboxylic acid; C₁₋₁₀carboxyl; straight chain or branched C₁₋₁₀alkyl, optionally containing unsaturation; straight chain or branched C₁₋₁₀alkyl amine, optionally containing unsaturation; a C₂₋₁₀cycloalkyl optionally containing unsaturation or one oxygen or nitrogen atom; heterocyclyl; straight chain or branched C₁₋₁₀alkyl amine; heterocyclic amine; and aryl comprising a phenyl; and heteroaryl containing from 1 to 4 N, O, or S atoms. Any of the above can be further optionally substituted.

The term “imine” or “imino”, as used herein, unless otherwise indicated, can include a functional group or chemical compound containing a carbon-nitrogen double bond. The expression “imino compound”, as used herein, unless otherwise indicated, refers to a compound that includes an “imine” or an “imino” group as defined herein. The “imine” or “imino” group can be optionally substituted.

The term “hydroxyl”, as used herein, unless otherwise indicated, can include —OH. The “hydroxyl” can be optionally substituted.

The terms “halogen” and “halo”, as used herein, unless otherwise indicated, include a chlorine, chloro, Cl; fluorine, fluoro, F; bromine, bromo, Br; or iodine, iodo, or I.

The term “acetamide”, as used herein, is an organic compound with the formula CH₃CONH₂. The “acetamide” can be optionally substituted.

The term “aryl”, as used herein, unless otherwise indicated, include a carbocyclic aromatic group. Examples of aryl groups include, but are not limited to, phenyl, benzyl, naphthyl, or anthracenyl. The “aryl” can be optionally substituted.

The terms “amine” and “amino”, as used herein, unless otherwise indicated, include a functional group that contains a nitrogen atom with a lone pair of electrons and wherein one or more hydrogen atoms have been replaced by a substituent such as, but not limited to, an alkyl group or an aryl group. The “amine” or “amino” group can be optionally substituted.

The term “alkyl”, as used herein, unless otherwise indicated, can include saturated monovalent hydrocarbon radicals having straight or branched moieties, such as but not limited to, methyl, ethyl, propyl, butyl, pentyl, hexyl, octyl groups, etc. Representative straight-chain lower alkyl groups include, but are not limited to, -methyl, -ethyl, -n-propyl, -n-butyl, -n-pentyl, -n-hexyl, -n-heptyl and -n-octyl; while branched lower alkyl groups include, but are not limited to, -isopropyl, -sec-butyl, -isobutyl, -tert-butyl, -isopentyl, 2-methylbutyl, 2-methylpentyl, 3-methylpentyl, 2,2-dimethylbutyl, 2,3-dimethylbutyl, 2,2-dimethylpentyl, 2,3-dimethylpentyl, 3,3-dimethylpentyl, 2,3,4-trimethylpentyl, 3-methylhexyl, 2,2-dimethylhexyl, 2,4-dimethylhexyl, 2,5-dimethylhexyl, 3,5-dimethylhexyl, 2,4-dimethylpentyl, 2-methylheptyl, 3-methylheptyl, unsaturated C₁₋₁₀ alkyls include, but are not limited to, -vinyl, -allyl, -1-butenyl, -2-butenyl, -isobutylenyl, -1-pentenyl, -2-pentenyl, -3-methyl-1-butenyl, -2-methyl-2-butenyl, -2,3-dimethyl-2-butenyl, 1-hexyl, 2-hexyl, 3-hexyl, -acetylenyl, -propynyl, -1-butynyl, -2-butynyl, -1-pentynyl, -2-pentynyl, or -3-methyl-1 butynyl. An alkyl can be saturated, partially saturated, or unsaturated. The “alkyl” can be optionally substituted.

The term “carboxyl”, as used herein, unless otherwise indicated, can include a functional group consisting of a carbon atom double bonded to an oxygen atom and single bonded to a hydroxyl group (—COOH). The “carboxyl” can be optionally substituted.

The term “alkenyl”, as used herein, unless otherwise indicated, can include alkyl moieties having at least one carbon-carbon double bond wherein alkyl is as defined above and including E and Z isomers of said alkenyl moiety. An alkenyl can be partially saturated or unsaturated. The “alkenyl” can be optionally substituted.

The term “alkynyl”, as used herein, unless otherwise indicated, can include alkyl moieties having at least one carbon-carbon triple bond wherein alkyl is as defined above. An alkynyl can be partially saturated or unsaturated.

The “alkynyl” can be optionally substituted.

The term “acyl”, as used herein, unless otherwise indicated, can include a functional group derived from an aliphatic carboxylic acid, by removal of the hydroxyl (—OH) group. The “acyl” can be optionally substituted.

The term “alkoxyl”, as used herein, unless otherwise indicated, can include O-alkyl groups wherein alkyl is as defined above, and O represents oxygen. Representative alkoxyl groups include, but are not limited to, —O-methyl, —O-ethyl, —O-n-propyl, —O-n-butyl, —O-n-pentyl, —O-n-hexyl, —O-n-heptyl, —O-n-octyl, —O-isopropyl, —O-sec-butyl, —O-isobutyl, —O-tert-butyl, —O-isopentyl, —O-2-methylbutyl, —O-2-methylpentyl, —O-3-methylpentyl, —O-2,2-dimethylbutyl, —O-2,3-dimethylbutyl, —O-2,2-dimethylpentyl, —O-2,3-dimethylpentyl, —O-3,3-dimethylpentyl, —O-2,3,4-trimethylpentyl, —O-3-methylhexyl, —O-2,2-dimethylhexyl, —O-2,4-dimethylhexyl, —O-2,5-dimethylhexyl, —O-3,5-dimethylhexyl, —O-2,4dimethylpentyl, —O-2-methylheptyl, —O-3-methylheptyl, —O-vinyl, —O-allyl, —O-1-butenyl, —O-2-butenyl, —O-isobutylenyl, —O-1-pentenyl, —O-2-pentenyl, —O-3-methyl-1-butenyl, —O-2-methyl-2-butenyl, —O-2,3-dimethyl-2-butenyl, —O-1-hexyl, —O-2-hexyl, —O-3-hexyl, —O-acetylenyl, —O-propynyl, —O-1-butynyl, —O-2-butynyl, —O-1-pentynyl, —O-2-pentynyl and —O-3-methyl-1-butynyl, —O-cyclopropyl, —O— cyclobutyl, —O-cyclopentyl, —O-cyclohexyl, —O-cycloheptyl, —O-cyclooctyl, —O— cyclononyl and —O-cyclodecyl, —O—CH₂-cyclopropyl, —O—CH₂-cyclobutyl, —O—CH₂-cyclopentyl, —O—CH₂-cyclohexyl, —O—CH₂-cycloheptyl, —O—CH₂-cyclooctyl, —O—CH₂-cyclononyl, —O—CH₂-cyclodecyl, —O—(CH₂)₂-cyclopropyl, —O—(CH₂)₂-cyclobutyl, —O—(CH₂)₂-cyclopentyl, —O—(CH₂)₂-cyclohexyl, —O—(CH₂)₂-cycloheptyl, —O—(CH₂)₂-cyclooctyl, —O—(CH₂)₂-cyclononyl, or —O—(CH₂)₂-cyclodecyl. An alkoxyl can be saturated, partially saturated, or unsaturated. The “alkoxyl” can be optionally substituted.

The term “cycloalkyl”, as used herein, unless otherwise indicated, can include an aromatic, a non-aromatic, saturated, partially saturated, or unsaturated, monocyclic or fused, spiro or unfused bicyclic or tricyclic hydrocarbon referred to herein containing a total of from 1 to 10 carbon atoms (e.g., 1 or 2 carbon atoms if there are other heteroatoms in the ring), preferably 3 to 8 ring carbon atoms. Examples of cycloalkyls include, but are not limited to, C₃₋₁₀ cycloalkyl groups include, but are not limited to, -cyclopropyl, -cyclobutyl, -cyclopentyl, -cyclopentadienyl, -cyclohexyl, -cyclohexenyl, -1,3-cyclohexadienyl, -1,4-cyclohexadienyl, -cycloheptyl, -1,3-cycloheptadienyl, -1,3,5-cycloheptatrienyl, -cyclooctyl, and -cyclooctadienyl. The term “cycloalkyl” also can include -lower alkyl-cycloalkyl, wherein lower alkyl and cycloalkyl are as defined herein. Examples of -lower alkyl-cycloalkyl groups include, but are not limited to, —CH₂-cyclopropyl, —CH₂-cyclobutyl, —CH₂-cyclopentyl, —CH₂-cyclopentadienyl, —CH₂-cyclohexyl, —CH₂-cycloheptyl, or —CH₂-cyclooctyl. The “cycloalkyl” can be optionally substituted. A “cycloheteroalkyl”, as used herein, unless otherwise indicated, can include any of the above with a carbon substituted with a heteroatom (e.g., O, S, N).

The term “heterocyclic” or “heteroaryl”, as used herein, unless otherwise indicated, can include an aromatic or non-aromatic cycloalkyl in which one to four of the ring carbon atoms are independently replaced with a heteroatom from the group consisting of O, S and N. Representative examples of a heterocycle include, but are not limited to, benzofuranyl, benzothiophene, indolyl, benzopyrazolyl, coumarinyl, isoquinolinyl, pyrrolyl, pyrrolidinyl, thiophenyl, furanyl, thiazolyl, imidazolyl, pyrazolyl, triazolyl, quinolinyl, pyrimidinyl, pyridinyl, pyridonyl, pyrazinyl, pyridazinyl, isothiazolyl, isoxazolyl, (1,4)-dioxane, (1,3)-dioxolane, 4,5-dihydro-1H-imidazolyl, or tetrazolyl. Heterocycles can be substituted or unsubstituted. Heterocycles can also be bonded at any ring atom (i.e., at any carbon atom or heteroatom of the heterocyclic ring). A heterocyclic can be saturated, partially saturated, or unsaturated. The “hetreocyclic” can be optionally substituted.

The term “indole”, as used herein, is an aromatic heterocyclic organic compound with formula C₈H₇N. It has a bicyclic structure, consisting of a six-membered benzene ring fused to a five-membered nitrogen-containing pyrrole ring. The “indole” can be optionally substituted.

The term “cyano”, as used herein, unless otherwise indicated, can include a —CN group. The “cyano” can be optionally substituted.

The term “alcohol”, as used herein, unless otherwise indicated, can include a compound in which the hydroxyl functional group (—OH) is bound to a carbon atom. In particular, this carbon center should be saturated, having single bonds to three other atoms. The “alcohol” can be optionally substituted.

The term “solvate” is intended to mean a solvate form of a specified compound that retains the effectiveness of such compound. Examples of solvates include compounds of the invention in combination with, for example: water, isopropanol, ethanol, methanol, dimethylsulfoxide (DMSO), ethyl acetate, acetic acid, or ethanolamine.

The term “mmol”, as used herein, is intended to mean millimole. The term “equiv”, as used herein, is intended to mean equivalent. The term “mL”, as used herein, is intended to mean milliliter. The term “g”, as used herein, is intended to mean gram. The term “kg”, as used herein, is intended to mean kilogram. The term “μg”, as used herein, is intended to mean micrograms. The term “h”, as used herein, is intended to mean hour. The term “min”, as used herein, is intended to mean minute. The term “M”, as used herein, is intended to mean molar. The term “μL”, as used herein, is intended to mean microliter. The term “μM”, as used herein, is intended to mean micromolar. The term “nM”, as used herein, is intended to mean nanomolar. The term “N”, as used herein, is intended to mean normal. The term “amu”, as used herein, is intended to mean atomic mass unit. The term “° C.”, as used herein, is intended to mean degree Celsius. The term “wt/wt”, as used herein, is intended to mean weight/weight. The term “v/v”, as used herein, is intended to mean volume/volume. The term “MS”, as used herein, is intended to mean mass spectroscopy. The term “HPLC”, as used herein, is intended to mean high performance liquid chromatograph. The term “RT”, as used herein, is intended to mean room temperature. The term “e.g.”, as used herein, is intended to mean example. The term “N/A”, as used herein, is intended to mean not tested.

As used herein, the expression “pharmaceutically acceptable salt” refers to pharmaceutically acceptable organic or inorganic salts of a compound of the invention. Preferred salts include, but are not limited, to sulfate, citrate, acetate, oxalate, chloride, bromide, iodide, nitrate, bisulfate, phosphate, acid phosphate, isonicotinate, lactate, salicylate, acid citrate, tartrate, oleate, tannate, pantothenate, bitartrate, ascorbate, succinate, maleate, gentisinate, fumarate, gluconate, glucaronate, saccharate, formate, benzoate, glutamate, methanesulfonate, ethanesulfonate, benzenesulfonate, p-toluenesulfonate, or pamoate (i.e., 1,1′-methylene-bis-(2-hydroxy-3-naphthoate)) salts. A pharmaceutically acceptable salt may involve the inclusion of another molecule such as an acetate ion, a succinate ion or other counterion. The counterion may be any organic or inorganic moiety that stabilizes the charge on the parent compound. Furthermore, a pharmaceutically acceptable salt may have more than one charged atom in its structure. Instances where multiple charged atoms are part of the pharmaceutically acceptable salt can have multiple counterions. Hence, a pharmaceutically acceptable salt can have one or more charged atoms and/or one or more counterion. As used herein, the expression “pharmaceutically acceptable solvate” refers to an association of one or more solvent molecules and a compound of the invention. Examples of solvents that form pharmaceutically acceptable solvates include, but are not limited to, water, isopropanol, ethanol, methanol, DMSO, ethyl acetate, acetic acid, and ethanolamine. As used herein, the expression “pharmaceutically acceptable hydrate” refers to a compound of the invention, or a salt thereof, that further can include a stoichiometric or non-stoichiometric amount of water bound by non-covalent intermolecular forces.

### Molecular Engineering

The following definitions and methods are provided to better define the present invention and to guide those of ordinary skill in the art in the practice of the present invention. Unless otherwise noted, terms are to be understood according to conventional usage by those of ordinary skill in the relevant art.

The terms “heterologous DNA sequence”, “exogenous DNA segment” or “heterologous nucleic acid,” as used herein, each refer to a sequence that originates from a source foreign to the particular host cell or, if from the same source, is modified from its original form. Thus, a heterologous gene in a host cell includes a gene that is endogenous to the particular host cell but has been modified through, for example, the use of DNA shuffling or cloning. The terms also include non-naturally occurring multiple copies of a naturally occurring DNA sequence. Thus, the terms refer to a DNA segment that is foreign or heterologous to the cell, or homologous to the cell but in a position within the host cell nucleic acid in which the element is not ordinarily found. Exogenous DNA segments are expressed to yield exogenous polypeptides. A “homologous” DNA sequence is a DNA sequence that is naturally associated with a host cell into which it is introduced.

Expression vector, expression construct, plasmid, or recombinant DNA construct is generally understood to refer to a nucleic acid that has been generated via human intervention, including by recombinant means or direct chemical synthesis, with a series of specified nucleic acid elements that permit transcription or translation of a particular nucleic acid in, for example, a host cell. The expression vector can be part of a plasmid, virus, or nucleic acid fragment. Typically, the expression vector can include a nucleic acid to be transcribed operably linked to a promoter.

A “promoter” is generally understood as a nucleic acid control sequence that directs transcription of a nucleic acid. An inducible promoter is generally understood as a promoter that mediates transcription of an operably linked gene in response to a particular stimulus. A promoter can include necessary nucleic acid sequences near the start site of transcription, such as, in the case of a polymerase II type promoter, a TATA element. A promoter can optionally include distal enhancer or repressor elements, which can be located as much as several thousand base pairs from the start site of transcription.

A “transcribable nucleic acid molecule” as used herein refers to any nucleic acid molecule capable of being transcribed into an RNA molecule. Methods are known for introducing constructs into a cell in such a manner that the transcribable nucleic acid molecule is transcribed into a functional mRNA molecule that is translated and therefore expressed as a protein product. Constructs may also be constructed to be capable of expressing antisense RNA molecules, in order to inhibit translation of a specific RNA molecule of interest. For the practice of the present disclosure, conventional compositions and methods for preparing and using constructs and host cells are well known to one skilled in the art (see e.g., Sambrook and Russel (2006) Condensed Protocols from Molecular Cloning: A Laboratory Manual, Cold Spring Harbor Laboratory Press, ISBN-10: 0879697717; Ausubel et al. (2002) Short Protocols in Molecular Biology, 5th ed., Current Protocols, ISBN-10: 0471250929; Sambrook and Russel (2001) Molecular Cloning: A Laboratory Manual, 3d ed., Cold Spring Harbor Laboratory Press, ISBN-10: 0879695773; Elhai, J. and Wolk, C. P. 1988. Methods in Enzymology 167, 747-754).

The “transcription start site” or “initiation site” is the position surrounding the first nucleotide that is part of the transcribed sequence, which is also defined as position+1. With respect to this site all other sequences of the gene and its controlling regions can be numbered. Downstream sequences (i.e., further protein encoding sequences in the 3′ direction) can be denominated positive, while upstream sequences (mostly of the controlling regions in the 5′ direction) are denominated negative.

“Operably linked” or “functionally linked” refers preferably to the association of nucleic acid sequences on a single nucleic acid fragment so that the function of one is affected by the other. For example, a regulatory DNA sequence is said to be “operably linked to” or “associated with” a DNA sequence that codes for an RNA or a polypeptide if the two sequences are situated such that the regulatory DNA sequence affects expression of the coding DNA sequence (i.e., that the coding sequence or functional RNA is under the transcriptional control of the promoter). Coding sequences can be operably linked to regulatory sequences in sense or antisense orientation. The two nucleic acid molecules may be part of a single contiguous nucleic acid molecule and may be adjacent. For example, a promoter is operably linked to a gene of interest if the promoter regulates or mediates transcription of the gene of interest in a cell.

A “construct” is generally understood as any recombinant nucleic acid molecule such as a plasmid, cosmid, virus, autonomously replicating nucleic acid molecule, phage, or linear or circular single-stranded or double-stranded DNA or RNA nucleic acid molecule, derived from any source, capable of genomic integration or autonomous replication, comprising a nucleic acid molecule where one or more nucleic acid molecule has been operably linked.

A construct of the present disclosure can contain a promoter operably linked to a transcribable nucleic acid molecule operably linked to a 3′ transcription termination nucleic acid molecule. In addition, constructs can include but are not limited to additional regulatory nucleic acid molecules from, e.g., the 3′-untranslated region (3′ UTR). Constructs can include but are not limited to the 5′ untranslated regions (5′ UTR) of an mRNA nucleic acid molecule which can play an important role in translation initiation and can also be a genetic component in an expression construct. These additional upstream and downstream regulatory nucleic acid molecules may be derived from a source that is native or heterologous with respect to the other elements present on the promoter construct.

The term “transformation” refers to the transfer of a nucleic acid fragment into the genome of a host cell, resulting in genetically stable inheritance. Host cells containing the transformed nucleic acid fragments are referred to as “transgenic” cells, and organisms comprising transgenic cells are referred to as “transgenic organisms”.

“Transformed,” “transgenic,” and “recombinant” refer to a host cell or organism such as a bacterium, cyanobacterium, animal or a plant into which a heterologous nucleic acid molecule has been introduced. The nucleic acid molecule can be stably integrated into the genome as generally known in the art and disclosed (Sambrook 1989; Innis 1995; Gelfand 1995; Innis & Gelfand 1999). Known methods of PCR include, but are not limited to, methods using paired primers, nested primers, single specific primers, degenerate primers, gene-specific primers, vector-specific primers, partially mismatched primers, and the like. The term “untransformed” refers to normal cells that have not been through the transformation process.

“Wild-type” refers to a virus or organism found in nature without any known mutation.

Design, generation, and testing of the variant nucleotides, and their encoded polypeptides, having the above required percent identities and retaining a required activity of the expressed protein is within the skill of the art. For example, directed evolution and rapid isolation of mutants can be according to methods described in references including, but not limited to, Link et al. (2007) Nature Reviews 5(9), 680-688; Sanger et al. (1991) Gene 97(1), 119-123; Ghadessy et al. (2001) Proc Natl Acad Sci USA 98(8) 4552-4557. Thus, one skilled in the art could generate a large number of nucleotide and/or polypeptide variants having, for example, at least 95-99% identity to the reference sequence described herein and screen such for desired phenotypes according to methods routine in the art.

Nucleotide and/or amino acid sequence identity percent (%) is understood as the percentage of nucleotide or amino acid residues that are identical with nucleotide or amino acid residues in a candidate sequence in comparison to a reference sequence when the two sequences are aligned. To determine percent identity, sequences are aligned and if necessary, gaps are introduced to achieve the maximum percent sequence identity. Sequence alignment procedures to determine percent identity are well known to those of skill in the art. Often publicly available computer software such as BLAST, BLAST2, ALIGN2 or Megalign (DNASTAR) software is used to align sequences. Those skilled in the art can determine appropriate parameters for measuring alignment, including any algorithms needed to achieve maximal alignment over the full-length of the sequences being compared. When sequences are aligned, the percent sequence identity of a given sequence A to, with, or against a given sequence B (which can alternatively be phrased as a given sequence A that has or comprises a certain percent sequence identity to, with, or against a given sequence B) can be calculated as: percent sequence identity=X/Y100, where X is the number of residues scored as identical matches by the sequence alignment program's or algorithm's alignment of A and B and Y is the total number of residues in B. If the length of sequence A is not equal to the length of sequence B, the percent sequence identity of A to B will not equal the percent sequence identity of B to A.

Generally, conservative substitutions can be made at any position so long as the required activity is retained. So-called conservative exchanges can be carried out in which the amino acid which is replaced has a similar property as the original amino acid, for example the exchange of Glu by Asp, Gln by Asn, Val by lie, Leu by lie, and Ser by Thr. For example, amino acids with similar properties can be Aliphatic amino acids (e.g., Glycine, Alanine, Valine, Leucine, Isoleucine); Hydroxyl or sulfur/selenium-containing amino acids (e.g., Serine, Cysteine, Selenocysteine, Threonine, Methionine); Cyclic amino acids (e.g., Proline); Aromatic amino acids (e.g., Phenylalanine, Tyrosine, Tryptophan); Basic amino acids (e.g., Histidine, Lysine, Arginine); or Acidic and their Amide (e.g., Aspartate, Glutamate, Asparagine, Glutamine). Deletion is the replacement of an amino acid by a direct bond. Positions for deletions include the termini of a polypeptide and linkages between individual protein domains. Insertions are introductions of amino acids into the polypeptide chain, a direct bond formally being replaced by one or more amino acids. Amino acid sequence can be modulated with the help of art-known computer simulation programs that can produce a polypeptide with, for example, improved activity or altered regulation. On the basis of this artificially generated polypeptide sequences, a corresponding nucleic acid molecule coding for such a modulated polypeptide can be synthesized in-vitro using the specific codon-usage of the desired host cell.

“Highly stringent hybridization conditions” are defined as hybridization at 65° C. in a 6×SSC buffer (i.e., 0.9 M sodium chloride and 0.09 M sodium citrate). Given these conditions, a determination can be made as to whether a given set of sequences will hybridize by calculating the melting temperature (Tₘ) of a DNA duplex between the two sequences. If a particular duplex has a melting temperature lower than 65° C. in the salt conditions of a 6×SSC, then the two sequences will not hybridize. On the other hand, if the melting temperature is above 65° C. in the same salt conditions, then the sequences will hybridize. In general, the melting temperature for any hybridized DNA:DNA sequence can be determined using the following formula: Tₘ=81.5° C.+16.6(log₁₀[Na⁺])+0.41 (fraction G/C content)−0.63(% formamide)−(600/l). Furthermore, the Tₘ of a DNA:DNA hybrid is decreased by 1-1.5° C. for every 1% decrease in nucleotide identity (see e.g., Sambrook and Russel, 2006).

Host cells can be transformed using a variety of standard techniques known to the art (see e.g., Sambrook and Russel (2006) Condensed Protocols from Molecular Cloning: A Laboratory Manual, Cold Spring Harbor Laboratory Press, ISBN-10: 0879697717; Ausubel et al. (2002) Short Protocols in Molecular Biology, 5th ed., Current Protocols, ISBN-10: 0471250929; Sambrook and Russel (2001) Molecular Cloning: A Laboratory Manual, 3d ed., Cold Spring Harbor Laboratory Press, ISBN-10: 0879695773; Elhai, J. and Wolk, C. P. 1988. Methods in Enzymology 167, 747-754). Such techniques include, but are not limited to, viral infection, calcium phosphate transfection, liposome-mediated transfection, microprojectile-mediated delivery, receptor-mediated uptake, cell fusion, electroporation, and the like. The transfected cells can be selected and propagated to provide recombinant host cells that comprise the expression vector stably integrated in the host cell genome.

Conservative Substitutions I

| Side Chain | |
|---|---|
| Characteristic | Amino Acid |
| Aliphatic Non-polar | G A P I L V |
| Polar-uncharged | C S T M N Q |
| Polar-charged | D E K R |
| Aromatic | H F W Y |
| Other | N Q D E |

Conservative Substitutions II

| Side Chain | |
|---|---|
| Characteristic | |
| Non-polar | |
| (hydrophobic) | Amino Acid |
| A. Aliphatic: | A L I V P |
| B. Aromatic: | F W |
| C. Sulfur-containing: | M |
| D. Borderline: | G |
| Uncharged-polar | |
| A. Hydroxyl: | S T Y |
| B. Amides: | N Q |
| C. Sulfhydryl: | C |
| D. Borderline: | G |
| Positively Charged | K R H |
| (Basic): | |
| Negatively Charged | D E |
| (Acidic): | |

Conservative Substitutions III

| Original Residue | Exemplary Substitution |
|---|---|
| Ala (A) | Val, Leu, Ile |
| Arg (R) | Lys, Gln, Asn |
| Asn (N) | Gln, His, Lys, Arg |
| Asp (D) | Glu |
| Cys (C) | Ser |
| Gln (Q) | Asn |
| Glu (E) | Asp |
| His (H) | Asn, Gln, Lys, Arg |
| Ile (I) | Leu, Val, Met, Ala, Phe, |
| Leu (L) | Ile, Val, Met, Ala, Phe |
| Lys (K) | Arg, Gln, Asn |
| Met (M) | Leu, Phe, Ile |
| Phe (F) | Leu, Val, Ile, Ala |
| Pro (P) | Gly |
| Ser (S) | Thr |
| Thr (T) | Ser |
| Trp (W) | Tyr, Phe |
| Tyr (Y) | Trp, Phe, Tur, Ser |
| Val (V) | lle, Leu, Met, Phe, Ala |

Exemplary nucleic acids which may be introduced to a host cell include, for example, DNA sequences or genes from another species, or even genes or sequences which originate with or are present in the same species but are incorporated into recipient cells by genetic engineering methods. The term “exogenous” is also intended to refer to genes that are not normally present in the cell being transformed, or perhaps simply not present in the form, structure, etc., as found in the transforming DNA segment or gene, or genes which are normally present and that one desires to express in a manner that differs from the natural expression pattern, e.g., to over-express. Thus, the term “exogenous” gene or DNA is intended to refer to any gene or DNA segment that is introduced into a recipient cell, regardless of whether a similar gene may already be present in such a cell. The type of DNA included in the exogenous DNA can include DNA which is already present in the cell, DNA from another individual of the same type of organism, DNA from a different organism, or a DNA generated externally, such as a DNA sequence containing an antisense message of a gene, or a DNA sequence encoding a synthetic or modified version of a gene.

Host strains developed according to the approaches described herein can be evaluated by a number of means known in the art (see e.g., Studier (2005) Protein Expr Purif. 41(1), 207-234; Gellissen, ed. (2005) Production of Recombinant Proteins: Novel Microbial and Eukaryotic Expression Systems, Wiley-VCH, ISBN-10: 3527310363; Baneyx (2004) Protein Expression Technologies, Taylor & Francis, ISBN-10: 0954523253).

Methods of down-regulation or silencing genes are known in the art. For example, expressed protein activity can be down-regulated or eliminated using antisense oligonucleotides (ASOs), protein aptamers, nucleotide aptamers, and RNA interference (RNAi) (e.g., small interfering RNAs (siRNA), short hairpin RNA (shRNA), and micro RNAs (miRNA) (see e.g., Rinaldi and Wood (2017) Nature Reviews Neurology 14, describing ASO therapies; Fanning and Symonds (2006) Handb Exp Pharmacol. 173, 289-303G, describing hammerhead ribozymes and small hairpin RNA; Helene, et al. (1992) Ann. N.Y. Acad. Sci. 660, 27-36; Maher (1992) Bioassays 14(12): 807-15, describing targeting deoxyribonucleotide sequences; Lee et al. (2006) Curr Opin Chem Biol. 10, 1-8, describing aptamers; Reynolds et al. (2004) Nature Biotechnology 22(3), 326-330, describing RNAi; Pushparaj and Melendez (2006) Clinical and Experimental Pharmacology and Physiology 33(5-6), 504-510, describing RNAi; Dillon et al. (2005) Annual Review of Physiology 67, 147-173, describing RNAi; Dykxhoorn and Lieberman (2005) Annual Review of Medicine 56, 401-423, describing RNAi). RNAi molecules are commercially available from a variety of sources (e.g., Ambion, TX; Sigma Aldrich, MO; Invitrogen). Several siRNA molecule design programs using a variety of algorithms are known to the art (see e.g., Cenix algorithm, Ambion; BLOCK-iT™ RNAi Designer, Invitrogen; siRNA Whitehead Institute Design Tools, Bioinformatics & Research Computing). Traits influential in defining optimal siRNA sequences include G/C content at the termini of the siRNAs, Tm of specific internal domains of the siRNA, siRNA length, position of the target sequence within the CDS (coding region), and nucleotide content of the 3′ overhangs.

### Genome Editing

As described herein, immune checkpoint signals can be modulated (e.g., reduced, eliminated, or enhanced) using genome editing. Processes for genome editing are well known; see e.g. Aldi 2018 Nature Communications 9(1911). Except as otherwise noted herein, therefore, the process of the present disclosure can be carried out in accordance with such processes.

For example, genome editing can comprise CRISPR/Cas9, CRISPR-Cpf1, TALEN, or ZNFs. Adequate engagement of immune checkpoint signals by genome editing can result in protection from autoimmune or inflammatory diseases.

As an example, clustered regularly interspaced short palindromic repeats (CRISPR)/CRISPR-associated (Cas) systems are a new class of genome-editing tools that target desired genomic sites in mammalian cells. Recently published type II CRISPR/Cas systems use Cas9 nuclease that is targeted to a genomic site by complexing with a synthetic guide RNA that hybridizes to a 20-nucleotide DNA sequence and immediately preceding an NGG motif recognized by Cas9 (thus, a (N)₂₀NGG target DNA sequence). This results in a double-strand break three nucleotides upstream of the NGG motif. The double strand break instigates either non-homologous end-joining, which is error-prone and conducive to frameshift mutations that knock out gene alleles, or homology-directed repair, which can be exploited with the use of an exogenously introduced double-strand or single-strand DNA repair template to knock in or correct a mutation in the genome. Thus, genomic editing, for example, using CRISPR/Cas systems could be useful tools for therapeutic applications for GI disorders to target cells by the removal of immune checkpoint signals.

For example, the methods as described herein can comprise a method for altering a target polynucleotide sequence in a cell comprising contacting the polynucleotide sequence with a clustered regularly interspaced short palindromic repeats-associated (Cas) protein.

### Formulation

The agents and compositions described herein can be formulated by any conventional manner using one or more pharmaceutically acceptable carriers or excipients as described in, for example, Remington's Pharmaceutical Sciences (A. R. Gennaro, Ed.), 21st edition, ISBN: 0781746736 (2005), incorporated herein by reference in its entirety. Such formulations will contain a therapeutically effective amount of a biologically active agent described herein, which can be in purified form, together with a suitable amount of carrier so as to provide the form for proper administration to the subject.

The term “formulation” refers to preparing a drug in a form suitable for administration to a subject, such as a human. Thus, a “formulation” can include pharmaceutically acceptable excipients, including diluents or carriers.

The term “pharmaceutically acceptable” as used herein can describe substances or components that do not cause unacceptable losses of pharmacological activity or unacceptable adverse side effects. Examples of pharmaceutically acceptable ingredients can be those having monographs in United States Pharmacopeia (USP 29) and National Formulary (NF 24), United States Pharmacopeial Convention, Inc, Rockville, Maryland, 2005 (“USP/NF”), or a more recent edition, and the components listed in the continuously updated Inactive Ingredient Search online database of the FDA. Other useful components that are not described in the USP/NF, etc. may also be used.

The term “pharmaceutically acceptable excipient,” as used herein, can include any and all solvents, dispersion media, coatings, antibacterial and antifungal agents, isotonic, or absorption delaying agents. The use of such media and agents for pharmaceutical active substances is well known in the art (see generally Remington's Pharmaceutical Sciences (A. R. Gennaro, Ed.), 21st edition, ISBN: 0781746736 (2005)). Except insofar as any conventional media or agent is incompatible with an active ingredient, its use in the therapeutic compositions is contemplated. Supplementary active ingredients can also be incorporated into the compositions.

A “stable” formulation or composition can refer to a composition having sufficient stability to allow storage at a convenient temperature, such as between about 0° C. and about 60° C., for a commercially reasonable period of time, such as at least about one day, at least about one week, at least about one month, at least about three months, at least about six months, at least about one year, or at least about two years.

The formulation should suit the mode of administration. The agents of use with the current disclosure can be formulated by known methods for administration to a subject using several routes which include, but are not limited to, parenteral, pulmonary, oral, topical, intradermal, intratumoral, intranasal, inhalation (e.g., in an aerosol), implanted, intramuscular, intraperitoneal, intravenous, intrathecal, intracranial, intracerebroventricular, subcutaneous, intranasal, epidural, intrathecal, ophthalmic, transdermal, buccal, and rectal. The individual agents may also be administered in combination with one or more additional agents or together with other biologically active or biologically inert agents. Such biologically active or inert agents may be in fluid or mechanical communication with the agent(s) or attached to the agent(s) by ionic, covalent, Van der Waals, hydrophobic, hydrophilic or other physical forces.

Controlled-release (or sustained-release) preparations may be formulated to extend the activity of the agent(s) and reduce dosage frequency. Controlled-release preparations can also be used to affect the time of onset of action or other characteristics, such as blood levels of the agent, and consequently affect the occurrence of side effects. Controlled-release preparations may be designed to initially release an amount of an agent(s) that produces the desired therapeutic effect, and gradually and continually release other amounts of the agent to maintain the level of therapeutic effect over an extended period of time. In order to maintain a near-constant level of an agent in the body, the agent can be released from the dosage form at a rate that will replace the amount of agent being metabolized or excreted from the body. The controlled release of an agent may be stimulated by various inducers, e.g., change in pH, change in temperature, enzymes, water, or other physiological conditions or molecules.

Agents or compositions described herein can also be used in combination with other therapeutic modalities, as described further below. Thus, in addition to the therapies described herein, one may also provide to the subject other therapies known to be efficacious for treatment of the disease, disorder, or condition.

### Therapeutic Methods

Also provided is a process of treating, preventing, or reversing a GI disorder in a subject in need of administration of a therapeutically effective amount of an ICI, so as to modulate the PD-1/PDL-1 signal pathway.

Methods described herein are generally performed on a subject in need thereof. A subject in need of the therapeutic methods described herein can be a subject having, diagnosed with, suspected of having, or at risk for developing a GI disorder, including but not limited to a GI tumor. A determination of the need for treatment will typically be assessed by a history, physical exam, or diagnostic tests consistent with the disease or condition at issue. Diagnosis of the various conditions treatable by the methods described herein is within the skill of the art. The subject can be an animal subject, including a mammal, such as horses, cows, dogs, cats, sheep, pigs, mice, rats, monkeys, hamsters, guinea pigs, and humans or chickens. For example, the subject can be a human subject.

Generally, a safe and effective amount of an immune checkpoint modulation agent is, for example, an amount that would cause the desired therapeutic effect in a subject while minimizing undesired side effects. In various embodiments, an effective amount of an immune checkpoint modulation agent described herein can substantially inhibit a GI disorder, slow the progress of a GI disorder, or limit the development of a GI disorder.

According to the methods described herein, administration can be parenteral, pulmonary, oral, topical, intradermal, intramuscular, intraperitoneal, intravenous, intratumoral, intrathecal, intracranial, intracerebroventricular, subcutaneous, intranasal, epidural, ophthalmic, buccal, or rectal administration.

When used in the treatments described herein, a therapeutically effective amount of an ICI can be employed in pure form or, where such forms exist, in pharmaceutically acceptable salt form and with or without a pharmaceutically acceptable excipient. For example, the compounds of the present disclosure can be administered, at a reasonable benefit/risk ratio applicable to any medical treatment, in a sufficient amount to treat a GI disorder.

The amount of a composition described herein that can be combined with a pharmaceutically acceptable carrier to produce a single dosage form will vary depending upon the subject or host treated and the particular mode of administration. It will be appreciated by those skilled in the art that the unit content of agent contained in an individual dose of each dosage form need not in itself constitute a therapeutically effective amount, as the necessary therapeutically effective amount could be reached by administration of a number of individual doses.

Toxicity and therapeutic efficacy of compositions described herein can be determined by standard pharmaceutical procedures in cell cultures or experimental animals for determining the LD₅₀ (the dose lethal to 50% of the population) and the ED₅₀, (the dose therapeutically effective in 50% of the population). The dose ratio between toxic and therapeutic effects is the therapeutic index that can be expressed as the ratio LD₅₀/ED₅₀, where larger therapeutic indices are generally understood in the art to be optimal.

The specific therapeutically effective dose level for any particular subject will depend upon a variety of factors including the disorder being treated and the severity of the disorder; activity of the specific compound employed; the specific composition employed; the age, body weight, general health, sex and diet of the subject; the time of administration; the route of administration; the rate of excretion of the composition employed; the duration of the treatment; drugs used in combination or coincidental with the specific compound employed; and like factors well known in the medical arts (see e.g., Koda-Kimble et al. (2004) Applied Therapeutics: The Clinical Use of Drugs, Lippincott Williams & Wilkins, ISBN 0781748453; Winter (2003) Basic Clinical Pharmacokinetics, 4^{th} ed., Lippincott Williams & Wilkins, ISBN 0781741475; Sharqel (2004) Applied Biopharmaceutics & Pharmacokinetics, McGraw-Hill/Appleton & Lange, ISBN 0071375503). For example, it is well within the skill of the art to start doses of the composition at levels lower than those required to achieve the desired therapeutic effect and to gradually increase the dosage until the desired effect is achieved. If desired, the effective daily dose may be divided into multiple doses for purposes of administration. Consequently, single dose compositions may contain such amounts or submultiples thereof to make up the daily dose. It will be understood, however, that the total daily usage of the compounds and compositions of the present disclosure will be decided by an attending physician within the scope of sound medical judgment.

Again, each of the states, diseases, disorders, and conditions, described herein, as well as others, can benefit from compositions and methods described herein. Generally, treating a state, disease, disorder, or condition includes preventing, reversing, or delaying the appearance of clinical symptoms in a mammal that may be afflicted with or predisposed to the state, disease, disorder, or condition but does not yet experience or display clinical or subclinical symptoms thereof. Treating can also include inhibiting the state, disease, disorder, or condition, e.g., arresting or reducing the development of the disease or at least one clinical or subclinical symptom thereof. Furthermore, treating can include relieving the disease, e.g., causing regression of the state, disease, disorder, or condition or at least one of its clinical or subclinical symptoms. A benefit to a subject to be treated can be either statistically significant or at least perceptible to the subject or to a physician.

Administration of an immune checkpoint modulation agent can occur as a single event or over a time course of treatment. For example, an immune checkpoint modulation agent can be administered daily, weekly, bi-weekly, or monthly. For treatment of acute conditions, the time course of treatment will usually be at least several days. Certain conditions could extend treatment from several days to several weeks. For example, treatment could extend over one week, two weeks, or three weeks. For more chronic conditions, treatment could extend from several weeks to several months or even a year or more.

Treatment in accord with the methods described herein can be performed prior to, concurrent with, or after conventional treatment modalities for a GI disorder.

An immune checkpoint modulation agent can be administered simultaneously or sequentially with another agent, such as an antibiotic, an anti-inflammatory, or another agent. For example, an immune checkpoint modulation agent can be administered simultaneously with another agent, such as an antibiotic or an anti-inflammatory. Simultaneous administration can occur through administration of separate compositions, each containing one or more of an immune checkpoint modulation agent, an antibiotic, an anti-inflammatory, or another agent. Simultaneous administration can occur through administration of one composition containing two or more of an immune checkpoint modulation agent, an antibiotic, an anti-inflammatory, or another agent. An immune checkpoint modulation agent can be administered sequentially with an antibiotic, an anti-inflammatory, or another agent. For example, an immune checkpoint modulation agent can be administered before or after administration of an antibiotic, an anti-inflammatory, or another agent.

### Administration

Agents and compositions described herein can be administered according to methods described herein in a variety of means known to the art. The agents and composition can be used therapeutically either as exogenous materials or as endogenous materials. Exogenous agents are those produced or manufactured outside of the body and administered to the body. Endogenous agents are those produced or manufactured inside the body by some type of device (biologic or other) for delivery within or to other organs in the body.

As discussed above, administration can be parenteral, pulmonary, oral, topical, intradermal, intratumoral, intranasal, inhalation (e.g., in an aerosol), implanted, intramuscular, intraperitoneal, intravenous, intrathecal, intracranial, intracerebroventricular, subcutaneous, intranasal, epidural, intrathecal, ophthalmic, transdermal, buccal, and rectal.

Agents and compositions described herein can be administered in a variety of methods well known in the arts. Administration can include, for example, methods involving oral ingestion, direct injection (e.g., systemic or stereotactic), implantation of cells engineered to secrete the factor of interest, drug-releasing biomaterials, polymer matrices, gels, permeable membranes, osmotic systems, multilayer coatings, microparticles, implantable matrix devices, mini-osmotic pumps, implantable pumps, injectable gels and hydrogels, liposomes, micelles (e.g., up to 30 μm), nanospheres (e.g., less than 1 μm), microspheres (e.g., 1-100 μm), reservoir devices, a combination of any of the above, or other suitable delivery vehicles to provide the desired release profile in varying proportions. Other methods of controlled-release delivery of agents or compositions will be known to the skilled artisan and are within the scope of the present disclosure.

Delivery systems may include, for example, an infusion pump which may be used to administer the agent or composition in a manner similar to that used for delivering insulin or chemotherapy to specific organs or tumors. Typically, using such a system, an agent or composition can be administered in combination with a biodegradable, biocompatible polymeric implant that releases the agent over a controlled period of time at a selected site. Examples of polymeric materials include polyanhydrides, polyorthoesters, polyglycolic acid, polylactic acid, polyethylene vinyl acetate, and copolymers and combinations thereof. In addition, a controlled release system can be placed in proximity of a therapeutic target, thus requiring only a fraction of a systemic dosage.

Agents can be encapsulated and administered in a variety of carrier delivery systems. Examples of carrier delivery systems include microspheres, hydrogels, polymeric implants, smart polymeric carriers, and liposomes (see generally, Uchegbu and Schatzlein, eds. (2006) Polymers in Drug Delivery, CRC, ISBN-10: 0849325331). Carrier-based systems for molecular or biomolecular agent delivery can: provide for intracellular delivery; tailor biomolecule/agent release rates; increase the proportion of biomolecule that reaches its site of action; improve the transport of the drug to its site of action; allow colocalized deposition with other agents or excipients; improve the stability of the agent in vivo; prolong the residence time of the agent at its site of action by reducing clearance; decrease the nonspecific delivery of the agent to nontarget tissues; decrease irritation caused by the agent; decrease toxicity due to high initial doses of the agent; alter the immunogenicity of the agent; decrease dosage frequency, improve taste of the product; or improve shelf life of the product.

### Screening

Also provided are methods for screening.

The subject methods find use in the screening of a variety of different candidate molecules (e.g., potentially therapeutic candidate molecules). Candidate substances for screening according to the methods described herein include, but are not limited to, fractions of tissues or cells, nucleic acids, polypeptides, siRNAs, antisense molecules, aptamers, ribozymes, triple helix compounds, antibodies, and small (e.g., less than about 2000 mw, or less than about 1000 mw, or less than about 800 mw) organic molecules or inorganic molecules including but not limited to salts or metals.

Candidate molecules encompass numerous chemical classes, for example, organic molecules, such as small organic compounds having a molecular weight of more than 50 and less than about 2,500 Daltons. Candidate molecules can comprise functional groups necessary for structural interaction with proteins, particularly hydrogen bonding, and typically include at least an amine, carbonyl, hydroxyl or carboxyl group, and usually at least two of the functional chemical groups. The candidate molecules can comprise cyclical carbon or heterocyclic structures and/or aromatic or polyaromatic structures substituted with one or more of the above functional groups.

A candidate molecule can be a compound in a library database of compounds. One of skill in the art will be generally familiar with, for example, numerous databases for commercially available compounds for screening (see e.g., ZINC database, UCSF, with 2.7 million compounds over 12 distinct subsets of molecules; Irwin and Shoichet (2005) J Chem Inf Model 45, 177-182). One of skill in the art will also be familiar with a variety of search engines to identify commercial sources or desirable compounds and classes of compounds for further testing (see e.g., ZINC database; eMolecules.com; and electronic libraries of commercial compounds provided by vendors, for example: ChemBridge, Princeton BioMolecular, Ambinter SARL, Enamine, ASDI, Life Chemicals etc.).

Candidate molecules for screening according to the methods described herein include both lead-like compounds and drug-like compounds. A lead-like compound is generally understood to have a relatively smaller scaffold-like structure (e.g., molecular weight of about 150 to about 350 kD) with relatively fewer features (e.g., less than about 3 hydrogen donors and/or less than about 6 hydrogen acceptors; hydrophobicity character xlogP of about −2 to about 4) (see e.g., Angewante (1999) Chemie Int. ed. Engl. 24, 3943-3948). In contrast, a drug-like compound is generally understood to have a relatively larger scaffold (e.g., molecular weight of about 150 to about 500 kD) with relatively more numerous features (e.g., less than about 10 hydrogen acceptors and/or less than about 8 rotatable bonds; hydrophobicity character xlogP of less than about 5) (see e.g., Lipinski (2000) J. Pharm. Tox. Methods 44, 235-249). Initial screening can be performed with lead-like compounds.

When designing a lead from spatial orientation data, it can be useful to understand that certain molecular structures are characterized as being “drug-like”. Such characterization can be based on a set of empirically recognized qualities derived by comparing similarities across the breadth of known drugs within the pharmacopoeia. While it is not required for drugs to meet all, or even any, of these characterizations, it is far more likely for a drug candidate to meet with clinical successful if it is drug-like.

Several of these “drug-like” characteristics have been summarized into the four rules of Lipinski (generally known as the “rules of fives” because of the prevalence of the number 5 among them). While these rules generally relate to oral absorption and are used to predict bioavailability of compound during lead optimization, they can serve as effective guidelines for constructing a lead molecule during rational drug design efforts such as may be accomplished by using the methods of the present disclosure.

The four “rules of five” state that a candidate drug-like compound should have at least three of the following characteristics: (i) a weight less than 500 Daltons; (ii) a log of P less than 5; (iii) no more than 5 hydrogen bond donors (expressed as the sum of OH and NH groups); and (iv) no more than 10 hydrogen bond acceptors (the sum of N and O atoms). Also, drug-like molecules typically have a span (breadth) of between about 8 Å to about 15 Å.

### Kits

Also provided are kits. Such kits can include an agent or composition described herein and, in certain embodiments, instructions for administration. Such kits can facilitate performance of the methods described herein. When supplied as a kit, the different components of the composition can be packaged in separate containers and admixed immediately before use. Components include, but are not limited to yeast that produce an immune checkpoint modulation agent. Such packaging of the components separately can, if desired, be presented in a pack or dispenser device which may contain one or more unit dosage forms containing the composition. The pack may, for example, comprise metal or plastic foil such as a blister pack. Such packaging of the components separately can also, in certain instances, permit long-term storage without losing activity of the components.

Kits may also include reagents in separate containers such as, for example, sterile water or saline to be added to a lyophilized active component packaged separately. For example, sealed glass ampules may contain a lyophilized component and in a separate ampule, sterile water, sterile saline each of which has been packaged under a neutral non-reacting gas, such as nitrogen. Ampules may consist of any suitable material, such as glass, organic polymers, such as polycarbonate, polystyrene, ceramic, metal or any other material typically employed to hold reagents. Other examples of suitable containers include bottles that may be fabricated from similar substances as ampules, and envelopes that may consist of foil-lined interiors, such as aluminum or an alloy. Other containers include test tubes, vials, flasks, bottles, syringes, and the like. Containers may have a sterile access port, such as a bottle having a stopper that can be pierced by a hypodermic injection needle. Other containers may have two compartments that are separated by a readily removable membrane that upon removal permits the components to mix. Removable membranes may be glass, plastic, rubber, and the like.

In certain embodiments, kits can be supplied with instructional materials. Instructions may be printed on paper or other substrate, and/or may be supplied as an electronic-readable medium or video. Detailed instructions may not be physically associated with the kit, instead, a user may be directed to an Internet web site specified by the manufacturer or distributor of the kit.

A control sample or a reference sample as described herein can be a sample from a healthy subject. A reference value can be used in place of a control or reference sample, which was previously obtained from a healthy subject or a group of healthy subjects. A control sample or a reference sample can also be a sample with a known amount of a detectable compound or a spiked sample.

The methods and algorithms of the invention may be enclosed in a controller or processor. Furthermore, methods and algorithms of the present invention, can be embodied as a computer implemented method or methods for performing such computer-implemented method or methods, and can also be embodied in the form of a tangible or non-transitory computer readable storage medium containing a computer program or other machine-readable instructions (herein “computer program”), wherein when the computer program is loaded into a computer or other processor (herein “computer”) and/or is executed by the computer, the computer becomes an apparatus for practicing the method or methods. Storage media for containing such computer program include, for example, floppy disks and diskettes, compact disk (CD)-ROMs (whether or not writeable), DVD digital disks, RAM and ROM memories, computer hard drives and back-up drives, external hard drives, “thumb” drives, and any other storage medium readable by a computer. The method or methods can also be embodied in the form of a computer program, for example, whether stored in a storage medium or transmitted over a transmission medium such as electrical conductors, fiber optics or other light conductors, or by electromagnetic radiation, wherein when the computer program is loaded into a computer and/or is executed by the computer, the computer becomes an apparatus for practicing the method or methods. The method or methods may be implemented on a general-purpose microprocessor or on a digital processor specifically configured to practice the process or processes. When a general-purpose microprocessor is employed, the computer program code configures the circuitry of the microprocessor to create specific logic circuit arrangements. Storage medium readable by a computer includes medium being readable by a computer per se or by another machine that reads the computer instructions for providing those instructions to a computer for controlling its operation. Such machines may include, for example, machines for reading the storage media mentioned above.

Compositions and methods described herein utilizing molecular biology protocols can be according to a variety of standard techniques known to the art (see e.g., Sambrook and Russel (2006) Condensed Protocols from Molecular Cloning: A Laboratory Manual, Cold Spring Harbor Laboratory Press, ISBN-10: 0879697717; Ausubel et al. (2002) Short Protocols in Molecular Biology, 5th ed., Current Protocols, ISBN-10: 0471250929; Sambrook and Russel (2001) Molecular Cloning: A Laboratory Manual, 3d ed., Cold Spring Harbor Laboratory Press, ISBN-10: 0879695773; Elhai, J. and Wolk, C. P. 1988. Methods in Enzymology 167, 747-754; Studier (2005) Protein Expr Purif. 41(1), 207-234; Gellissen, ed. (2005) Production of Recombinant Proteins: Novel Microbial and Eukaryotic Expression Systems, Wiley-VCH, ISBN-10: 3527310363; Baneyx (2004) Protein Expression Technologies, Taylor & Francis, ISBN-10: 0954523253).

Definitions and methods described herein are provided to better define the present disclosure and to guide those of ordinary skill in the art in the practice of the present disclosure. Unless otherwise noted, terms are to be understood according to conventional usage by those of ordinary skill in the relevant art.

In some embodiments, numbers expressing quantities of ingredients, properties such as molecular weight, reaction conditions, and so forth, used to describe and claim certain embodiments of the present disclosure are to be understood as being modified in some instances by the term “about.” In some embodiments, the term “about” is used to indicate that a value includes the standard deviation of the mean for the device or method being employed to determine the value. In some embodiments, the numerical parameters set forth in the written description and attached claims are approximations that can vary depending upon the desired properties sought to be obtained by a particular embodiment. In some embodiments, the numerical parameters should be construed in light of the number of reported significant digits and by applying ordinary rounding techniques. Notwithstanding that the numerical ranges and parameters setting forth the broad scope of some embodiments of the present disclosure are approximations, the numerical values set forth in the specific examples are reported as precisely as practicable. The numerical values presented in some embodiments of the present disclosure may contain certain errors necessarily resulting from the standard deviation found in their respective testing measurements. The recitation of ranges of values herein is merely intended to serve as a shorthand method of referring individually to each separate value falling within the range. Unless otherwise indicated herein, each individual value is incorporated into the specification as if it were individually recited herein. The recitation of discrete values is understood to include ranges between each value.

In some embodiments, the terms “a” and “an” and “the” and similar references used in the context of describing a particular embodiment (especially in the context of certain of the following claims) can be construed to cover both the singular and the plural, unless specifically noted otherwise. In some embodiments, the term “or” as used herein, including the claims, is used to mean “and/or” unless explicitly indicated to refer to alternatives only or the alternatives are mutually exclusive.

The terms “comprise,” “have” and “include” are open-ended linking verbs. Any forms or tenses of one or more of these verbs, such as “comprises,” “comprising,” “has,” “having,” “includes” and “including,” are also open-ended. For example, any method that “comprises,” “has” or “includes” one or more steps is not limited to possessing only those one or more steps and can also cover other unlisted steps. Similarly, any composition or device that “comprises,” “has” or “includes” one or more features is not limited to possessing only those one or more features and can cover other unlisted features.

All methods described herein can be performed in any suitable order unless otherwise indicated herein or otherwise clearly contradicted by context. The use of any and all examples, or exemplary language (e.g., “such as”) provided with respect to certain embodiments herein is intended merely to better illuminate the present disclosure and does not pose a limitation on the scope of the present disclosure otherwise claimed. No language in the specification should be construed as indicating any non-claimed element essential to the practice of the present disclosure.

Groupings of alternative elements or embodiments of the present disclosure disclosed herein are not to be construed as limitations. Each group member can be referred to and claimed individually or in any combination with other members of the group or other elements found herein. One or more members of a group can be included in, or deleted from, a group for reasons of convenience or patentability. When any such inclusion or deletion occurs, the specification is herein deemed to contain the group as modified thus fulfilling the written description of all Markush groups used in the appended claims.

All publications, patents, patent applications, and other references cited in this application are incorporated herein by reference in their entirety for all purposes to the same extent as if each individual publication, patent, patent application or other reference was specifically and individually indicated to be incorporated by reference in its entirety for all purposes. Citation of a reference herein shall not be construed as an admission that such is prior art to the present disclosure.

Having described the present disclosure in detail, it will be apparent that modifications, variations, and equivalent embodiments are possible without departing the scope of the present disclosure defined in the appended claims. Furthermore, it should be appreciated that all examples in the present disclosure are provided as non-limiting examples.

### EXAMPLES

The following non-limiting examples are provided to further illustrate the present disclosure. It should be appreciated by those of skill in the art that the techniques disclosed in the examples that follow represent approaches the inventors have found function well in the practice of the present disclosure, and thus can be considered to constitute examples of modes for its practice. However, those of skill in the art should, in light of the present disclosure, appreciate that many changes can be made in the specific embodiments that are disclosed and still obtain a like or similar result without departing from the spirit and scope of the present disclosure.

### Example 1—A Yeast-Based Oral Therapeutic Platform for Delivery of Immune Checkpoint Inhibitors Reduces Intestinal 2 Tumor Burden

To develop and validate an engineered ICI secretion system in the yeast Saccharomyces boulardii (Sb) to treat intestinal tumors via oral administration, the following experiments were conducted.

16S rRNA gene sequencing data obtained as described below was deposited at NCBI SRA (accession number: PRJNA1040306).

### Microbial Strains

For production of ICI payloads, E. coli DH5α-E was used for subcloning and E. coli Star BL21 was used as a recombinant expression vector. These strains are commercially available and commonly used for cloning and production of recombinant proteins. They were cultured in standard LB at 37° C. according to the manufacturer guidelines.

For development of the yeast-based secretion system, Saccharomyces cerevisiae var. boulardii was used as a chassis. Unless otherwise stated, Sb constructs were grown in YP broth with 2% glucose and 50 μg/mL blasticidin S in aerobic conditions at 30° C. Sb seed cultures were grown for approximately 30 hours before subculturing to an OD₆₀₀ of 0.1, followed by an additional 16 hours of growth prior to use in subsequent assays.

### PD-1/PD-L1 Blockade Assay

The PD-1/PD-L1 blockade assay used to quantify immune checkpoint blockade of microbially-derived ICI payloads is a commercially available assay (Promega, J1250) that relies on a luminescent reporter linked to PD-1/PD-L1 inhibition between two cell types. The cell lines included in this assay are PD-1 effector cells (Jurkat T cells stably expressing PD-1 and NFAT-induced luciferase) and PD-1 aAPC/CHO-K1 cells (CHO-K1 cells stably expressing PD-L1 and a cell surface protein designed to activate cognate T-cell receptors in an antigen-independent manner). These thaw-and-use cells were cultured and conditioned according to the manufacturer's instructions and were not further propagated.

### Animal Models

Three different mouse models were used to characterize Sb constructs and their recombinant payloads. All mouse husbandry and experimental procedures were performed at Washington University in St. Louis School of Medicine (WUSM) with institutional approval and oversight by the WUSM Institutional Animal Care and Use Committee (IACUC, Protocol 21-0160). All mice are fed a standard mouse chow (*****) and water ad libitium except as noted. Mice were housed in a (****cage-racks****) within a specific pathogen free BSL-1 facility and maintained on a 12 hour light/dark cycle at 30-70% humidity at 68-79° F. Cages were cleaned weekly with additional, daily cage changes following completion of therapeutic intervention. Mice were carefully monitored throughout experiments for signs of irreversible decline and moribund mice were ethically euthanized in accordance with IACUC and American Veterinary Medical Association (AVMA) guidelines.

We maintain an onsite APC^{min} mouse breeding colony that breeds C57BL/6J-APC^{min}/J sires (sourced directly from Jackson) with C57BL/6J dams (sourced from Jackson or WT offspring from our breeding colony). Pups were genotyped as APC^{min}-Het or APC^{min}-WT and separated by sex and genotype following weaning. Due to the propensity for adult male mice to fight non-littermates, we were unable to inter-mix mice from different cages following tumor induction. Mice were allowed a minimum two-week acclimation period following their arrival to our facility prior to starting experiments. Whenever feasible, mouse experiments were conducted using mice from our breeding colony (APC^{min}-Hets and WT littermates) as well as mice ordered directly from Jackson Laboratory. All experiments included mice of both sexes and all manipulations were executed by multiple personnel to avoid experimental bias.

For gut transit, in vivo plasmid retention, ex vivo GLuc detection, and PK/PD experiments, healthy WT C57BL-6J mice 8-12 weeks of age were used. For gut transit and PK/PD experiments, sample collection entailed collection of tissue or large volumes of blood. As such, terminal timepoints were used and individual data points represent samples collected from different mice. For in vivo plasmid retention and ex vivo GLuc detection experiments, only stool was collected and therefore samples were repeatedly collected from the same mouse for the duration of each experiment.

For therapeutic efficacy experiments, we utilized male and female APC^{min} mice, which develop spontaneous tumors throughout their small intestine, in combination with AOM challenge, which induces tumor formation in the colon [17638923]. AOM-directed tumor induction began when mice were aged 7-12 weeks and treatment intervention commenced 5 weeks later, allowing for robust tumor development to occur prior to treatment [17638923].

### Method Details

### Recombinant Protein Expression and Purification

haPD-1 and rPD-L1 nb were expressed and purified recombinantly from E. coli as an expression host. haPD-1 purification was outsourced through GenScript and rPD-L1 nb was purified in-house. The gene encoding haPD-1 with N-terminal 6×His and HA epitope tags was codon optimized for E. coli expression, subcloned into the pET30a vector, and transformed into E. coli. Following IPTG induction, inclusion bodies were extracted from E. coli and haPD-1 was purified via Nickel and Q column chromatography and stored in 50 mM Tris-HCl, 150 mM NaCl, 10% Glycerol, pH 8.0.

The gene for rPD-L1 nb with an N-terminal HA tag and C-terminal 6×His tag was codon-optimized for E. coli expression, subcloned into the pET28b(+) vector, and transformed into BL21 Star™ (DE3) E. coli. Mid-log cultures were induced with 1 mM IPTG overnight at 20° C. The following day, cultures were spun down and the pellets were stored at −80° C. until ready for lysis. Thawed pellets were lysed in B-PER™ Complete supplemented with 1 mM EDTA, Pierce Protease Inhibitor Cocktail, and 1 mg/mL lysozyme at room temperature for 1 hour with rotation. Lysates were centrifuged at 16,000×G for 20 minutes at 4° C. Supernatants were passed through a 0.22 μm filter, mixed 1:1 with Buffer A (100 mM sodium phosphate, 300 mM NaCl, pH 8.0), and incubated with Ni-NTA beads overnight at 4° C. with gentle rotation. The following day, rPD-L1 nb was eluted from the resin using a stepwise elution gradient of 25-, 50-, 100-, 150-, and 250-mM imidazole in Buffer A. The purity of each fraction was assessed using SDS-PAGE gel electrophoresis followed by total protein stain using Coomassie total protein stain. Fractions containing >95% purity of rPD-L1 nb were pooled, buffer-exchanged, and concentrated in PBS.

Genetic Engineering of S. boulardii

The OGS539 vector was purchased directly from Sigma Aldrich (OGS539) and transformed into E. coli DH5a. Genes encoding Gaussia luciferase (GLuc), haPD-1, and rPD-L1 nb with N-terminal mating factor α secretion signal and the HA epitope tag were synthesized as gBlocks™ from IDT and cloned into OGS539 using Gibson Assembly [19363495]. Following transformation into E. coli DH5a, colonies were screened for insertion of the genes using colony PCR with the CP-F1/CP-R1 primers and validated via sequencing by Plasmidsaurus. The plasmids were transformed in S. boulardii MYA-797 following the lithium acetate transformation procedure [17401334] and plated on yeast peptone (YP) agar containing 2% glucose (w/v) and 50 μg/mL blasticidin S. Yeast colonies were screened using the CP-F1/CP-R1 colony PCR primer set (SEQ ID NOS: 24 and 25). PCR reactions producing positive bands were purified and sequenced using Plasmidsaurus.

### Western Blot

Purified proteins or 48-hour Sb construct culture broths were diluted in Laemmli buffer with β-mercaptoethanol followed by 95° C. denaturation. Samples were size separated via 12% Tris-Glycine gel electrophoresis and transferred to nitrocellulose membranes using the iBlot® 2 system. Blots were blocked for 1 hour in 5% w/v dry milk in TBST. An αHA primary antibody (ab236632) was spiked 1:5000 into the blocking solution and incubated overnight. Blots were washed three times with TBST and incubated with horse radish peroxidase-conjugated goat anti-rabbit secondary antibody (Invitrogen 31460) diluted 1:5000 in TBST with 15 mg/mL BSA for 1 hour. Blots were again washed with TBST and then incubated with Pierce™ ECL Western Blotting Substrate. Images were captured on the Bio-Rad Chemi Doc XRS+ Imaging system.

### ELISA-Based PD-L1 Binding Assays

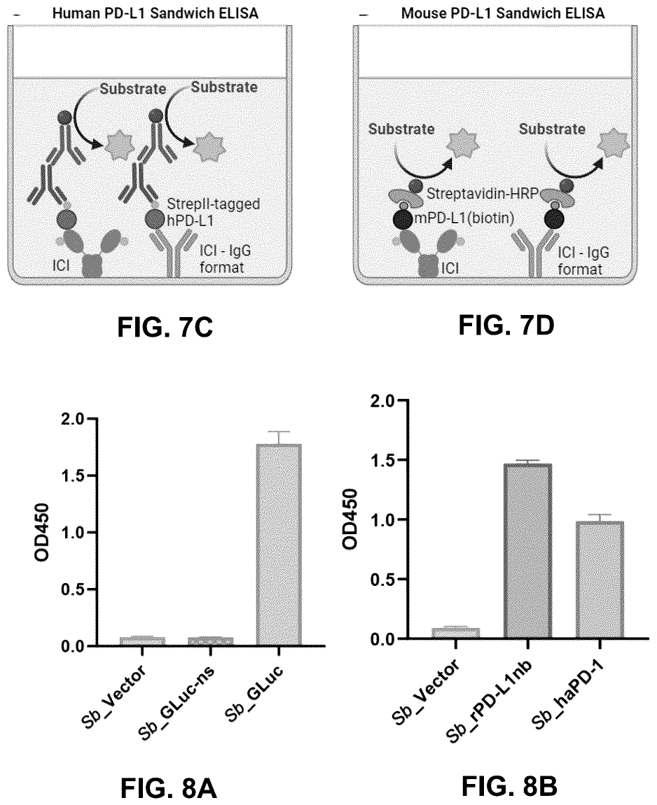

Four ELISA formats were utilized in this study. To assay absolute protein payloads produced from the Sb secretion systems, we employed an indirect ELISA (FIG. 7A) wherein culture broths collected from Sb constructs grown for 48 hours were mixed 1:1 with 100 mM sodium carbonate buffer, applied directly to a 96-well polystyrene plate (Costar 3370), allowed to bind overnight at 4° C., and blocked overnight with 1% BSA in PBS-T at 4° C. Indirect immunodetection of the HA epitope tag was performed by incubation with an αHA primary antibody (Abcam ab236632) diluted 1:5000 in 1% BSA in PBS-T for 1 hour followed by goat-anti-rabbit HRP conjugate secondary antibody (Invitrogen 31460) diluted 1:5000 in 1% BSA in PBS-T for 1 hour. The plates were incubated with TMB substrate (Thermo 34028) for 30 minutes at room temperature and an equivalent volume of 2M sulfuric acid was added to each well, after which OD₄₅₀ values were determined on a BioTek PowerWave HT plate reader.

Sandwich ELISAs were also performed in three formats, including an ICI sandwich ELISA, human PD-L1 (hPD-L1) sandwich ELISA, and mouse PD-L1 (mPD-L1) sandwich ELISA (FIG. 7B-D). For the ICI sandwich ELISA, plates were coated with 1 μg/mL PD-L1 (ab167713) in 100 mM sodium carbonate buffer overnight at 4° C. and blocked overnight with 1% BSA in PBS-T at 4° C. Sb culture broths and other samples containing haPD-1 or rPD-L1 nb were applied to the plates for 1 hour at 37° C. αHA tag immunodetection was performed as described for the indirect ELISA above with the same antibody set.

For the hPD-L1 sandwich ELISA, plates were coated with dilution series of the ICIs (haPD-1, rPD-L1nb, and conventional IgG ICIs) in 100 mM sodium carbonate buffer overnight at 4° C. and blocked overnight as described above. 1 μg/mL StrepII-tagged hPD-L1 (ab182689) in blocking buffer was applied to the plate for 1 hour at 37° C., followed by incubation with a αStrepII-tag primary antibody (ab180957) and the same secondary antibody/development procedure as above.

For the mPD-L1 sandwich ELISA, plates were coated and blocked as described above for the hPD-L1 sandwich ELISA. 1 μg/mL biotinylated mPD-L1 (ab216261) in blocking buffer was applied to the plate for 1 hour at 37° C. Streptavidin-HRP conjugate protein (Thermo Scientific N100) was diluted 1:20,000 in blocking buffer and applied to the plate for 1 hour, followed by incubation with TMB substrate and OD₄₅₀ measurement as described above.

For all sandwich ELISAs, raw OD₄₅₀ values generated from the ELISA were corrected using a set of wells with identical steps of the ELISA performed with the exception of the initial antigen binding step to account for nonspecific binding of PD-L1 or sample to the assay plate. K_{D} values were determined using corrected OD₄₅₀ curves or percent maximum signal curves using the Specific binding with Hill Slope equation in Prism.

### Yeast Culturing

Plasmid retention was measured by growing constructs in YP+2% glucose+/−50 μg/mL blasticidin S followed by enumeration on plates containing YP with 2% glucose+/−50 μg/mL blasticidin S. To enumerate Sb titers in mouse stool, stool was collected and weighed, diluted in 1 mL sterile PBS, and vortexed at maximum speed for 5 min. Stool solution was serially diluted on Sabouraud dextrose agar (SDA) supplemented with chloramphenicol (50 mg/L), gentamycin (5 mg/L), and 50 μg/mL blasticidin S for selective growth of Sb constructs.

### Secretion Assays

To perform growth and secretion assays of Sb constructs, Sb cultures were back diluted to an OD₆₀₀ of 0.1 and grown for 8 hours in YP broth with 2% glucose and 50 μg/mL blasticidin S in aerobic conditions at 30° C. unless otherwise specified. Growth was measured via OD₆₀₀ absorbance and/or CFU enumeration on selective plates as described above. To assess secretion, a culture aliquot was collected at each timepoint, centrifuged at maximum speed for 1 minute, and the supernatants were stored at −80° C. for subsequent analysis via indirect or ICI sandwich ELISA as described above.

To assess growth and secretion using simulated physiological conditions, Sb was cultured via five unique culturing conditions. First, the optimal growth condition is described above (30° C., aerobic with agitation, 2% glucose). The next 3 culturing conditions (37° C., anaerobic, and 0.1% glucose) each altered a single variable while the other conditions remained identical to the optimal growth conditions. For anaerobic growth assays, cultures were incubated in an anaerobic Coy chamber without agitation. The final condition (“physiological”) entails changing all of the parameters (37° C., anaerobic, and 0.1% glucose) at the same time. Each condition was tested with a minimum of four replicates and the optimal culturing condition was included in each experiment to control for experimental variability. Growth and secretion were quantified as described above.

### PD-1/PD-L1 Blockade Assays

Sb_haPD-1, Sb_rPDL1-nb and Sb_Vector strains were cultured for 30 hours, sub-cultured to OD₆₀₀ 0.1 and then grown for 16 hours. Supernatants were collected and concentrated using Amicon™ Ultra-15 10 kDa centrifugal filters (Millipore Sigma UFC9010). To measure antibody function, Sb-secreted supernatants, purified haPD-1, purified rPD-L1 nb and commercial controls (anti-human PD-1-Promega J1201, Ultra-Leaf™ anti-mouse PD-1-BioLegend 135247) were utilized in human or mouse blockade assays. Briefly, mouse or human PD-L1 aAPC/CHO-K1 cells in 100 μL cell recovery media (90% Hams F-12/10% FBS) were added to 96 well tissue culture plates (Corning 3917) and incubated for 20 hours at 37° C. After incubation, media were removed from wells and 40 μL 2.5-fold serial dilutions of antibodies in assay buffer (99% RPMI 1640/1% FBS) were added to the wells containing cells immediately followed by adding 40 μL of PD-1 effector cells. Cells and antibody mix were incubated for 6 hours at 37° C. Following the incubation, 80 μL Bio-Glo™ Reagent was added to the wells, allowed to incubate for 20 minutes and imaged using a luminometer.

### Gut Transit Dynamics

Healthy WT C57BL/6J mice received oral gavages with 100 μL PBS containing 107 CFU of Sb_haPD-1. Mice were sacrificed at various timepoints and the GI tract was harvested. Whole contents were collected from the distal 10 cm of the SI, cecum, and colon, along with terminal stool. Contents were diluted in 1 mL PBS, vortexed at maximum speed, and solution was serially diluted on SDA supplemented with chloramphenicol (50 mg/L), gentamycin (5 mg/L), and 50 μg/mL blasticidin S for selective growth of Sb constructs.

### In Vivo Plasmid Stability

Healthy WT C57BL/6J mice received oral gavages with 10⁸ CFU of Sb_Vector or Sb_haPD-1 in 100 μL of PBS. Stool was collected from mice 24 hours post-gavage, diluted in 1 mL PBS, vortexed at maximum speed, and plated on SDA. SDA plates were supplemented with chloramphenicol (50 mg/L) and gentamycin (5 mg/L) but lacked blasticidin S, which was purposely excluded to allow for growth of Sb that no longer maintained their plasmid. Following a 48 hour incubation period, 24 colonies from each treatment group were picked and grown in 96 deep well plates containing YP+2% glucose with 50 μg/mL blasticidin S for 24 hours. OD₆₀₀ was measured to assess residual blasticidin resistance, followed by centrifugation and collection of supernatants for subsequent haPD-1 detection via hPD-L1 sandwich ELISA as described above.

### Ex Vivo Detection of GLuc

Healthy WT C57BL/6J mice received oral gavages with 10⁸ CFU of Sb_Vector or Sb_GLuc in 100 μL of PBS. Stool was collected from mice every 2 hours for 8 hours and stored at −80 C. Frozen stools were thawed and immediately resuspended in 500 μL PBS with a protease inhibitor cocktail (Pierce A32953) by vortexing for 10 minutes. Each stool homogenate was transferred to an opaque 96-well plate. Coelenterazine (CEZ) was prepared at 100 μM in PBS and stored in the dark for 30 minutes. The CEZ solution was mixed with stool homogenates 1:1 and incubated with shaking in the dark for 10 minutes. Following luminescence reads on a Biotek Synergy H1, the final luminescence readout for each sample was computed as a signal fold-change relative to the baseline of each individual mouse.

### PK/PD Study

Healthy WT C57BL/6J mice received IP injections of purified, E. coli-derived haPD-1 or rPD-L1 nb (100 μg). At each timepoint, a terminal submandibular bleed was performed and blood was collected in EDTA-coated tubes (BD Microtainer, 365974). Blood was mixed 1:1 with 100 mM sodium carbonate buffer and ICIs were quantified via indirect ELISA as described above.

### APCₘᵢₙ+AOM Tumor Induction

Male and female C57BL/6J-APC^{min}/J mice aged 7-12 weeks were administered AOM (8 mg/kg) on days 1, 8, and 15. Throughout the induction period, mice were observed for excessive weight loss, prolapsed rectum, and other signs of moribundity. On day 36, mice that maintained at least 90% of their initial bodyweight moved onto the treatment phase of the experiment.

### Probiotic Treatment

Following the induction period, cages were randomized across four treatment groups: (1) PBS, (2) Sb_Vector, (3) Sb_haPD-1, (4) α-mPD-L1. Mice in groups 1, 2, and 3 received 100 μL of therapy via oral gavage every other day for a total of 7 doses. Probiotic inoculums were prepared from overnight (˜16 hour) cultures resuspended in PBS at a concentration of 1×10⁸ CFU/mL. Mice in group 4 received 100 μg of α-mPD-L1 in 100 μL PBS via IP injection every 3 days for a total of 5 doses. Stool was collected from all mice immediately prior to beginning treatment and two days after the final probiotic treatment and stored at −80° C. for subsequent microbiome analysis. Two weeks after the first treatment, stool was collected daily from groups 2 and 3 and plated on SDA plates supplemented with chloramphenicol (50 mg/L), gentamycin (5 mg/L), and 50 μg/mL blasticidin S for Sb enumeration. Cages were changed daily for the first two days of stool collection to reduce re-exposure via coprophagy. Once Sb titers fell below 1 CFU/mg, the probiotic was considered cleared and the mouse was sacrificed, along with randomly selected mice from groups 1 and 4 as time-matched controls. Mice with low levels of residual Sb were sacrificed one week after the final gavage.

### Tumor Enumeration and Biospecimen Harvesting

At time of sacrifice, ˜50 μL of blood was collected via submandibular puncture and stored in EDTA-coated serum separator tubes (BD Microtainer, 365978). Samples were centrifuged at 10,000×g for 10 minutes and serum was transferred into new microcentrifuge tubes and stored at −80° C. for subsequent analysis via Bio-Plex. The GI tract was harvested and the colon, SI (distal 15 cm), and mesenteric lymph nodes (MLNs) were collected. The distal-most 10 cm was used for tumor enumeration and the remaining 5 cm was used for flow cytometry. The colon and distal 10 cm of the SI were washed thoroughly with PBS, cut longitudinally with eyelet scissors, and spread onto a light box lumen side up. Alcian blue dye was used to enhance contrast and all tumor counting was performed by the same person who was blinded to the mouse treatment groups.

### Cytokine Analysis

Serum cytokines were measured using the Bio-Plex Pro Mouse Cytokine 23-Plex Assay (Biorad, M60009RDPD) on the Bio-Plex 200 machine. Each serum sample was analyzed across two technical replicates. Cytokine concentrations were derived from kit-included controls analyzed alongside serum samples and the lowest concentration calculated using the Bio-Plex software was designated as the limit of detection (LOD).

### Microbiome Characterization

Metagenomic DNA was extracted from stools collected immediately before and after treatment using the DNeasy PowerSoil Pro Kit (Qiagen, 47014). Targeted amplification of the V4 region (2053442) of the 16S rRNA gene was performed in triplicate with a DNA-free template negative control reaction for each sample. Amplicons were visualized via agarose gel electrophoresis to confirm correct amplicon size and rule out contamination in negative control reactions. Amplicons were pooled at an equimolar ratio and sequenced with the paired-end 2×250 Illumina MiSeq platform.

Paired-end reads were trimmed and merged using the BioConductor DADA2 package in R and taxonomically assigned from the phylum to genus level using the Silva version 138.1 reference database (27214047, 23193283). For rarefaction analysis, samples in the top 10% of total read counts were analyzed to determine a minimum read count for all samples. Reads were incrementally subsampled from 2,500 to 50,000 reads and relative abundances were calculated by transforming read counts. Low abundance taxa (mean relative abundance of <0.1% across included samples) were filtered and excluded from the rarefaction analysis. Shannon alpha diversity scores were calculated for all subsample depths using the Vegan package in R and compared by pairwise Dunn tests with Benjamini-Hochberg adjustment. A read count minimum of 5,000 was determined via rarefaction as no significant differences in Shannon diversity were observed between 5,000 reads and any higher read depth. Samples that passed the empirically determined read threshold were included in subsequent analyses (n=99).

Read count transformed phylum, family, and genus relative abundances were calculated for remaining samples, again with low abundance taxa removed. The Vegan package in R was used to calculate Shannon index alpha diversity and perform principal coordinate analysis (PCoA) using between-sample Bray-Curtis dissimilarities to visualize beta diversity. Significant differences across treatment groups and timepoints were tested via PERMANOVA of between-sample Bray-Curtis dissimilarities using the adonis2 function in the Vegan package in R (36704711). To identify bacterial genera that significantly increase or decrease in relative abundance from d36 to d50 within each treatment group, MaAsLin2 was used to fit linear models to the count transformed genus relative abundance data (34784344). A linear model was generated for each treatment group, with pre-treatment as the fixed effect within each model. Taxa with a p-value <0.05 following Benjamini-Hochberg adjustment were considered statistically significant.

### Flow Cytometry

MLNs were immediately placed in DMEM with 5% FBS, 50 μM β-mercaptoethanol, nonessential amino acids, penicillin (100 U/mL), streptomycin (100 μg/mL), 1 mM sodium pyruvate, and 1% glutaMAX. The MLNs were dissociated by mashing the tissue between frosted ends of two microscope slides and strained through a 100 μm cell strainer. Following centrifugation, MLN cells were resuspended in FACS buffer (3% FBS, 10 mM EDTA, 20 mM HEPES, 10 μg/mL polymyxin B, 1 mM sodium pyruvate, penicillin (100 U/mL), and streptomycin (100 μg/mL) in PBS).

Small intestine tumors were dissected and placed in R10 media (RPMI 1640 with 10% FBS, 1% glutaMAX, penicillin (100 U/mL), streptomycin (100 μg/mL), and 10 mM HEPES). The tumors were thoroughly minced with curved scissors and digested in R0 with 1 mg/mL collagenase and 0.5 μg/mL DNAse 1 for 45 minutes at 37° C. The digested tumors were strained through a 100 μM filter, washed with R10, and resuspended in FACS buffer.

For intracellular cytokine stimulation, cells were incubated with eBioscience™ cell stimulation cocktail at 37° C. for 3.5 hours, followed by cell surface stain (CD4, CD3, CD8, CD44, CD62L) and intracellular stain (TNFα, IFNγ) using the Foxp3 fixation/permeabilization buffer from the eBioscience™ Foxp3/Transcription Factor Staining Buffer Set performed overnight at 4° C. For intracellular transcription factor staining, cells were surface stained (as above), fixed/permeabilized with permeabilization buffer from the eBioscience™ Foxp3/Transcription Factor Staining Buffer Set and intracellular transcription factor stain (Foxp3) performed overnight at 4° C. Following cytokine and transcription factor staining, the cells were washed in permeabilization buffer and resuspended in FACS buffer for flow analysis on the Attune NxT flow cytometer.

### Quantification and Statistical Analysis

The details of all statistical tests are available in respective figure legends. Statistical tests for all analyses, with the exception of those pertaining to microbiome community structure, were performed in GraphPad Prism v.10.0.3. Statistically significant outliers were determined using the Grubbs' test within Prism and removed from analyses. For analyses comparing outcomes of categorical nature, distribution normality of each category was assessed via the Shapiro Wilks test. If all categories were found to be normally distributed (p≥0.05), either a t-test (2 categories) or an ordinary one-way ANOVA (3 or more categories) was used and Dunnett's multiple comparison test was applied with either PBS or optimal culturing conditions designated as the control category. If one or more categories were found to be non-normally distributed (p<0.05), either a Mann-Whitney test (2 categories) or a Kruskal Wallis ANOVA (3 or more categories) was used and Dunn's multiple comparison test was applied using PBS or optimal culturing conditions as the control category. For analyses comparing outcomes overtime, distribution normality was again measured using Shapiro Wilks test, with normal data sets being analyzed via multiple t-tests and non-normal data sets analyzed via multiple Mann-Whitney tests. For analyses comparing survival over time, the log-rank Mantel Cox test was applied. All p-values were corrected via false discovery rate (FDR) as appropriate for each statistical test.

### Results

### Candidate Antibody Variants for Probiotic Platform Perform Effective Immune Checkpoint Blockade

To evaluate candidate ICI payloads for integration into our probiotic therapeutic delivery platform, we identified two previously developed ICI variants: a high-affinity PD-1 microbody (haPD-1) consisting of a PD-1 ectodomain fused to a human IgG1 dimeric CH3 domain (26604307) and a PD-L1-targeted nanobody (camelid single-domain antibody) (rPD-L1 nb) (32051224). haPD-1 was previously demonstrated to have potent interactions with both mouse and human PD-L1, reduce tumor burdens in syngeneic CT26 murine tumor models, and to have superior tumor penetrance and induce less T-cell exhaustion in comparison to a conventional PD-L1 antibody (26604307). The rPD-L1 nb was previously integrated into an engineered Escherichia coli Nissle 1917 delivery system with efficacy against multiple tumor types in a murine flank syngeneic model following intratumoral injection (32051224). Both proteins have substantially lower molecular weights in comparison to conventional ICI antibodies (30.1 kDa for haPD-1 monomer and 16 kDa for rPD-L1nb; ˜150 kDa for IgG antibodies)[26604307; 32051224], making them favorable candidates for robust expression and secretion from yeast.

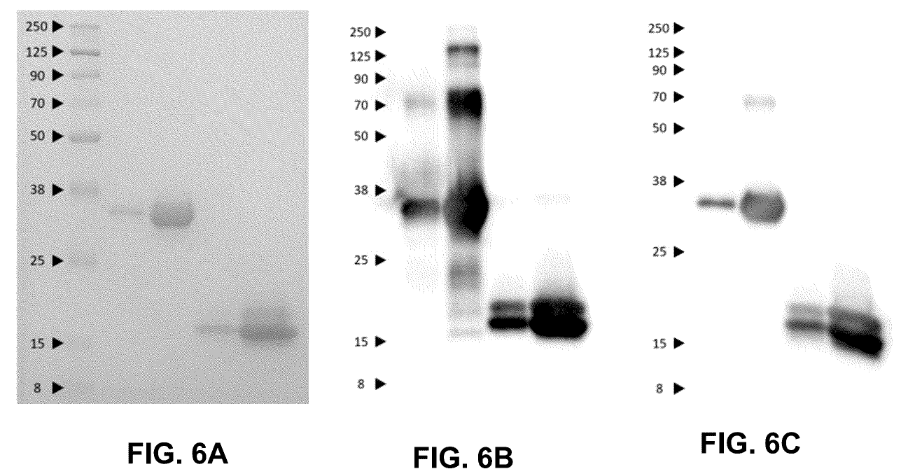

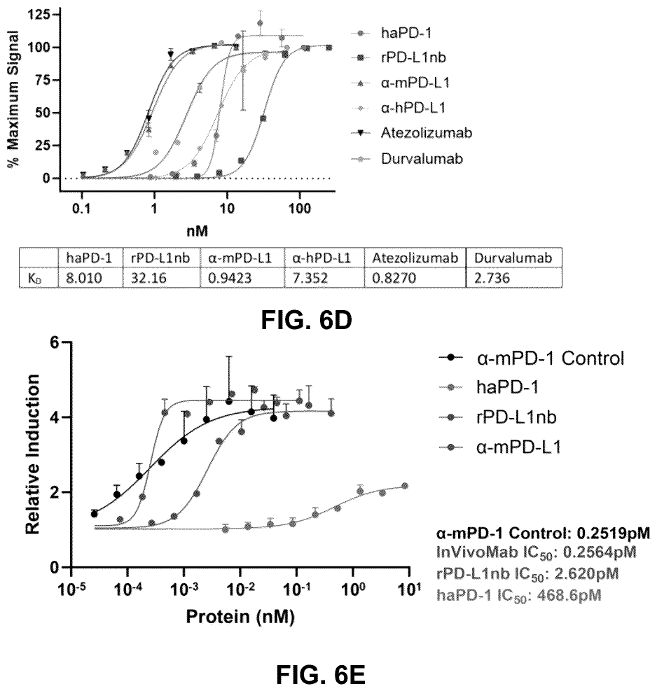

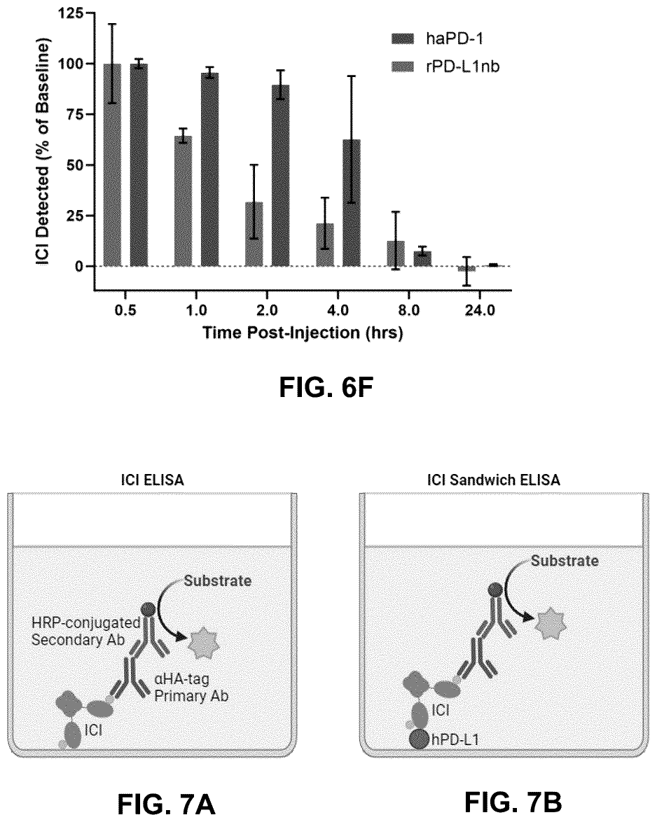

haPD-1 and rPD-L1 nb with 6×Histidine and hemagglutinin (HA) tags were recombinantly expressed with and purified from E. coli BL21(DE3) using Nickel affinity chromatography. We analyzed the proteins for correct size and purity using SDS-PAGE followed by total protein stain and western blot. We observed bands at the predicted sizes for haPD-1 and rPD-L1 nb at 30.1 and 16 kDa, respectively [FIG. 6A]. Furthermore, both proteins were detectable using αHis and αHA western blotting [FIG. 6B,C]].

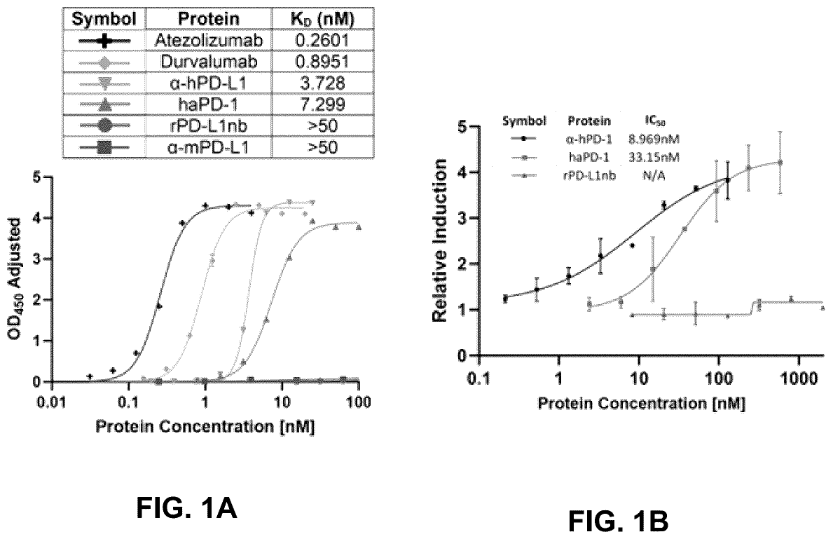

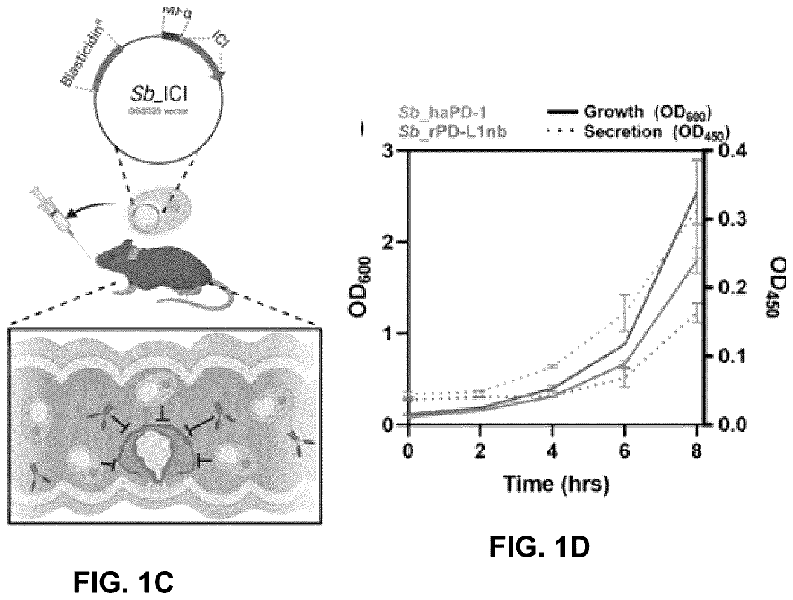

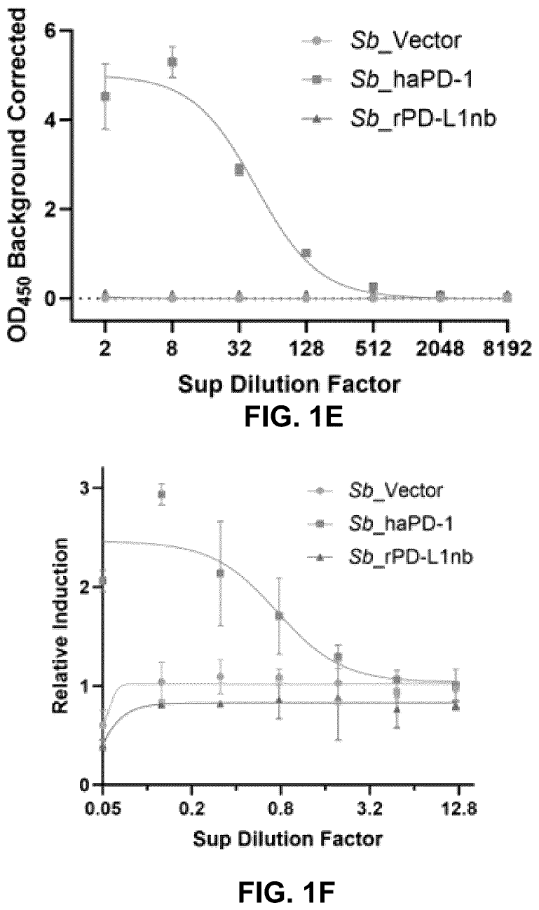

To confirm and compare the PD-L1 binding capacity of haPD-1 and rPD-L1 nb, we developed a series of sandwich ELISAs to assess interactions with PD-L1 [FIG. 7]. In a human PD-L1 (hPD-L1) binding assay, we observed a dose-dependent interaction of haPD-1 with hPD-L1, which was also observed for three conventional PD-L1-directed IgG antibodies, including InVivoMab α-hPD-L1 and clinically utilized antibodies, durvalumab and atezolizumab [FIG. 1A]. The K_{D} value for haPD-1 was calculated using the specific binding with Hill Slope equation (GraphPad Prism 10.1.0), and was found to be comparable to InVivoMab α-hPD-L1 (7.3 and 3.7 nM, respectively) and was within approximately an order of magnitude of durvalumab and atezolizumab (0.90 and 0.26 nM, respectively). Although rPD-L1nb and InVivoMab α-mPD-L1 showed no appreciable binding to hPD-L1, we observed dose-dependent binding in a similar assay with mouse PD-L1 (mPD-L1), wherein haPD-1 maintained a comparable K_{D} (32.16 and 8.01 nM for rPD-L1nb and haPD-1, respectively) [FIG. 6D], showing that haPD-1 binds both hPD-L1 and mPD-L1.

As an orthogonal and more functional measure of the intended PD-1/PD-L1 blockade activity, we tested purified haPD-1 and rPD-L1 nb in a human PD-1/PD-L1 blockade assay, wherein blockage of the interaction between PD-1 effector cells and PD-L1αAPC/CHO-K1 cells results in luciferase expression (Promega J1250)(10.1158/1538-7445.AM2015-5440). haPD-1 demonstrated an IC₅₀ value of 33.15 nM (GraphPad Prism 10.1.0, Sigmoidal 4 parameter logistic curve) in this assay [FIG. 1B]. Although rPD-L1 nb did not appear to induce blockade in this assay, we observed activity in the mouse PD-1/PD-L1 blockade assay, indicating rPD-L1 nb has an apparent species specificity towards the mouse PD-1/PD-L1 axis [FIG. 6E].

We next evaluated the pharmacokinetics of the purified ICI variants in healthy mice. 100 μg of haPD-1 and rPD-L1nb was administered via intraperitoneal (IP) injection, and blood was collected at 0.5, 2, 4, 8, and 24 hours post-injection. Both proteins were detectable at the highest signal at 0.5 hours post-injection and dropped to roughly half of baseline concentrations within 2 and 4 hours post-injection in rPD-L1nb and haPD-1 groups, respectively. By 8 hours post-injection, both proteins were >90% cleared and were undetectable at 24 hours post-injection [FIG. 6F], indicating a transient pharmacokinetic profile in comparison to conventional IgG antibodies, which have reported half-lives of 6-8 days in mice[3350037].

### Engineered Sb can Secrete Functional ICI Proteins

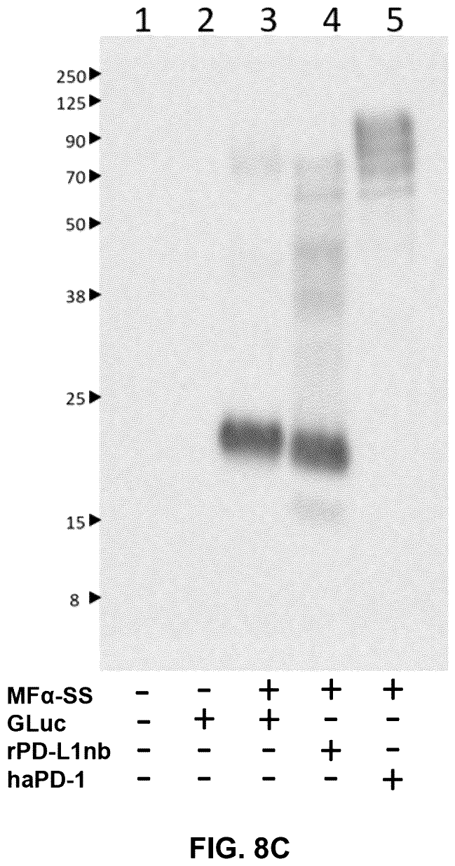

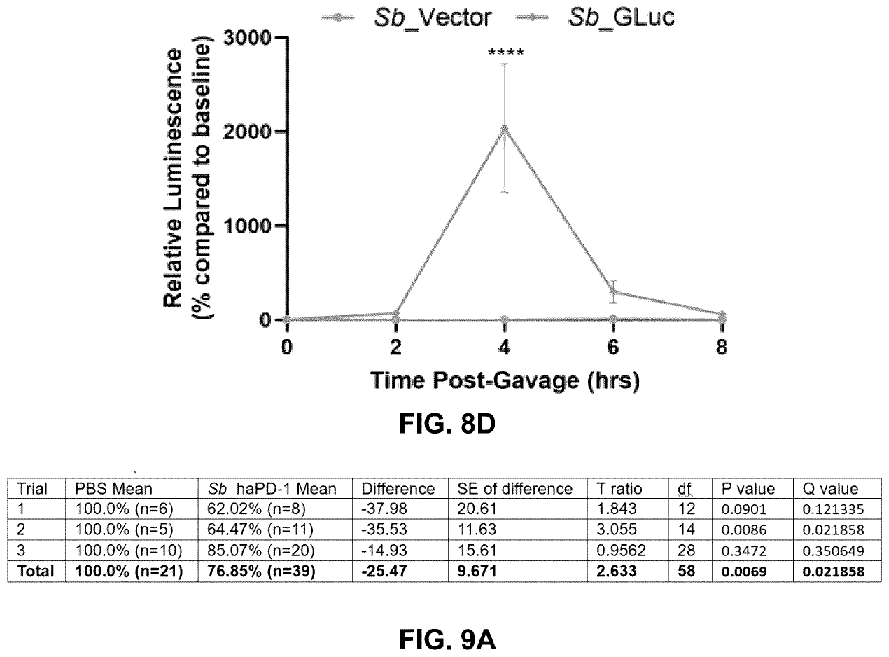

To enable robust secretion of protein-based ICI therapies from Sb directly within the mammalian gut, we developed a constitutive yeast secretion construct in the OGS539 vector [32567021][FIG. 1C]. This vector backbone contains a yeast high-copy 2μ origin of replication and a blasticidin resistance marker for selection in yeast. The secretion construct is composed of an N-terminal mating factor α secretion signal (MFα-SS) followed by an HA epitope tag and the payload protein. The gene circuit is regulated by the constitutive pTEF1 yeast promoter. Both haPD-1 and rPD-L1 nb were cloned into this secretion circuit, and we also engineered constructs that included the reporter protein Gaussia luciferase (GLuc) with and without MFα-SS (GLuc and GLuc-ns, respectively) as additional controls [FIG. 8]. Sb strains transformed with these plasmids were grown for 48 hours. We observed that the presence of MFα-SS was essential for observing signal in 48 hour Sb_GLuc cultures via ELISA [FIG. 8A] and αHA western blot [FIG. 8C] and that both ICI payloads were successfully secreted [FIG. 8B-8C].

To obtain a robust comparison of growth and secretion between Sb_haPD-1 and Sb_rPD-L1 nb, both strains were grown for 8 hours with periodic sampling of culture broths followed by αHA ELISA and cell density (OD₆₀₀) reads. Although Sb_haPD-1 had a slightly lower density over the time-course, this strain exhibited higher secretion capacity [FIG. 1D]. To measure binding capacity of Sb-secreted haPD-1 and rPD-L1 nb, 48-hour culture broths were assayed in a hPD-L1 binding ELISA [FIG. 1E]. We observed robust binding of haPD-1 to hPD-L1, but no apparent activity of Sb-secreted rPD-L1 nb in this assay, consistent with rPD-L1 nb binding specificity towards mPD-L1 [FIG. 6E]. Sb_haPD-1 culture broths also maintained dose-dependent activity in the human PD-1/PD-L1 blockade assay [FIG. 1F]. Due to the robust performance of purified, E. coli-derived haPD-1 and Sb-secreted haPD-1, as well as the novelty of haPD-1 as a probiotic-carried payload, we chose to further characterize haPD-1 in subsequent analyses.

Sb haPD-1 Transiently Traverses the GI Tract

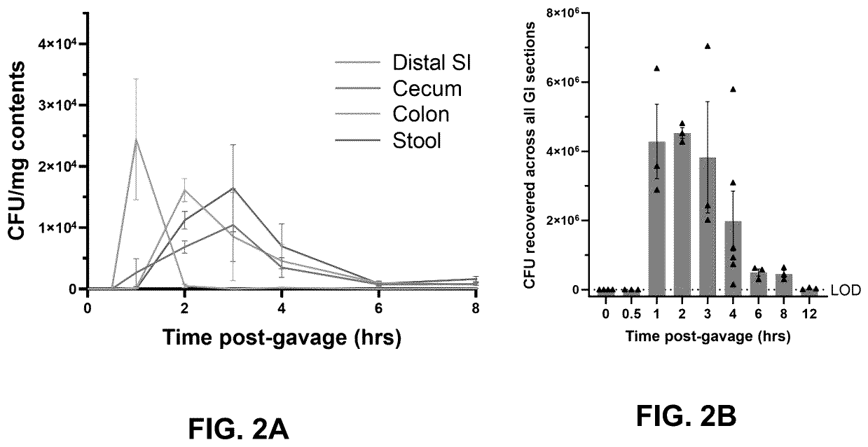

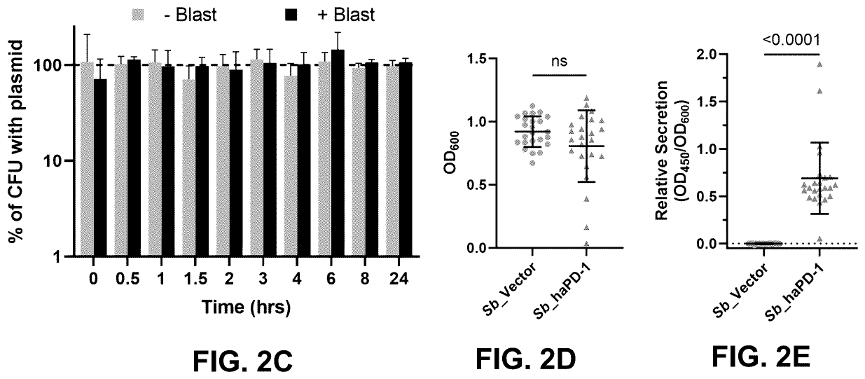

Sb does not readily colonize the mammalian GI tract in the absence of antibiotic intervention or other perturbations (10.1038/s41598-022-12806-0). Although it has a reportedly short gut transit time (10.1038/s41598-022-12806-0), we sought a more detailed, high-resolution characterization of its GI transit dynamics. To address this, we tracked the presence of orally administered Sb_haPD-1 at various timepoints across the different compartments of the lower GI tract in healthy mice [FIG. 2A,B]. We observed a sequential transition of Sb_haPD-1 titers that corresponded to the biogeographical order of compartments, with titers peaking in the distal small intestine (SI) and colon at 1- and 2-hours post-gavage, respectively [FIG. 2A]. Importantly, Sb_haPD-1 maintains the transient GI transit dynamics of its wildtype (WT) counterpart and is virtually undetectable by 12 hours post-gavage, with maximal titers observed at 2 hours post-gavage [FIG. 2B]. At this maximal point, we recovered 45.30% of the administered bolus, which is likely an underestimate as yeast recovery was only measured in a single stool pellet and yeasts were already being shed in the feces by this time [FIG. 2A]. This high recovery proportion suggests a large proportion of the inoculated yeast survive gut transit. Together, these data demonstrate that Sb_haPD-1 efficiently transits and clears the GI tract while maintaining viability.

### Sb can Stably Express and Secrete Recombinant Proteins in the GI Tract

Due to its toxicity to mice [13525246], blasticidin supplementation was not an option to promote retention of the OGS539 plasmid during animal experiments. As such, we sought to quantify plasmid loss rates in vitro in the absence of blasticidin-directed selective pressure. Reassuringly, we observed no appreciable loss of plasmid-dependent blasticidin resistance in vitro in a 24-hour period [FIG. 2C], suggesting that the plasmid is stably maintained for longer than the expected duration of Sb transit through the mouse GI tract. To test this in vivo, we administered Sb_haPD-1 to healthy mice and probed their stool for viable Sb_haPD-1 that maintained blasticidin resistance and the capacity to secrete haPD-1. Of the colonies screened, the vast majority of Sb_Vector and Sb_haPD-1 colonies maintained the ability to grow in the presence of blasticidin. There were no statistically significant differences in growth (p<0.2162, Mann-Whitney test) between the two constructs, suggesting the presence of a functional payload does not impart a fitness cost or increase plasmid loss [FIG. 2D]. Importantly, most of the recovered Sb_haPD-1 colonies maintained the capacity for robust haPD-1 secretion as indicated by significantly elevated ELISA signal of the Sb_haPD-1 culture broths compared to the Sb_Vector culture broths (p<0.0001, Mann-Whitney test), demonstrating that the plasmid-based secretion system remained intact for the duration of transit through the GI tract [FIG. 2E]. Based on these results, this plasmid-based secretion system is sufficiently stable within Sb to traverse the murine GI tract while maintaining its functionality.

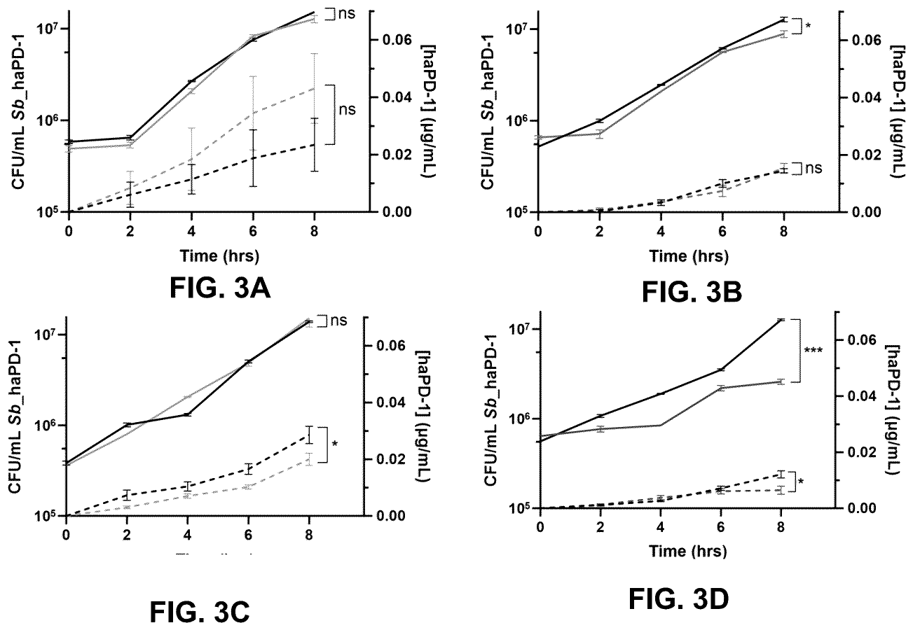

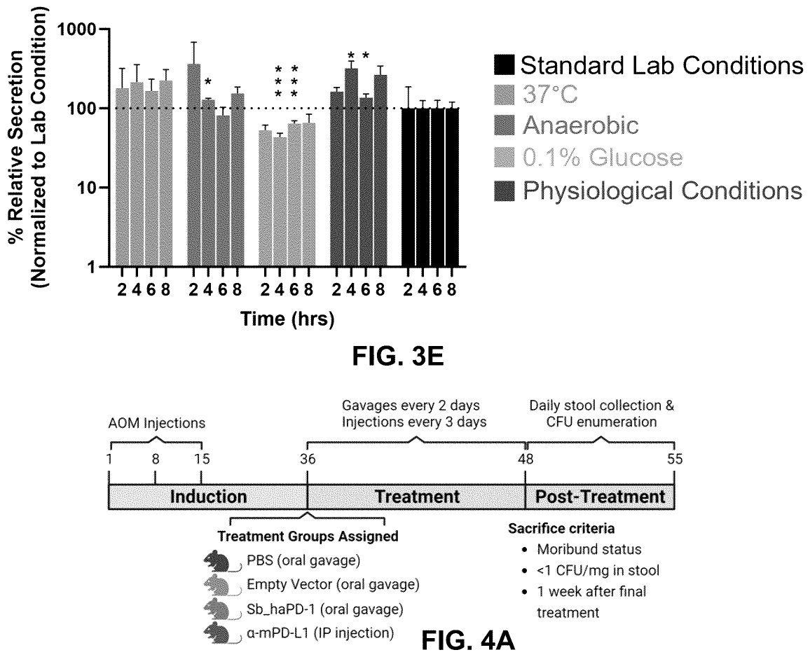

Although we have demonstrated the stability of Sb_haPD-1 and its recombinant secretion system, it is possible that the harsh conditions imposed by the GI environment could negatively impact secretion during its transit. In particular, the increased temperature, decreased glucose availability, and anaerobic nature of the GI tract are environmental challenges that could hinder Sb_haPD-1 replication, haPD-1 secretion, or both. To systematically quantify the impacts of each of these variables, we first measured growth and secretion via culture broth ELISA in each physiological condition individually (37° C., 0.1% glucose, or anaerobic), then collectively in a single condition (physiological) [FIG. 3A-C]. The only single variable condition that resulted in significant changes to Sb_haPD-1 growth was oxygen limitation (p<0.0424, extra sum-of-squares F test with Bonferroni correction). The physiological condition imposed the largest growth defect, resulting in a significant increase in doubling time (1.246 hours for optimal, 3.417 hours for physiological; p<0.0004, extra sum-of-squares F test with Bonferroni correction) [FIG. 3D]. Final haPD-1 concentration was significantly decreased in 0.1% glucose (0.02857 μg/mL for optimal, 0.01955 μg/mL for 0.1% glucose; p<0.0148, multiple t-tests with Bonferroni correction) and physiological (0.01209 μg/mL for optimal, 0.006472 μg/mL for physiological; p<0.005112, multiple t-tests with Bonferroni correction) conditions. When secretion is normalized to growth, the majority of conditions and timepoints are not significantly different compared to optimal lab condition controls [FIG. 3E]. Although there are a few exceptions wherein relative secretion significantly increases (e.g., anaerobic, 4 hours) or decreases (e.g., 0.1% glucose, 4 hours and 6 hours), the net outcome when these variables are combined is an increase in relative secretion, which is statistically significant at the 4- and 6-hour timepoints (220.41% increase and 36.26% increase, respectively; p<0.0243 and p<0.0229, respectively, mixed-effect analysis with Dunnett's multiple comparisons test) [FIG. 3E]. These data support the capacity for Sb_haPD-1 to secrete haPD-1 in the GI tract, despite the environmental challenges imposed by the GI tract on the probiotic.

As a final proof-of-concept demonstrating Sb's capacity for in vivo secretion, we used GLuc as a reporter payload to measure in vivo secretion by Sb. Unlike haPD-1, GLuc is not known to bind any native targets in the murine gut, making it ideal as a proxy payload that is readily shed and detectable in the stool. Mice were orally administered Sb_GLuc and their stool was collected for luminescence quantification. GLuc-mediated luminescence was detected in their stool at significantly higher levels than the Sb_Vector control (p<0.00001, multiple t-tests with Bonferroni correction) [FIG. 8D]. Luminescence was observed as early as 2 hours post-gavage and peaked at 4 hours post-gavage. In sum, these data illustrate Sb's capacity to stably express and secrete functional proteins in the murine GI tract.

Sb haPD-1 is Well Tolerated and Reduces Intestinal Tumors in APC^{min} Mice

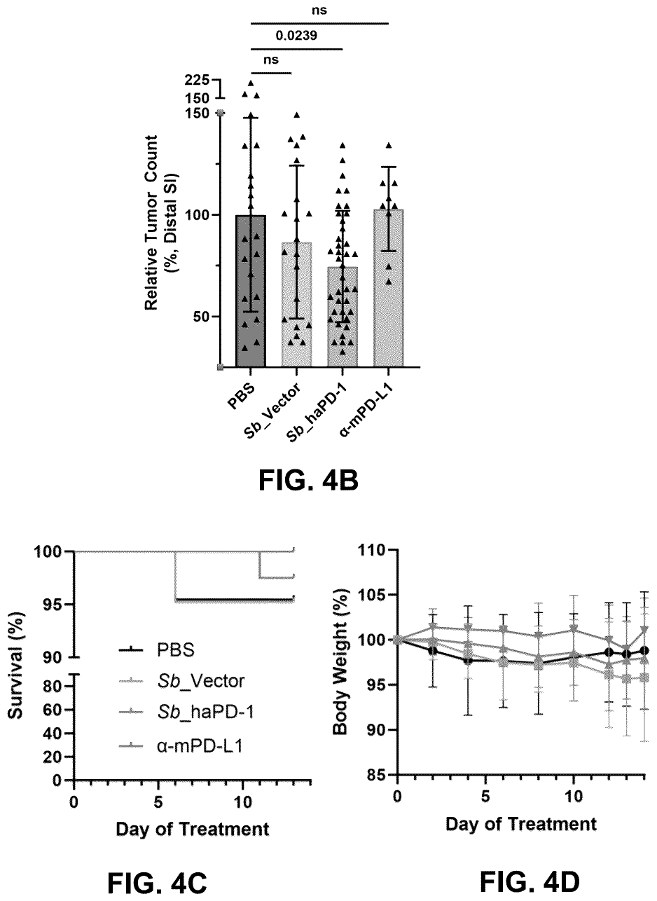

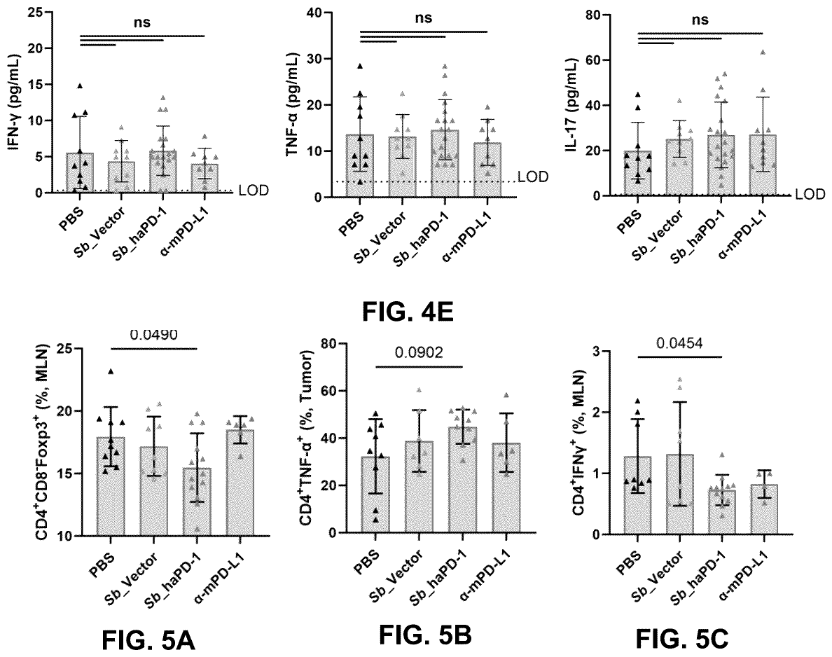

Following our extensive in vitro characterization and preliminary testing of Sb_haPD-1 in healthy mice, we then tested its tolerability and therapeutic efficacy in a murine model of CRC. We utilized APC^{min} mice, a mouse model of multiple intestinal neoplasia that also develops colonic tumors when challenged with azoxymethane [17638923]. Following a 5-week induction period, mice were treated with repeated doses of Sb_haPD-1 or relevant controls (PBS, Sb_Vector, or α-mPD-L1) for approximately two weeks [FIG. 4A]. Although this mouse model is considered a microsatellite stable (MSS) model of CRC [18403596] with limited responsiveness to PD-1/PD-L1-directed monotherapy[32185408, 10.1136/jitc-2020-001895corr1], we employed a conventional α-mPD-L1 dosing regimen with demonstrated efficacy in other models [10.1126/science.abc3421].

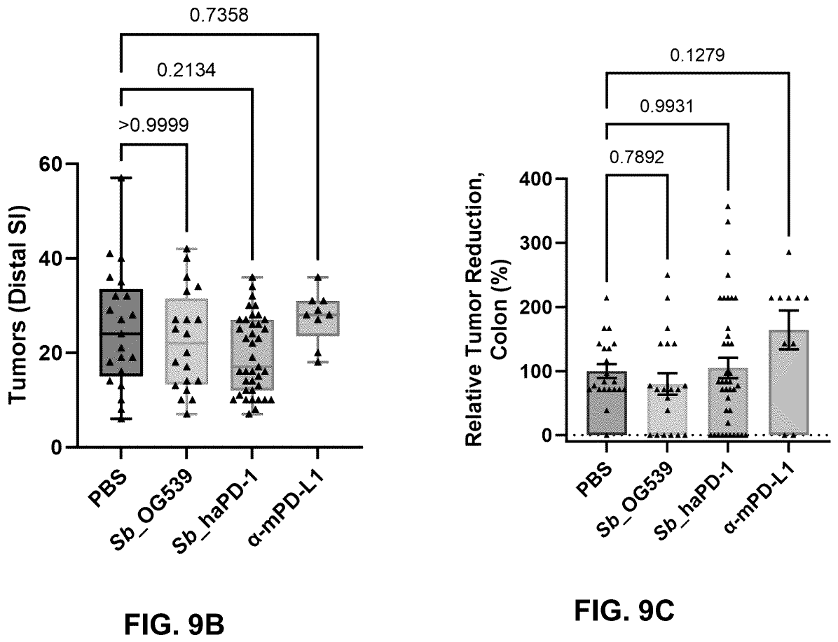

Upon sacrifice, tumors in the distal SI and colon were enumerated to evaluate the therapeutic efficacy of Sb_haPD-1. Mice treated with Sb_haPD-1 had a statistically significant decrease (p<0.0239, ordinary one-way ANOVA with Dunnett's multiple comparisons test) in distal SI tumor burden [FIG. 4B]. On average, mice treated with Sb_haPD-1 had 37.90 distal SI tumors compared to 50.50 in the PBS control, a 24.95% reduction [FIG. 9A,B]. This reduced SI tumor burden was observed across three independent experiments [FIG. 9A], encompassing both male and female mice supplied from different breeding colonies, suggesting a stable phenotypic penetrance. There was no observable tumor reduction in the colon [FIG. 9C]. Notably, this mouse model remained recalcitrant to conventional α-mPD-L1 therapy, despite α-mPD-L1 outperforming haPD-1 in vitro with regard to mouse PD-L1 binding and mouse PD-1/PD-L1 blockade (FIG. 6D,E).

Regarding tolerability of the Sb strains, there were no significant differences in survival (p<0.8768, Mantel-Cox text) or weight loss between Sb_haPD-1 and any other treatment group (n.s., multiple Mann-Whitney tests with Bonferroni correction) [FIG. 4C,D]. Furthermore, we performed quantification of key cytokines associated with inflammation (interferon gamma (IFN-γ), tumor necrosis factor alpha (TNF-α), and interleukin 17 (IL-17)) on post-treatment serum to rule out any treatment-induced systemic inflammation. We found no significant changes (p<0.9954, p<0.9573, and p<0.3237, respectively, ordinary one-way ANOVA with Dunnett's multiple comparison test for IFN-γ and TNF-α and Kruskal-Wallis ANOVA with Dunn's multiple comparison test for IL-17) in these cytokine levels in Sb_haPD-1-treated mice [FIG. 4E], illustrating that the Sb_haPD-1 regimen was well tolerated in this disease model.

Sb haPD-1 Modulates Immune Cell Profiles in APC^{min} Mice

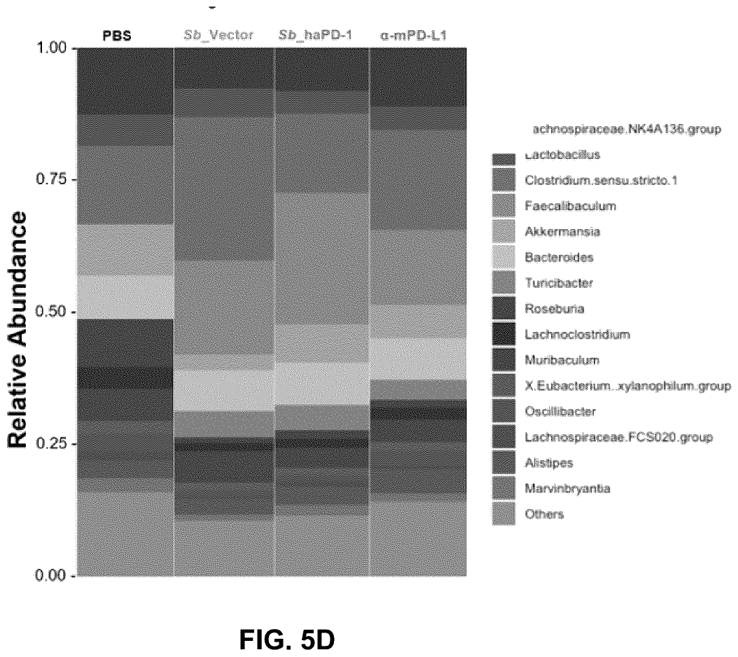

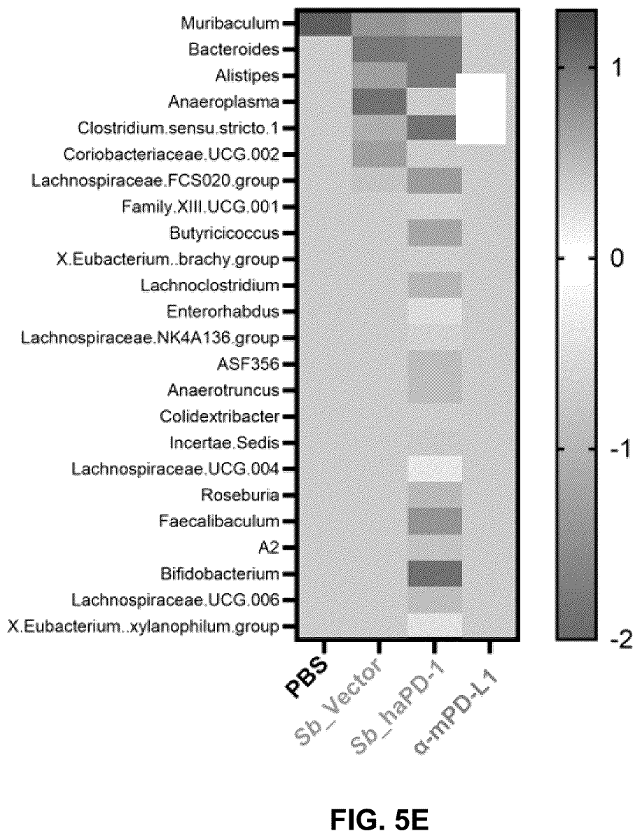

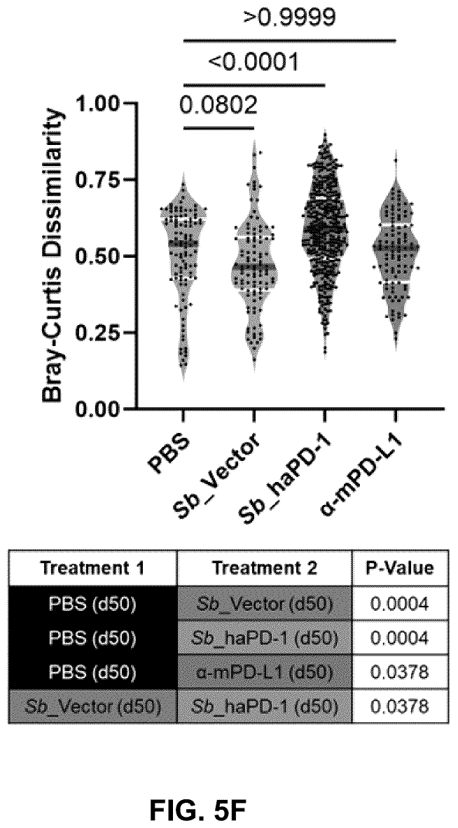

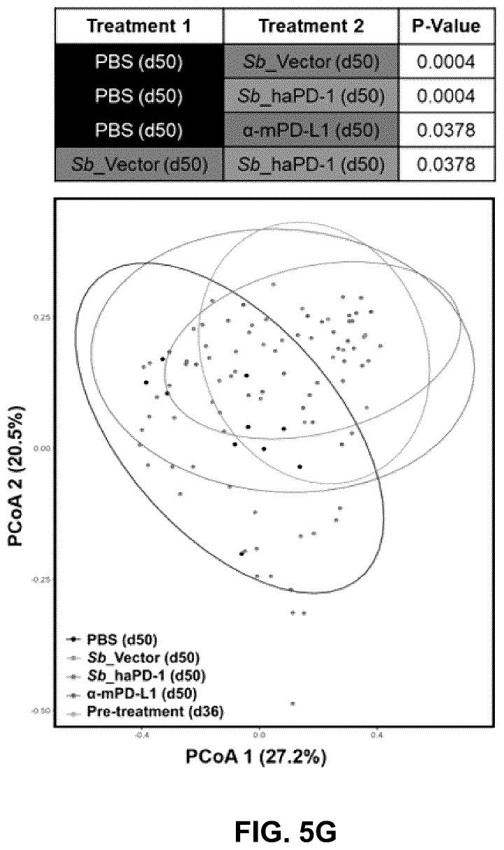

To elucidate immunomodulatory shifts associated with Sb_haPD-1 treatment, we performed flow cytometry on immune cells isolated from mesenteric lymph nodes (MLNs) and distal SI tumors harvested from treated mice. Specifically, we sought to probe T cell populations that could be affected by PD-1/PD-L1 blockade and subsequently influence antitumor immunity. We observed a significant decrease (p<0.0490; ordinary one-way ANOVA with Dunnett's multiple comparisons test) in tolerogenic CD4⁺CD8⁻Foxp3⁺ T cells (Tregs) in the MLNs [FIG. 5A] of mice treated with Sb_haPD-1. We also noted a substantial trend (p<0.0902; ordinary one-way ANOVA with Dunnett's multiple comparisons test) towards increased CD4⁺TNF-α⁺ T cells in tumors and significantly decreased (p<0.0454; Kruskal-Wallis ANOVA with Dunn's multiple comparisons test) CD4⁺IFNγ⁺ T cells in the MLNs of Sb_haPD-1-treated mice [FIG. 5B,C]. Notably, we did not observe any significant differences in the immune cell populations tested in α-mPD-L1-treated mice, indicating that the Sb_haPD-1 treatment alone was associated with substantial shifts in immune cell populations within both MLNs and tumors.

Sb haPD-1 Therapy Significantly Shifts the Gut Microbiome Composition

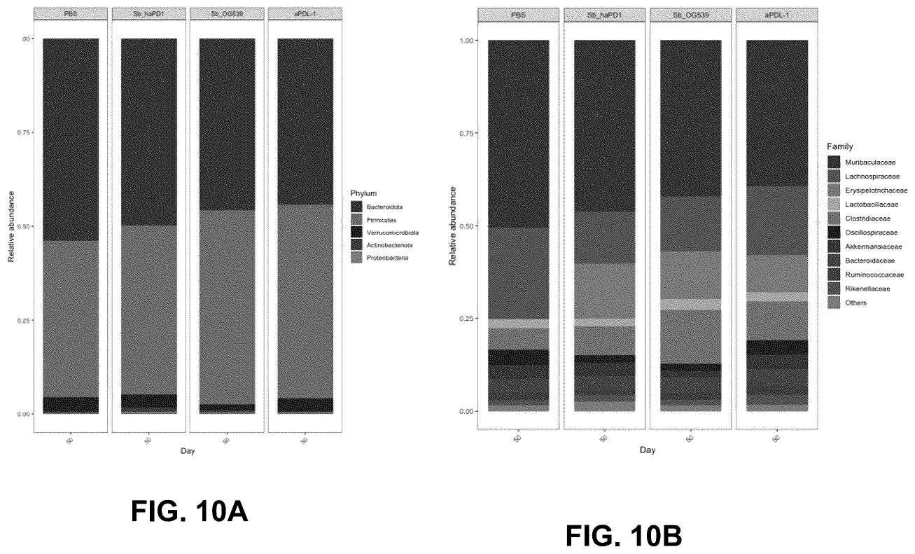

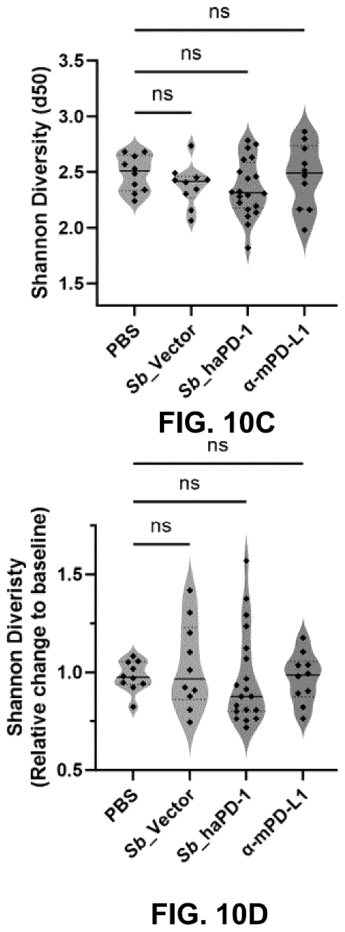

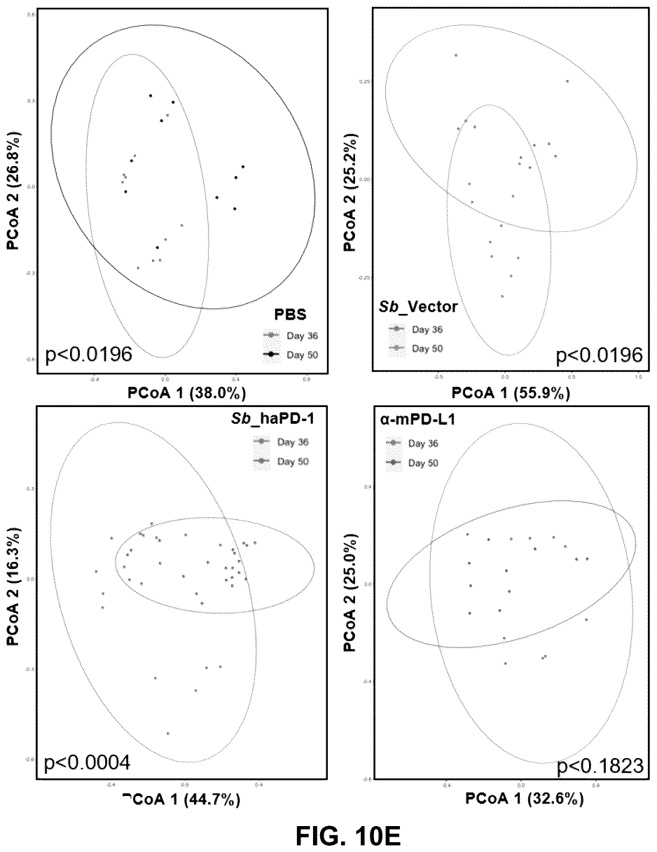

To investigate how Sb_haPD-1 impacts the bacterial component of the gut microbiome, we performed 16S rRNA gene amplicon sequencing of gDNA extracted from stool pellets collected immediately before (d36) and after (d50) treatment. The relative abundance of the 15 most abundant bacterial genera [FIG. 5D], phyla [FIG. 10A], and families [FIG. 10B] after treatment are typical of a murine microbiome dominated by genera belonging to the Firmicutes and Bacteroidetes phyla[35892142]. To examine which bacterial genera were differentially abundant at d50 compared to the d36 pre-treatment, we performed microbiome multivariate association modeling using MaAsLin2[10.1371/journal.pcbi.1009442] within each treatment group [FIG. 5E]. Altogether, we identified 23 bacterial genera that were differentially present (Linear Model, Total Sum Scaling, Benjamini-Hochberg corrected) in Sb_haPD-1 d50 samples relative to Sb_haPD-1 d36 samples, and 18 of these significant depletions or enrichments were unique to Sb_haPD-1. In contrast, we only identified one and six differentially present genera in PBS and Sb_Vector-treated mice, respectively, and no genera were significantly enriched or depleted in α-mPD-L1-treated mice.

We next evaluated diversity metrics within pre- and post-treatment samples to assess changes to community composition. To quantify differences in alpha diversity of the gut before and after treatment, we computed Shannon diversity of d36 and d50 samples. There were no significant differences (n.s., ordinary one-way ANOVA with Dunnett's multiple comparisons test) in Shannon diversity between treatment groups at d50 [FIG. 10C]. We also quantified overall change in Shannon diversity before and after treatment within each mouse (d50/d36), which also showed no significant differences across treatment groups (n.s., Kruskal-Wallis ANOVA with Dunn's multiple comparisons test). To quantify differences in the beta diversity of the gut microbiome between pre- and post-treatment samples within each treatment group, we computed Bray-Curtis dissimilarities between each mouse at d36 and each mouse (including itself) within its own treatment group at d50. Mice treated with Sb_haPD-1 had a significantly higher mean Bray-Curtis dissimilarity between pre- and post-treatment compared to that of PBS-treated mice (0.5867 and 0.5039, respectively; p<0.001 Kruskal-Wallis ANOVA with Dunn's multiple corrections) [FIG. 5F]. In contrast, the mean Bray-Curtis dissimilarities between pre- and post-treatment of Sb_Vector- and α-mPDL-1-treated mice were not significantly different (p<0.0802 and p>0.9999, respectively; Kruskal-Wallis ANOVA with Dunn's multiple corrections) from PBS-treated mice. These results indicate that Sb_haPD-1 treatment induced the most substantial change in the bacterial gut microbiome relative to PBS treatment.

To further investigate differences in community composition among treated mice, principal coordinate analysis (PCoA) using between-sample Bray-Curtis dissimilarities revealed significant differences (p<0.0004, p<0.0004, p<0.0378 for Sb_Vector, Sb_haPD-1, and α-mPD-L1, respectively; PERMANOVA with Benjamini-Hochberg correction) in post-treatment bacterial profiles between each treatment group and PBS [FIG. 5G]. We also observed prominent shifts between d36 and d50 samples within each treatment group (p<0.0196, p<0.0196, p<0.0004, and p<0.1823 for PBS, Sb_Vector, Sb_haPD-1, and α-mPD-L1, respectively; PERMANOVA with Benjamini-Hochberg correction) [FIG. 10E]. These results demonstrate that each treatment induced unique shifts in global gut bacterial compositions. This was further validated by a significant difference comparing Sb_haPD-1 and Sb_Vector Bray-Curtis dissimilarity PCoAs (p<0.0378; PERMANOVA with Benjamini-Hochberg correction) suggesting that despite their similar exposure to Sb, payload production differentiates the gut bacterial profile between these treatment groups. Collectively, these data illustrate that in addition to substantially influencing the immune cell landscape, Sb_haPD-1 also significantly impacts the gut microbiota composition, which may contribute to Sb_haPD-1 antitumor effects.

This study develops and characterizes the first reported use of a yeast probiotic as an orally administered carrier for cancer therapy to treat GI tumors. We show that Sb_haPD-1 can express and secrete functional ICI variant proteins via a plasmid-based secretion circuit, which demonstrates robust stability and function in vitro and in vivo. Oral administration of Sb_haPD-1 alleviates tumor burdens in APC^{min} mice and induces a distinct immune profile within both tumors and MLNs accompanied by shifts in the bacterial component of the gut microbiome.

This study also presents promising data supporting the stability of therapeutic secretion circuits in the genetic context of plasmid-based systems without the need for constant antibiotic exposure to ensure plasmid retention. We demonstrate that gene circuits developed in the OGS539 plasmid are functional and stable for at least 24 hours, which exceeds the expected transit time of Sb throughout the mouse gut. Based on our in vitro and in vivo demonstrations of the stability of OGS539-based gene circuits in such conditions [FIGS. 2C-E & 8D], such plasmid-based systems are functional and sufficient for therapeutic delivery during the brief murine gut transit time in the absence of traditional antimicrobial selection agents.

Although ICI therapies are effective for a subset of patients with CRC, their current use is limited to patients with deficient mismatch repair (dMMR) and microsatellite instability high (MSI-H) cancers [10.1038/s41575-019-0126-x]. The successful reduction of tumor burden in APC^{min} mice, which is an MSS, ICI-refractory mouse model[18403596, 32185408, 10.1136/jitc-2020-001895corr1], is a promising indication that our probiotic platform could have therapeutic efficacy in an expanded patient population. ICI therapy can also be precluded by immune-related adverse events (irAEs), which manifest in diverse off-target body sites and systems [32158597]. Further studies are warranted in additional mouse models with various mismatch repair and microsatellite statuses and mouse models that develop measurable, IC-induced toxicities to assess whether these limitations of ICI therapy can be overcome with our platform.

The host immune system is inextricably linked to the gut microbiome and plays an integral role in preventing or promoting tumor development [22356853, 30275043]. We observed a significant decrease in relative tumor burden [FIG. 4B] that was accompanied by a decrease in Tregs [FIG. 5A] in Sb_haPD-1-treated mice. Tregs activate immunosuppressive pathways that can limit ICI efficacy[10.1038/bjc.2017.434] and their reduced prevalence in Sb_haPD-1-treated mice is promising evidence that the therapy modulates the immune landscape to promote antitumor immunity. We also observed a significant decrease of CD4⁺IFNγ⁺ T cell prevalence [FIG. 5C] in the Sb_haPD-1 treatment group. Although IFNγ is canonically associated with antitumor immunity, there is a growing body of evidence that shows that the role of IFNγ is context-dependent and it can often promote tumor progression[29283429, 30388040], including in the context of PD-1 therapy[33606996]. Future efforts will focus on in-depth mechanistic studies to elucidate how Sb_haPD-1 treatment modulates T cells and other immune cell populations that can influence microbiota composition and promote tumor reduction.

The importance of the host microbiome in cancer development and response to therapy is an increasingly appreciated field of study[33253684, 34895176, 34506740]. Distinct taxa have been noted to have either beneficial or detrimental roles in ICI response[29097493, 10.1126/science.aan3706, 29302014]; however, no consensus “responder” microbiome has been identified [36622383, 30339501]. In addition to serving as a measurable biomarker that may predict therapeutic outcome, the microbiome has also been shown to be a target for manipulation to improve ICI response [34941392, 33303685]. The goal of this study was a preliminary characterization of this therapeutic platform as a proof-of-concept. As such, our focus in evaluating the gut microbiota was to assess tolerability and rule out undesirable perturbations that could result from probiotic supplementation or the presence of enteral ICI variants. Although we did observe significant changes in community composition following Sb_haPD-1 treatment [FIG. 5D-G], these shifts were not accompanied by increased weight loss or death [FIG. 4C-D], suggesting that these changes were tolerable to the host. We also observed changes in pre- and post-treatment community structure in PBS mice, which can be explained by changes in microbiome composition that occur as cancer progresses, a previously documented occurrence [10.1038/s41564-019-0649-5, 34249773]. Additionally, we also observed significant differences in post-treatment community composition between Sb_Vector and Sb_haPD-1 [FIG. 5G], indicative of haPD-1-specific effects on the microbiota. Further work is ongoing to assess whether these probiotic-driven changes in the microbiome are correlated with tumor reduction and whether baseline microbiota is predictive of treatment success or failure in this model. If the microbiota is found to be an important determinant in treatment outcome, direct manipulation of the microbiota through FMT, targeted antibiotics, or prebiotic supplementation could be used in combination with our therapeutic platform to enhance therapeutic response.

The use of Sb as an in situ biomanufacturer and delivery vehicle for protein-based therapies has immense potential therapeutic benefit. Protein-based biologic therapies including ICIs have demonstrated stark growth in recent decades, however the manufacturing processes and storage requirements of such therapies impose high cost, qualified personnel, and appropriate facilities, which limits broad access to these therapies[33536635, 29883853]. Yeast-based therapeutic production and delivery platforms could represent a less costly and labor-intensive platform in comparison to traditional methods to manufacture biologics [20140428], and the ease of storage through desiccation and safe administration of Sb in high doses makes a probiotic-mediated delivery system a compelling improvement to conventional biologic therapies including ICIs. Lower regions of the GI tract are typically not amenable to orally-administered, protein-based therapies secondary to degradation by proteases and the harsh physiochemical GI environment. Targeted therapeutic delivery coupled with the short half-lives of antibody variants and other miniaturized therapeutic payloads could maximize delivery of the therapeutic to the site of action while also minimizing off-target effects in other body sites.

The capacity of Sb for extensive genetic engineering allows for the future development of gene circuits that produce alternative ICIs or other anticancer therapies alone and in combination, an effective approach that has often been limited by tolerability [32257944]. Furthermore, we anticipate these gene circuits could be developed as ‘sense-and-respond’ systems that specifically release therapies in the presence of a disease-specific marker (10.1021/acssynbio.1c00384). Such expansions on constitutively expressed therapeutic delivery systems have exciting potential for further improving therapeutic outcome, safety, and specificity of the therapy towards the disease site. Although the effects we observed in tumor reduction and immune modulation were statistically significant, their modest effect sizes leave much room for improvement in future designs. In addition to the future directions described above, alternative delivery routes, such as enema, could also be employed to see if targeted administration to the colon could improve therapeutic effect in that region of the GI tract. Whether this platform can be repurposed to treat other GI malignancies, such as gastric or pancreatic cancers, is yet to be determined. Beyond cancer, we believe this platform could also be adapted to treat other GI diseases, including inflammatory bowel disease and metabolic disorders, expanding the utility of the probiotic platform to provide GI patients new therapeutic options.

In summary, we developed a first-in-class ICI delivery system in the probiotic yeast Sb, expanding the arsenal of available microbial chassis to carry anticancer therapies which has historically been dominated by bacteria. Ultimately, we believe this orally administered, yeast-based therapy prototype provides compelling evidence that supports the utility of engineered yeasts as remediators of cancer and other GI diseases.

### Example 2—Construction and Characterization of Modified Sb Strain Library

To assemble and characterize a Sb strain library that produces a range of haPD-1 therapeutic outputs, the following experiments were conducted. The Sb strain library was constructed to enable selection of an Sb strain with favorable dose (highest therapeutic effect with lowest off-target effects/toxicity which represents a common obstacle in immune checkpoint inhibitor therapy compositions. A combinatorial construct library (SEQ ID NOS:1-23) was developed and transformed into Sb probiotic yeast. The transformed Sb were subjected to further analysis as described below to optimize the therapeutic dose of haPD-1 within a wide range of expression.

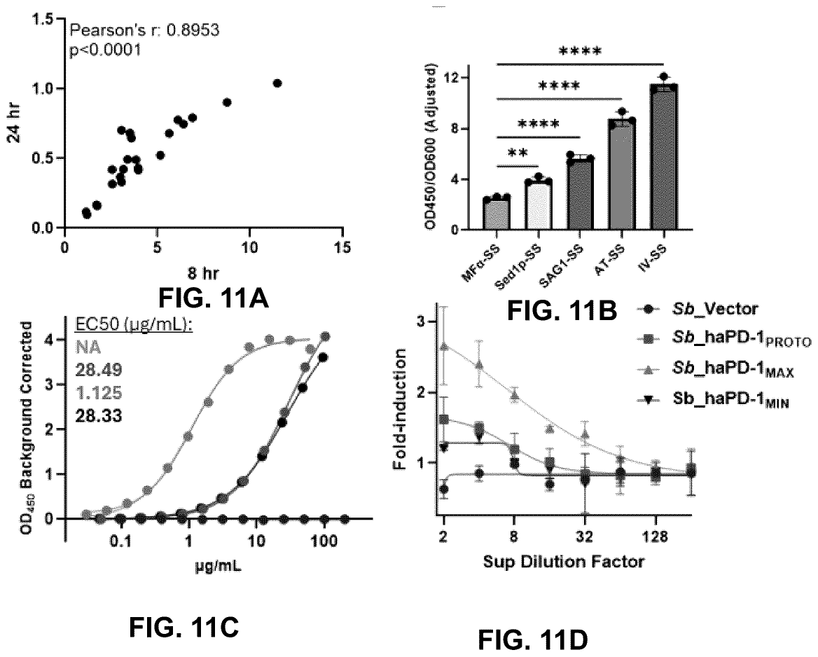

Cultured yeast was subjected to PD-L1 binding ELISA analysis to quantify expression rates. The expression rates were positively correlated between two timepoints tested in vitro. FIG. 11A shows ELISA OD450 values adjusted by cell density (OD600), demonstrating that the Sb_haPD-1 strains have a wide range of expression.

FIG. 11B compares different Sb secretion signals demonstrating that secretion signal identity has a significant impact on haPD-1 expression. The IV secretion signal (right bar) had the highest signal.

FIG. 11C is graph comparing dose-response curves showing that Sb_haPD-1max (MJW126) has a 28-fold improvement in EC50 in PD-L1 binding ELISA assay (4PL logistic curve).

FIG. 11D is a graph comparing the results of a PD-1/PD-L1 blockade assay for various Sb strains. FIG. 11D demonstrated that Sb_haPD-1 max culture broths exhibited elevated activity in the PD-1/PD-L1 blockade assay (Promega J1250).

Healthy male and female mice were administered a single gavage of Sb_haPD-1proto (original strain) and Sb_haPD-1max (MJW126, Table 1, highest secreting strain)(n=5 for each sex and each group). Stools were collected from each mouse prior to gavage (TO) and at 2, 4, 6, 8, and 24 hours post-gavage. PD-L1 binding ELISA and Sb CFU titering was used to determine haPD-1 detection and Sb CFU/mg, respectively.

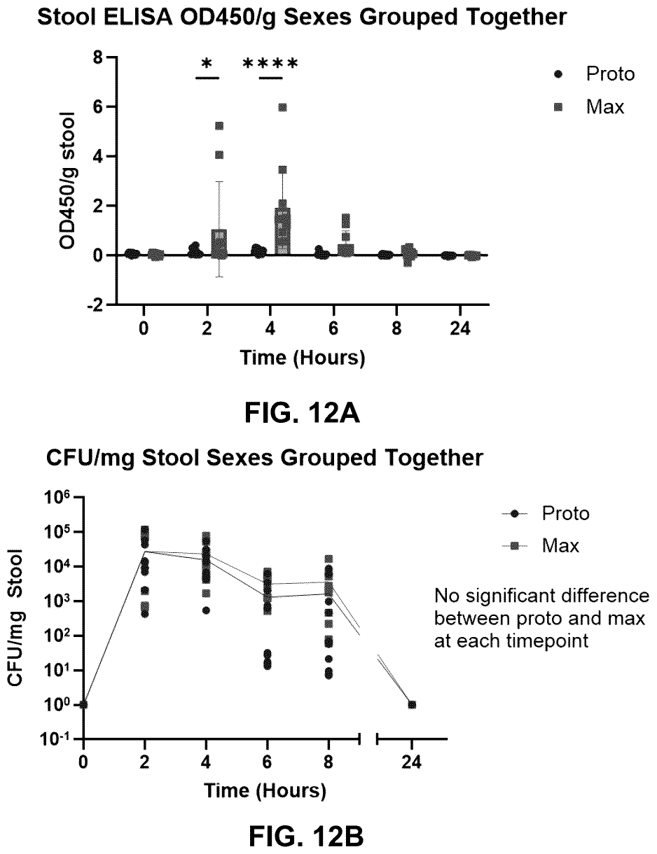

FIG. 12A summarizes the amount of haPD-1 in the mouse stools at various times. Sb_haPD-1 max is now detectable in stools, whereas Sb_haPD-1 proto continued to be undetectable in stools. The Sb_haPD-1 max Sb strain secreted significantly more therapeutic haPD-1 in vivo.

FIG. 12B compares the Sb CFU titering of Sb_haPD and Sb_haPD-1 max strains. Both strains had similar Sb colonization profiles.

What is claimed is:

1. A composition to suppress or reduce tumors in a patient, the composition comprising an engineered yeast comprising a nucleotide sequence encoding a high-affinity programmed cell death 1 ectodomain protein (haPD-1) comprising a PD-1 ectodomain fused to a IgG1 CH₃ domain, wherein the engineered yeast is configured to secrete the haPD-1.

2. The composition of claim 1, wherein the nucleotide sequence is selected from SEQ ID NOS:1-23.

3. The composition of claim 2, wherein the nucleotide sequence is SEQ ID NO:17.

4. The composition of claim 1, wherein the engineered yeast comprises a yeast species consisting of Saccharomyces cerevisiae var. boulardii (Sb).

5. The composition of claim 1, wherein the composition is further configured for oral administration and for release of the haPD-1 into the patient's gastrointestinal tract.

6. The composition of claim 1, wherein the haPD-1 is configured to bind the patient's programmed death ligand 1 (PD-L1) with high affinity.

7. The composition of claim 1, wherein the composition acts as an immune checkpoint inhibitor (ICI) for reducing gastrointestinal tumors.

8. A method of suppressing or reducing gastrointestinal tumors in a patient, the method comprising administering a therapeutically effective amount of a composition comprising an engineered yeast comprising a nucleotide sequence encoding a high-affinity programmed cell death 1 ectodomain protein (haPD-1) comprising a PD-1 ectodomain fused to a IgG1 CH3 domain, wherein the engineered yeast is configured to secrete the haPD-1.

9. The method of claim 8, wherein the nucleotide sequence is selected from SEQ ID NOS:1-26.

10. The method of claim 9, wherein the nucleotide sequence is SEQ ID NO:17.

11. The method of claim 8, wherein the engineered yeast comprises a yeast species consisting of Saccharomyces cerevisiae var. boulardii (Sb).

12. The method of claim 8, wherein the composition is further configured for oral administration and for release of the haPD-1 into the patient's gastrointestinal tract.

13. The method of claim 8, wherein the haPD-1 is configured to bind the patient's programmed death ligand 1 (PD-L1) with high affinity.

14. The method of claim 8, wherein the composition acts as an immune checkpoint inhibitor (ICI) for reducing gastrointestinal tumors.
